(19) United States
(12) Patent Application Publication
(10) Pub. No.: US 2026/0315592 A1
(43) Pub. Date: Oct. 15, 2026

(54) ENPP3-BINDING MOLECULES, CHIMERIC ANTIGEN RECEPTORS FORMED THEREFROM, AND METHODS OF USE THEREOF FOR THE TREATMENT OF CANCER

(71) Applicants: YALE UNIVERSITY, New Haven, CT (US); CELLINFINITY BIO, INC., West Haven, CT (US)

(72) Inventors: Sidi CHEN, New Haven, CT (US); Shan XIN, New Haven, CT (US); Manuel MOHR, West Haven, CT (US); Alex YEMELYANOV, West Haven, CT (US); Geeta RAM, West Haven, CT (US); Narges TAFRESHI, New Haven, CT (US); Sunil PANIGRAHI, West Haven, CT (US); Venkata Naga Chandrasekhar BODDUPALLI, West Haven, CT (US); Jun Liu, WEST HAVEN, CT (US)

(21) Appl. No.: 19/478,832

(22) PCT Filed: Apr. 25, 2024
(86) PCT No.: PCT/US2024/026310

§ 371 Date: Oct. 27, 2025

Related U.S. Application Data
(60) Provisional application No. 63/498,473, filed on Apr. 26, 2023.

(51) Int. Cl. A61K 40/11; A61K 40/31; A61K 40/42; A61P 35/00; C07K 14/705; C07K 16/28; C12N 5/0783

(52) U.S. Cl. CPC A61K 40/11; A61K 40/31; A61K 40/4202; A61K 40/4252; A61P 35/00; C07K 14/7051; C07K 16/2896; C12N 5/0636; A61K 2239/17; A61K 2239/56; C07K 2317/565; C07K 2317/622; C07K 2317/732; C07K 2317/734; C07K 2317/92

(57) ABSTRACT

Antibodies, chimeric antigen receptors (CARs) and other molecules that specifically bind ENPP3 are provided. Host cells, such as immune cells, including the molecules, antibodies, fusion proteins, CARs, nucleic acids encoding them, are also provided. In some forms, the cells are CAR immune cells. In some forms, the CAR immune cells maintain the ability to kill ENPP3+ cancer cells after multiple stimulations. Pharmaceutical compositions including the antibodies, CARs or T cells are also provided. In some forms, the molecule or antibody includes drugs conjugated thereto, has antibody-dependent cell-mediated cytotoxicity (ADCC) activity, complement-dependent cytotoxicity activity (CDC), and/or is a bi-specific engager optionally a T cell engager or NK cell engager. Methods of using the described molecules to treat ENPP3+ cancers are also described.

### CROSS-REFERENCE TO RELATED APPLICATIONS

This application claims the benefit of and priority to U.S. Provisional Application No. 63/498,473 filed Apr. 26, 2023, which is hereby incorporated by reference in its entirety.

### STATEMENT REGARDING FEDERALLY SPONSORED RESEARCH

This invention was made with government support under CA238295 and CA231112 awarded by National Institutes of Health and W81XWH-20-1-0072 and W81XWH-21-1-0514 awarded by the Department of Defense. The government has certain rights in the invention.

### REFERENCE TO THE SEQUENCE LISTING

The Sequence Listing XML submitted as a file named “YU_8521_PCT_ST26.xml” and having a size of 147,411 bytes is hereby incorporated by reference pursuant to 37 C.F.R. § 1.834 (c) (1).

### FIELD OF THE INVENTION

The invention is generally related to the field of immunotherapy, and more particularly to binder polypeptides, chimeric antigen receptors (CAR) formed therefrom, and cells expressing the same that effectively target and kill solid tumors, and methods of making and using thereof.

### BACKGROUND OF THE INVENTION

Chimeric antigen receptor (CAR)-T cell therapy is a new treatment paradigm that redirects genetically engineered T cells to specifically recognize and lyse oncogenic cells in an MHC-independent manner. Chimeric antigen receptor T cells (CAR-Ts) have been proven as powerful cancer therapeutics, and many CAR-T cell candidates have achieved astonishing clinical results in hematological malignancies. It is worth noting that six CAR-T cell-based products have been approved by the United States Food and Drug Administration (US FDA) since 2017. However, unfavorable efficacy, safety issues, exhaustion, limited infiltration, and poor persistence are among the current major obstacles that hinder the clinical success of CAR-T therapy in refractory or resistant hematological malignancies, as well as solid tumors. Furthermore, current FDA-approved CAR-T cells are personalized (autologous) and restricted with single antigen-specific CARs, making the treatment more vulnerable to antigen loss. A number of studies have observed that antigen loss-associated tumor relapse remains a major challenge for maintaining long-term remission in a substantial fraction of CAR-T administered patients, which makes treatments of post-CAR relapse more difficult. CAR-T therapy does not show strong efficacy in solid tumors. Thus, the development of more potent, adaptable and time/cost-effective T cell therapies is needed. Despite its current success, there remains major challenges for CAR-T therapy. No CAR T therapy has been approved by the FDA for solid tumors so far. Thus, engineering better CAR-Ts is important to allow CAR-Ts to achieve their full potential against solid tumor and there is a need for enhanced artificial T cells that are broadly effective against solid tumors.

Therefore, it is an object of the invention to provide new immunoglobulins and immunoglobulin-like binding molecules specific for cancer antigens present on solid tumors, and binder-based therapies based thereon.

It is also an object of the invention to provide artificial immune cells with chimeric immunoreceptors presenting the binders having anti-tumor activity and methods thereof for treating cancers including those with solid tumors.

### SUMMARY OF THE INVENTION

Antigen binding domains and antibodies and other molecules including the same that immunochemically bind to SEQ ID NOS: 1 and/or 2 are provided. In some embodiments, the antigen binding domain includes six complementarity determining regions (CDRs), wherein the CDRs include at least one consensus CDR of the CDRs of anti-ENPP3 antibodies SX13-1, SX13-2, SX13-5, SX13-6, SX13-7, SX13-8.1, SX13-10 and SX13-12 with all remaining CDRs selected from: (A) the three light chain and the three heavy chain CDRs of anti-ENPP3 antibody SX13-1; (B) the three light chain and the three heavy chain CDRs of anti-ENPP3 antibody SX13-2; (C) the three light chain and the three heavy chain CDRs of anti-ENPP3 antibody SX13-5; (D) the three light chain and the three heavy chain CDRs of anti-ENPP3 antibody SX13-6; (E) the three light chain and the three heavy chain CDRs of anti-ENPP3 antibody SX13-7; (F) the three light chain and the three heavy chain CDRs of anti-ENPP3 antibody SX13-8.1; (G) the three light chain and the three heavy chain CDRs of anti-ENPP3 antibody SX13-10; or (H) the three light chain and the three heavy chain CDRs of anti-ENPP3 antibody SX13-12.

In some embodiments, the six CDRs are: (A) the three light chain and the three heavy chain CDRs of anti-ENPP3 antibody SX13-1; (B) the three light chain and the three heavy chain CDRs of anti-ENPP3 antibody SX13-2; (C) the three light chain and the three heavy chain CDRs of anti-ENPP3 antibody SX13-5; (D) the three light chain and the three heavy chain CDRs of anti-ENPP3 antibody SX13-6; (E) the three light chain and the three heavy chain CDRs of anti-ENPP3 antibody SX13-7; (F) the three light chain and the three heavy chain CDRs of anti-ENPP3 antibody SX13-8.1; (G) the three light chain and the three heavy chain CDRs of anti-ENPP3 antibody SX13-10; or (H) the three light chain and the three heavy chain CDRs of anti-ENPP3 antibody SX13-12.

In some embodiments, the antigen binding domain includes one or both of the light and heavy chain variable domain(s) of clone SX13-1, SX13-2, SX13-5, SX13-6, SX13-7, SX13-8.1, SX13-10 or SX13-12.

For example, in some embodiments, the antigen binding domain includes six complementarity-determining regions (CDRs), wherein the CDRs include at least one consensus CDR of the CDRs of the amino acid sequence of SEQ ID NOS: 3 or 4, SEQ ID NOS: 5 or 6, SEQ ID NOS: 7 or 8, SEQ ID NOS: 9 or 10, SEQ ID NOS: 11 or 12, SEQ ID NOS: 13 or 14, SEQ ID NOS: 15 or 16, and SEQ ID NOS: 17 or 18, with all remaining CDRs selected from: (A) the three light chain and the three heavy chain CDRs of the amino acid sequence of SEQ ID NOS: 4 or 3; (B) the three light chain and the three heavy chain CDRs of the amino acid sequence of SEQ ID NOS: 6 or 5; (C) the three light chain and the three heavy chain CDRs of the amino acid sequence of SEQ ID NOS: 8 or 7; (D) the three light chain and the three heavy chain CDRs of the amino acid sequence of SEQ ID NOS: 10 or 9; (E) the three light chain and the three heavy chain CDRs of the amino acid sequence of SEQ ID NOS: 12 or 11; (F) the three light chain and the three heavy chain CDRs of the amino acid sequence of SEQ ID NOS: 14 or 13; (G) the three light chain and the three heavy chain CDRs of the amino acid sequence of SEQ ID NOS: 16 or 15; or (H) the three light chain and the three heavy chain CDRs of the amino acid sequence of SEQ ID NOS: 18 or 17.

In some embodiments, the six CDRs are: (A) the three light chain and the three heavy chain CDRs of the amino acid sequence of SEQ ID NOS: 4 and 3; (B) the three light chain and the three heavy chain CDRs of the amino acid sequence of SEQ ID NOS: 6 and 5; (C) the three light chain and the three heavy chain CDRs of the amino acid sequence of SEQ ID NOS: 8 and 7; (D) the three light chain and the three heavy chain CDRs of the amino acid sequence of SEQ ID NOS: 10 and 9; (E) the three light chain and the three heavy chain CDRs of the amino acid sequence of SEQ ID NOS: 12 and 11; (F) the three light chain and the three heavy chain CDRs of the amino acid sequence of the amino acid sequence of SEQ ID NOS: 14 and 13; (G) the three light chain and the three heavy chain CDRs of the amino acid sequence of SEQ ID NOS: 16 and 15; or (H) the three light chain and the three heavy chain CDRs of the amino acid sequence of SEQ ID NOS: 18 and 17.

In some embodiments, the antigen binding domain includes one or both of the light and heavy chain variable domain(s) of the amino acid sequence of SEQ ID NOS: 4 and/or 3, respectively with or without the signal sequence (e.g., SEQ ID NOS: 71 and/or 70), SEQ ID NOS: 6 and/or 5, respectively with or without the signal sequence (e.g., SEQ ID NOS: 73 and/or 72), SEQ ID NOS: 8 and/or 7, respectively with or without the signal sequence (e.g., SEQ ID NOS: 75 and/or 74), SEQ ID NOS: 10 and/or 9, respectively with or without the signal sequence (e.g., SEQ ID NOS: 77 and/or 76), SEQ ID NOS: 12 and/or 11, respectively with or without the signal sequence (e.g., SEQ ID NOS: 79 and/or 78), SEQ ID NOS: 14 and/or 13, respectively with or without the signal sequence (e.g., SEQ ID NOS: 81 and/or 80), SEQ ID NOS: 16 and/or 15, respectively with or without the signal sequence (e.g., SEQ ID NOS: 83 and/or 82), or SEQ ID NOS: 18 and/or 17, respectively with or without the signal sequence (e.g., SEQ ID NOS: 85 and/or 84).

In some embodiments, the antibody or molecule includes the amino acid sequence of any one of SEQ ID NOS: 19-26, or variant thereof with at least 75% sequence identity thereto, optionally wherein the variant includes the CDRs of SEQ ID NOS: 4 and 3, respectively, SEQ ID NOS: 6 and 5, respectively, SEQ ID NOS: 8 and 7, respectively, SEQ ID NOS: 10 and 9, respectively, SEQ ID NOS: 12 and 11, respectively, SEQ ID NOS: 14 and 13, respectively, SEQ ID NOS: 16 and 15, respectively or SEQ ID NOS: 18 and 17, respectively without variation.

In some embodiments, the antibody is an intact antibody and functional antibody fragment or fusion protein. Functional fragments or fusion proteins include, for example, Fab fragments, F(ab′)2 fragments, Fab′ fragments, Fv fragments, recombinant IgG (rlgG) fragments, single chain antibody fragments optionally single chain variable fragments (scFv), and single domain antibodies optionally selected from sdAb, sdFv, and nanobody fragments.

In some embodiments, the antibody is selected from intrabodies, peptibodies, chimeric antibodies, fully human antibodies, humanized antibodies, and heteroconjugate antibodies, and multispecific antibodies optionally selected bispecific antibodies, diabodies, triabodies, tetrabodies, tandem di-scFv, and tandem tri-scFv.

The antibody can be an IgM, IgE, IgA, IgD, or IgG optionally an IgG1, IgG2, lgG3, or IgG4.

The molecule or antibody can be detectably labeled or include a conjugated toxin, drug, receptor, enzyme, receptor ligand. Fusion proteins including the provide antigen binding domains and a heterologous amino acid sequence are also provided. In some embodiments, the fusion protein is a chimeric antigen receptor (CAR) polypeptide. Such CARs can include extracellular antigen binding domain, a spacer domain, a transmembrane domain, and intracellular signaling domain. In an exemplary embodiment, the structure of CAR includes a disclosed antigen binding domain linked to CD8hinge linked to CD8TM linked to 41BBcostim linked to CD3z optionally including the amino acid sequence of SEQ ID NO:28.

In some forms, the molecule or antibody includes six complementarity-determining regions (CDRs), wherein the CDRs include at least one consensus CDR of the CDRs of a heavy chain variable domain including the amino acid sequence of SEQ ID NO: 78, and a light chain variable domain including the amino acid sequence of SEQ ID NO: 79. In some forms, the molecule or antibody includes heavy chain CDR1, heavy chain CDR2 and heavy chain CDR3 of SEQ ID NO:78, and light chain CDR1, light chain CDR2 and light chain CDR3 of SEQ ID NO:79. In some forms, the molecule or antibody includes the heavy chain CDR1 includes SEQ ID NO:46, heavy chain CDR2 includes SEQ ID NO:36, and heavy chain CDR3 includes SEQ ID NO:31, light chain CDR1 includes SEQ ID NO:52, light chain CDR2 includes SEQ ID NO:53 and light chain CDR3 includes SEQ ID NO:34. In some forms, the molecule or antibody includes the molecule includes the amino acid sequence of SEQ ID NO:23. In some forms, the molecule or antibody includes the molecule includes a chimeric antigen receptor (CAR). In some forms, the CAR includes the amino acid sequence of any one of SEQ ID NOs: 28, 99, 109, 111, 113, 115, 117 and 119.

In some forms, the molecule or antibody includes six complementarity-determining regions (CDRs), wherein the CDRs include at least one consensus CDR of the CDRs of a heavy chain variable domain including the amino acid sequence of SEQ ID NO: 78, and a light chain variable domain including the amino acid sequence of SEQ ID NO: 79. includes six CDRs, wherein the CDRs include at least one consensus CDR of the CDRs of the heavy chain variable domain amino acid sequence of SEQ ID NO:82, and the light chain variable domain amino acid sequence of SEQ ID NO:83. In some forms, the antigen binding domain includes heavy chain CDR1, heavy chain CDR2 and heavy chain CDR3 of SEQ ID NO:82, and light chain CDR1, light chain CDR2 and light chain CDR3 of SEQ ID NO:83. In some forms, the heavy chain CDR1 includes SEQ ID NO: 60, heavy chain CDR2 includes SEQ ID NO:61, and heavy chain CDR3 includes SEQ ID NO:62, light chain CDR1 includes SEQ ID NO:63, light chain CDR2 includes SEQ ID NO:64 and light chain CDR3 includes SEQ ID NO:65. In some forms, the molecule includes the amino acid sequence of SEQ ID NO:25. In some forms, the molecule includes a chimeric antigen receptor (CAR). In some forms, the CAR includes the amino acid sequence of SEQ ID NO:28 or 103.

In some forms, the molecule or antibody includes six complementarity-determining regions (CDRs), wherein the CDRs include at least one consensus CDR of the CDRs of a heavy chain variable domain including the amino acid sequence of SEQ ID NO: 78, and a light chain variable domain including the amino acid sequence of SEQ ID NO: 79. antigen binding domain includes six CDRs, wherein the CDRs include at least one consensus CDR of the CDRs of the heavy chain variable domain amino acid sequence of SEQ ID NO:70, and the light chain variable domain amino acid sequence of SEQ ID NO:71. In some forms, the antigen binding domain includes heavy chain CDR1, heavy chain CDR2 and heavy chain CDR3 of SEQ ID NO:70, and light chain CDR1, light chain CDR2 and light chain CDR3 of SEQ ID NO:71. In some forms, the heavy chain CDR1 includes SEQ ID NO:29, heavy chain CDR2 includes SEQ ID NO:30, and heavy chain CDR3 includes SEQ ID NO:31, light chain CDR1 includes SEQ ID NO:32, light chain CDR2 includes SEQ ID NO:33 and light chain CDR3 includes SEQ ID NO: 34. In some forms, the molecule includes the amino acid sequence of SEQ ID NO: 19. In some forms, the molecule includes a chimeric antigen receptor (CAR). In some forms, the CAR includes the amino acid sequence of SEQ ID NO:28 or 91.

In some forms, the molecule or antibody includes six CDRs, wherein the CDRs include at least one consensus CDR of the CDRs of a heavy chain variable domain including the amino acid sequence of SEQ ID NO:72, and a light chain variable domain including the amino acid sequence of SEQ ID NO:73. In some forms, the antigen binding domain includes heavy chain CDR1, heavy chain CDR2 and heavy chain CDR3 of SEQ ID NO: 72, and light chain CDR1, light chain CDR2 and light chain CDR3 of SEQ ID NO: 73. In some forms, the heavy chain CDR1 includes SEQ ID NO:35, heavy chain CDR2 includes SEQ ID NO:36, and heavy chain CDR3 includes SEQ ID NO:37, light chain CDR1 includes SEQ ID NO:38, light chain CDR2 includes SEQ ID NO:39 and light chain CDR3 includes SEQ ID NO:37. In some forms, the molecule includes the amino acid sequence of SEQ ID NO:20. In some forms, the molecule includes a chimeric antigen receptor (CAR). In some forms, the CAR further includes the amino acid sequence of SEQ ID NO:28 or 93.

In some forms, the molecule or antibody includes six CDRs, wherein the CDRs include at least one consensus CDR of the CDRs of a heavy chain variable domain amino acid sequence of SEQ ID NO:74, and a light chain variable domain amino acid sequence of SEQ ID NO:75. In some forms, the antigen binding domain includes heavy chain CDR1, heavy chain CDR2 and heavy chain CDR3 of SEQ ID NO:74, and light chain CDR1, light chain CDR2 and light chain CDR3 of SEQ ID NO:75. In some forms, the heavy chain CDR1 includes SEQ ID NO:41, heavy chain CDR2 includes SEQ ID NO: 42, and heavy chain CDR3 includes SEQ ID NO:43, light chain CDR1 includes SEQ ID NO: 44, light chain CDR2 includes SEQ ID NO:45 and light chain CDR3 includes SEQ ID NO:46. In some forms, the molecule includes the amino acid sequence of SEQ ID NO: 21. In some forms, the molecule includes a chimeric antigen receptor (CAR). In some forms, the CAR further includes the amino acid sequence of any one of SEQ ID NO: 28 or 95.

In some forms, the molecule or antibody includes six CDRs, wherein the CDRs include at least one consensus CDR of the CDRs of a heavy chain variable domain amino acid sequence of SEQ ID NO:76, and a light chain variable domain amino acid sequence of SEQ ID NO:77. In some forms, the antigen binding domain includes heavy chain CDR1, heavy chain CDR2 and heavy chain CDR3 of SEQ ID NO:76, and light chain CDR1, light chain CDR2 and light chain CDR3 of SEQ ID NO:77. In some forms, the heavy chain CDR1 includes SEQ ID NO:46, heavy chain CDR2 includes SEQ ID NO: 47, and heavy chain CDR3 includes SEQ ID NO:48, light chain CDR1 includes SEQ ID NO: 49, light chain CDR2 includes SEQ ID NO:50 and light chain CDR3 includes SEQ ID NO:51. In some forms, the molecule includes the amino acid sequence of SEQ ID NO: 22. In some forms, the molecule includes a chimeric antigen receptor (CAR). In some forms, the CAR further includes the amino acid sequence of any one of SEQ ID NO: 28 or 97.

In some forms, the molecule or antibody includes six CDRs, wherein the CDRs include at least one consensus CDR of the CDRs of a heavy chain variable domain amino acid sequence of SEQ ID NO:80 and a light chain variable domain amino acid sequence of SEQ ID NO:81. In some forms, the antigen binding domain includes heavy chain CDR1, heavy chain CDR2 and heavy chain CDR3 of SEQ ID NO:80, and light chain CDR1, light chain CDR2 and light chain CDR3 of SEQ ID NO:81. In some forms, the heavy chain CDR1 includes SEQ ID NO:54, heavy chain CDR2 includes SEQ ID NO: 55, and heavy chain CDR3 includes SEQ ID NO:56, light chain CDR 1 includes SEQ ID NO: 57, light chain CDR2 includes SEQ ID NO:58 and light chain CDR3 includes SEQ ID NO:59. In some forms, the molecule includes the amino acid sequence of SEQ ID NO: 24. In some forms, the molecule includes a chimeric antigen receptor (CAR). In some forms, the CAR further includes the amino acid sequence of any one of SEQ ID NO: 28 or 101.

In some forms, the molecule or antibody includes six CDRs, wherein the CDRs include at least one consensus CDR of the CDRs of a heavy chain variable domain amino acid sequence of SEQ ID NO:84, and a light chain variable domain amino acid sequence of SEQ ID NO:85. In some forms, the antigen binding domain includes heavy chain CDR1, heavy chain CDR2 and heavy chain CDR3 of SEQ ID NO:84, and light chain CDR1, light chain CDR2 and light chain CDR3 of SEQ ID NO:85. In some forms, the heavy chain CDR1 includes SEQ ID NO:46, heavy chain CDR2 includes SEQ ID NO: 66, and heavy chain CDR3 includes SEQ ID NO:67, light chain CDR1 includes SEQ ID NO: 68, light chain CDR2 includes SEQ ID NO:69 and light chain CDR3 includes SEQ ID NO:34. In some forms, the molecule includes the amino acid sequence of SEQ ID NO: 26. In some forms, molecule includes a chimeric antigen receptor (CAR). In some forms, the CAR includes the amino acid sequence of any one of SEQ ID NOs: 28, and 105. In some forms, the chimeric antigen receptor (CAR) includes a framework including any one of SEQ ID NOs: 28, 120, 121, 122, 123, 124, or 125.

In some forms, the CAR includes (i) an antigen binding domain, optionally an anti-ENPP3 antigen binding domain, optionally an scFv or other construct, and (ii) a variant spacer domain, transmembrane domain, and/or intracellular/costimulatory (“costim”) domains. In some such embodiments, the CAR include the amino acid sequence of SEQ ID NOS: 120-125. In some forms, the CAR includes the amino acid sequence of any one of SEQ ID NOS: 91, 93, 95, 97, 99, 101, 103, 105, 107, 109, 111, 113, 115, 117, and 119 without SEQ ID NO: 126, any one of SEQ ID NOS: 91, 93, 95, 97, 99, 101, 103, 105, 107, 109, 111, 113, 115, 117, and 119, or a fragment or variant thereof with at least 70% sequence identity thereto.

Nucleic acids encoding the molecules and antibodies, the fusion proteins, and the CARs are also provided. In some embodiments, the nucleic acids include an expression control sequence operably linked thereto, optionally wherein the expression control sequence includes a promoter optionally selected SFFV promoter or EFS promotor. In some embodiments, the nucleic acid is a vector.

Host cells including the molecules, antibodies, fusion proteins, CARs, nucleic acids, etc. are also provided. In some embodiments, the host cells are CAR immune cells. Such immune cells can be, for example, a T cell, natural killer (NK) cell, or macrophage (MA). In some embodiments, the CAR immune cell maintains the ability to kill cancer ENPP3+ cancer cells after 1, 2, 3, 4 or more stimulations. In some embodiments, the CAR immune cell is resistant to exhaustion.

Pharmaceutical compositions including the provided molecules and T cells optionally wherein the molecule or antibody includes a drug conjugated thereto and/or has antibody-dependent cell-mediated cytotoxicity (ADCC) activity, or complement-dependent cytotoxicity activity (CDC), and/or is a bispecific engager optionally a T cell engager or NK cell engager are also provided, as are methods of use thereof.

For example, a method of treating cancer can include administering a subject in need thereof an effective amount of a disclosed composition, for example a pharmaceutical composition. In some embodiments, the subject has a cancer including cancer cells including expression of ENPP3. In some embodiments, the expression of ENPP3 on the surface of the cancer cells is elevated compared to non-cancer cells, optionally non-cancer cells of the same cell type. Exemplary preferred cancers include renal cell carcinomas (RCC), optionally wherein the RCC is clear cell RCC or a non-clear cell RCC optionally selected papillary renal cell carcinoma, chromophobe renal cell carcinoma, collecting duct RCC, multilocular cystic RCC, medullary carcinoma, mucinous tubular and spindle cell carcinoma, and neuroblastoma-associated RCC. In some embodiments, the cancer includes solid tumors.

### BRIEF DESCRIPTION OF THE DRAWINGS

The accompanying drawings, which are incorporated in and constitute a part of this specification, illustrate several forms of the disclosed method and compositions and together with the description, explain the principles of the disclosed methods and compositions.

FIG. 1A is a graph of ENPP3 expression across TCGA cancer, showing log 2 (TPM+1) over different tissue types (normal tissue and cancer tissue: Bladder Urothelial Carcinoma (BLCA), Breast invasive carcinoma (BRCA), Cervical squamous cell carcinoma and endocervical adenocarcinoma (CESC), Cholangiocarcinoma (CHOL), Colon adenocarcinoma (COAD), Esophageal carcinoma (ESCA), Glioblastoma multiforme (GBM), Head and Neck squamous cell carcinoma (HNSC), Kidney Chromophobe (KICH), Kidney renal clear cell carcinoma (KIRC), Kidney renal papillary cell carcinoma (KIRP), Liver hepatocellular carcinoma (LIHC), Lung adenocarcinoma (LUAD), Lung squamous cell carcinoma (LUSC), Pancreatic adenocarcinoma (PAAD), prostate adenocarcinoma (PRAD), Pheochromocytoma and Paraganglioma (PCPG), Rectum adenocarcinoma (READ), Sarcoma (SARC), Skin Cutaneous Melanoma (SKCM), Thyroid carcinoma (THCA), Thymoma (THYM), Stomach adenocarcinoma (STAD), Uterine Corpus Endometrial Carcinoma (UCEC)). The first plot is normal and second plot is tumor for each tumor type. FIG. 1B is also a graph of ENPP3 expression across TCGA cancers.

FIG. 2 is a schematic showing the organization of cassette and knock-in strategy for preparing ENPP3-targeting T cells expressing a heterologous Chimeric Antigen Receptor (CAR) from the TRAC locus.

FIGS. 3A and 3B show SX13-1, 13-7 and 13-8 ENPP3 CARs in vitro cytotoxicity assay at ratio of 1:1 E:T for A498, 786-O and ACHN kidney cancer cell lines. FIGS. 3C-3G show SX13-1, 13-2, 13-5, 13-6, 13-7, 13-8.1, 13-10,13-12, and pos-ct in different kidney cancer cell lines at ratio of 1:1 E:T used for RTCA (real time killing assay) with different level of ENPP3 expression from A498-ENPP3 the highest to 786-O the lowest level: A498-ENPP3 (3C), A498 (3D), ACHN (3E), SW156 (3F) and 786-O (3G).

FIG. 4 is a graph showing SX13-1 (13-1) and SX13-7 (13-7) ENPP3 CARS, 204C (Un-transduced T cells from donor 204C) and PBS control group in vivo tumor elimination assay; 786-O cells were injected into NSG mice subcutaneously followed by CAR T cells injection intravenously, showing volume (mm³) over dpi. Tumor size was measured weekly.

FIG. 5 is a graph showing volume (mm³) over dpi for each of SX13-1 (13-1) and SX13-7 (13-7) ENPP3 CARs, 204C (Un-transduced T cells from donor 204C) and untreated (untr) PBS control group in vivo tumor elimination assay. 786-O cells were injected into NBSGW mice subcutaneously, followed by CAR T cells injection intravenously, showing. Tumor size was measured weekly.

FIG. 6 is a graph showing SX13-1 (13-1) and SX13-7 (13-7) ENPP3 CARS, 204C (Un-transduced T cells from donor 204C) and Untr (PBS control group) in vivo tumor elimination assay. ACHN cells were injected into NSG mice subcutaneously followed by CAR T cells injection intravenously. Tumor size was measured weekly.

FIG. 7 is an illustration of CAR T evolution in a serial co-culture assay.

FIG. 8A shows CAR-T expression (using FLAG) in different time points after serial killing (no stimulation, stimulation 1, 3 and 6). FIG. 8B shows a cytokine profile of ENPP3 CARs in the serial killing assay after stimulation 4. CAR 13-7, 13-10, and 13-12 showed as good as positive control CAR for GM-CSF secretion and Granzyme B.

FIG. 8C shows the IL-4 profile after various stimulations.

FIGS. 9A-9D show the profiles of four exhaustion markers (PD1, TIGIT, KAG3 and CD39) for the donor 3 ENPP3 CARs (FIGS. 9A and 9B) and the donor 4 ENPP3 CARs (FIGS. 9C and 9D) unstimulated (9A and 9C) and after 6 stimulations (FIGS. 9B and 9D) with A498 kidney cancer cells.

FIG. 10 shows the EC50 for killing (single killing, just one time stimulation) for the final candidate ENPP3 CARs (13-1, 13-7, 13-10 and 13-12) at 24 h time point (24 h after adding CARs to target cells which is A498 kidney cancer cells). The EC50 was calculated as following 0.97, 1.76, 0.66 and 1.26 for 13-1, 13-7, 13-10 and 13-12, respectively.

FIG. 11 is a plot showing relative binding affinity of CAR42, CAR46, CAR48 and CAR49 to ENPP3.

FIG. 12 is a plot showing RTCA assay (killing assay) on ENPP3 KO A498 and ENPP3 over-expressing (OE) A498 cells using ENPP3 CARscFv 13-7 and 13-10 demonstrating high specificity of the scFvs (a lack of cytolytic activity against A498 ENPP3 KO cells).

FIGS. 13A-13B are a pair of plot showing Tumor growth of A498 and ACHN human kidney tumor with ENPP3 CAR 13-7 treatment. Mice were first subcutaneously injected with 4×10⁶ tumor cells; five million of the ENPP3 CAR 13-7 cells, or non-transduced CD3+ T cells were infused into mice at day 10. Two-way ANOVA was used to assess significance. **** p<0.0001. CAR scFv 13-7 in vivo in an A498 (FIG. 13A) and ACHN (FIG. 13B) xenograft models demonstrated superior anti-tumor response for CAR scFv 13-7.

FIGS. 14A-14D demonstrate cytolysis assays with ACHN and SW156 cancer cell lines using CAR42, CAR46, CAR48, CAR49, CAR50. CAR46 and CAR48 showed better killing efficacy against ACHN, while all CARs showed strong elimination of SW156. FIGS. 14E-14H are plots showing quantification of Enpp3 CAR killing efficiency across different kidney cancer cell lines, E:T 2:1 at 48 h (FIGS. 14E and 14F) and 72 h (FIGS. 14G and 14H), after adding the CAR-Ts using Luc assay. FIGS. 14I-14L are plots showing phenotyping of ENPP3 CAR-T in two different donors (donor 5 and 7), after three times stimulations with ACHN (FIG. 14I and FIG. 14L) and two times stimulations with SW156 (FIGS. 14J-14K).

FIGS. 15A-15F are plots showing anti-tumor cytokine induction in different donors. scFV46 and scFV48 showed strong anti-tumor cytokine induction.

FIG. 16A-16C are plots showing anti-tumor cytokine induction (TNF-alpha, Cgranzyme B, and INF gamma) in donor 5.

FIGS. 17A-17E show domain maps of CAR46 (PL46, WT CAR) and its variants PL199 (CD28 Hinge+CD28Tm), PL203 (IgG4m Hinge+CD8 Tm), PL205 (IgG4m Hinge+CD28 Tm) in the spacer regions. PL46 is used as a control.

FIGS. 18A-18B show CAR % and VCN for PL199, PL203, PL205, PL46 from donor 3 (FIG. 18A) and donor 9 (FIG. 18B).

FIGS. 19A-19H are plots showing luciferase assay for cytolysis of SW156, 786-O cells, A498, ACHN cells with CARs: PL199, PL203, PL205, PL46. FIGS. 19I-19L are plots showing the results of an RTCA killing assay of ENPP3 CARs (all using scFv 46) on SW156 cells in two different donors (D3 and D9) at E:T 1:1 (FIGS. 19I-19J) and on 786-O cells in two different donors (D3 and D9) at E:T 1:1 (FIGS. 19K-19L).

FIG. 20 shows constructs of CAR46 spacer and co-stimulation domain variants.

FIGS. 21A-21B show expression profiles of variants of CAR-46, PL046 (CD8-CD8-4-1BB), PL203 (IgG4m-CD8-4-1BB), PL216 (CD28-CD28-CD28), PL217 (IgG4-CD8-4-1BB), and PL219 (IgG4-CD28-CD28) from donor 5 (FIG. 21A) and donor 9 (FIG. 21B).

FIGS. 22A-22F are plots showing luciferase assay for cytolysis of SW156, 786-O cells, A498 using variants of CAR-46, PL046 (CD8-CD8-4-1BB), PL203 (IgG4m-CD8-4-1BB), PL216 (CD28-CD28-CD28), PL217 (IgG4-CD8-4-1BB), and PL219 (IgG4-CD28-CD28) from donor 5 (FIGS. 21A-21C) and donor 9 (FIGS. 21D-21F).

FIGS. 23A-23B show CAR expression profile after ST1 (stimulation 1) with SW156 target cells for PL046 (CD8-CD8-4-1BB), PL203 (IgG4m-CD8-4-1BB), PL216 (CD28-CD28-CD28), PL217 (IgG4-CD8-4-1BB), and PL219 (IgG4-CD28-CD28) from donor 5 (FIG. 23A) and donor 9 (FIG. 23B). FIGS. 23C-23J are plots showing RTCA killing assay of ENPP3 CARs (all using scFv 46) on SW156 cells in two different donors D5 (FIGS. 23C-23F) and D9 (FIGS. 23G-23J) at E:T 1:1 in serial killing assay: Stimulation 1, 2, 3 and 4.

### DETAILED DESCRIPTION OF THE INVENTION

The disclosed method and compositions can be understood more readily by reference to the following detailed description of forms and the Examples included therein and to the Figures and their previous and following description.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise,” or variations such as “comprises” or “comprising,” will be understood to imply the inclusion of a stated element, integer or step, or group of elements, integers or steps, but not the exclusion of any other element, integer or step, or group of elements, integers or steps.

### I. Definitions

“Introduce” in the context of genome modification refers to bringing in to contact. For example, to introduce a gene editing composition to a cell is to provide contact between the cell and the composition. The term encompasses penetration of the contacted composition to the interior of the cell by any suitable means, e.g., via transfection, electroporation, transduction, gene gun, nanoparticle delivery, etc.

“Endogenous” refers to any material from or produced inside an organism, cell, tissue or system. “Exogenous” refers to any material introduced from or produced outside an organism, cell, tissue or system.

The term “antibody” is used in the broadest sense unless clearly indicated otherwise. Therefore, an “antibody” can be naturally occurring or man-made such as monoclonal antibodies produced by conventional hybridoma technology. Antibodies include monoclonal and polyclonal antibodies as well as fragments and polymers containing the antigen binding domain and/or one or more complementarity determining regions of these antibodies. As used herein, the term “antibody” refers to any form of antibody or antigen binding fragment or recombinant protein, and specifically covers monoclonal antibodies (including full length monoclonal antibodies), polyclonal antibodies, multi-specific antibodies (e.g., bispecific antibodies), and antibody fragments so long as they specifically bind the target antigen. Any specific antibody can be used in the methods and compositions provided herein. Thus, in one embodiment the term “antibody” encompasses a molecule including at least one variable region from a light chain immunoglobulin molecule and at least one variable region from a heavy chain molecule that in combination form a specific binding site for the target antigen. The term “variable region” is intended to distinguish such domain of the immunoglobulin from domains that are broadly shared by antibodies (such as an antibody Fc domain). The variable region includes a “hypervariable region” whose residues are responsible for antigen binding. The hypervariable region includes amino acid residues from a “Complementarity Determining Region” or “CDR” (i.e., typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, MD. (1991)) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Chothia, C. et al. (1987) “Canonical Structures For The Hypervariable Regions Of Immunoglobulins,” J. Mol. Biol. 196:901-917). “Framework Region” or “FR” residues are those variable domain residues other than the hypervariable region residues as herein defined. The term antibody includes monoclonal antibodies, multi-specific antibodies, human antibodies, humanized antibodies, synthetic antibodies, chimeric antibodies, camelized antibodies (See e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Publication Nos. WO 94/04678 and WO 94/25591; U.S. Pat. No. 6,005,079), single-chain Fvs (scFv) (see, e.g., see Pluckthun in The Pharmacology of Monoclonal Antibodies, vol. 113, Rosenburg and Moore eds. Springer-Verlag, New York, pp. 269-315 (1994)), single chain antibodies, disulfide-linked Fvs (sdFv), intrabodies, and anti-idiotypic (anti-Id) antibodies (including, e.g., anti-Id and anti-anti-Id antibodies to antibodies of the invention). In particular, such antibodies include immunoglobulin molecules of any type (e.g., IgG, IgE, IgM, IgD, IgA and IgY), class (e.g., IgG₁, IgG₂, IgG₃, IgG₄, IgA₁ and IgA₂) or subclass. An “antibody fragment” or “antigen binding fragment” of an antibody is defined as at least a portion of the variable region of the immunoglobulin molecule that binds to its target, i.e., the antigen binding region (also antigen binding domain). In one embodiment it specifically covers single antibodies and clones thereof and anti-antibody compositions with polyepitopic specificity. The antibody of the present methods and compositions can be monoclonal or polyclonal. An antibody can be in the form of an antigen binding antibody fragment including a Fab fragment, F(ab′)₂ fragment, a single chain variable region, and the like. Fragments of intact molecules can be generated using methods well known in the art and include enzymatic digestion and recombinant means. Thus, the “fragment” may be a recombinant protein, e.g., a fusion protein.

As used herein, any form of the “antigen” can be used to generate an antibody that is specific for the target antigen. Thus, the eliciting antigen may be a single epitope, multiple epitopes, or the entire protein alone or in combination with one or more immunogenicity enhancing agents known in the art. The eliciting antigen may be an isolated full-length protein, a cell surface protein (e.g., immunizing with cells transfected with at least a portion of the antigen), or a soluble protein (e.g., immunizing with only the extracellular domain portion of the protein). The antigen may be produced in a genetically modified cell. The DNA encoding the antigen may genomic or non-genomic (e.g., cDNA) and encodes at least a portion of the extracellular domain. As used herein, the term “portion” refers to the minimal number of amino acids or nucleic acids, as appropriate, to constitute an immunogenic epitope of the antigen of interest. Any genetic vectors suitable for transformation of the cells of interest may be employed, including but not limited to adenoviral vectors, plasmids, and non-viral vectors, such as cationic lipids. In one embodiment, the antibody of the methods and compositions herein specifically bind at least a portion of the extracellular domain of the target antigen of interest.

The antibodies or antigen binding fragments thereof provided herein may be conjugated to a “bioactive agent.” As used herein, the term “bioactive agent” refers to any synthetic or naturally occurring compound that binds the antigen and/or enhances or mediates a desired biological effect.

In one embodiment, the binding fragments useful in the present invention are biologically active fragments. As used herein, the term “biologically active” refers to an antibody or antibody fragment that is capable of binding the desired the antigenic epitope and directly or indirectly exerting a biologic effect.

“Bispecific” antibodies are also useful in the present methods and compositions. As used herein, the term “bispecific antibody” refers to an antibody, typically a monoclonal antibody, having binding specificities for at least two different antigenic epitopes. In one embodiment, the epitopes are from the same antigen. In another embodiment, the epitopes are from two different antigens. Methods for making bispecific antibodies are known in the art. For example, bispecific antibodies can be produced recombinantly using the co-expression of two immunoglobulin heavy chain/light chain pairs. See, e.g., Milstein et al., Nature 305:537-39 (1983). Alternatively, bispecific antibodies can be prepared using chemical linkage. See, e.g., Brennan, et al., Science 229:81 (1985). Bispecific antibodies include bispecific antibody fragments. See, e.g., Hollinger, et al., Proc. Natl. Acad. Sci. U.S.A. 90:6444-48 (1993), Gruber, et al., J. Immunol. 152:5368 (1994).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they specifically bind the target antigen and/or exhibit the desired biological activity (U.S. Pat. No. 4,816,567; and Morrison et al., Proc. Natl. Acad. Sci. USA 81:6851-6855 (1984)).

The term “specifically binds” or “immuno-specifically binds” refers to the binding of an antibody to its cognate antigen, while not significantly binding to other antigens. Preferably, an antibody “specifically binds” to an antigen with an affinity constant (Ka) greater than about 10⁵ mol⁻¹ (e.g., 10⁶ mol⁻¹, 10⁷ mol⁻¹, 10⁸ mol⁻¹, 10⁹ mol⁻¹, 10¹⁰ mol⁻¹, 10¹¹ mol⁻¹, and 10¹² mol⁻¹ or more) with that second molecule.

The term “monoclonal antibody” or “mAb” refers to an antibody obtained from a substantially homogeneous population of antibodies, i.e., the individual antibodies within the population are identical except for possible naturally occurring mutations that may be present in a small subset of the antibody molecules.

“Bi-specific chimeric antigen receptor” refers to a CAR that includes two domains, wherein the first domain is specific for a first ligand/antigen/target, and wherein the second domain is specific for a second ligand/antigen/target. In some forms, the ligand is a B-cell specific protein, a tumor-specific ligand/antigen/target, a tumor associated ligand/antigen/target, or combinations thereof. A bispecific CAR is specific to two different antigens. A multi-specific or multivalent CAR is specific to more than one different antigen, e.g., 2, 3, 4, 5, or more. In some forms, a multi-specific or multivalent CAR targets and/or binds three or more different antigens.

“Encoding” or “encode” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “locus” is the specific physical location of a DNA sequence (e.g., of a gene) on a chromosome. It is understood that a locus of interest can not only qualify a nucleic acid sequence that exists in the main body of genetic material (i.e., in a chromosome) of a cell but also a portion of genetic material that can exist independently to said main body of genetic material such as plasmids, episomes, virus, transposons or in organelles such as mitochondria as non-limiting examples.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. An “isolated nucleic acid” refers to a nucleic acid segment or fragment which has been separated from sequences which flank it in a naturally occurring state, e.g., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes: a recombinant DNA which is part of a hybrid gene encoding additional polypeptide sequence, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like.

In the context of cells, the term “isolated” also refers to a cell altered or removed from its natural state. That is, the cell is in an environment different from that in which the cell naturally occurs, e.g., separated from its natural milieu such as by concentrating to a concentration at which it is not found in nature. “Isolated cell” is meant to include cells that are within samples that are substantially enriched for the cell of interest and/or in which the cell of interest is partially or substantially purified.

As used herein, “transformed,” “transduced,” and “transfected” encompass the introduction of a nucleic acid or other material into a cell by one of a number of techniques known in the art.

A “vector” is a composition of matter which includes an isolated nucleic acid and which can be used to deliver the isolated nucleic acid to the interior of a cell. Examples of vectors include but are not limited to, linear polynucleotides, polynucleotides associated with ionic or amphiphilic compounds, plasmids, and viruses. Thus, the term “vector” encompasses an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus (AAV) vectors, retroviral vectors, and the like.

“Tumor burden” or “tumor load” as used herein, refers to the number of cancer cells, the size or mass of a tumor, or the total amount of tumor/cancer in a particular region of a subject. Methods of determining tumor burden for different contexts are known in the art, and the appropriate method can be selected by the skilled person. For example, in some forms tumor burden can be assessed using guidelines provided in the Response Evaluation Criteria in Solid Tumors (RECIST).

As used herein, “subject” includes, but is not limited to, animals, plants, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects. In some forms, the subject can be any organism in which the disclosed method can be used to genetically modify the organism or cells of the organism.

The term “inhibit” or other forms of the word such as “inhibiting” or “inhibition” means to decrease, hinder or restrain a particular characteristic such as an activity, response, condition, disease, or other biological parameter. It is understood that this is typically in relation to some standard or expected value, i.e., it is relative, but that it is not always necessary for the standard or relative value to be referred to. “Inhibits” can also mean to hinder or restrain the synthesis, expression or function of a protein relative to a standard or control. Inhibition can include, but is not limited to, the complete ablation of the activity, response, condition, or disease. “Inhibits” can also include, for example, a 10% reduction in the activity, response, condition, disease, or other biological parameter as compared to the native or control level. Thus, the reduction can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any amount of reduction in between as compared to native or control levels. For example, “inhibits expression” means hindering, interfering with or restraining the expression and/or activity of the gene/gene product pathway relative to a standard or a control.

“Treatment” or “treating” means to administer a composition to a subject or a system with an undesired condition (e.g., cancer). The condition can include one or more symptoms of a disease, pathological state, or disorder. Treatment includes medical management of a subject with the intent to cure, ameliorate, stabilize, or prevent a disease, pathological condition, or disorder. This includes active treatment, that is, treatment directed specifically toward the improvement of a disease, pathological state, or disorder, and also includes causal treatment, that is, treatment directed toward removal of the cause of the associated disease, pathological state, or disorder. In addition, this term includes palliative treatment, that is, treatment designed for the relief of symptoms rather than the curing of the disease, pathological state, or disorder; preventative treatment, that is, treatment directed to minimizing or partially or completely inhibiting the development of the associated disease, pathological state, or disorder; and supportive treatment, that is, treatment employed to supplement another specific therapy directed toward the improvement of the associated disease, pathological state, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount. “Prevention” or “preventing” means to administer a composition to a subject or a system at risk for an undesired condition (e.g., cancer). The condition can include one or more symptoms of a disease, pathological state, or disorder. The condition can also be a predisposition to the disease, pathological state, or disorder. The effect of the administration of the composition to the subject can be the cessation of a particular symptom of a condition, a reduction or prevention of the symptoms of a condition, a reduction in the severity of the condition, the complete ablation of the condition, a stabilization or delay of the development or progression of a particular event or characteristic, or reduction of the chances that a particular event or characteristic will occur.

As used herein, the terms “effective amount” or “therapeutically effective amount” means a quantity sufficient to alleviate or ameliorate one or more symptoms of a disorder, disease, or condition being treated, or to otherwise provide a desired pharmacologic and/or physiological effect. Such amelioration only requires a reduction or alteration, not necessarily elimination. The precise quantity will vary according to a variety of factors such as subject-dependent variables (e.g., age, immune system health, weight, etc.), the disease or disorder being treated, as well as the route of administration, and the pharmacokinetics and pharmacodynamics of the agent being administered.

By “pharmaceutically acceptable” is meant a material that is not biologically or otherwise undesirable, i.e., the material can be administered to a subject along with the selected compound without causing any undesirable biological effects or interacting in a deleterious manner with any of the other components of the pharmaceutical composition in which it is contained.

As used herein, the term “polypeptides” includes proteins and functional fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, the term “functional fragment” as used herein is a fragment of a full-length protein retaining one or more function properties of the full-length protein.

As used herein, the terms “variant” or “active variant” refers to a polypeptide or polynucleotide that differs from a reference polypeptide or polynucleotide, but retains essential properties (e.g., functional or biological activity). A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally. Modifications and changes can be made in the structure of the polypeptides of the disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological or functional activity, certain amino acid sequence substitutions can be made in a polypeptide sequence and nevertheless obtain a polypeptide with like properties (e.g., functional or biological activity).

Modifications and changes can be made in the structure of the polypeptides of in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within +2 is preferred, those within +1 are particularly preferred, and those within +0.5 are even more particularly preferred.

Substitution of like amino acids can also be made on the basis of hydrophilicity, particularly, where the biological functional equivalent polypeptide or peptide thereby created is intended for use in immunological forms. The following hydrophilicity values have been assigned to amino acid residues: arginine (+3.0); lysine (+3.0); aspartate (+3.0±1); glutamate (+3.0±1); serine (+0.3); asparagine (+0.2); glutamnine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within +2 is preferred, those within +1 are particularly preferred, and those within +0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Forms of this disclosure thus contemplate functional or biological equivalents of a polypeptide as set forth above. In particular, forms of the polypeptides can include variants having about 50%, 60%, 70%, 80%, 90%, and 95% sequence identity to the polypeptide of interest.

As used herein, “conservative” amino acid substitutions are substitutions wherein the substituted amino acid has similar structural or chemical properties.

As used herein, “non-conservative” amino acid substitutions are those in which the charge, hydrophobicity, or bulk of the substituted amino acid is significantly altered.

As used herein, the term “identity,” as known in the art, is a relationship between two or more polypeptide sequences, as determined by comparing the sequences. In the art, “identity” also means the degree of sequence relatedness between polypeptide as determined by the match between strings of such sequences. “Identity” can also mean the degree of sequence relatedness of a polypeptide compared to the full-length of a reference polypeptide. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48:1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48:443-453, 1970) algorithm (e.g., NBLAST, and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific form or combination of forms of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the forms and does not pose a limitation on the scope of the forms unless otherwise claimed. No language in the specification should be construed as indicating any non-claimed element as essential to the practice of the invention.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values can range in value either above or below the stated value in a range of approx. +/−5%; in other forms the values can range in value either above or below the stated value in a range of approx. +/−2%; in other forms the values can range in value either above or below the stated value in a range of approx. +/−1%. The preceding ranges are intended to be made clear by context, and no further limitation is implied.

### II. Compositions

Disclosed are antibodies, as well as fragments and antigen-binding components thereof, and fusion proteins including antigen binding fragments that are capable of immuno-specifically binding to Ectonucleotide pyrophosphatase/phosphodiesterase 3 (ENPP3 or CD203c) (also referred to herein as ENPP3-binding polypeptides). As discussed elsewhere herein in more detail, in non-limiting embodiments, such molecules can be, for example, a monoclonal antibody, a human antibody, a chimeric antibody or a humanized antibody, or a fragment thereof, and fusion proteins formed therefrom. In some forms, the fusion protein is a chimeric antigen receptor (CAR). The antibodies and antigen binding fragments can be monospecific, bispecific, trispecific or multispecific. In some forms, chimeric antigen receptors (CARs) include the antigen-binding component of an ENPP3-specific antibody. In some forms, the CARs include one or more additional modifications within the non-antigen binding component of the CAR. Exemplary non-antigen binding components of a CAR include the extracellular region, the transmembrane domain and the intracellular domain. Compositions including nucleic acids expressing the antibodies and antigen binding fragments and fusion proteins, such as CARs, and cells having the CARs are also provided. Also provided are pharmaceutical compositions including these molecules and cells, for example, for use in the diagnosis and treatment of cancer and other diseases.

### A. ENPP3-Binding Polypeptides

Polypeptides that selectively bind Ectonucleotide pyrophosphatase/phosphodiesterase 3 (ENPP3 or CD203c) are provided. ENPP3 is a type II transmembrane protein that belongs to the ectonucleotide pyrophosphatase/phosphodiesterase 3 (E-NPP3) family of enzymes involved in hydrolysis of oligonucleotides, nucleoside phosphates, and NAD. CD203c is expressed on resting cells at low levels and its expression is rapidly up-regulated following activation. ENPP3 increases expansion and survival of cancer kidney cells. ENPP3 is also a tumor associated antigen (TAA) for renal cell carcinoma (RCC) that is highly expressed in RCC with limited expression in normal tissue.

An exemplary consensus amino acid sequence
for the mature human ENPP3 polypeptide is:
(SEQ ID NO: 1)
MESTLTLATEQPVKKNTLKKYKIACIVLLALLVIMSLGLGLGLGL
RKLEKQGSCRKKCFDASFRGLENCRCDVACKDRGDCCWDFEDTCV
ESTRIWMCNKFRCGETRLEASLCSCSDDCLQRKDCCADYKSVCQG
ETSWLEENCDTAQQSQCPEGFDLPPVILFSMDGFRAEYLYTWDTL
MPNINKLKTCGIHSKYMRAMYPTKTFPNHYTIVTGLYPESHGIID
NNMYDVNLNKNFSLSSKEQNNPAWWHGQPMWLTAMYQGLKAATYF
WPGSEVAINGSFPSIYMPYNGSVPFEERISTLLKWLDLPKAERPR
FYTMYFEEPDSSGHAGGPVSARVIKALQVVDHAFGMLMEGLKQRN
LHNCVNIILLADHGMDQTYCNKMEYMTDYFPRINFFYMYEGPAPR
IRAHNIPHDFFSFNSEEIVRNLSCRKPDQHFKPYLTPDLPKRLHY
AKNVRIDKVHLFVDQQWLAVRSKSNTNCGGGNHGYNNEFRSMEAI
FLAHGPSFKEKTEVEPFENIEVYNLMCDLLRIQPAPNNGTEGSLN
HLLKVPFYEPSHAEEVSKFSVCGFANPLPTESLDCFCPHLQNSTQ
LEQVNQMLNLTQEEITATVKVNLPFGRPRVLQKNVDHCLLYHREY
VSGFGKAMRMPMWSSYTVPQLGDTSPLPPTVPDCLRADVRVPPSE
SQKCSFYLADKNITHGFLYPPASNRTSDSQYDALITSNLVPMYEE
FRKMWDYFHSVLLIKHATERNGVNVVSGPIFDYNYDGHFDAPDEI
TKHLANTDVPIPTHYFVVLTSCKNKSHTPENCPGWLDVLPFIIPH
RPINVESCPEGKPEALWVEERFTAHIARVRDVELLTGLDFYQDKV
QPVSEILQLKTYLPTFETTI.
In other forms, the amino acid sequence for the
mature human ENPP3 polypeptide is:
(SEQ ID NO: 2)
LLVIMSLGLGLGLGLRKLEKQGSCRKKCFDASFRGLENCRCDVAC
KDRGDCCWDFEDTCVESTRIWMCNKFRCGETRLEASLCSCSDDCL
QRKDCCADYKSVCQGETSWLEENCDTAQQSQCPEGFDLPPVILFS
MDGFRAEYLYTWDTLMPNINKLKTCGIHSKYMRAMYPTKTFPNKY
TIVTGLYPESHGIIDNNMYDVNLNKNFSLSSKEQNNPAWWHGQPM
WLTAMYQGLKAATYFWPGSEVAINGSFPSIYMPYNGSVPFEERIS
TLLKWLDLPKAERPRFYTMYFEEPDSSGHAGGPVSARVIKALQVV
DHAFGMLMEGLKQRNLHNCVNIILLADHGMDQTYCNKMEYMTDYF
PRINFFYMYEGPAPRIRAHNIPHDFFSENSEEIVRNLSCRKPDQH
FKPYLTPDLPKRLHYAKNVRIDKVHLEVDQQWLAVRSKSNTNCGG
GNAGYNNEFRSMEAIFLAHGPSFKEKTEVEPFENIEVYNLMCDLL
RIQPAPNNGTHGSLNHLLKVPFYEPSHAEEVSKESVCGFANPLPT
ESLDCFCPHLQNSTQLEQVNQMLNLTQEEITATVKVNLPFGRPRV
LQKNVDHCLLYHREYVSGFGKAMRMPMWSSYTVPQLGDTSPLPPT
VPDCLRADVRVPPSESQKCSFYLADKNITHGFLYPPASNRTSDSQ
YDALITSNLVPMYEEFRKMWDYFHSVLLIKHATERNGVNVVSGPI
FDYNYDGHEDAPDEITKHLANIDVPIPTHYFVVLISCKNKSHTPE
NCPGWLDVLPFIIPHRPTNVESCPEGKPEALWVEERFTAHIARVR
DVELLTGLDFYQDKVQPVSBILQLKTYLPTFETTI.

In some embodiments, the disclosed ENPP3-binding polypeptides bind to the ENPP3 of SEQ ID NO:1 and/or SEQ ID NO:2, or a homologue, paralogue, or ortholog thereof. In some embodiments, the ENPP3-binding polypeptides bind to a variant having at least 70%, up to 99% identity to SEQ ID NOS: 1 and/or 2. For example in some forms, the variant sequence has at least about 70%, 75%, 80%, 85%, 90%, or 95% identity to SEQ ID NO:2. Therefore, in some forms, the variant consensus amino acid sequence for the mature ENPP3 polypeptide has an amino acid sequence that has one or more amino acids different to SEQ ID NOS: 1 and/or 2, such as one or more substitutions, deletions or additions at any one of the amino acid positions of SEQ ID NOS: 1 and/2.

### B. Exemplary ENPP3-Binding Polypeptide Sequences

As demonstrated in the examples, a panel of fully-human, anti-ENPP3 immunoglobulins has been developed.

As introduced above, the term antibody herein refers to natural or synthetic polypeptides that bind a target antigen. The term includes polyclonal and monoclonal antibodies, including intact antibodies and functional (e.g., antigen-binding) antibody fragments, including Fab fragments, F(ab′)2 fragments, Fab′ fragments, Fv fragments, recombinant IgG (rlgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific antibodies, diabodies, triabodies, and tetrabodies, tandem di-scFv, tandem tri-scFv. The term also encompasses intact or full-length antibodies, including antibodies of any class or subclass, including IgG and sub classes thereof, IgM, IgE, IgA, and IgD. Thus, although typically discussed in the context of IgG, the target antibody of the Ig-Fc-specific immunoglobulin variable domain can be IgM, IgE, IgA, or IgD.

Each of 8 different IgG candidate clones were identified, named SX13-1, SX13-2, SX13-5, SX13-6, SX13-7, SX13-8.1, SX13-10 and SX13-12, respectively.

Provided are molecules or antibodies that bind to ENPP3, and wherein the antigen binding domain includes six CDRs, wherein the CDRs include at least one consensus CDR of the CDRs of any of the anti-ENPP3 antibodies SX13-1, SX13-2, SX13-5, SX13-6, SX13-7, SX13-8.1, SX13-10 and SX13-12, with all remaining CDRs selected from:
(A) the three light chain CDRs and/or the three heavy chain CDRs of anti-ENPP3 antibody SX13-1;
(B) the three light chain CDRs and/or the three heavy chain CDRs of anti-ENPP3 antibody SX13-2;
(C) the three light chain CDRs and/or the three heavy chain CDRs of anti-ENPP3 antibody SX13-5;
(D) the three light chain CDRs and/or the three heavy chain CDRs of anti-ENPP3 antibody SX13-6;
(E) the three light chain CDRs and/or the three heavy chain CDRs of anti-ENPP3 antibody SX13-7;
(F) the three light chain CDRs and/or the three heavy chain CDRs of anti-ENPP3 antibody SX13-8.1;
(G) the three light chain CDRs and/or the three heavy chain CDRs of anti-ENPP3 antibody SX13-10; or
(H) the three light chain CDRs and/or the three heavy chain CDRs of anti-ENPP3 antibody SX13-12.

In some embodiments, the molecules or antibodies which binds to ENPP3, and wherein the antigen binding domain includes six CDRs, wherein the six CDRs are:
(A) the three light chain and the three heavy chain CDRs of anti-ENPP3 antibody SX13-1;
(B) the three light chain and the three heavy chain CDRs of anti-ENPP3 antibody SX13-2;
(C) the three light chain and the three heavy chain CDRs of anti-ENPP3 antibody SX13-5;
(D) the three light chain and the three heavy chain CDRs of anti-ENPP3 antibody SX13-6;
(E) the three light chain and the three heavy chain CDRs of anti-ENPP3 antibody SX13-7;
(F) the three light chain and the three heavy chain CDRs of anti-ENPP3 antibody SX13-8.1;
(G) the three light chain and the three heavy chain CDRs of anti-ENPP3 antibody SX13-10; or
(H) the three light chain and the three heavy chain CDRs of anti-ENPP3 antibody SX13-12.

In some embodiments, the CDRs in the light and heavy chains are in the same order and/or orientation as in SX13-1, SX13-2, SX13-5, SX13-6, SX13-7, SX13-8.1, SX13-10 or SX13-12.

In some embodiments, the antibody binds to ENPP3 and includes one or both of the light and heavy chain variable domain(s) of clone SX13-1, SX13-2, SX13-5, SX13-6, SX13-7, SX13-8.1, SX13-10 or SX13-12.

The amino acid sequences for the heavy and light chain variable regions of SX13-1, SX13-2, SX13-5, SX13-6, SX13-7, SX13-8.1, SX13-10 and SX13-12 are provided below as SEQ ID NOS: 3-18 and include the signal sequence in italic font and CDRs (according to the Chothia numbering system) in bold. Also expressly provided are SEQ ID NOS: 3-18 without the signal sequence, e.g., as SEQ ID NOS: 70-85.

Variants of SEQ ID NOS: 3-18 with and without the signal sequence (e.g., SEQ ID NOS: 70-85) that bind ENPP3 and have at least 70% and up to 99.9% sequence identity to any of SEQ ID NOS: 3-18 and 70-85 are also provided, and can be used as the heavy chain (i.e., variants of SEQ ID NOS: 3, 5, 7, 9, 11, 13, 15, 17, 71, 73, 75, 77, 79, 81, 83, 85) and/or light chain (i.e., variants of SEQ ID NOS: 4, 6, 8, 10, 12, 14, 16, 18, 70, 72, 74, 76, 78, 80, 82, 84) variable regions of the disclosed antigen binding domains (i.e., in place the correspondence sequence of SEQ ID NOS: 3-18 or 70-85). For example, in some forms, the variant sequence has at least about 70%, 75%, 80%, 85%, 90%, or 95% identity to any one of SEQ ID NOS: 3-18 with or without the signal sequence (e.g., SEQ ID NOS: 70-85). Therefore, in some forms, the amino acid sequence has one or more amino acids different, such as one or more substitutions, deletions or additions at any one of the amino acid positions of its corresponding sequence of any one of SEQ ID NOS: 3-18 or 70-85. In some embodiments, variants of SEQ ID NOS: 3-18 or 70-85 include the CDRs of SEQ ID NOS: 3-18 or 70-85, respectively (i.e., without variation).

1.
SX13-1 H/L Subunits
An exemplary consensus amino acid sequence for the mature variable heavy
subunit of SX13-1 is:
(SEQ ID NO: 3)
MELGLRWVFLVAILEGVQCEVQLVESGGGLVKPGGSLRLSCAASGFTFSDYSMNWVRQA
PGKGLEWVSSISSSSRYKYYADSMKGRFTISRDNAKNSLYLQMNSLRVEDTAVYYCTRG
IDYFDYWGQGTLVTVSS,
or
(SEQ ID NO: 70)
QLVESGGGLVKPGGSLRLSCAASGFTFSDYSMNWVRQAPGKGLEWVSSISSSSRYKYYA
DSMKGRFTISRDNAKNSLYLQMNSLRVEDTAVYYCTRGIDYFDYWGQGTLVTVSS,
without the signal sequence.
Chothia CDR-H1:
(SEQ ID NO: 29)
FTFSDY
Chothia CDR-H2:
(SEQ ID NO: 30)
SSSSRY
Chothia CDR-H3:
(SEQ ID NO: 31)
GIDYFDY
An exemplary consensus amino acid sequence for the mature variable light
subunit of SX13-1 is:
(SEQ ID NO: 4)
MDMRVPAQLLGLLLLWLPGARCDIQLTQSPSFLSASVGDRVTITCRASQDINNYLAWYQ
QKPGKAPKFLIHVASTLQSGVPSRFSGSGSGTEFTLTISSLQPEDSATYYCQQLNSYPR
TFGQGTKVEIK,
or
(SEQ ID NO: 71)
DIQLTQSPSFLSASVGDRVTITCRASQDINNYLAWYQQKPGKAPKFLIHVASTLQSGVP
SRFSGSGSGTEFTLTISSLQPEDSATYYCQQLNSYPRTFGQGTKVEIK,
without the signal sequence.
Chothia CDR-L1:
(SEQ ID NO: 32)
RASQDINNYLA
Chothia CDR-L2:
(SEQ ID NO: 33)
VASTLQS
Chothia CDR-L3:
(SEQ ID NO: 34)
QQLNSYPRT
2.
SX13-2 H/L Subunits
An exemplary consensus amino acid sequence for the mature variable heavy
subunit of SX13-2 is:
(SEQ ID NO: 5)
MELGLRWVFLVAILEGVQCEVQLVESGGGLVKPGGSLRLSCAASGFTFSSYSMNWVRQA
PGKGLEWVTYISSSSSYRYYADSVKGRFTISRDNAKNSLNLQMNTLRPEDTAVYYCARV
GAITGYFDFWGQGTLVTVSS,
or
(SEQ ID NO: 72)
QLVESGGGLVKPGGSLRLSCAASGFTFSSYSMNWVRQAPGKGLEWVTYISSSSSYRYYA
DSVKGRFTISRDNAKNSLNLQMNTLRPEDTAVYYCARVGAITGYFDFWGQGTLVTVSS,
without the signal sequence.
Chothia CDR-H1:
(SEQ ID NO: 35)
GFTFSSY
Chothia CDR-H2:
(SEQ ID NO: 36)
SSSSSY
Chothia CDR-H3:
(SEQ ID NO: 37)
VGAITGYEDF
An exemplary consensus amino acid sequence for the mature variable light
subunit of SX13-2 is:
(SEQ ID NO: 6)
MDMRVPAQLLGLLLLWFPGARCDIQMTQSPSSLSASVGDRVTITCRASQGIRNDLDWFQ
QKPGKAPKRLIYAASSLQSGVPSRFSGSGSGTEFTLTVSSLQPEDFATYYCLQYNGYPL
TFGGGTKVEIK,
or
(SEQ ID NO: 73)
DIQMTQSPSSLSASVGDRVTITCRASQGIRNDLDWFQQKPGKAPKRLIYAASSLQSGVP
SRFSGSGSGTEFTLTVSSLQPEDFATYYCLQYNGYPLTFGGGTKVEIK,
without the signal sequence.
Chothia CDR-L1:
(SEQ ID NO: 38)
RASQGIRNDLD
Chothia CDR-L2:
(SEQ ID NO: 39)
AASSLQS
Chothia CDR-L3:
(SEQ ID NO: 40)
LQYNGYPLT
3.
SX13-5 H/L Subunits
An exemplary consensus amino acid sequence for the mature variable heavy
subunit of SX13-5 is:
(SEQ ID NO: 7)
MELGLRWVFLVAILEGVQCEVQLVESGGGLVKPGGSLRLSCAASGFTFSTFSMNWVRQA
PGKGLEWVSYISSYSSYIYYADSVKGRFTISRDNAKKSLYLQMNSLRAEDTAVYYCSRV
GAITGYFDYWGQGTLVTVSS,
or
(SEQ ID NO: 74)
QLVESGGGLVKPGGSLRLSCAASGFTFSTFSMNWVRQAPGKGLEWVSYISSYSSYIYYA
DSVKGRFTISRDNAKKSLYLQMNSLRAEDTAVYYCSRVGAITGYFDYWGQGTLVTVSS,
without the signal sequence.
Chothia CDR-H1:
(SEQ ID NO: 41)
GFTFSTF
Chothia CDR-H2:
(SEQ ID NO: 42)
SSYSSY
Chothia CDR-H3:
(SEQ ID NO: 43)
VGAITGYFDY
An exemplary consensus amino acid sequence for the mature variable light
subunit of SX13-5 IgG is:
(SEQ ID NO: 8)
MDMRVPAQLLGLLLLWFPGARCGIQMTQSPSSLSASVGDRVTITCRASQGIRNDLGWYQ
QTPGKAPKRLMYTASSLHSGVPSRFSGSGSGTEFTLTISSLQPEDFATYYCLQYNGYPL,
TFGGGTKVEIK,
or
(SEQ ID NO: 75)
GIQMTQSPSSLSASVGDRVTITCRASQGIRNDLGWYQQTPGKAPKRLMYTASSLHSGVP
SRFSGSGSGTEFTLTISSLQPEDFATYYCLQYNGYPLTFGGGTKVEIK,
without the signal sequence.
Chothia CDR-L1:
(SEQ ID NO: 44)
RASQGIRNDLG
Chothia CDR-L2:
(SEQ ID NO: 45)
TASSLHS
Chothia CDR-L3:
(SEQ ID NO: 40)
LQYNGYPLT
4.
SX13-6 H/L Subunits
An exemplary consensus amino acid sequence for the mature variable heavy
subunit of SX13-6 is:
(SEQ ID NO: 9)
MEFGLSWVFLVAIIKGVQCQVQLVESGGDLVKPGGSLRLSCAASGFTFSDYYMNWIRQA
PEKGLEWISYISSSGFIMDSADSVKGRFTISRDNAKNSLFLQMNSLRAEDTAVYYCARG
NWNYPFDYWGQGTLVTVSS,
or
(SEQ ID NO: 76)
QLVESGGDLVKPGGSLRLSCAASGFTFSDYYMNWIRQAPEKGLEWISYISSSGFIMDSA
DSVKGRFTISRDNAKNSLFLQMNSLRAEDTAVYYCARGNWNYPFDYWGQGTLVTVSS,
withoutthesignalsequence.
Chothia CDR-H1:
(SEQ ID NO: 46)
GFTFSDY
Chothia CDR-H2:
(SEQ ID NO: 47)
SSSGFI
Chothia CDR-H3:
(SEQ ID NO: 48)
GNWNYPFDY
An exemplary consensus amino acid sequence for the mature variable light
subunit of SX13-6 IgG is:
(SEQ ID NO: 10)
MDMRVPAQLLGLLLLWLPATRCDIQMTQSPSSLSASIGDRVTITCRASQGISHYLAWYQ
QRPGKVPKFLIYAASTLQSGVPSRFSGRGSGTDFTLTISSLQPEDIATYYCQMYKNVPF
TFGPGTKVDIK,
or
(SEQ ID NO: 77)
DIQMTQSPSSLSASIGDRVTITCRASQGISHYLAWYQQRPGKVPKFLIYAASTLQSGVP
SRFSGRGSGTDFTLTISSLQPEDIATYYCQMYKNVPFTFGPGTKVDIK,
without the signal sequence.
Chothia CDR-L1:
(SEQ ID NO: 49)
RASQGISHYLA
Chothia CDR-L2:
(SEQ ID NO: 50)
AASTLQS
Chothia CDR-L3:
(SEQ ID NO: 51)
QMYKNVPFT
5.
SX13-7 H/L Subunits
An exemplary consensus amino acid sequence for the mature variable heavy
subunit of SX13-7 is:
(SEQ ID NO: 11)
MELGLRWVELVALLEGVQCEVQLVESGGGLVKPGGSLRLSCAASGFTFSDYSMNWVRQA
PGKGLEWVSSISSSSSYKKYADSVKGRLTISRDNAKNSLYLQMNSLRAEDTAVYYCARG
IDYFDYWGQGTLVTVSS,
or
(SEQ ID NO: 78)
QLVESGGGLVKPGGSLRLSCAASGFTFSDYSMNWVRQAPGKGLEWVSSISSSSSYKKYA
DSVKGRLTISRDNAKNSLYLQMNSLRAEDTAVYYCARGIDYFDYWGQGTLVTVSS,
without the signal sequence.
Chothia CDR-H1:
(SEQ ID NO: 46)
GFTFSDY
Chothia CDR-H2:
(SEQ ID NO: 36)
SSSSSY
Chothia CDR-H3:
(SEQ ID NO: 31)
GIDYFDY
An exemplary consensus amino acid sequence for the mature variable light
subunit of SX13-7 is:
(SEQ ID NO: 12)
MDMRVPAQLLGLLLLWLPGARCDIQLTQSPSFLSASVGDRVTITCRASOGISSNLAWYQ
QKPGKDPKVLIYGASTLQSGVPSRFSGSGSGTEFTLTISSLQPEDFATYYCQQLNSYPR
TFGQGTKVEIK,
or
(SEQ ID NO: 79)
DIQLTQSPSFLSASVGDRVTITCRASQGISSNLAWYQQKPGKDPKVLIYGASTLQSGVP
SRFSGSGSGTEFTLTISSLQPEDFATYYCQQLNSYPRTFGQGTKVEIK,
without the signal sequence.
Chothia CDR-L1:
(SEQ ID NO: 52)
RASQGISSNLA
Chothia CDR-L2:
(SEQ ID NO: 53)
GASTLQS
Chothia CDR-L3:
(SEQ ID NO: 34)
QQLNSYPRT
6.
SX13-8.1 H/L Subunits
An exemplary consensus amino acid sequence for the mature variable heavy
subunit of SX13-8.1 is:
(SEQ ID NO: 13)
MSVSFLIFLPVLGLPWGVLSQVQLQQSGPGLVKPSQTLSLTCAISGDSVSSNRAAWNWI
RQPPSRGLEWLGRTYYRSNWYNDYAVSVKSRITIKPDTSKNQFSLQLNSVTPEDTAVYY
CARDRGTYYYYYYMDVWDKGTTVTVSS,
or
(SEQ ID NO: 80)
VQLQQSGPGLVKPSQTLSLTCAISGDSVSSNRAAWNWIRQPPSRGLEWLGRTYYRSNWY
NDYAVSVKSRITIKPDTSKNQFSLQLNSVTPEDTAVYYCARDRGTYYYYYYMDVWDKGT
TVTVSS,
without the signal sequence.
Chothia CDR-H1:
(SEQ ID NO: 54)
GDSVSSNRA
Chothia CDR-H2:
(SEQ ID NO: 55)
YYRSNWY
Chothia CDR-H3:
(SEQ ID NO: 56)
RGTYYYYYYMDV
An exemplary consensus amino acid sequence for the mature variable light
subunit of SX13-8.1 is:
(SEQ ID NO: 14)
METPAQLLLLLLWLPDTTGEIVLTQSPGTLSLSPGERATLSCRASQSVSSSYLVWYQQ
KAGQAPRILIYGASNRATDIPVRFSGSGSGTDFTLTISRLEPEDFGVYFCQHYGGSYTF
GQGTKLEIK,
or
(SEQ ID NO: 81)
EIVLTQSPGTLSLSPGERATLSCRASQSVSSSYLVWYQQKAGQAPRILIYGASNRATDI
PVRFSGSGSGTDFTLTISRLEPEDFGVYFCQHYGGSYTFGQGTKLEIK,
without the signal sequence.
Chothia CDR-L1:
(SEQ ID NO: 57)
RASQSVSSSYLV
Chothia CDR-L2:
(SEQ ID NO: 58)
GASNRAT
Chothia CDR-L3:
(SEQ ID NO: 59)
QHYGGSYT
7.
SX13-10 H/L Subunits
An exemplary consensus amino acid sequence for the mature variable heavy
subunit of SX13-10 is:
(SEQ ID NO: 15)
MELGLRWVFLVAILEGVQCEVQLLESGGGLVKPGGSLRLSCAASGFTFSRYNMNWVRQA
PGKGLEWVSFISGSSSYVYYADSVKGRFTISKDNAKNSLYLQMNSLRAEDTAVYYCARS
GYCTSTTCYNYYYYYMDVWGKGTTVTVSS,
or
(SEQ ID NO: 82)
QLLESGGGLVKPGGSLRLSCAASGFTFSRYNMNWVRQAPGKGLEWVSFISGSSSYVYYA
DSVKGRFTISKDNAKNSLYLQMNSLRAEDTAVYYCARSGYCTSTTCYNYYYYYMDVWGK
GTTVTVSS,
without the signal sequence.
Chothia CDR-H1:
(SEQ ID NO: 60)
GFTFSRY
Chothia CDR-H2:
(SEQ ID NO: 61)
SGSSSY
Chothia CDR-H3:
(SEQ ID NO: 62)
SGYCTSTTCYNYYYYYMDV
An exemplary consensus amino acid sequence for the mature variable light
subunit of SX13-10 is:
(SEQ ID NO: 16)
METPAQLLFLLLLWLPDTTGEIVLMQSPGTLSLSPGERATLSCRASQSVSNSYLAWYQQ
KPGQAPRLLIYGASSRATGIPDRFSGSGSGTDFTLTISRLEPEDFAVFYCQQFGSSPLT
FGGGTKVEIK
or
(SEQ ID NO: 83)
EIVLMQSPGTLSLSPGERATLSCRASQSVSNSYLAWYQQKPGQAPRLLIYGASSRATGI
PDRFSGSGSGTDFTLTISRLEPEDFAVFYCQQFGSSPLTFGGGTKVEIK
without the signal sequence.
Chothia CDR-L1:
(SEQ ID NO: 63)
RASQSVSNSYLA
Chothia CDR-L2:
(SEQ ID NO: 64)
GASSRAT
Chothia CDR-L3:
(SEQ ID NO: 65)
QQFGSSPLT
8.
SX13-12 H/L Subunits
An exemplary consensus amino acid sequence for the mature variable heavy
subunit of SX13-12 is:
(SEQ ID NO: 17)
MELGLRWVFLVAILEGVQCELQLVESGGGLVKPGGSLRLSCAASGFTFSDYSMNWVRQA
PGKGLNWVASISSSSHYKYYADSVKGRFTISRDNAKNSLYLQMNSLRAEDTAVYYCARG
IDFFDIWGQGTMVTVSS,
or
(SEQ ID NO: 84)
QLVESGGGLVKPGGSLRLSCAASGFTFSDYSMNWVRQAPGKGLNWVASISSSSHYKYYA
DSVKGRFTISRDNAKNSLYLQMNSLRAEDTAVYYCARGIDFFDIWGQGTMVTVSS,
without the signal sequence.
Chothia CDR-H1:
(SEQ ID NO: 46)
GFTFSDY
Chothia CDR-H2:
(SEQ ID NO: 66)
SSSSHY
Chothia CDR-H3:
(SEQ ID NO: 67)
GIDFFDI
An exemplary consensus amino acid sequence for the mature variable light
subunit of SX13-12 is:
(SEQ ID NO: 18)
MDMRVPAQLLGLLLLWLPGARCDIQLTQSPSFLSASVGDRVTITCRASQDINSYLVWYQ
QKPGKAPKLLIYTASTLQSGVPSRFSGSGSGTEFTLTIRSLQPEDFATYYCQQLNSYPR
TFGQGTKVEIK
or
(SEQ ID NO: 85)
DIQLTQSPSFLSASVGDRVTITCRASQDINSYLVWYQQKPGKAPKLLIYTASTLQSGVP
SRFSGSGSGTEFTLTIRSLQPEDFATYYCQQLNSYPRTFGQGTKVEIK,
without the signal sequence.
Chothia CDR-L1:
(SEQ ID NO: 68)
RASQDINSYLV
Chothia CDR-L2:
(SEQ ID NO: 69)
TASTLQS
Chothia CDR-L3:
(SEQ ID NO: 34)
QQLNSYPRT

### C. Anti-ENPP3 Single Chain Variable Fragment (scFV)

Also disclosed are single chain variable (scFv) fragments of anti-ENPP3 antibodies which have bioactivity.

The scFvs, whether attached to other sequences or not, can include insertions, deletions, substitutions, or other selected modifications of particular regions or specific amino acids residues, provided the activity of the fragment is not significantly altered or impaired compared to the non-modified antibody or antibody fragment.

Methods for the production of single-chain antibodies are well known to those of skill in the art. Techniques can be adapted for the production of single-chain antibodies specific to an antigenic protein. A single chain antibody can be created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding. The linker is chosen to permit the heavy chain and light chain to bind together in their proper conformational orientation.

In some embodiments, the scFv includes the heavy and light chain variable regions CDRs of SX13-1, SX13-2, SX13-5, SX13-6, SX13-7, SX13-8.1, SX13-10 or SX13-12, or the heavy and light chain variable regions of SX13-1, SX13-2, SX13-5, SX13-6, SX13-7, SX13-8.1, SX13-10 or SX13-12, optionally, but preferably connected by a linker.

The anti-ENPP3 antibodies can be modified to improve their therapeutic potential. For example, in some embodiments, the anti-ENPP3 antibody is conjugated to another antibody specific for a second therapeutic target in the nucleus of the cancer cell. For example, the anti-ENPP3 antibody can be a fusion protein containing an scFv corresponding to any one of SX13-1, SX13-2, SX13-5, SX13-6, SX13-7, SX13-8.1, SX13-10 and SX13-12, respectively, and a single chain variable fragment of a monoclonal antibody that specifically binds the second therapeutic target, or has an amino acid sequence of any one or more of corresponding to any one of SX13-1, SX13-2, SX13-5, SX13-6, SX13-7, SX13-8.1, SX13-10 and SX13-12. In other embodiments, the anti-ENPP3 antibody is a bispecific antibody having a first heavy chain and a first light chain from any one of SX13-1, SX13-2, SX13-5, SX13-6, SX13-7, SX13-8.1, SX13-10 and SX13-12, and a second heavy chain and a second light chain from a monoclonal antibody that specifically binds the second therapeutic target.

Divalent single-chain variable fragments (di-scFvs) can be engineered by linking two scFvs. This can be done by producing a single peptide chain with two VH and two VL regions, yielding tandem scFvs. ScFvs can also be designed with linker peptides that are too short for the two variable regions to fold together (about five amino acids), forcing scFvs to dimerize. This type is known as diabodies. Diabodies have been shown to have dissociation constants up to 40-fold lower than corresponding scFvs, meaning that they have a much higher affinity to their target. Still shorter linkers (one or two amino acids) lead to the formation of trimers (triabodies or tribodies). Tetrabodies have also been produced. They exhibit an even higher affinity to their targets than diabodies.

The therapeutic function of the antibody can be enhanced by coupling the antibody or a fragment thereof with a therapeutic agent. Such coupling of the antibody or fragment with the therapeutic agent can be achieved by making an immunoconjugate or by making a fusion protein, or by linking the antibody or fragment to a nucleic acid such as an siRNA or to a small molecule, including the antibody or antibody fragment and the therapeutic agent.

A recombinant fusion protein is a protein created through genetic engineering of a fusion gene. This typically involves removing the stop codon from a cDNA sequence coding for the first protein, then appending the cDNA sequence of the second protein in frame through ligation or overlap extension PCR. The DNA sequence will then be expressed by a cell as a single protein. The protein can be engineered to include the full sequence of both original proteins, or only a portion of either. If the two entities are proteins, often linker (or “spacer”) peptides are also added which make it more likely that the proteins fold independently and behave as expected.

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Antibody humanization techniques generally involve the use of recombinant DNA technology to manipulate the DNA sequence encoding one or more polypeptide chains of an antibody molecule.

In some embodiments, the anti-ENPP3 antibody is modified to alter its half-life. In some embodiments, it is desirable to increase the half-life of the antibody so that it is present in the circulation or at the site of treatment for longer periods of time. For example, where the anti-ENPP3 antibodies are being used alone to treat cancer, it may be desirable to maintain titers of the antibody in the circulation or in the location to be treated for extended periods of time. In other embodiments, the half-life of the anti-ENPP3 antibody is decreased to reduce potential side effects. For example, where the antibody is being used in conjunction with radiotherapy or chemotherapy, the antibody is preferably present in the circulation at high doses during the treatment with radiation or antineoplastic drug but is otherwise quickly removed from the circulation. Antibody fragments, such as an scFv of any one of SX13-1, SX13-2, SX13-5, SX13-6, SX13-7, SX13-8.1, SX13-10 and SX13-12, are expected to have a shorter half-life than full size antibodies. Other methods of altering half-life are known and can be used in the described methods. For example, antibodies can be engineered with Fc variants that extend half-life, e.g., using Xtend™ antibody half-life prolongation technology (Xencor, Monrovia, CA).

In some embodiments, binding proteins are formed by the pair of SEQ ID NOS: 3 and 4, or SEQ ID NOs: 5 and 6, or SEQ ID NOs: 7 and 8, or SEQ ID NOs: 9 and 10, or SEQ ID NOs: 11 and 12, or SEQ ID NOs: 13 and 14, or SEQ ID NOs: 15 and 16, or SEQ ID NOs: 17 and 18, with or without the signal sequence. Exemplary ENPP3-binding scFvs, including an amino acid sequence corresponding to any one of SX13-1, SX13-2, SX13-5, SX13-6, SX13-7, SX13-8.1, SX13-10 and SX13-12 are provided below, as SEQ ID NOS: 19-26, wherein a linker is depicted in italic font, and CDRs (according to the Chothia numbering system are depicted in bold font. Typically, the scFvs include an amino acid sequence corresponding to the heavy (H) and light (L) variable subunits of any one of SX13-1, SX13-2, SX13-5, SX13-6, SX13-7, SX13-8.1, SX13-10 and SX13-12, coupled by a linker. An exemplary linker is SSSGGGGGGGGSGGGGS (SEQ ID NO: 27).

Also provided are variants of SEQ ID NOS: 19-26 that bind ENPP3 and have at least 70% and up to 99% sequence identity to any of SEQ ID NOS: 19-26. For example, in some forms, the variant sequence has at least about 70%, 75%, 80%, 85%, 90%, or 95% identity to any one of SEQ ID NOS: 19-26. Therefore, in some forms, the amino acid sequence has one or more amino acids different, such as one or more substitutions, deletions or additions at any one of the amino acid positions of its corresponding sequence of SEQ ID NOS: 19-26. In some embodiments, variants of SEQ ID NOS: 19-26 include the CDRs of SEQ ID NOS: 3-18, respectively (i.e., without variation).

1.
SX13-1 scFV
An exemplary SX13-1 scFv has the amino acid
sequence:
(SEQ ID NO: 19)
QLVESGGGLVKPGGSLRLSCAASGFTFSDYSMNWVRQAPGKGLEWVS
SISSSSRYKYYADSMKGRFTISRDNAKNSLYLQMNSLRVEDTAVYYC
TRGIDYFDYWGQGTLVTVSSSGGGGSGGGGSGGGGSDIQLTQSPSFL
SASVGDRVTITCRASQDINNYLAWYQQKPGKAPKFLIHVASTLQSGV
PSRFSGSGSGTEFTLTISSLQPEDSATYYCQQLNSYPRTFGQGTKVE
IK.
2.
SX13-2 scFv
An exemplary SX13-2 IgG scFv has the amino acid
sequence:
(SEQ ID NO: 20)
QLVESGGGLVKPGGSLRLSCAASGFTFSSYSMNWVRQAPGKGLEWVT
YISSSSSYRYYADSVKGRFTISRDNAKNSLNLQMNTLRPEDTAVYYC
ARVGAITGYFDFWGQGTLVTVSSSGGGGSGGGGSGGGGSDIQMTQSP
SSLSASVGDRVTITCRASQGIRNDLDWFQQKPGKAPKRLIYAASSLQ
SGVPSRFSGSGSGTEFTLTVSSLQPEDFATYYCLQYNGYPLTFGGGT
KVEIK.
3.
SX13-5 scFv
An exemplary SX13-5 scFv has the amino acid
sequence:
(SEQ ID NO: 21)
QLVESGGGLVKPGGSLRLSCAASGFTFSTFSMNWVRQAPGKGLEWVS
YISSYSSYIYYADSVKGRFTISRDNAKKSLYLQMNSLRAEDTAVYYC
SRVGAITGYFDYWGQGTLVTVSSSGGGGSGGGGSGGGGSGIQMTQSP
SSLSASVGDRVTITCRASQGIRNDLGWYQQTPGKAPKRLMYTASSLH
SGVPSRFSGSGSGTEFTLTISSLQPEDFATYYCLQYNGYPLTFGGGT
KVEIK.
4.
SX13-6 scFv
An exemplary SX13-6 scFv has the amino acid
sequence:
(SEQ ID NO: 22)
QLVESGGDLVKPGGSLRLSCAASGFTFSDYYMNWIRQAPEKGLEWIS
YISSSGFIMDSADSVKGRFTISRDNAKNSLFLQMNSLRAEDTAVYYC
ARGNWNYPFDYWGQGTLVTVSSSGGGGSGGGGSGGGGSDIQMTQSPS
SLSASIGDRVTITCRASQGISHYLAWYQQRPGKVPKFLIYAASTLQS
GVPSRFSGRGSGTDFTLTISSLQPEDIATYYCQMYKNVPFTFGPGTK
VDIK.
5.
SX13-7 scFv
An exemplary SX13-7 scFv has the amino acid
sequence:
(SEQ ID NO: 23)
QLVESGGGLVKPGGSLRLSCAASGFTFSDYSMNWVRQAPGKGLEWVS
SISSSSSYKKYADSVKGRLTISRDNAKNSLYLQMNSLRAEDTAVYYC
ARGIDYFDYWGQGTLVTVSSSGGGGSGGGGSGGGGSDIQLTQSPSFL
SASVGDRVTITCRASQGISSNLAWYQQKPGKDPKVLIYGASTLQSGV
PSRFSGSGSGTEFTLTISSLQPEDFATYYCQQLNSYPRTFGQGTKVE
IK.
6.
SX13-8.1 scFv
An exemplary SX13-8 scFv has the amino acid
sequence:
(SEQ ID NO: 24)
VQLQQSGPGLVKPSQTLSLTCAISGDSVSSNRAAWNWIRQPPSRGLE
WLGRTYYRSNWYNDYAVSVKSRITIKPDTSKNQFSLQLNSVTPEDTA
VYYCARDRGTYYYYYYMDVWDKGTTVTVSSSGGGGSGGGGSGGGGSE
IVLTQSPGTLSLSPGERATLSCRASQSVSSSYLVWYQQKAGQAPRIL
IYGASNRATDIPVRFSGSGSGTDFTLTISRLEPEDFGVYFCQHYGGS
YTFGQGTKLEIK.
7.
SX13-10 scFv
An exemplary SX13-10 scFv has the amino acid
sequence:
(SEQ ID NO: 25)
QLLESGGGLVKPGGSLRLSCAASGFTFSRYNMNWVRQAPGKGLEWVS
FISGSSSYVYYADSVKGRFTISKDNAKNSLYLQMNSLRAEDTAVYYC
ARSGYCTSTTCYNYYYYYMDVWGKGTTVTVSSSGGGGSGGGGSGGGG
SEIVLMQSPGTLSLSPGERATLSCRASQSVSNSYLAWYQQKPGQAPR
LLIYGASSRATGIPDRFSGSGSGTDFTLTISRLEPEDFAVFYCQQFG
SSPLTFGGGTKVEIK.
8.
SX13-12 scFv
An exemplary SX13-12 scFv has the amino acid
sequence:
(SEQ ID NO: 26)
QLVESGGGLVKPGGSLRLSCAASGFTFSDYSMNWVRQAPGKGLNWVA
SISSSSHYKYYADSVKGRFTISRDNAKNSLYLQMNSLRAEDTAVYYC
ARGIDFFDIWGQGTMVTVSSSGGGGSGGGGSGGGGSDIQLTQSPSFL
SASVGDRVTITCRASQDINSYLVWYQQKPGKAPKLLIYTASTLQSGV
PSRFSGSGSGTEFTLTIRSLQPEDFATYYCQQLNSYPRTFGQGTKVE
IK.

### D. Functional Forms of Anti-ENPP3 Antibodies

The disclosure particularly contemplates the production and use of “derivatives” of any of the above-described antibodies and their antigen-binding fragments. The term “derivative” refers to an antibody or antigen-binding fragment thereof that immunospecifically binds to an antigen but which incudes, one, two, three, four, five or more amino acid substitutions, additions, deletions or modifications relative to a “parental” (or wild-type) molecule. Such amino acid substitutions or additions may introduce naturally occurring (i.e., DNA-encoded) or non-naturally occurring amino acid residues. Such amino acids may be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment the altered carbohydrate modifications enhance antibody mediated effector function relative to the antibody lacking the carbohydrate modification. Carbohydrate modifications that lead to altered antibody mediated effector function are well known in the art (for example, see Shields, R. L. et al. (2002) “Lack Of Fucose On Human IgG N-Linked Oligosaccharide Improves Binding To Human Fc gamma RIII And Antibody-Dependent Cellular Toxicity.,” J. Biol. Chem. 277(30): 26733-26740; Davies J. et al. (2001) “Expression Of GnTIII In A Recombinant Anti-CD20 CHO Production Cell Line: Expression Of Antibodies With Altered Glycoforms Leads To An Increase In ADCC Through Higher Affinity For FC Gamma RIII,” Biotechnology & Bioengineering 74(4): 288-294). Methods of altering carbohydrate contents are known to those skilled in the art, see, e.g., Wallick, S. C. et al. (1988) “Glycosylation Of A VH Residue Of A Monoclonal Antibody Against Alpha (1----6) Dextran Increases Its Affinity For Antigen,” J. Exp. Med. 168(3): 1099-1109; Tao, M. H. et al. (1989) “Studies Of Aglycosylated Chimeric Mouse-Human IgG. Role Of Carbohydrate In The Structure And Effector Functions Mediated By The Human IgG Constant Region,” J. Immunol. 143(8): 2595-2601; Routledge, E. G. et al. (1995) “The Effect Of Aglycosylation On The Immunogenicity Of A Humanized Therapeutic CD3 Monoclonal Antibody,” Transplantation 60(8):847-53; Elliott, S. et al. (2003) “Enhancement Of Therapeutic Protein In Vivo Activities Through Glycoengineering,” Nature Biotechnol. 21:414-21; Shields, R. L. et al. (2002) “Lack Of Fucose On Human IgG N-Linked Oligosaccharide Improves Binding To Human Fcgamma RIII And Antibody-Dependent Cellular Toxicity.,” J. Biol. Chem. 277(30): 26733-26740).

In some embodiments, a humanized antibody is a derivative. Such a humanized antibody can include amino acid residue substitutions, deletions or additions in one or more non-human CDRs. The humanized antibody derivative may have substantially the same binding, better binding, or worse binding when compared to a non-derivative humanized antibody. In specific embodiments, one, two, three, four, or five amino acid residues of the CDR have been substituted, deleted or added (i.e., mutated).

A derivative antibody or antibody fragment can be modified by chemical modifications using techniques known to those of skill in the art, including, but not limited to, specific chemical cleavage, acetylation, formulation, metabolic synthesis of tunicamycin, etc. In one embodiment, an antibody derivative possess a similar or identical function as the parental antibody. In another embodiment, an antibody derivative exhibits an altered activity relative to the parental antibody. For example, a derivative antibody (or fragment thereof) can bind to its epitope more tightly or be more resistant to proteolysis than the parental antibody.

Substitutions, additions or deletions in the derivatized antibodies may be in the Fc region of the antibody and may thereby serve to modify the binding affinity of the antibody to one or more FcγR. Methods for modifying antibodies with modified binding to one or more FcγR are known in the art, see, e.g., PCT Publication Nos. WO 04/029207, WO 04/029092, WO 04/028564, WO 99/58572, WO 99/51642, WO 98/23289, WO 89/07142, WO 88/07089, and U.S. Pat. Nos. 5,843,597 and 5,642,821. In some embodiments, antibodies are provided that have altered affinity for an activating FcγR, e.g., FcγRIIIA. Preferably such modifications also have an altered Fc-mediated effector function. Modifications that affect Fc-mediated effector function are well known in the art (see U.S. Pat. No. 6,194,551, and WO 00/42072). In one particular embodiment, the modification of the Fc region results in an antibody with an altered antibody-mediated effector function, an altered binding to other Fc receptors (e.g., Fc activation receptors), an altered antibody-dependent cell-mediated cytotoxicity (ADCC) activity, an altered C1q binding activity, an altered complement-dependent cytotoxicity activity (CDC), a phagocytic activity, or any combination thereof.

Derivatized antibodies can be used to alter the half-lives (e.g., serum half-lives) of parental antibodies in a mammal, preferably a human. Preferably such alteration will result in a half-life of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the humanized antibodies of the disclosed antibodies or fragments thereof in a mammal, preferably a human, results in a higher serum titer of the antibodies or antibody fragments in the mammal, and thus, reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor. The antibodies of can be engineered to increase biological half-lives (see, e.g. U.S. Pat. No. 6,277,375). For example, antibodies can be engineered in the Fc-hinge domain to have increased in vivo or serum half-lives.

Antibodies or fragments thereof with increased in vivo half-lives can be generated by attaching to the antibodies or antibody fragments polymer molecules such as high molecular weight polyethylene glycol (PEG). PEG can be attached to said antibodies or antibody fragments with or without a multifunctional linker either through site-specific conjugation of the PEG to the N- or C-terminus of said antibodies or antibody fragments or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography.

The antibodies can also be modified by the methods and coupling agents described by Davis et al. (See U.S. Pat. No. 4,179,337) in order to provide compositions that can be injected into the mammalian circulatory system with substantially no immunogenic response.

The disclosed antibodies encompasses modification of framework residues of the humanized antibodies. Framework residues in the framework regions may be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., U.S. Pat. No. 5,585,089; and Riechmann, L. et al. (1988) “Reshaping Human Antibodies For Therapy,” Nature 332:323-327).

The Fc portion of an antibody be varied by isotype or subclass, can be a chimeric or hybrid, and/or can be modified, for example to improve effector functions, control of half-life, tissue accessibility, augment biophysical characteristics such as stability, and improve efficiency of production (and less costly). Many modifications useful in construction of disclosed proteins and methods for making them are known in the art, see for example Mueller, J. P. et al. (1997) “Humanized Porcine VCAM-Specific Monoclonal Antibodies With Chimeric Igg2/G4 Constant Regions Block Human Leukocyte Binding To Porcine Endothelial Cells,” Mol. Immun. 34(6):441-452, Swann, P. G. (2008) “Considerations For The Development Of Therapeutic Monoclonal Antibodies,” Curr. Opin. Immun. 20:493-499 (2008), and Presta, L. G. (2008) “Molecular Engineering And Design Of Therapeutic Antibodies,” Curr. Opin. Immun. 20:460-470. In some embodiments the Fc region is the native IgG1, IgG2, or IgG4 Fc region. In some embodiments the Fc region is a hybrid, for example a chimeric consisting of IgG2/IgG4 Fc constant regions. Modifications to the Fc region include, but are not limited to, IgG4 modified to prevent binding to Fc gamma receptors and complement, IgG1 modified to improve binding to one or more Fc gamma receptors, IgG1 modified to minimize effector function (amino acid changes), IgG1 with altered/no glycan (typically by changing expression host), and IgG1 with altered pH-dependent binding to FcRn. The Fc region can include the entire hinge region, or less than the entire hinge region.

The therapeutic outcome in patients treated with rituximab (a chimeric mouse/human IgG1 monoclonal antibody against CD20) for non-Hodgkin's lymphoma or Waldenstrom's macroglobulinemia correlated with the individual's expression of allelic variants of Fcγ receptors with distinct intrinsic affinities for the Fc domain of human IgG1. In particular, patients with high affinity alleles of the low affinity activating Fc receptor CD16A (FcγRIIIA) showed higher response rates and, in the cases of non-Hodgkin's lymphoma, improved progression-free survival. In another embodiment, the Fc domain may contain one or more amino acid insertions, deletions or substitutions that reduce binding to the low affinity inhibitory Fc receptor CD32B (FcγRIIB) and retain wild-type levels of binding to or enhance binding to the low affinity activating Fc receptor CD16A (FcγRIIIA).

Another embodiment includes IgG2-4 hybrids and IgG4 mutants that have reduce binding to FcR which increase their half-life. Representative IG2-4 hybrids and IgG4 mutants are described in Angal, S. et al. (1993) “A Single Amino Acid Substitution Abolishes The Heterogeneity Of Chimeric Mouse/Human (IgG4) Antibody,” Molec. Immunol. 30(1):105-108; Mueller, J. P. et al. (1997) “Humanized Porcine VCAM-Specific Monoclonal Antibodies With Chimeric IgG2/G4 Constant Regions Block Human Leukocyte Binding To Porcine Endothelial Cells,” Mol. Immun. 34(6):441-452; and U.S. Pat. No. 6,982,323. In some embodiments the IgG1 and/or IgG2 domain is deleted for example, Angal et al. describe IgG1 and IgG2 having serine 241 replaced with a proline

In a preferred embodiment, the Fc domain contains amino acid insertions, deletions or substitutions that enhance binding to CD16A. A large number of substitutions in the Fc domain of human IgG1 that increase binding to CD16A and reduce binding to CD32B are known in the art and are described in Stavenhagen, J. B. et al. (2007) “Fc Optimization Of Therapeutic Antibodies Enhances Their Ability To Kill Tumor Cells In Vitro And Controls Tumor Expansion In Vivo Via Low-Affinity Activating Fcgamma Receptors,” Cancer Res. 57(18):8882-8890. Exemplary variants of human IgG1 Fc domains with reduced binding to CD32B and/or increased binding to CD16A contain F243L, R929P, Y300L, V305I or P296L substitutions. These amino acid substitutions can be present in a human IgG1 Fc domain in any combination. In one embodiment, the human IgG1 Fc domain variant contains a F243L, R929P and Y300L substitution. In another embodiment, the human IgG1 Fc domain variant contains a F243L, R929P, Y300L, V305I and P296L substitution. In another embodiment, the human IgG1 Fc domain variant contains an N297Q substitution, as this mutation abolishes FcR binding.

Also provided are antibodies with a heterologous molecule fused, conjugated, or otherwise attached thereto. In some forms, such antibodies are referred to as antibody-drug conjugates (ADCs). In some embodiments, heterologous molecules are polypeptides having at least 10, at least 20, at least 30, at least 40, at least 50, at least 60, at least 70, at least 80, at least 90 or at least 100 amino acids. Such heterologous molecules may alternatively be enzymes, hormones, cell surface receptors, drug moieties, such as: toxins (such as abrin, ricin A, pseudomonas exotoxin (i.e., PE-40), diphtheria toxin, ricin, gelonin, or pokeweed antiviral protein), proteins (such as tumor necrosis factor, interferon (e.g., α-interferon, β-interferon), nerve growth factor, platelet derived growth factor, tissue plasminogen activator, or an apoptotic agent (e.g., tumor necrosis factor-α, tumor necrosis factor-β)), biological response modifiers (such as, for example, a lymphokine (e.g., interleukin-1 (“IL-1”), interleukin-2 (“IL-2”), interleukin-6 (“IL-6”)), granulocyte macrophage colony stimulating factor (“GM-CSF”), granulocyte colony stimulating factor (“G-CSF”), or macrophage colony stimulating factor, (“M-CSF”)), or growth factors (e.g., growth hormone (“GH”))), cytotoxins (e.g., a cytostatic or cytocidal agent, such as paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, monomethyl auristatin F (MMAF), monomethyl auristatin E (MMAE; e.g., vedotin) and puromycin and analogs or homologs thereof), antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, BiCNU® (carmustine; BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), or anti-mitotic agents (e.g., vincristine and vinblastine).

Techniques for conjugating such therapeutic moieties to antibodies are well known; see, e.g., Arnon et al., “Monoclonal Antibodies For Immunotargeting Of Drugs In Cancer Therapy”, in MONOCLONAL ANTIBODIES AND CANCER THERAPY, Reisfeld et al. (eds.), 1985, pp. 243-56, Alan R. Liss, Inc.); Hellstrom et al., “Antibodies For Drug Delivery”, in CONTROLLED DRUG DELIVERY (2nd Ed.), Robinson et al. (eds.), 1987, pp. 623-53, Marcel Dekker, Inc.); Thorpe, “Antibody Carriers Of Cytotoxic Agents In Cancer Therapy: A Review”, in MONOCLONAL ANTIBODIES '84: BIOLOGICAL AND CLINICAL APPLICATIONS, Pinchera et al. (eds.), 1985, pp. 475-506); “Analysis, Results, And Future Prospective Of The Therapeutic Use Of Radiolabeled Antibody In Cancer Therapy”, in MONOCLONAL ANTIBODIES FOR CANCER DETECTION AND THERAPY, Baldwin et al. (eds.), 1985, pp. 303-16, Academic Press; Thorpe et al. (1982) “The Preparation And Cytotoxic Properties Of Antibody-Toxin Conjugates,” Immunol. Rev. 62:119-158; Carter, P. J. et al. (2008) “Antibody-Drug Conjugates for Cancer Therapy,” Cancer J. 14(3):154-169; Alley, S. C. et al. (2010) “Antibody-Drug Conjugates: Targeted Drug Delivery For Cancer,” Curr. Opin. Chem. Biol. 14(4):529-537; Carter, P. et al. (2005) “Designer Antibody-Based Therapeutics For Oncology,” Amer. Assoc. Cancer Res. Educ. Book. 2005(1):147-154; Carter, P. J. et al. (2008) “Antibody-Drug Conjugates For Cancer Therapy,” Cancer J. 14(3):154-169; Chari, R. V. J. (2008) “Targeted Cancer Therapy: Conferring Specificity To Cytotoxic Drugs,” Acc. Chem Res. 41(1):98-107; Doronina, S. O. et al. (2003) “Development Of Potent Monoclonal Antibody Auristatin Conjugates For Cancer Therapy,” Nat. Biotechnol. 21(7):778-784; Ducry, L. et al. (2010) “Antibody-Drug Conjugates: Linking Cytotoxic Payloads To Monoclonal Antibodies,” Bioconjug Chem. 21(1):5-13; Senter, P. D. (2009) “Potent Antibody Drug Conjugates For Cancer Therapy,” Curr. Opin. Chem. Biol. 13(3):235-244; and Teicher, B. A. (2009) “Antibody-Drug Conjugate Targets,” Curr Cancer Drug Targets. 9(8):982-1004.

Any of the molecules can be fused to marker sequences, such as a peptide, to facilitate purification. In some embodiments, the marker amino acid sequence is a hexa-histidine peptide, the hemagglutinin “HA” tag, which corresponds to an epitope derived from the influenza hemagglutinin protein (Wilson, I. A. et al. (1984) “The Structure Of An Antigenic Determinant In A Protein,” Cell, 37:767-778) and the “flag” tag (Knappik, A. et al. (1994) “An Improved Affinity Tag Based On The FLAG Peptide For The Detection And Purification Of Recombinant Antibody Fragments,” Biotechniques 17(4):754-761).

Also encompassed are antibodies or their antigen-binding fragments that are conjugated to a diagnostic or therapeutic agent or any other molecule for which serum half-life is desired to be increased. The antibodies can be used diagnostically (in vivo, in situ or in vitro) to, for example, monitor the development or progression of a disease, disorder or infection as part of a clinical testing procedure to, e.g., determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive materials, positron emitting metals, and nonradioactive paramagnetic metal ions. The detectable substance may be coupled or conjugated either directly to the antibody or indirectly, through an intermediate (such as, for example, a linker known in the art) using techniques known in the art. See, for example, U.S. Pat. No. 4,741,900 for metal ions which can be conjugated to antibodies for use as diagnostics according to the present invention. Such diagnosis and detection can be accomplished by coupling the antibody to detectable substances including, but not limited to, various enzymes, enzymes including, but not limited to, horseradish peroxidase, alkaline phosphatase, beta-galactosidase, or acetylcholinesterase; prosthetic group complexes such as, but not limited to, streptavidin/biotin and avidin/biotin; fluorescent materials such as, but not limited to, umbelliferone, fluorescein, fluorescein isothiocyanate, rhodamine, dichlorotriazinylamine fluorescein, dansyl chloride or phycoerythrin; luminescent material such as, but not limited to, luminol; bioluminescent materials such as, but not limited to, luciferase, luciferin, and aequorin; radioactive material such as, but not limited to, bismuth (213Bi), carbon (14C), chromium (51Cr), cobalt (57Co), fluorine (18F), gadolinium (153Gd, 159Gd), gallium (68Ga, 67Ga), germanium (68Ge), holmium (166Ho), indium (115In, 113In, 112In, 111In), iodine (131I, 125I, 123I, 121I), lanthanium (140La), lutetium (177Lu), manganese (54Mn), molybdenum (99Mo), palladium (103Pd), phosphorous (32P), praseodymium (142Pr), promethium (149Pm), rhenium (186Re, 188Re), rhodium (105Rh), ruthemium (97Ru), samarium (153Sm), scandium (47Sc), selenium (75Se), strontium (85Sr), sulfur (35S), technetium (99Tc), thallium (201Ti), tin (113Sn, 117Sn), tritium (3H), xenon (133Xe), ytterbium (169Yb, 175Yb), yttrium (90Y), zinc (65Zn); positron emitting metals using various positron emission tomographies, and nonradioactive paramagnetic metal ions.

The molecules of the disclose can be conjugated to a second antibody to form an antibody heteroconjugate as described by Segal in U.S. Pat. No. 4,676,980. Such heteroconjugate antibodies may additionally bind to haptens (such as fluorescein, etc.), or to cellular markers (e.g., 4-1-BB, B7-H4, CD4, CD8, CD14, CD25, CD27, CD40, CD68, CD163, CTLA4, GITR, LAG-3, OX40, TIM3, TIM4, TLR2, LIGHT, ICOS, B7-H3, B7-H7, B7-H7CR, CD70, CD47, etc.) or to cytokines (e.g., IL-7, IL-15, IL-12, IL-4 TGF-beta, IL-10, IL-17, IFNγ, Flt3, BLys) or chemokines (e.g., CCL21), etc.

Bispecific and multispecific antibodies that bind to ENPP3 and e.g., a second cancer antigen or immune cell antigen are provided. For example, exemplary second cancer antigens specific for cancer that could be targeted by the bispecific/multispecific antibody bispecific or multispecific format along with ENPP3, include, but are not limited to, 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgGl, LI-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, vimentin, and combinations thereof.

Exemplary immune antigens include, but are not limited to, CD3, CD4, CD8, CD2, CD5, CD7, CD28, 41BB, CD16, NKp46/CD335, CD11b, CD11c, etc.

In some embodiments, the bispecific/multispecific antibodies targeting ENPP3 and at least one immune-related molecule can be referred to as a T cell engager (BITE) or NK cell engager or macrophage engager. See, e.g., Tian, et al., “Bispecific T cell engagers: an emerging therapy for management of hematologic malignancies,” J Hematol Oncol. 2021 May 3; 14(1):75. doi: 10.1186/s13045-021-01084-4. PMID: 33941237; PMCID: PMC8091790, Zhou, et al. “The landscape of bispecific T cell engager in cancer treatment,” Biomark Res 9, 38 (2021).

The molecules of the disclosure can be attached to solid supports, which are particularly useful for immunoassays or purification of the target antigen or of other molecules that are capable of binding to target antigen that has been immobilized to the support via binding to an antibody or antigen-binding fragment of the present invention. Such solid supports include, but are not limited to, glass, cellulose, polyacrylamide, nylon, polystyrene, polyvinyl chloride or polypropylene.

As discussed elsewhere in more detail, also provided are nucleic acid molecules (DNA or RNA) that encode any such antibodies, fusion proteins or fragments, as well as vector molecules (such as plasmids) that are capable of transmitting or of replication such nucleic acid molecules. The nucleic acids can be single-stranded, double-stranded, may contain both single-stranded and double-stranded portions.

### E. Chimeric Antigen Receptor (CARs) Including Anti-ENPP3

Chimeric antigen receptors (CARs) including the anti-ENPP3 binding polypeptides are also provided.

Immunotherapy using T cells recombinant nucleic acid and genetic engineering techniques to express chimeric antigen receptors (CAR) are rapidly emerging as a promising new treatment for hematological and non-hematological malignancies. CARs are engineered receptors that possess both antigen-binding and T-cell-activating functions. Based on the location of the CAR in the membrane of the cell, the CAR can be divided into three main distinct domains, including an extracellular antigen binding domain, followed by a space region, a transmembrane domain, and an intracellular signaling domain.

### 1. Extracellular Domains

Typically, the described ENPP-3 binding CARs include an extracellular domain that includes the ENPP-3 binding domain.

The ENPP-3 binding function of the described CAR is implemented within the intracellular components of the CAR molecule. Therefore, an exemplary CAR includes an extracellular (e.g., antigen binding) domain, conjugated directly or indirectly to a trans-membrane domain. In some forms, an ENPP-3 binding CAR includes an extracellular domain including an ENPP-3 binding domain and optionally one or more additional extracellular domains conjugated to the trans-membrane domain, such as a “spacer” domain. An exemplary spacer domain and one or more intracellular domains. In some forms, the extracellular domain of the described CARs includes at least one extracellular component of a mature human TCR co-receptor protein, such as an extracellular polypeptide derived from the mature human CD28 protein, or an extracellular polypeptide derived from the mature human CD8 protein.

i. Antigen-Binding Domain

The extracellular component of the described CARs includes an antigen binding domain, such as an ENPP-3 binding domain. Typically the ENPP-3 binding domain is composed of two immunoglobulin domains.

The antigen binding domain, most commonly derived from variable regions of immunoglobulins, typically contains V_{H} and V_{L} immunoglobulin domains (also referred to as “chains”) that are joined up by a linker to form the so-called “scFv.”

In some forms, the antigen binding domain is derived from an antibody. As discussed in more detail elsewhere in herein, the term antibody herein refers to natural or synthetic polypeptides that bind a target antigen. The term includes polyclonal and monoclonal antibodies, including intact antibodies and functional (e.g., antigen-binding) antibody fragments, including Fab fragments, F(ab′)₂ fragments, Fab′ fragments, Fv fragments, recombinant IgG (rlgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. The term also encompasses intact or full-length antibodies, including antibodies of any class or subclass, including IgG and sub-classes thereof, IgM, IgE, IgA, and IgD. The antigen binding domain of a CAR can contain complementary determining regions (CDR) of an antibody, variable regions of an antibody, and/or antigen binding fragments thereof. For example, the antigen binding domain for a CD19 CAR can be derived from a human monoclonal antibody to CD19, such as those described in U.S. Pat. No. 7,109,304. Likewise, the disclosed CAR can include an antigen binding domain from a anti-ENPP3 binding polypeptide provided herein. In some forms, the antigen binding domain can include an F(ab′)2, Fab′, Fab, Fv or scFv.

In some forms, the CAR is a bispecific CAR, i.e., that selectively binds to more than a single antigen. In some forms, the CAR can be multivalent. Bispecific or multi-specific (multivalent) CARs, e.g., including, but not limited to, CARs described in WO 2014/4011988 and US20150038684, are contemplated for use in the disclosed methods and compositions.

Exemplary antigens specific for cancer that could be targeted by the CAR in a bispecific or multi-specific format along with ENPP3 are mentioned above.

In some forms, the CAR includes an ENPP-3 binding domain fused directly to a spacer or transmembrane domain. An exemplary ENPP-3 binding domain includes a V_{H} domain including an amino acid sequence of between about 100 and about 130 residues, inclusive, derived from the V_{H} domain of any one of the described ENPP-3 binding antibodies.

In some forms, a CAR includes the CDRs of a V_{H} domain and/or V_{L} domain as set forth in any one of the described ENNP-3 binding antibodies.

a. Antibody SX13-1 Antigen-Binding Domain

In some forms, a CAR includes all or part of the V_{H} domain and/or V_{L} domain of the SX13-1 antibody. For example in some forms, a CAR includes all or part of the SX13-1 antibody V_{H} domain. In some forms, the CAR includes all or part of the amino acid sequence of SEQ ID NO:70. For example, in some forms, a CAR includes the CDRs of the V_{H} domain of SEQ ID NO:70. In some forms, a CAR includes one or more of the V_{H} CDRs 1-3 including the amino acid sequences set forth in SEQ ID NOs: 29, 30, and/or 31.

In some forms, a CAR includes all or part of the SX13-1 antibody V_{L} domain including all or part of the amino acid sequence of SEQ ID NO:71. For example, in some forms, a CAR includes the CDRs of the V_{L} domain of SEQ ID NO:71. In some forms, a CAR includes one or more of the V_{L} CDRs 1-3 including the amino acid sequences set forth in SEQ ID NOs: 32, 33, and/or 34.

In Exemplary forms, a CAR includes all the CDRs of the SX13-1 antibody. For example, in some forms, a CAR includes all of the V_{H} CDRs 1-3 including the amino acid sequences set forth in SEQ ID NOs: 29, 30, and 31, and all of the V_{L} CDRs 1-3 including the amino acid sequences set forth in SEQ ID NOs: 32, 33, and 34.

In some forms, a CAR includes a single chain FV (ScFV) of the SX13-1 antibody. For example, in some forms, a CAR includes an amino acid sequence as set forth in SEQ ID NO:19, or a functional variant thereof having at least 80% sequence identity with SEQ ID NO:19.

b. Antibody SX13-2 Antigen-Binding Domain

In some forms, a CAR includes all or part of the V_{H} domain and/or V_{L} domain of the SX13-2 antibody. For example in some forms, a CAR includes all or part of the SX13-2 antibody V_{H} domain. In some forms, the CAR includes all or part of the amino acid sequence of SEQ ID NO:72. For example, in some forms, a CAR includes the CDRs of the V_{H} domain of SEQ ID NO:72. In some forms, a CAR includes one or more of the V_{H} CDRs 1-3 including the amino acid sequences set forth in SEQ ID NOs: 35, 36, and/or 37.

In some forms, a CAR includes all or part of the SX13-2 antibody V_{L} domain including all or part of the amino acid sequence of SEQ ID NO:73. For example, in some forms, a CAR includes the CDRs of the V_{L} domain of SEQ ID NO:73. In some forms, a CAR includes one or more of the V_{L} CDRs 1-3 including the amino acid sequences set forth in SEQ ID NOs: 38, 39, and/or 40.

In Exemplary forms, a CAR includes all the CDRs of the SX13-2 antibody. For example, in some forms, a CAR includes all of the V_{H} CDRs 1-3 including the amino acid sequences set forth in SEQ ID NOs: 35, 36, and 37, and all of the V_{L} CDRs 1-3 including the amino acid sequences set forth in SEQ ID NOs: 38, 39, and 40.

In some forms, a CAR includes a single chain FV (ScFV) of the SX13-2 antibody. For example, in some forms, a CAR includes an amino acid sequence as set forth in SEQ ID NO:20, or a functional variant thereof having at least 80% sequence identity with SEQ ID NO:20.

c. Antibody SX13-5 Antigen-Binding Domain

In some forms, a CAR includes all or part of the V_{H} domain and/or V_{L} domain of the SX13-5 antibody. For example in some forms, a CAR includes all or part of the SX13-5 antibody V_{H} domain. In some forms, the CAR includes all or part of the amino acid sequence of SEQ ID NO:74. For example, in some forms, a CAR includes the CDRs of the V_{H} domain of SEQ ID NO:74. In some forms, a CAR includes one or more of the V_{H} CDRs 1-3 including the amino acid sequences set forth in SEQ ID NOs: 41, 42, and/or 43.

In some forms, a CAR includes all or part of the SX13-5 antibody V_{L} domain including all or part of the amino acid sequence of SEQ ID NO:75. For example, in some forms, a CAR includes the CDRs of the V_{L} domain of SEQ ID NO:75. In some forms, a CAR includes one or more of the V_{L} CDRs 1-3 including the amino acid sequences set forth in SEQ ID NOs: 44, 45, and/or 46.

In Exemplary forms, a CAR includes all the CDRs of the SX13-5 antibody. For example, in some forms, a CAR includes all of the V_{H} CDRs 1-3 including the amino acid sequences set forth in SEQ ID NOs: 41, 42, and 43, and all of the V_{L} CDRs 1-3 including the amino acid sequences set forth in SEQ ID NOs: 44, 45, and 46.

In some forms, a CAR includes a single chain FV (ScFV) of the SX13-5 antibody. For example, in some forms, a CAR includes an amino acid sequence as set forth in SEQ ID NO:21, or a functional variant thereof having at least 80% sequence identity with SEQ ID NO:21.

d. Antibody SX13-6 Antigen-Binding Domain

In some forms, a CAR includes all or part of the V_{H} domain and/or V_{L} domain of the SX13-6 antibody. For example in some forms, a CAR includes all or part of the SX13-6 antibody V_{H} domain. In some forms, the CAR includes all or part of the amino acid sequence of SEQ ID NO:76. For example, in some forms, a CAR includes the CDRs of the V_{H} domain of SEQ ID NO:76. In some forms, a CAR includes one or more of the V_{H} CDRs 1-3 including the amino acid sequences set forth in SEQ ID NOs: 46, 47, and/or 48.

In some forms, a CAR includes all or part of the SX13-6 antibody V_{L} domain including all or part of the amino acid sequence of SEQ ID NO:77. For example, in some forms, a CAR includes the CDRs of the V_{L} domain of SEQ ID NO:77. In some forms, a CAR includes one or more of the V_{L} CDRs 1-3 including the amino acid sequences set forth in SEQ ID NOs: 49, 50, and/or 51.

In Exemplary forms, a CAR includes all the CDRs of the SX13-6 antibody. For example, in some forms, a CAR includes all of the V_{H} CDRs 1-3 including the amino acid sequences set forth in SEQ ID NOs: 46, 47, and 48, and all of the V_{L} CDRs 1-3 including the amino acid sequences set forth in SEQ ID NOs: 49, 50, and 51. In some forms, a CAR includes a single chain FV (ScFV) of the SX13-6 antibody. For example, in some forms, a CAR includes an amino acid sequence as set forth in SEQ ID NO:22, or a functional variant thereof having at least 80% sequence identity with SEQ ID NO:22.

e. Antibody SX13-7 Antigen-Binding Domain

In some forms, a CAR includes all or part of the V_{H} domain and/or V_{L} domain of the SX13-7 antibody. For example in some forms, a CAR includes all or part of the SX13-7 antibody V_{H} domain. In some forms, the CAR includes all or part of the amino acid sequence of SEQ ID NO:78. For example, in some forms, a CAR includes the CDRs of the V_{H} domain of SEQ ID NO:79. In some forms, a CAR includes one or more of the V_{H} CDRs 1-3 including the amino acid sequences set forth in SEQ ID NOs: 46, 36, and/or 31.

In some forms, a CAR includes all or part of the SX13-7 antibody V_{L} domain including all or part of the amino acid sequence of SEQ ID NO:79. For example, in some forms, a CAR includes the CDRs of the V_{L} domain of SEQ ID NO:79. In some forms, a CAR includes one or more of the V_{L} CDRs 1-3 including the amino acid sequences set forth in SEQ ID NOs: 52, 53, and/or 34.

In Exemplary forms, a CAR includes all the CDRs of the SX13-7 antibody. For example, in some forms, a CAR includes all of the V_{H} CDRs 1-3 including the amino acid sequences set forth in SEQ ID NOs: 46, 36, and 31, and all of the V_{L} CDRs 1-3 including the amino acid sequences set forth in SEQ ID NOs: 52, 53, and 34.

In some forms, a CAR includes a single chain FV (ScFV) of the SX13-7 antibody. For example, in some forms, a CAR includes an amino acid sequence as set forth in SEQ ID NO:23, or a functional variant thereof having at least 80% sequence identity with SEQ ID NO:23.

f. Antibody SX13-8 Antigen-Binding Domain

In some forms, a CAR includes all or part of the V_{H} domain and/or V_{L} domain of the SX13-8 antibody. For example in some forms, a CAR includes all or part of the SX13-8 antibody V_{H} domain. In some forms, the CAR includes all or part of the amino acid sequence of SEQ ID NO:80. For example, in some forms, a CAR includes the CDRs of the V_{H} domain of SEQ ID NO:80. In some forms, a CAR includes one or more of the V_{H} CDRs 1-3 including the amino acid sequences set forth in SEQ ID NOs: 54, 55, and/or 56.

In some forms, a CAR includes all or part of the SX13-8 antibody V_{L} domain including all or part of the amino acid sequence of SEQ ID NO:81. For example, in some forms, a CAR includes the CDRs of the V_{L} domain of SEQ ID NO:81. In some forms, a CAR includes one or more of the V_{L} CDRs 1-3 including the amino acid sequences set forth in SEQ ID NOs: 57, 58, and/or 59.

In Exemplary forms, a CAR includes all the CDRs of the SX13-8 antibody. For example, in some forms, a CAR includes all of the V_{H} CDRs 1-3 including the amino acid sequences set forth in SEQ ID NOs: 54, 55, and 56, and all of the V_{L} CDRs 1-3 including the amino acid sequences set forth in SEQ ID NOs: 57, 58, and 59.

In some forms, a CAR includes a single chain FV (ScFV) of the SX13-8 antibody. For example, in some forms, a CAR includes an amino acid sequence as set forth in SEQ ID NO:24, or a functional variant thereof having at least 80% sequence identity with SEQ ID NO:24.

g. Antibody SX13-10 Antigen-Binding Domain

In some forms, a CAR includes all or part of the V_{H} domain and/or V_{L} domain of the SX13-10 antibody. For example in some forms, a CAR includes all or part of the SX13-10 antibody V_{H} domain. In some forms, the CAR includes all or part of the amino acid sequence of SEQ ID NO:82. For example, in some forms, a CAR includes the CDRs of the V_{H} domain of SEQ ID NO:82. In some forms, a CAR includes one or more of the V_{H} CDRs 1-3 including the amino acid sequences set forth in SEQ ID NOs: 60, 61, and/or 62.

In some forms, a CAR includes all or part of the SX13-10 antibody V_{L} domain including all or part of the amino acid sequence of SEQ ID NO:83. For example, in some forms, a CAR includes the CDRs of the V_{L} domain of SEQ ID NO:83. In some forms, a CAR includes one or more of the V_{L} CDRs 1-3 including the amino acid sequences set forth in SEQ ID NOs: 63, 64, and/or 65.

In Exemplary forms, a CAR includes all the CDRs of the SX13-10 antibody. For example, in some forms, a CAR includes all of the V_{H} CDRs 1-3 including the amino acid sequences set forth in SEQ ID NOs: 60, 61, and 62, and all of the V_{L} CDRs 1-3 including the amino acid sequences set forth in SEQ ID NOs: 63, 64, and 65.

In some forms, a CAR includes a single chain FV (ScFV) of the SX13-10 antibody. For example, in some forms, a CAR includes an amino acid sequence as set forth in SEQ ID NO:25, or a functional variant thereof having at least 80% sequence identity with SEQ ID NO:25.

h. Antibody SX13-12 Antigen-Binding Domain

In some forms, a CAR includes all or part of the V_{H} domain and/or V_{L} domain of the SX13-12 antibody. For example in some forms, a CAR includes all or part of the SX13-12 antibody V_{H} domain. In some forms, the CAR includes all or part of the amino acid sequence of SEQ ID NO:84. For example, in some forms, a CAR includes the CDRs of the V_{H} domain of SEQ ID NO:84. In some forms, a CAR includes one or more of the V_{H} CDRs 1-3 including the amino acid sequences set forth in SEQ ID NOs: 46, 66, and/or 67.

In some forms, a CAR includes all or part of the SX13-12 antibody V_{L} domain including all or part of the amino acid sequence of SEQ ID NO:85. For example, in some forms, a CAR includes the CDRs of the V_{L} domain of SEQ ID NO:85. In some forms, a CAR includes one or more of the V_{L} CDRs 1-3 including the amino acid sequences set forth in SEQ ID NOs: 68, 69, and/or 34.

In Exemplary forms, a CAR includes all the CDRs of the SX13-12 antibody. For example, in some forms, a CAR includes all of the V_{H} CDRs 1-3 including the amino acid sequences set forth in SEQ ID NOs: 46, 66, and/or 67, and all of the V_{L} CDRs 1-3 including the amino acid sequences set forth in SEQ ID NOs: 68, 69, and 34.

In some forms, a CAR includes a single chain FV (ScFV) of the SX13-12 antibody. For example, in some forms, a CAR includes an amino acid sequence as set forth in SEQ ID NO:26, or a functional variant thereof having at least 80% sequence identity with SEQ ID NO:26.

ii. Spacer Domain

In some forms, the extracellular component of the described CARs includes a spacer domain.

The segment interposing between the antigen binding domain (e.g., scFv) and the transmembrane domain is a “spacer domain.” The spacer domain can include, for example, the constant IgG1 hinge-CH2-CH3 Fc domain. In some cases, the spacer domain and the transmembrane domain are derived from CD8.

In some forms, the CAR includes one or more spacer domain(s) (also referred to as hinge domain) that is located between the extracellular antigen binding domain and the transmembrane domain. A spacer domain is an amino acid segment that is generally found between two domains of a protein and may allow for flexibility of the protein and movement of one or both of the domains relative to one another. Any amino acid sequence that provides such flexibility and movement of the extracellular antigen binding domain relative to the transmembrane domain can be used. The spacer domain can be a spacer or hinge domain of a naturally occurring protein. In some forms, the hinge domain is derived from CD8a, such as, a portion of the hinge domain of CD8a, e.g., a fragment containing at least 5 (e.g., 5, 10, 15, 20, 25, 30, 35, or 40) consecutive amino acids of the hinge domain of CD8a. Hinge domains of antibodies, such as an IgG, IgA, IgM, IgE, or IgD antibodies can also be used. In some forms, the hinge domain is the hinge domain that joins the constant CH1 and CH2 domains of an antibody. Non-naturally occurring peptides may also be used as spacer domains. For example, the spacer domain can be a peptide linker, such as a (GxS)n linker, wherein x and n, independently can be an integer of 3 or more, including 3, 4, 5, 6, 7, 8, 9, 10, 11, 12, or more.

As described in the Examples, in some forms, the CAR includes an ENPP-3 binding domain fused directly to a “CD8 spacer domain”. An exemplary CD8 spacer domain includes an amino acid sequence of between about 30 and about 60 residues, inclusive, derived from the extracellular component of the mature human CD8 alpha protein or an amino acid sequence of between about 30 and about 60 residues, inclusive, derived from the extracellular component of the mature human CD8 beta protein. In some forms, the spacer domain includes an amino acid sequence of about 45 residues derived from the extracellular component of the mature human CD8 alpha protein.

An exemplary amino acid sequence for the transmembrane domain derived from the mature human CD8 alpha subunit includes:

(SEQ ID NO: 86)
TTTPAPRPPTPAPTIASQPLSLRPEACRPAAGGAVHTRGLDFACD.

As described in the Examples, in some forms, the CAR includes an ENPP-3 binding domain fused directly to a “CD28 spacer domain”. An exemplary CD28 spacer domain includes an amino acid sequence of between about 30 and about 60 residues, inclusive, derived from the extracellular component of the mature human CD28 protein. In some forms, the spacer domain includes an amino acid sequence of about 39 residues derived from the extracellular component of the mature human CD28 protein.

In other forms, the CAR includes a spacer domain derived from the “hinge” region of an immunoglobulin (Ig), such as a human Ig. An exemplary human Ig hinge includes the hinge of IgG4m. In some forms, the spacer domain includes an amino acid sequence of between about 10 and about 20 residues, inclusive, derived from the extracellular component of the mature human IgG4m protein. In some forms, the spacer domain includes an amino acid sequence of about 12 residues derived from the hinge region of the mature human IgG4m protein.

Typically, a spacer domain is fused directly to a trans-membrane domain of a CAR.

### 2. Trans-Membrane Domain

In some forms, the described CARs include a transmembrane domain.

In some forms the intracellular signaling domains mediating T cell activation can include a CD3ζ co-receptor signaling domain derived from C-region of the TCR a and B chains and one or more costimulatory domains. In some forms, the CAR includes a transmembrane domain that can be directly or indirectly fused to the antigen binding domain. The transmembrane domain may be derived either from a natural or a synthetic source. As used herein, a “transmembrane domain” refers to any protein structure that is thermodynamically stable in a cell membrane, preferably a eukaryotic cell membrane. In some forms, the transmembrane domain of the CAR includes a transmembrane domain of an alpha, beta or zeta chain of a T-cell receptor, CD8, CD4, CD28, CD137, CD80, CD86, CD152 or PD1, or a portion thereof. Transmembrane domains can also contain at least a portion of a synthetic, non-naturally occurring protein segment. In some forms, the transmembrane domain is a synthetic, non-naturally occurring alpha helix or beta sheet. In some forms, the protein segment is at least about 15 amino acids, e.g., at least 15, 16, 17, 18, 19, 20, 21, 22, 23, 24, 25, 26, 27, 28, 29, 30, or more amino acids. Examples of synthetic transmembrane domains are known in the art, for example in U.S. Pat. No. 7,052,906 and PCT Publication No. WO 2000/032776.

As described in the Examples, in some forms, the CAR includes a transmembrane domain derived from a TCR co-receptor such as CD8 or CD28.

An exemplary CD28 transmembrane domain includes an amino acid sequence of between about 20 and about 30 residues, inclusive, derived from the transmembrane component of the mature human CD28 protein. In some forms, the transmembrane domain includes an amino acid sequence of about 27 residues derived from the transmembrane component of the mature human CD28 protein.

An exemplary CD8 transmembrane domain includes an amino acid sequence of between about 20 and about 30 residues, inclusive, derived from the transmembrane component of the mature human CD8 protein. In some forms, the transmembrane domain includes an amino acid sequence of about 23 residues derived from the transmembrane component of the mature human CD8 protein.

An exemplary amino acid sequence for the transmembrane domain derived from the mature human CD8 alpha subunit includes:

(SEQ ID NO: 87)
IYIWAPLAGTCGVLLLSLVITLY.

### 3. Intracellular Domains

In some forms, the intracellular domain of the described CARs includes at least one intracellular component of a mature human CD3 subunit protein, such as the intracellular polypeptide of the human CD3 zeta subunit. Intracellular signaling via the tumor necrosis factor receptor superfamily 4-1BB improves T cell persistence even in the context of CAR activation, which indicates distinct pro-survival signals mediated by the 4-1BB cytoplasmic domain. Therefore, in some forms, the intracellular domain of the described CARs includes at least one intracellular domain of either of the costimulatory molecules, CD28 or 4-1BB. In some forms, the intracellular domain of the described CARs includes at least one intracellular component of a mature human CD3 subunit protein, such as the intracellular polypeptide of the human CD3 zeta subunit and at least one intracellular domain of either of the costimulatory molecule 4-1BB. The intracellular signaling domain is responsible for activation of at least one of the normal effector functions of the immune effector cell expressing the CAR. The term effector function refers to a specialized function of a cell. Effector function of a T cell, for example, may be cytolytic activity or helper activity including the secretion of cytokines. In some forms, an intracellular signaling domain includes the zeta chain of the T cell receptor or any of its homologs (e.g., eta, delta, gamma or epsilon), MBI chain, B29, Fc RIII, Fc RI and combinations of signaling molecules such as CD3ζ and CD28, 4-1BB, OX40 and combination thereof, as well as other similar molecules and fragments. Intracellular signaling portions of other members of the families of activating proteins can be used, such as FcγRIII and FcεRI.

An exemplary 4-1BB intracellular (cytoplasmic) domain includes an amino acid sequence of between about 30 and about 60 residues, inclusive, derived from the intracellular (cytoplasmic) domain of the mature human 4-1BB protein. In some forms, the transmembrane domain includes an amino acid sequence of about 42 residues derived from the transmembrane component of the mature human 4-1BB protein.

An exemplary amino acid sequence for the transmembrane domain derived from the mature human 4-1BB protein includes:

(SEQ ID NO: 88)
CKRGRKKLLYIFKQPFMRPVQTTQEEDGCSCRFPEEEEGGCEL

An exemplary CD3 zeta intracellular (cytoplasmic) domain includes an amino acid sequence of between about 100 and about 130 residues, inclusive, derived from the intracellular (cytoplasmic) domain of the mature human CD3 zeta protein. In some forms, the transmembrane domain includes an amino acid sequence of about 112 residues derived from the transmembrane component of the mature human CD3 zeta protein.

An exemplary amino acid sequence for the transmembrane domain derived from the mature human CD3 zeta protein includes:

(SEQ ID NO: 89)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR.

### 4. Exemplary CARs

In specific exemplary embodiments, the CAR includes the following structure: anti-ENPP3 antigen binding domain-CD8hinge-CD8TM-41BBcostim-CD3z.

A non-limiting exemplary amino acid sequence for CD8hinge-CD8TM-41BBcostim-CD3z is (italic=CD8hinge, bold+italic=CD8TM, lowercase=41BBcostim, bold=CD3z):

(SEQ ID NO: 28)
AKPTTTPAPRPPTPAPTIASQPLSLRPEACRPAAGGAVHTRGLDFACDI
YIWAPLAGTCGVLLLSLVITLYckrgrkkllyifkqpfmrpvqttqeed
gcscrfpeeeeggcelRVKFSRSADAPAYQQGQNQLYNELNLGRREEYD
VLDKRRGRDPEMGGKPRRKNPQEGLYNELQKDKMAEAYSEIGMKGERRR
GKGHDGLYQGLSTATKDTYDALHMQALPPR.

In some embodiments, SEQ ID NO:28 is preceded by a disclosed anti-ENPP3 antibody. In some embodiments, the anti-ENPP3 is an scFv. In some embodiments, the anti-ENPP3 scFv has the amino acid sequence of any one of SEQ ID NOS: 19-26.

Many immune effector cells require co-stimulation, in addition to stimulation of an antigen-specific signal, to promote cell proliferation, differentiation and survival, as well as to activate effector functions of the cell. Therefore, in some forms, the CAR includes at least one co-stimulatory signaling domain. The term co-stimulatory signaling domain, refers to at least a portion of a protein that mediates signal transduction within a cell to induce an immune response such as an effector function. The co-stimulatory signaling domain can be a cytoplasmic signaling domain from a co-stimulatory protein, which transduces a signal and modulates responses mediated by immune cells, such as T cells, NK cells, macrophages, neutrophils, or eosinophils. In some forms, the co-stimulatory signaling domain is derived from a co-stimulatory molecule selected from CD27, CD28, CD137, 0X40, CD30, CD40, CD3, LFA-1, ICOS, CD2, CD7, LIGHT, NKG2C, B7-H3, ligands of CD83 and combinations thereof.

CARs can be used in order to generate immuno-responsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and PCT Publication WO 9215322). Alternative CAR constructs can be characterized as belonging to successive generations. First-generation CARs typically include a single-chain variable fragment of an antibody specific for an antigen, for example including a VL linked to a VH of a specific antibody, linked by a flexible linker, for example by a CD8α hinge domain and a CD8α transmembrane domain, to the transmembrane and intracellular signaling domains of either CD3ζ or FcRγ (scFv-CD3ζ or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD32; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GDI 1a-CD18, CD2, ICOS, CD27, CD154, CDS, OX40, 4-1BB, or CD28 signaling domains (for example scFv-CD28-4-1BB-CD3ζ or scFv-CD28-OX40-CD3ζ; see U.S. Pat. Nos. 8,906,682; 8,399,645; 5,686,281; PCT Publication No. WO2014134165; PCT Publication No. WO2012079000). Alternatively, co-stimulation can be orchestrated by expressing CARs in antigen-specific T cells, chosen so as to be activated and expanded following engagement of their native αβTCR, for example by antigen on professional antigen-presenting cells, with attendant co-stimulation. Any of the first, second, or third generation CARs described above can be used in accordance with the disclosed compositions and methods.

Heterologous elements that can be associated with, linked, conjugated, or otherwise attached directly or indirectly to the disclosed sequence(s), or nucleic acids expressing the disclosed polypeptides are also provided. Such molecules include, but are not limited to, protein domains, such as transduction domains, fusogenic peptides, targeting molecules, and sequences that enhance protein expression and/or isolation.

In some forms, the disclosed polypeptides include one or more heterologous peptide domains, such as receptors at the surface of a cell, optionally including a transmembrane domain that anchors or connects the ectodomain to the cell surface and connects with the one or more intracellular domains. Exemplary cell surface receptors coordinate the activity of cells upon interaction with other cells, such as immune cells, such as T cells. For example, in some forms, the heterologous domain is a recombinant or engineered Programmed death protein 1 (PD1) domain.

In some forms, the disclosed molecules include one or more additional elements that are designed to assist and/or enhance the expression or production of the disclose polypeptide. Although many proteins with therapeutic or commercial uses can be produced by recombinant organisms, the yield and quality of the expressed protein are variable due to many factors. For example, heterologous protein expression by genetically engineered organisms can be affected by the size and source of the protein to be expressed, the presence of an affinity tag linked to the protein to be expressed, codon biasing, the strain of the microorganism, the culture conditions of microorganism, and the in vivo degradation of the expressed protein. Some of these problems can be mitigated by fusing the protein of interest to an expression or solubility enhancing amino acid sequence. Exemplary expression or solubility enhancing amino acid sequences include maltose-binding protein (MBP), glutathione S-transferase (GST), thioredoxin (TRX), NUS A, ubiquitin (Ub), and a small ubiquitin-related modifier (SUMO).

In some forms, the compositions disclosed herein include expression or solubility enhancing amino acid sequence. In some forms, the expression or solubility enhancing amino acid sequence is cleaved prior administration of the composition to a subject in need thereof. The expression or solubility enhancing amino acid sequence can be cleaved in the recombinant expression system, or after the expressed protein in purified.

i. CAR Proteins and Constructs

In some forms, a CAR includes an ScFv derived from one of the described anti-ENPP3 antigen binding antibodies, together with a CD8 hinge region polypeptide, CD8TM domain, 4-1BB costim domain and CD3zeta intracellular domain. Exemplary CARs are demonstrated in the Examples.

Exemplary CARs are provided below as SEQ ID NOS: 91, 93, 95, 97, 99, 101, 103, 105, 107, 109, 111, 113, 115, 117, and 119. Also provided are fragments and variants thereof. For example, each of SEQ ID NOS: 91, 93, 95, 97, 99, 101, 103, 105, 107, 109, 111, 113, 115, 117, and 119 are expressly disclosed without the N-terminal sequence MALPVTALLLPLALLLHAARPDYKDDDDK (SEQ ID NO:126). Also provided are variants having at least 70, 75, 80, 85, 90, or 95 percent sequence identity to SEQ ID NOS: 91, 93, 95, 97, 99, 101, 103, 105, 107, 109, 111, 113, 115, 117, and 119, with or without the SEQ ID NO:126.

a. CAR42 (Antibody SX13-1)

In some forms, a CAR includes an scFv derived from the anti-ENPP3 antibody SX13-1 (see, e.g., “CAR42” demonstrated in the Examples).

An exemplary nucleic acid sequence for a CAR including an scFv derived
derived from the anti-ENPP3 antibody SX13-1 is:
(SEQ ID NO: 90)
ATGGCCTTACCAGTGACCGCCTTGCTCCTGCCGCTGGCCTTGCTGCTCCACGCCGCCAG
GCCGGATTACAAAGACGATGACGATAAGCAACTGGTGGAGTCTGGGGGAGGCCTGGTCA
AGCCTGGGGGGTCCCTGAGACTCTCCTGTGCAGCCTCTGGATTCACCTTCAGTGACTAT
AGCATGAACTGGGTCCGCCAGGCTCCAGGAAAGGGGCTGGAGTGGGTCTCATCCATTAG
TAGTAGTAGTAGATACAAATACTACGCAGACTCAATGAAGGGCCGATTCACCATCTCCA
GAGACAACGCCAAGAACTCACTGTATCTGCAAATGAACAGCCTGAGAGTCGAGGACACA
GCTGTATATTACTGTACGAGAGGGATTGACTACTTTGACTACTGGGGCCAGGGAACCCT
GGTCACCGTCTCCTCAAGTGGTGGCGGTGGCTCGGGCGGTGGTGGGTCGGGTGGCGGAG
GCTCTGACATCCAGTTGACCCAGTCTCCATCCTTCCTGTCTGCATCTGTAGGAGACAGA
GTCACCATCACTTGCCGGGCCAGTCAGGACATTAACAATTATTTAGCCTGGTATCAGCA
AAAACCAGGGAAAGCCCCTAAGTTCCTGATCCATGTTGCATCCACTTTGCAAAGTGGGG
TCCCATCAAGGTTCAGCGGCAGTGGATCTGGGACAGAGTTCACTCTCACAATCAGCAGT
TTGCAGCCTGAAGATTCTGCAACTTATTACTGTCAACAGCTTAATAGTTACCCGCGGAC
GTTCGGCCAAGGGACCAAGGTGGAGATTAAAGCCAAGCCTACCACGACGCCAGCGCCGC
GACCACCAACACCGGCGCCCACCATCGCGTCGCAGCCCCTGTCCCTGCGCCCAGAGGCA
TGCCGGCCAGCAGCAGGGGGCGCAGTGCACACGAGGGGGCTGGACTTCGCCTGTGATAT
CTACATCTGGGCGCCCTTGGCCGGGACTTGTGGGGTCCTTCTCCTGTCACTGGTTATCA
CCCTTTACTGCAAACGGGGCAGAAAGAAACTCCTGTATATATTCAAACAACCATTTATG
AGACCAGTACAAACTACTCAAGAGGAAGATGGCTGTAGCTGCCGATTTCCAGAAGAAGA
AGAAGGAGGATGTGAACTGAGAGTGAAGTTCAGCAGGAGCGCAGACGCCCCCGCGTACC
AGCAGGGCCAGAACCAGCTCTATAACGAGCTCAATCTAGGACGAAGAGAGGAGTACGAT
GTTTTGGACAAGCGACGTGGCCGGGACCCTGAGATGGGGGGAAAGCCGAGAAGGAAGAA
CCCTCAGGAAGGCCTGTACAATGAACTGCAGAAAGATAAGATGGCGGAGGCCTACAGTG
AGATTGGGATGAAAGGCGAGCGCCGGAGGGGCAAGGGGCACGATGGCCTTTACCAGGGT
CTCAGTACAGCCACCAAGGACACCTACGACGCCCTTCACATGCAGGCCCTGCCCCCTCG
CTGA.
An exemplary amino acid sequence for a CAR including an scFv derived
from the anti-ENPP3 antibody SX13-1 is:
(SEQ ID NO: 91)
MALPVTALLLPLALLLHAARPDYKDDDDKQLVESGGGLVKPGGSLRLSCAASGFTFSDY
SMNWVRQAPGKGLEWVSSISSSSRYKYYADSMKGRFTISRDNAKNSLYLQMNSLRVEDT
AVYYCTRGIDYFDYWGQGTLVTVSSSGGGGSGGGGSGGGGSDIQLTQSPSFLSASVGDR
VTITCRASQDINNYLAWYQQKPGKAPKFLIHVASTLQSGVPSRFSGSGSGTEFTLTISS
LQPEDSATYYCQQLNSYPRTFGQGTKVEIKAKPTTTPAPRPPTPAPTIASQPLSLRPEA
CRPAAGGAVHTRGLDFACDIYIWAPLAGTCGVLLLSLVITLYCKRGRKKLLYIFKQPFM
RPVQTTQEEDGCSCRFPEEEEGGCELRVKFSRSADAPAYQQGQNQLYNELNLGRREEYD
VLDKRRGRDPEMGGKPRRKNPQEGLYNELQKDKMAEAYSEIGMKGERRRGKGHDGLYQG
LSTATKDTYDALHMQALPPR.

In some forms, the disclosed CAR of SEQ ID NO:91 binds to an epitope on a protein having an amino acid sequence set forth in SEQ ID NO:1 and/or SEQ ID NO:2, or a homologue, paralogue, or ortholog thereof. In some forms, the CAR is a variant having at least 70%, up to 99% identity to SEQ ID NO:91. For example in some forms, the variant sequence has at least about 70%, 75%, 80%, 85%, 90%, or 95% identity to SEQ ID NO:91. Typically, the variant CAR has an amino acid sequence that has one or more amino acids different to SEQ ID NO:91, such as one or more substitutions, deletions or additions at any one of the amino acid positions of SEQ ID NO:91, but retains the ability to specifically bind to an epitope on a protein having an amino acid sequence set forth in SEQ ID NO:1 and/or SEQ ID NO:2.

b. CAR43 (Antibody SX13-2)

In some forms, a CAR includes an scFv derived from the anti-ENPP3 antibody SX13-2 (see, e.g., “CAR43” demonstrated in the Examples).

An exemplary nucleic acid sequence for a CAR including an scFv derived from
the anti-ENPP3 antibody SX13-2 is:
(SEQ ID NO: 92)
ATGGCCTTACCAGTGACCGCCTTGCTCCTGCCGCTGGCCTTGCTGCTCCACGCCGCCAG
GCCGGATTACAAAGACGATGACGATAAGCAGCTGGTGGAGTCTGGGGGAGGCCTGGTCA
AGCCTGGGGGGTCCCTGAGACTCTCCTGTGCAGCCTCTGGATTCACCTTCAGTAGTTAT
AGCATGAACTGGGTCCGCCAGGCTCCAGGGAAGGGGCTGGAGTGGGTCACATATATTAG
TAGTAGTAGTAGTTACAGATACTACGCAGACTCAGTGAAGGGCCGGTTTACCATCTCCA
GAGACAACGCCAAGAACTCACTGAATCTGCAAATGAACACCCTGAGACCCGAGGACACA
GCTGTGTACTACTGTGCGAGAGTGGGAGCTATTACGGGGTACTTTGACTTTTGGGGCCA
GGGAACCCTGGTCACCGTCTCCTCAAGTGGTGGCGGTGGCTCGGGCGGTGGTGGGTCGG
GTGGCGGAGGCTCTGACATCCAGATGACCCAGTCTCCATCCTCCCTGTCTGCATCTGTA
GGAGACAGAGTCACCATCACTTGCCGGGCAAGTCAGGGCATTAGAAATGATTTAGACTG
GTTTCAGCAGAAACCAGGGAAAGCCCCTAAGCGCCTGATCTATGCTGCATCCAGTTTGC
AAAGTGGGGTCCCATCAAGGTTCAGCGGCAGTGGATCTGGGACAGAATTCACTCTCACA
GTCAGCAGCCTGCAGCCTGAAGATTTTGCAACTTATTACTGTCTACAGTATAATGGTTA
TCCGCTCACTTTCGGCGGAGGGACCAAGGTGGAGATCAAAGCCAAGCCTACCACGACGC
CAGCGCCGCGACCACCAACACCGGCGCCCACCATCGCGTCGCAGCCCCTGTCCCTGCGC
CCAGAGGCATGCCGGCCAGCAGCAGGGGGCGCAGTGCACACGAGGGGGCTGGACTTCGC
CTGTGATATCTACATCTGGGCGCCCTTGGCCGGGACTTGTGGGGTCCTTCTCCTGTCAC
TGGTTATCACCCTTTACTGCAAACGGGGCAGAAAGAAACTCCTGTATATATTCAAACAA
CCATTTATGAGACCAGTACAAACTACTCAAGAGGAAGATGGCTGTAGCTGCCGATTTCC
AGAAGAAGAAGAAGGAGGATGTGAACTGAGAGTGAAGTTCAGCAGGAGCGCAGACGCCC
CCGCGTACCAGCAGGGCCAGAACCAGCTCTATAACGAGCTCAATCTAGGACGAAGAGAG
GAGTACGATGTTTTGGACAAGCGACGTGGCCGGGACCCTGAGATGGGGGGAAAGCCGAG
AAGGAAGAACCCTCAGGAAGGCCTGTACAATGAACTGCAGAAAGATAAGATGGCGGAGG
CCTACAGTGAGATTGGGATGAAAGGCGAGCGCCGGAGGGGCAAGGGGCACGATGGCCTT
TACCAGGGTCTCAGTACAGCCACCAAGGACACCTACGACGCCCTTCACATGCAGGCCCT
GCCCCCTCGCTGA.
An exemplary amino acid sequence for a CAR including an scFv derived from
the anti-ENPP3 antibody SX13-2 is:
(SEQ ID NO: 93)
MALPVTALLLPLALLLHAARPDYKDDDDKQLVESGGGLVKPGGSLRLSCAASGFTFSSY
SMNWVRQAPGKGLEWVTYISSSSSYRYYADSVKGRFTISRDNAKNSLNLQMNTLRPEDT
AVYYCARVGAITGYFDFWGQGTLVTVSSSGGGGSGGGGSGGGGSDIQMTQSPSSLSASV
GDRVTITCRASQGIRNDLDWFQQKPGKAPKRLIYAASSLQSGVPSRFSGSGSGTEFTLT
VSSLQPEDFATYYCLQYNGYPLIFGGGTKVEIKAKPTTTPAPRPPTPAPTIASQPLSLR
PEACRPAAGGAVHTRGLDFACDIYIWAPLAGTCGVLLLSLVITLYCKRGRKKLLYIFKQ
PFMRPVQTTQEEDGCSCRFPEEEEGGCELRVKFSRSADAPAYQQGQNQLYNELNLGRRE
EYDVLDKRRGRDPEMGGKPRRKNPQEGLYNELQKDKMAEAYSEIGMKGERRRGKGHDGL
YQGLSTATKDTYDALHMQALPPR.

In some forms, the disclosed CAR of SEQ ID NO:93 binds to an epitope on a protein having an amino acid sequence set forth in SEQ ID NO:1 and/or SEQ ID NO:2, or a homologue, paralogue, or ortholog thereof. In some forms, the CAR is a variant having at least 70%, up to 99% identity to SEQ ID NO:93. For example in some forms, the variant sequence has at least about 70%, 75%, 80%, 85%, 90%, or 95% identity to SEQ ID NO:93. Typically, the variant CAR has an amino acid sequence that has one or more amino acids different to SEQ ID NO:93, such as one or more substitutions, deletions or additions at any one of the amino acid positions of SEQ ID NO:93, but retains the ability to specifically bind to an epitope on a protein having an amino acid sequence set forth in SEQ ID NO:1 and/or SEQ ID NO:2.

c. CAR44 (Antibody SX13-5)

In some forms, a CAR includes an scFv derived from the anti-ENPP3 antibody SX13-5 (see, e.g., “CAR44” demonstrated in the Examples).

An exemplary nucleic acid sequence for a CAR including an scFv derived from
the anti-ENPP3 antibody SX13-5 is:
(SEQ ID NO: 94)
ATGGCCTTACCAGTGACCGCCTTGCTCCTGCCGCTGGCCTTGCTGCTCCACGCCGCCAG
GCCGGATTACAAAGACGATGACGATAAGCAATTGGTGGAGTCTGGGGGAGGCCTGGTCA
AGCCTGGGGGGTCCCTGAGACTCTCCTGTGCAGCCTCTGGATTCACCTTCAGTACCTTT
AGCATGAACTGGGTCCGCCAGGCTCCAGGGAAGGGGCTGGAATGGGTTTCATACATTAG
TAGTTATAGTAGTTACATATACTACGCAGACTCAGTGAAGGGCCGATTCACCATCTCCA
GAGACAACGCCAAGAAATCACTGTATCTGCAAATGAACAGCCTGAGAGCCGAAGACACA
GCTGTCTATTACTGTTCGCGAGTGGGAGCTATTACGGGGTACTTTGACTATTGGGGCCA
GGGAACCCTGGTCACCGTCTCCTCAAGTGGTGGCGGTGGCTCGGGCGGTGGTGGGTCGG
GTGGCGGAGGCTCTGGCATCCAGATGACCCAGTCTCCATCCTCCCTGTCTGCATCTGTA
GGAGACAGAGTCACCATCACTTGCCGGGCAAGTCAGGGCATTAGAAATGATTTAGGCTG
GTATCAGCAGACACCAGGAAAAGCCCCTAAGCGCCTGATGTATACTGCATCCAGTTTGC
ACAGTGGGGTCCCCTCAAGGTTCAGCGGCAGTGGATCTGGGACAGAATTCACTCTCACA
ATCAGCAGCCTGCAGCCTGAAGATTTTGCAACTTATTACTGTCTACAGTATAATGGTTA
CCCGCTCACTTTCGGCGGAGGGACCAAGGTGGAGATCAAGGCCAAGCCTACCACGACGC
CAGCGCCGCGACCACCAACACCGGCGCCCACCATCGCGTCGCAGCCCCTGTCCCTGCGC
CCAGAGGCATGCCGGCCAGCAGCAGGGGGCGCAGTGCACACGAGGGGGCTGGACTTCGC
CTGTGATATCTACATCTGGGCGCCCTTGGCCGGGACTTGTGGGGTCCTTCTCCTGTCAC
TGGTTATCACCCTTTACTGCAAACGGGGCAGAAAGAAACTCCTGTATATATTCAAACAA
CCATTTATGAGACCAGTACAAACTACTCAAGAGGAAGATGGCTGTAGCTGCCGATTTCC
AGAAGAAGAAGAAGGAGGATGTGAACTGAGAGTGAAGTTCAGCAGGAGCGCAGACGCCC
CCGCGTACCAGCAGGGCCAGAACCAGCTCTATAACGAGCTCAATCTAGGACGAAGAGAG
GAGTACGATGTTTTGGACAAGCGACGTGGCCGGGACCCTGAGATGGGGGGAAAGCCGAG
AAGGAAGAACCCTCAGGAAGGCCTGTACAATGAACTGCAGAAAGATAAGATGGCGGAGG
CCTACAGTGAGATTGGGATGAAAGGCGAGCGCCGGAGGGGCAAGGGGCACGATGGCCTT
TACCAGGGTCTCAGTACAGCCACCAAGGACACCTACGACGCCCTTCACATGCAGGCCCT
GCCCCCTCGCTGA.
An exemplary amino acid sequence for a CAR including an scFv derived from
the anti-ENPP3 antibody SX13-5 is:
(SEQ ID NO: 95)
MALPVTALLLPLALLLHAARPDYKDDDDKQLVESGGGLVKPGGSLRLSCAASGFTFSTF
SMNWVRQAPGKGLEWVSYISSYSSYIYYADSVKGRFTISRDNAKKSLYLQMNSLRAEDT
AVYYCSRVGAITGYFDYWGQGTLVTVSSSGGGGSGGGGSGGGGSGIQMTQSPSSLSASV
GDRVTITCRASQGIRNDLGWYQQTPGKAPKRLMYTASSLHSGVPSRFSGSGSGTEFTLT
ISSLQPEDFATYYCLQYNGYPLTFGGGTKVEIKAKPTTTPAPRPPTPAPTIASQPLSLR
PEACRPAAGGAVHTRGLDFACDIYIWAPLAGTCGVLLLSLVITLYCKRGRKKLLYIFKQ
PFMRPVQTTQEEDGCSCRFPEEEEGGCELRVKFSRSADAPAYQQGQNQLYNELNLGRRE
EYDVLDKRRGRDPEMGGKPRRKNPQEGLYNELQKDKMAEAYSEIGMKGERRRGKGHDGL
YQGLSTATKDTYDALHMQALPPR.

In some forms, the disclosed CAR of SEQ ID NO:95 binds to an epitope on a protein having an amino acid sequence set forth in SEQ ID NO:1 and/or SEQ ID NO:2, or a homologue, paralogue, or ortholog thereof. In some forms, the CAR is a variant having at least 70%, up to 99% identity to SEQ ID NO:95. For example in some forms, the variant sequence has at least about 70%, 75%, 80%, 85%, 90%, or 95% identity to SEQ ID NO:95. Typically, the variant CAR has an amino acid sequence that has one or more amino acids different to SEQ ID NO:95, such as one or more substitutions, deletions or additions at any one of the amino acid positions of SEQ ID NO:95, but retains the ability to specifically bind to an epitope on a protein having an amino acid sequence set forth in SEQ ID NO:1 and/or SEQ ID NO:2.

d. CAR45 (Antibody SX13-6)

In some forms, a CAR includes an scFv derived from the anti-ENPP3 antibody SX13-6 (see, e.g., “CAR45” demonstrated in the Examples).

An exemplary nucleic acid sequence for a CAR including an scFv derived from
the anti-ENPP3 antibody SX13-6 is:
(SEQ ID NO: 96)
ATGGCCTTACCAGTGACCGCCTTGCTCCTGCCGCTGGCCTTGCTGCTCCACGCCGCCAG
GCCGGATTACAAAGACGATGACGATAAGCAGCTGGTGGAGTCTGGGGGAGACTTGGTCA
AGCCTGGAGGGTCCCTGAGACTCTCCTGTGCAGCCTCTGGATTCACCTTCAGTGACTAT
TACATGAACTGGATCCGCCAGGCTCCAGAGAAGGGACTGGAGTGGATTTCATACATTAG
TAGTAGTGGTTTTATCATGGACTCCGCAGACTCTGTGAAGGGCCGATTCACCATCTCCA
GGGACAACGCCAAGAACTCACTGTTTCTGCAAATGAACAGCCTGAGAGCCGAGGACACG
GCCGTGTATTACTGTGCGAGAGGTAACTGGAACTACCCTTTTGACTACTGGGGCCAGGG
AACCCTGGTCACCGTCTCCTCAAGTGGTGGCGGTGGCTCGGGCGGTGGTGGGTCGGGTG
GCGGAGGCTCTGACATCCAGATGACCCAGTCTCCATCCTCCCTGTCTGCATCTATAGGA
GACAGAGTCACCATCACTTGCCGGGCGAGTCAGGGCATTAGCCATTATTTAGCCTGGTA
TCAGCAGAGACCAGGGAAAGTTCCTAAGTTCCTGATCTATGCTGCATCCACTTTACAAT
CAGGGGTCCCATCTCGGTTCAGTGGCCGTGGATCTGGGACAGATTTCACTCTCACCATC
AGCAGCCTGCAGCCTGAAGATATTGCGACTTATTACTGTCAGATGTATAAAAATGTCCC
ATTCACTTTCGGCCCTGGGACCAAAGTGGATATCAAAGCCAAGCCTACCACGACGCCAG
CGCCGCGACCACCAACACCGGCGCCCACCATCGCGTCGCAGCCCCTGTCCCTGCGCCCA
GAGGCATGCCGGCCAGCAGCAGGGGGCGCAGTGCACACGAGGGGGCTGGACTTCGCCTG
TGATATCTACATCTGGGCGCCCTTGGCCGGGACTTGTGGGGTCCTTCTCCTGTCACTGG
TTATCACCCTTTACTGCAAACGGGGCAGAAAGAAACTCCTGTATATATTCAAACAACCA
TTTATGAGACCAGTACAAACTACTCAAGAGGAAGATGGCTGTAGCTGCCGATTTCCAGA
AGAAGAAGAAGGAGGATGTGAACTGAGAGTGAAGTTCAGCAGGAGCGCAGACGCCCCCG
CGTACCAGCAGGGCCAGAACCAGCTCTATAACGAGCTCAATCTAGGACGAAGAGAGGAG
TACGATGTTTTGGACAAGCGACGTGGCCGGGACCCTGAGATGGGGGGAAAGCCGAGAAG
GAAGAACCCTCAGGAAGGCCTGTACAATGAACTGCAGAAAGATAAGATGGCGGAGGCCT
ACAGTGAGATTGGGATGAAAGGCGAGCGCCGGAGGGGCAAGGGGCACGATGGCCTTTAC
CAGGGTCTCAGTACAGCCACCAAGGACACCTACGACGCCCTTCACATGCAGGCCCTGCC
CCCTCGCTGA.
An exemplary amino acid sequence for a CAR including an scFv derived from
the anti-ENPP3 antibody SX13-6 is:
(SEQ ID NO: 97)
MALPVTALLLPLALLLHAARPDYKDDDDKQLVESGGDLVKPGGSLRLSCAASGFTFSDY
YMNWIRQAPEKGLEWISYISSSGFIMDSADSVKGRFTISRDNAKNSLFLQMNSLRAEDT
AVYYCARGNWNYPFDYWGQGTLVTVSSSGGGGSGGGGSGGGGSDIQMTQSPSSLSASIG
DRVTITCRASQGISHYLAWYQQRPGKVPKFLIYAASTLQSGVPSRFSGRGSGTDFTLTI
SSLQPEDIATYYCQMYKNVPFTFGPGTKVDIKAKPTTTPAPRPPTPAPTIASQPLSLRP
EACRPAAGGAVHTRGLDFACDIYIWAPLAGTCGVLLLSLVITLYCKRGRKKLLYIFKQP
FMRPVQTTQEEDGCSCRFPEEEEGGCELRVKFSRSADAPAYQQGQNQLYNELNLGRREE
YDVLDKRRGRDPEMGGKPRRKNPQEGLYNELQKDKMAEAYSEIGMKGERRRGKGHDGLY
QGLSTATKDTYDALHMQALPPR.

In some forms, the disclosed CAR of SEQ ID NO:97 binds to an epitope on a protein having an amino acid sequence set forth in SEQ ID NO:1 and/or SEQ ID NO:2, or a homologue, paralogue, or ortholog thereof. In some forms, the CAR is a variant having at least 70%, up to 99% identity to SEQ ID NO:97. For example in some forms, the variant sequence has at least about 70%, 75%, 80%, 85%, 90%, or 95% identity to SEQ ID NO:97. Typically, the variant CAR has an amino acid sequence that has one or more amino acids different to SEQ ID NO:97, such as one or more substitutions, deletions or additions at any one of the amino acid positions of SEQ ID NO:97, but retains the ability to specifically bind to an epitope on a protein having an amino acid sequence set forth in SEQ ID NO:1 and/or SEQ ID NO:2.

e. CAR46 (Antibody SX13-7)

In some forms, a CAR includes an scFv derived from the anti-ENPP3 antibody SX13-7 (see, e.g., “CAR46” demonstrated in the Examples).

An exemplary nucleic acid sequence for a CAR including an scFv derived from
the anti-ENPP3 antibody SX13-7 is:
(SEQ ID NO: 98)
ATGGCCTTACCAGTGACCGCCTTGCTCCTGCCGCTGGCCTTGCTGCTCCACGCCGCCAG
GCCGGATTACAAAGACGATGACGATAAGCAGCTGGTGGAGTCTGGGGGAGGCCTGGTCA
AGCCTGGGGGGTCCCTGAGACTCTCCTGTGCAGCCTCTGGATTCACCTTCAGTGACTAT
AGCATGAACTGGGTCCGCCAGGCTCCAGGGAAGGGGCTGGAGTGGGTCTCATCCATTAG
TAGTAGTAGTAGTTACAAAAAGTACGCAGACTCAGTGAAGGGCCGCCTCACCATCTCCA
GAGACAACGCCAAGAATTCATTGTATCTGCAGATGAACAGCCTGAGAGCCGAGGACACA
GCTGTGTATTACTGTGCGAGAGGGATTGACTACTTTGATTACTGGGGCCAGGGAACCCT
GGTCACCGTCTCCTCAAGTGGTGGCGGTGGCTCGGGCGGTGGTGGGTCGGGTGGCGGAG
GCTCTGACATCCAGTTGACCCAGTCTCCATCCTTCCTGTCTGCATCTGTAGGAGACAGA
GTCACCATCACTTGCCGGGCCAGTCAGGGCATTAGCAGTAACTTAGCCTGGTATCAGCA
GAAACCAGGGAAAGACCCTAAGGTCCTGATCTATGGTGCATCCACTTTGCAAAGTGGGG
TCCCATCAAGGTTCAGCGGCAGTGGATCTGGGACAGAATTCACTCTCACAATCAGCAGC
CTGCAGCCTGAAGATTTTGCAACTTATTACTGTCAACAGCTTAATAGTTACCCGAGGAC
GTTCGGCCAAGGGACCAAGGTGGAAATCAAAGCCAAGCCTACCACGACGCCAGCGCCGC
GACCACCAACACCGGCGCCCACCATCGCGTCGCAGCCCCTGTCCCTGCGCCCAGAGGCA
TGCCGGCCAGCAGCAGGGGGCGCAGTGCACACGAGGGGGCTGGACTTCGCCTGTGATAT
CTACATCTGGGCGCCCTTGGCCGGGACTTGTGGGGTCCTTCTCCTGTCACTGGTTATCA
CCCTTTACTGCAAACGGGGCAGAAAGAAACTCCTGTATATATTCAAACAACCATTTATG
AGACCAGTACAAACTACTCAAGAGGAAGATGGCTGTAGCTGCCGATTTCCAGAAGAAGA
AGAAGGAGGATGTGAACTGAGAGTGAAGTTCAGCAGGAGCGCAGACGCCCCCGCGTACC
AGCAGGGCCAGAACCAGCTCTATAACGAGCTCAATCTAGGACGAAGAGAGGAGTACGAT
GTTTTGGACAAGCGACGTGGCCGGGACCCTGAGATGGGGGGAAAGCCGAGAAGGAAGAA
CCCTCAGGAAGGCCTGTACAATGAACTGCAGAAAGATAAGATGGCGGAGGCCTACAGTG
AGATTGGGATGAAAGGCGAGCGCCGGAGGGGCAAGGGGCACGATGGCCTTTACCAGGGT
CTCAGTACAGCCACCAAGGACACCTACGACGCCCTTCACATGCAGGCCCTGCCCCCTCG
CTGA.
An exemplary amino acid sequence for a CAR including an scFv derived from
the anti-ENPP3 antibody SX13-7 is:
(SEQ ID NO: 99)
MALPVTALLLPLALLLHAARPDYKDDDDKQLVESGGGLVKPGGSLRLSCAASGFTFSDY
SMNWVRQAPGKGLEWVSSISSSSSYKKYADSVKGRLTISRDNAKNSLYLQMNSLRAEDT
AVYYCARGIDYFDYWGQGTLVTVSSSGGGGSGGGGSGGGGSDIQLTQSPSFLSASVGDR
VTITCRASQGISSNLAWYQQKPGKDPKVLIYGASTLQSGVPSRFSGSGSGTEFTLTISS
LQPEDFATYYCQQLNSYPRTFGQGTKVEIKAKPTTTPAPRPPTPAPTIASQPLSLRPEA
CRPAAGGAVHTRGLDFACDIYIWAPLAGTCGVLLLSLVITLYCKRGRKKLLYIFKQPFM
RPVQTTQEEDGCSCRFPEEEEGGCELRVKFSRSADAPAYQQGQNQLYNELNLGRREEYD
VLDKRRGRDPEMGGKPRRKNPQEGLYNELQKDKMAEAYSEIGMKGERRRGKGHDGLYQG
LSTATKDTYDALHMQALPPR.

In some forms, the disclosed CAR of SEQ ID NO:99 binds to an epitope on a protein having an amino acid sequence set forth in SEQ ID NO: 1 and/or SEQ ID NO:2, or a homologue, paralogue, or ortholog thereof. In some forms, the CAR is a variant having at least 70%, up to 99% identity to SEQ ID NO:99. For example in some forms, the variant sequence has at least about 70%, 75%, 80%, 85%, 90%, or 95% identity to SEQ ID NO:99. Typically, the variant CAR has an amino acid sequence that has one or more amino acids different to SEQ ID NO:99, such as one or more substitutions, deletions or additions at any one of the amino acid positions of SEQ ID NO:99, but retains the ability to specifically bind to an epitope on a protein having an amino acid sequence set forth in SEQ ID NO:1 and/or SEQ ID NO:2.

f. CAR47 (Antibody SX13-8.1)

In some forms, a CAR includes an scFv derived from the anti-ENPP3 antibody SX13-8.1 (see, e.g., “CAR47” demonstrated in the Examples).

An exemplary nucleic acid sequence for a CAR including an scFv derived from
the anti-ENPP3 antibody SX13-8.1 is:
(SEQ ID NO: 100)
ATGGCCTTACCAGTGACCGCCTTGCTCCTGCCGCTGGCCTTGCTGCTCCACGCCGCCAG
GCCGGATTACAAAGACGATGACGATAAGGTACAGCTGCAGCAGTCAGGTCCAGGACTGG
TGAAGCCCTCGCAGACCCTCTCACTCACCTGTGCCATCTCCGGGGACAGTGTCTCTAGC
AACAGAGCTGCTTGGAACTGGATCAGGCAGCCCCCATCGAGAGGCCTTGAGTGGCTGGG
AAGGACATACTACAGGTCCAACTGGTATAATGATTATGCAGTTTCTGTGAAGAGTCGAA
TAACCATCAAACCAGACACATCCAAGAACCAGTTCTCCCTGCAGCTGAACTCTGTGACT
CCCGAGGACACGGCTGTTTATTACTGTGCAAGAGATCGTGGGACCTACTACTACTACTA
CTACATGGACGTCTGGGACAAAGGGACCACGGTCACCGTCTCCTCAAGTGGTGGCGGTG
GCTCGGGCGGTGGTGGGTCGGGTGGCGGAGGCTCTGAAATTGTGTTGACGCAGTCTCCA
GGCACCCTGTCTTTGTCTCCAGGGGAAAGAGCCACCCTCTCCTGCAGGGCCAGTCAGAG
TGTTAGTAGCAGCTACTTAGTCTGGTACCAACAGAAAGCTGGCCAGGCTCCCAGGATCC
TCATCTATGGTGCCTCCAACAGGGCCACTGACATCCCAGTCAGGTTCAGTGGCAGTGGG
TCTGGGACAGACTTCACTCTCACCATCAGCAGACTGGAGCCTGAGGATTTTGGAGTGTA
TTTCTGTCAGCATTATGGTGGCTCATACACTTTTGGCCAGGGGACCAAGCTGGAGATCA
AAGCCAAGCCTACCACGACGCCAGCGCCGCGACCACCAACACCGGCGCCCACCATCGCG
TCGCAGCCCCTGTCCCTGCGCCCAGAGGCATGCCGGCCAGCAGCAGGGGGCGCAGTGCA
CACGAGGGGGCTGGACTTCGCCTGTGATATCTACATCTGGGCGCCCTTGGCCGGGACTT
GTGGGGTCCTTCTCCTGTCACTGGTTATCACCCTTTACTGCAAACGGGGCAGAAAGAAA
CTCCTGTATATATTCAAACAACCATTTATGAGACCAGTACAAACTACTCAAGAGGAAGA
TGGCTGTAGCTGCCGATTTCCAGAAGAAGAAGAAGGAGGATGTGAACTGAGAGTGAAGT
TCAGCAGGAGCGCAGACGCCCCCGCGTACCAGCAGGGCCAGAACCAGCTCTATAACGAG
CTCAATCTAGGACGAAGAGAGGAGTACGATGTTTTGGACAAGCGACGTGGCCGGGACCC
TGAGATGGGGGGAAAGCCGAGAAGGAAGAACCCTCAGGAAGGCCTGTACAATGAACTGC
AGAAAGATAAGATGGCGGAGGCCTACAGTGAGATTGGGATGAAAGGCGAGCGCCGGAGG
GGCAAGGGGCACGATGGCCTTTACCAGGGTCTCAGTACAGCCACCAAGGACACCTACGA
CGCCCTTCACATGCAGGCCCTGCCCCCTCGCTGA.
An exemplary amino acid sequence for a CAR including an scFv derived from
the anti-ENPP3 antibody SX13-8.1 is:
(SEQ ID NO: 101)
MALPVTALLLPLALLLHAARPDYKDDDDKVQLQQSGPGLVKPSQTLSLTCAISGDSVSS
NRAAWNWIRQPPSRGLEWLGRTYYRSNWYNDYAVSVKSRITIKPDTSKNQFSLQLNSVT
PEDTAVYYCARDRGTYYYYYYMDVWDKGTTVTVSSSGGGGSGGGGSGGGGSEIVLTQSP
GTLSLSPGERATLSCRASQSVSSSYLVWYQQKAGQAPRILIYGASNRATDIPVRFSGSG
SGTDFTLTISRLEPEDFGVYFCQHYGGSYTFGQGTKLEIKAKPTTTPAPRPPTPAPTIA
SQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNE
LNLGRREEYDVLDKRRGRDPEMGGKPRRKNPQEGLYNELQKDKMAEAYSEIGMKGERRR
GKGHDGLYQGLSTATKDTYDALHMQALPPR.

In some forms, the disclosed CAR of SEQ ID NO: 101 binds to an epitope on a protein having an amino acid sequence set forth in SEQ ID NO:1 and/or SEQ ID NO:2, or a homologue, paralogue, or ortholog thereof. In some forms, the CAR is a variant having at least 70%, up to 99% identity to SEQ ID NO:101. For example in some forms, the variant sequence has at least about 70%, 75%, 80%, 85%, 90%, or 95% identity to SEQ ID NO:101. Typically, the variant CAR has an amino acid sequence that has one or more amino acids different to SEQ ID NO:101, such as one or more substitutions, deletions or additions at any one of the amino acid positions of SEQ ID NO:101, but retains the ability to specifically bind to an epitope on a protein having an amino acid sequence set forth in SEQ ID NO:1 and/or SEQ ID NO:2.

g. CAR48 (Antibody SX13-10)

In some forms, a CAR includes an scFv derived from the anti-ENPP3 antibody SX13-10 (see, e.g., “CAR48” demonstrated in the Examples).

An exemplary nucleic acid sequence for a CAR including an
scFv derived from the anti-ENPP3 antibody SX13-10 is:
(SEQ ID NO: 102)
ATGGCCTTACCAGTGACCGCCTTGCTCCTGCCGCTGGCCTTGCTGCTCCACGCCGCCAG
GCCGGATTACAAAGACGATGACGATAAGCAGCTGCTGGAGTCTGGGGGAGGCCTGGTCA
AGCCTGGGGGGTCCCTGAGACTCTCCTGTGCGGCCTCTGGATTCACGTTCAGTAGATAT
AACATGAACTGGGTCCGCCAGGCTCCAGGGAAGGGGCTGGAGTGGGTCTCATTCATCAG
TGGCAGTAGTAGTTACGTATACTACGCAGACTCAGTGAAGGGCCGATTCACCATCTCCA
AAGACAACGCCAAGAACTCACTGTATCTGCAAATGAACAGCCTGAGAGCCGAGGACACA
GCTGTATATTATTGTGCGAGATCGGGGTATTGTACTAGTACCACCTGCTATAATTACTA
CTACTACTACATGGACGTCTGGGGCAAAGGGACCACGGTCACCGTCTCCTCAAGTGGTG
GCGGTGGCTCGGGCGGTGGTGGGTCGGGTGGCGGAGGCTCTGAAATTGTGTTGATGCAG
TCTCCAGGCACCCTGTCTTTGTCTCCAGGGGAAAGAGCCACCCTCTCCTGCAGGGCCAG
TCAGAGTGTTAGCAACAGCTACTTAGCCTGGTACCAGCAGAAACCTGGCCAGGCTCCCA
GGCTCCTCATCTATGGTGCATCCAGCAGGGCCACTGGCATCCCAGACAGGTTCAGTGGC
AGTGGGTCTGGGACAGACTTCACTCTCACCATCAGCAGACTGGAGCCTGAAGATTTTGC
AGTGTTTTACTGTCAGCAGTTTGGTAGCTCACCGCTCACTTTCGGCGGAGGGACCAAGG
TGGAGATCAAAGCCAAGCCTACCACGACGCCAGCGCCGCGACCACCAACACCGGCGCCC
ACCATCGCGTCGCAGCCCCTGTCCCTGCGCCCAGAGGCATGCCGGCCAGCAGCAGGGGG
CGCAGTGCACACGAGGGGGCTGGACTTCGCCTGTGATATCTACATCTGGGCGCCCTTGG
CCGGGACTTGTGGGGTCCTTCTCCTGTCACTGGTTATCACCCTTTACTGCAAACGGGGC
AGAAAGAAACTCCTGTATATATTCAAACAACCATTTATGAGACCAGTACAAACTACTCA
AGAGGAAGATGGCTGTAGCTGCCGATTTCCAGAAGAAGAAGAAGGAGGATGTGAACTGA
GAGTGAAGTTCAGCAGGAGCGCAGACGCCCCCGCGTACCAGCAGGGCCAGAACCAGCTC
TATAACGAGCTCAATCTAGGACGAAGAGAGGAGTACGATGTTTTGGACAAGCGACGTGG
CCGGGACCCTGAGATGGGGGGAAAGCCGAGAAGGAAGAACCCTCAGGAAGGCCTGTACA
ATGAACTGCAGAAAGATAAGATGGCGGAGGCCTACAGTGAGATTGGGATGAAAGGCGAG
CGCCGGAGGGGCAAGGGGCACGATGGCCTTTACCAGGGTCTCAGTACAGCCACCAAGGA
CACCTACGACGCCCTTCACATGCAGGCCCTGCCCCCTCGCTGA.
An exemplary amino acid sequence for a CAR including an
scFv derived from the anti-ENPP3 antibody SX13-10 is:
(SEQ ID NO: 103)
MALPVTALLLPLALLLHAARPDYKDDDDKQLLESGGGLVKPGGSLRLSCAASGFTFSRY
NMNWVRQAPGKGLEWVSFISGSSSYVYYADSVKGRFTISKDNAKNSLYLQMNSLRAEDT
AVYYCARSGYCTSTTCYNYYYYYMDVWGKGTTVTVSSSGGGGSGGGGSGGGGSEIVLMQ
SPGTLSLSPGERATLSCRASQSVSNSYLAWYQQKPGQAPRLLIYGASSRATGIPDRFSG
SGSGTDFTLTISRLEPEDFAVFYCQQFGSSPLTFGGGTKVEIKAKPTTTPAPRPPTPAP
TIASQPLSLRPEACRPAAGGAVHTRGLDFACDIYIWAPLAGTCGVLLLSLVITLYCKRG
RKKLLYIFKQPFMRPVQTTQEEDGCSCRFPEEEEGGCELRVKFSRSADAPAYQQGQNQL
YNELNLGRREEYDVLDKRRGRDPEMGGKPRRKNPQEGLYNELQKDKMAEAYSEIGMKGE
RRRGKGHDGLYQGLSTATKDTYDALHMQALPPR.

In some forms, the disclosed CAR of SEQ ID NO:103 binds to an epitope on a protein having an amino acid sequence set forth in SEQ ID NO:1 and/or SEQ ID NO:2, or a homologue, paralogue, or ortholog thereof. In some forms, the CAR is a variant having at least 70%, up to 99% identity to SEQ ID NO:103. For example, in some forms, the variant sequence has at least about 70%, 75%, 80%, 85%, 90%, or 95% identity to SEQ ID NO:103. Typically, the variant CAR has an amino acid sequence that has one or more amino acids different to SEQ ID NO:103, such as one or more substitutions, deletions or additions at any one of the amino acid positions of SEQ ID NO:103, but retains the ability to specifically bind to an epitope on a protein having an amino acid sequence set forth in SEQ ID NO:1 and/or SEQ ID NO:2.

h. CAR49 (Antibody SX13-12)

In some forms, a CAR includes an scFv derived from the anti-ENPP3 antibody SX13-12 (see, e.g., “CAR49” demonstrated in the Examples).

An exemplary nucleic acid sequence for a CAR including an
scFv derived from the anti-ENPP3 antibody SX13-10 is:
(SEQ ID NO: 104)
ATGGCCTTACCAGTGACCGCCTTGCTCCTGCCGCTGGCCTTGCTGCTCCACGCCGCCAG
GCCGGATTACAAAGACGATGACGATAAGCAGCTGGTGGAGTCTGGGGGAGGCCTGGTCA
AGCCTGGGGGGTCCCTGAGACTCTCCTGTGCAGCCTCTGGATTCACCTTCAGTGACTAC
AGCATGAACTGGGTCCGCCAGGCTCCAGGAAAGGGGCTGAATTGGGTCGCATCCATTAG
TAGTAGTAGTCATTACAAATACTACGCAGACTCAGTGAAGGGCCGATTCACCATCTCCA
GAGACAACGCCAAGAACTCACTGTATCTGCAAATGAACAGCCTGAGAGCCGAGGACACA
GCTGTGTATTATTGTGCGAGAGGGATTGATTTTTTTGATATTTGGGGCCAAGGGACAAT
GGTCACCGTCTCTTCAAGTGGTGGCGGTGGCTCGGGCGGTGGTGGGTCGGGTGGCGGAG
GCTCTGACATCCAGTTGACCCAGTCTCCATCCTTCCTGTCTGCATCTGTAGGAGACAGA
GTCACCATCACTTGCCGGGCCAGTCAGGACATTAACAGTTATTTAGTTTGGTATCAGCA
AAAACCAGGGAAAGCCCCTAAACTCCTGATCTATACTGCATCCACTTTGCAAAGCGGGG
TCCCATCAAGGTTCAGCGGCAGTGGATCTGGGACAGAATTCACTCTCACAATCAGAAGC
CTGCAGCCTGAAGATTTTGCAACTTATTACTGTCAACAGCTTAATAGTTACCCTCGGAC
GTTCGGCCAAGGGACCAAGGTGGAAATCAAAGCCAAGCCTACCACGACGCCAGCGCCGC
GACCACCAACACCGGCGCCCACCATCGCGTCGCAGCCCCTGTCCCTGCGCCCAGAGGCA
TGCCGGCCAGCAGCAGGGGGCGCAGTGCACACGAGGGGGCTGGACTTCGCCTGTGATAT
CTACATCTGGGCGCCCTTGGCCGGGACTTGTGGGGTCCTTCTCCTGTCACTGGTTATCA
CCCTTTACTGCAAACGGGGCAGAAAGAAACTCCTGTATATATTCAAACAACCATTTATG
AGACCAGTACAAACTACTCAAGAGGAAGATGGCTGTAGCTGCCGATTTCCAGAAGAAGA
AGAAGGAGGATGTGAACTGAGAGTGAAGTTCAGCAGGAGCGCAGACGCCCCCGCGTACC
AGCAGGGCCAGAACCAGCTCTATAACGAGCTCAATCTAGGACGAAGAGAGGAGTACGAT
GTTTTGGACAAGCGACGTGGCCGGGACCCTGAGATGGGGGGAAAGCCGAGAAGGAAGAA
CCCTCAGGAAGGCCTGTACAATGAACTGCAGAAAGATAAGATGGCGGAGGCCTACAGTG
AGATTGGGATGAAAGGCGAGCGCCGGAGGGGCAAGGGGCACGATGGCCTTTACCAGGGT
CTCAGTACAGCCACCAAGGACACCTACGACGCCCTTCACATGCAGGCCCTGCCCCCTCG
CTGA.
An exemplary amino acid sequence for a CAR including an
scFv derived from the anti-ENPP3 antibody SX13-12 is:
(SEQ ID NO: 105)
MALPVTALLLPLALLLHAARPDYKDDDDKQLVESGGGLVKPGGSLRLSCAASGFTFSDY
SMNWVRQAPGKGLNWVASISSSSHYKYYADSVKGRFTISRDNAKNSLYLQMNSLRAEDT
AVYYCARGIDFFDIWGQGTMVTVSSSGGGGSGGGGSGGGGSDIQLTQSPSFLSASVGDR
VTITCRASQDINSYLVWYQQKPGKAPKLLIYTASTLQSGVPSRFSGSGSGTEFTLTIRS
LQPEDFATYYCQQLNSYPRTFGQGTKVEIKAKPTTTPAPRPPTPAPTIASQPLSLRPEA
CRPAAGGAVHTRGLDFACDIYIWAPLAGTCGVLLLSLVITLYCKRGRKKLLYIFKQPFM
RPVQTTQEEDGCSCREPEEEEGGCELRVKFSRSADAPAYQQGQNQLYNELNLGRREEYD
VLDKRRGRDPEMGGKPRRKNPQEGLYNELQKDKMAEAYSEIGMKGERRRGKGHDGLYQG
LSTATKDTYDALHMQALPPR.

In some forms, the disclosed CAR of SEQ ID NO:105 binds to an epitope on a protein having an amino acid sequence set forth in SEQ ID NO: 1 and/or SEQ ID NO:2, or a homologue, paralogue, or ortholog thereof. In some forms, the CAR is a variant having at least 70%, up to 99% identity to SEQ ID NO:105. For example in some forms, the variant sequence has at least about 70%, 75%, 80%, 85%, 90%, or 95% identity to SEQ ID NO:105. Typically, the variant CAR has an amino acid sequence that has one or more amino acids different to SEQ ID NO:105, such as one or more substitutions, deletions or additions at any one of the amino acid positions of SEQ ID NO:105, but retains the ability to specifically bind to an epitope on a protein having an amino acid sequence set forth in SEQ ID NO:1 and/or SEQ ID NO:2.

i. CAR50 (Control Antibody)

In some forms, a CAR includes an scFv derived from the control anti-ENPP3 antibody (see, e.g., “CAR50” demonstrated in the Examples).

An exemplary nucleic acid sequence for a CAR including an
scFv derived from the control anti-ENPP3 antibody is:
(SEQ ID NO: 106)
ATGGCCTTACCAGTGACCGCCTTGCTCCTGCCGCTGGCCTTGCTGCTCCACGCCGCCAG
GCCGGATTACAAAGACGATGACGATAAGATGGAGGTGCTGGTGGAGAGCGGCGGCGGCC
TGGTGCAGCCCGGCGGCAGCCTGAGGCTGAGCTGCGCCGCCAGCGGCTTCGCCTTCAGC
AACTACGGCATGAGCTGGGTGAGGCAGGCCCCCGGCCAGAGGCTGGAGTGGGTGAGCTA
CATCAGCAGCGGCGGCGGCACCACCTACTACCTGGACAGCGTGACCGGCAGGTTCACCA
TCAGCAGGGACAACGCCAAGAACAGCCTGTACCTGCAGATGAACAGCCTGAGGGCCGAG
GACACCGCCGTGTACTACTGCGCCAGGCACAGGAGGGTGTACTACGGCTACTACTTCGA
CTACTGGGGCCAGGGCACCCTGGTGACCGTGAGCAGCAGTGGTGGCGGTGGCTCGGGCG
GTGGTGGGTCGGGTGGCGGAGGCTCTATGGACATCCTGCTGACCCAGAGCCCCGACTTC
CAGAGCGTGACCCCCAAGGAGAAGGTGACCATCACCTGCAGGGCCAGCCAGAGCATCGG
CACCAGCATCCACTGGTACCAGCAGAAGCCCGACCAGAGCCCCAAGCTGCTGATCGAGT
ACGCCAGCGAGAGCATCAGCGGCATCCCCAGCAGGTTCAGCGGCAGCGGCAGCGGCACC
GACTTCACCCTGACCATCAACAGCCTGGAGGCCGAGGACGCCGCCACCTACTACTGCCA
GCAGAGCAACAGCTGGCCCTACACCTTCGGCGGCGGCACCAAGCTGGAGATCAAGGCCA
AGCCTACCACGACGCCAGCGCCGCGACCACCAACACCGGCGCCCACCATCGCGTCGCAG
CCCCTGTCCCTGCGCCCAGAGGCATGCCGGCCAGCAGCAGGGGGCGCAGTGCACACGAG
GGGGCTGGACTTCGCCTGTGATATCTACATCTGGGCGCCCTTGGCCGGGACTTGTGGGG
TCCTTCTCCTGTCACTGGTTATCACCCTTTACTGCAAACGGGGCAGAAAGAAACTCCTG
TATATATTCAAACAACCATTTATGAGACCAGTACAAACTACTCAAGAGGAAGATGGCTG
TAGCTGCCGATTTCCAGAAGAAGAAGAAGGAGGATGTGAACTGAGAGTGAAGTTCAGCA
GGAGCGCAGACGCCCCCGCGTACCAGCAGGGCCAGAACCAGCTCTATAACGAGCTCAAT
CTAGGACGAAGAGAGGAGTACGATGTTTTGGACAAGCGACGTGGCCGGGACCCTGAGAT
GGGGGGAAAGCCGAGAAGGAAGAACCCTCAGGAAGGCCTGTACAATGAACTGCAGAAAG
ATAAGATGGCGGAGGCCTACAGTGAGATTGGGATGAAAGGCGAGCGCCGGAGGGGCAAG
GGGCACGATGGCCTTTACCAGGGTCTCAGTACAGCCACCAAGGACACCTACGACGCCCT
TCACATGCAGGCCCTGCCCCCTCGCTGA.
An exemplary amino acid sequence for a CAR including an
scFv derived from the control anti-ENPP3 antibody is:
(SEQ ID NO: 107)
MALPVTALLLPLALLLHAARPDYKDDDDKMEVLVESGGGLVQPGGSLRLSCAASGFAFS
NYGMSWVRQAPGQRLEWVSYISSGGGTTYYLDSVTGRFTISRDNAKNSLYLQMNSLRAE
DTAVYYCARHRRVYYGYYFDYWGQGTLVTVSSSGGGGSGGGGSGGGGSMDILLTQSPDF
QSVTPKEKVTITCRASQSIGTSIHWYQQKPDQSPKLLIEYASESISGIPSRFSGSGSGT
DFTLTINSLEAEDAATYYCQQSNSWPYTFGGGTKLEIKAKPTTTPAPRPPTPAPTIASQ
PLSLRPEACRPAAGGAVHTRGLDFACDIYIWAPLAGTCGVLLLSLVITLYCKRGRKKLL
YIFKQPFMRPVQTTQEEDGCSCRFPEEEEGGCELRVKFSRSADAPAYQQGQNQLYNELN
LGRREEYDVLDKRRGRDPEMGGKPRRKNPQEGLYNELQKDKMAEAYSEIGMKGERRRGK
GHDGLYQGLSTATKDTYDALHMQALPPR.

In some forms, the disclosed CAR of SEQ ID NO:107 binds to an epitope on a protein having an amino acid sequence set forth in SEQ ID NO:1 and/or SEQ ID NO:2, or a homologue, paralogue, or ortholog thereof. In some forms, the CAR is a variant having at least 70%, up to 99% identity to SEQ ID NO:107. For example in some forms, the variant sequence has at least about 70%, 75%, 80%, 85%, 90%, or 95% identity to SEQ ID NO:107. Typically, the variant CAR has an amino acid sequence that has one or more amino acids different to SEQ ID NO:107, such as one or more substitutions, deletions or additions at any one of the amino acid positions of SEQ ID NO:107, but retains the ability to specifically bind to an epitope on a protein having an amino acid sequence set forth in SEQ ID NO:1 and/or SEQ ID NO:2.

ii. Variant Domain CAR Proteins and Constructs

In some forms, a CAR includes an antigen binding domain, typically an anti-ENPP3 antigen binding domain, optionally an scFv or other construct, optionally including the CDRs derived from one of the described anti-ENPP3 antigen binding antibodies, together with a variant spacer domain, transmembrane domain, and/or intracellular/costimulatory (“costim”) domains. Exemplary variants include swapping one or more of the CD8hinge region polypeptide, CD8TM domain, 4-1BBcostim domain and CD3zeta intracellular domain with a spacer domain from human IgG4, and/or CD28, the transmembrane domain of CD8 alpha or CD28, the intracellular domain of CD28, or 4-1BB. Typically, all variant domain CARs include the intracellular CD3 zeta component. Exemplary variant domain CARs are demonstrated in the Examples, including CARs prepared with different “framework” domain structures, including CAR 199, CAR 203, CAR 205, CAR 216, CAR 217 and CAR 219 framework domain structures.

### CAR 199 Framework

In some forms, a domain variant CAR includes an antigen binding domain, such as an IgG ScFv, fused to a CAR framework including a polypeptide derived from the extracellular (hinge) domain of human CD8 alpha coreceptor, together with a polypeptide derived from the transmembrane region of the human CD8 alpha coreceptor, and a cytoplasmic region derived from the cytoplasmic component of 4-1BB and CD3 zeta. Typically, the CAR framework does not include the antigen binding component, but can be used to form a function CAR by conjugation with any suitable antigen-specific domain. An exemplary antigen-specific domain is an antigen binding domain of an anti-ENPP3 antibody, such as an scFv of an anti-ENPP3 antibody. An exemplary amino acid sequence for a CAR 199 framework includes:

(SEQ ID NO: 120)
KIEVMYPPPYLDNEKSNGTIIHVKGKHLCPSPLFPGPSKPFWVLVV
VGGVLACYSLLVTVAFIIFWVKRGRKKLLYIFKQPFMRPVQTTQEE
DGCSCRFPEEEEGGCELRVKFSRSADAPAYQQGQNQLYNELNLGRR
EEYDVLDKRRGRDPEMGGKPRRKNPQEGLYNELQKDKMAEAYSEIG
MKGERRRGKGHDGLYQGLSTATKDTYDALHMQALPPR.

### CAR 203 Framework

In some forms, a domain variant CAR includes an antigen binding domain, such as an IgG ScFv, fused to a CAR framework including a polypeptide derived from the extracellular (hinge) domain of human IgG4m, together with a polypeptide derived from the transmembrane region of the human CD8 alpha coreceptor, and a cytoplasmic region derived from the cytoplasmic component of 4-1BB and CD3 zeta. Typically, a CAR framework does not include the antigen binding component, but can be used to form a functional CAR by conjugation with any suitable antigen-specific domain. An exemplary antigen-specific domain is an antigen binding domain of an anti-ENPP3 antibody, such as an scFv of an anti-ENPP3 antibody. An exemplary amino acid sequence for a CAR 203 framework includes:

(SEQ ID NO: 121)
KESKYGPPCPPCPIYIWAPLAGTCGVLLLSLVITLYCKRGRKKLLY
IFKQPFMRPVQTTQEEDGCSCRFPEEEEGGCELRVKFSRSADAPAY
QQGQNQLYNELNLGRREEYDVLDKRRGRDPEMGGKPRRKNPQEGLY
NELQKDKMAEAYSEIGMKGERRRGKGHDGLYQGLSTATKDTYDALH
MQALPPR.

### CAR 205 Framework

In some forms, a domain variant CAR includes an antigen binding domain, such as an IgG ScFv, fused to a CAR framework including a polypeptide derived from the extracellular (hinge) domain of human IgG4m, together with a polypeptide derived from the transmembrane region of the human CD28, and a cytoplasmic region derived from the cytoplasmic component of 4-1BB and CD3 zeta. Typically, the framework does not include the antigen binding component, but can be used to form a functional CAR by conjugation with any suitable antigen-specific domain. An exemplary antigen-specific domain is an antigen binding domain of an anti-ENPP3 antibody, such as an scFv of an anti-ENPP3 antibody. An exemplary amino acid sequence for a CAR 205 framework includes:

(SEQ ID NO: 122)
KESKYGPPCPPCPFWVLVVVGGVLACYSLLVTVAFIIFWVKRGRKK
LLYIFKQPFMRPVQTTQEEDGCSCRFPEEEEGGCELRVKFSRSADA
PAYQQGQNQLYNELNLGRREEYDVLDKRRGRDPEMGGKPRRKNPQE
GLYNELQKDKMAEAYSEIGMKGERRRGKGHDGLYQGLSTATKDTYD
ALHMQALPPR.

### CAR 216 Framework

In some forms, a domain variant CAR includes an antigen binding domain, such as an IgG ScFv, fused to a CAR framework including a polypeptide derived from the extracellular (hinge) domain of human CD28, together with a polypeptide derived from the transmembrane region of the human CD28, and a cytoplasmic region derived from the cytoplasmic component of human CD28 and CD3 zeta. Typically, the framework does not include the antigen binding component, but can be used to form a functional CAR by conjugation with any suitable antigen-specific domain. An exemplary antigen-specific domain is an antigen binding domain of an anti-ENPP3 antibody, such as an scFv of an anti-ENPP3 antibody. An exemplary amino acid sequence for a CAR 216 framework includes:

(SEQ ID NO: 123)
KIEVMYPPPYLDNEKSNGTIIHVKGKHLCPSPLFPGPSKPFWVLVV
VGGVLACYSLLVTVAFIIFWVRSKRSRLLHSDYMNMTPRRPGPTRK
HYQPYAPPRDFAAYRSRVKFSRSADAPAYQQGQNQLYNELNLGRRE
EYDVLDKRRGRDPEMGGKPRRKNPQEGLYNELQKDKMAEAYSEIGM
KGERRRGKGHDGLYQGLSTATKDTYDALHMQALPPR.

### CAR 217 Framework

In some forms, a domain variant CAR includes an antigen binding domain, such as an IgG ScFv, fused to a CAR framework including a polypeptide derived from the extracellular (hinge) domain of human IgG4m, together with a polypeptide derived from the transmembrane region of the human CD8 coreceptor, and a cytoplasmic region derived from the cytoplasmic component of 4-1BB and CD3 zeta. Typically, the framework does not include the antigen binding component, but can be used to form a functional CAR by conjugation with any suitable antigen-specific domain. An exemplary antigen-specific domain is an antigen binding domain of an anti-ENPP3 antibody, such as an scFv of an anti-ENPP3 antibody. An exemplary amino acid sequence for a CAR 217 framework includes:

(SEQ ID NO: 124)
KESKYGPPCPSCPIYIWAPLAGTCGVLLLSLVITLYCKRGRKKLLY
IFKQPFMRPVQTTQEEDGCSCRFPEEEEGGCELRVKFSRSADAPAY
QQGQNQLYNELNLGRREEYDVLDKRRGRDPEMGGKPRRKNPQEGLY
NELQKDKMAEAYSEIGMKGERRRGKGHDGLYQGLSTATKDTYDALH
MQALPPR.

### CAR 219 Framework

In some forms, a domain variant CAR includes an antigen binding domain, such as an IgG ScFv, fused to a CAR framework including a polypeptide derived from the extracellular (hinge) domain of human IgG4m, together with a polypeptide derived from the transmembrane region of the human CD28, and a cytoplasmic region derived from the cytoplasmic component of human CD28 and CD3 zeta. Typically, the framework does not include the antigen binding component, but can be used to form a functional CAR by conjugation with any suitable antigen-specific domain. An exemplary antigen-specific domain is an antigen binding domain of an anti-ENPP3 antibody, such as an scFv of an anti-ENPP3 antibody. An exemplary amino acid sequence for a CAR 219 framework includes:

(SEQ ID NO: 125)
KESKYGPPCPSCPFWVLVVVGGVLACYSLLVTVAFIIFWVRSKRSR
LLHSDYMNMTPRRPGPTRKHYQPYAPPRDFAAYRSRVKFSRSADAP
AYQQGQNQLYNELNLGRREEYDVLDKRRGRDPEMGGKPRRKNPQEG
LYNELQKDKMAEAYSEIGMKGERRRGKGHDGLYQGLSTATKDTYDA
LHMQALPPR.

In some forms, the variant domain CARs include CDRs derived from, a scFv of the SX13-7 or SX13-10 antibody, together with a spacer domain from human IgG4, and/or the transmembrane domain of CD8 alpha, fused to the intracellular domain of 4-1BB and CD3 zeta.

For example, in some forms, a variant domain CAR includes all the CDRs of the SX13-7 antibody, such as all the V_{H} CDRs 1-3 including the amino acid sequences set forth in SEQ ID NOs: 46, 36, and 31, and all of the V_{L} CDRs 1-3 including the amino acid sequences set forth in SEQ ID NOs: 52, 53, and 34; in some forms as a CAR including a single chain FV (ScFV) of the SX13-7 antibody, for example, having an amino acid sequence as set forth in SEQ ID NO:23, or a functional variant thereof having at least 80% sequence identity with SEQ ID NO:23, fused to “hinge” domain derived from human IgG4, or a spacer domain derived from human CD-28, fused to a transmembrane domain derived from human CD8 or from human CD28, fused to an extracellular domain including a cytoplasmic domain derived from human 4-1BB or human CD28, and a cytoplasmic domain derived from human CD3 zeta. For example, in some forms, a CAR includes all the CDRs of the SX13-10 antibody, such as all the V_{H} CDRs 1-3 including the amino acid sequences set forth in SEQ ID NOs: 60, 61, and 62, and all of the V_{L} CDRs 1-3 including the amino acid sequences set forth in SEQ ID NOs: 63, 64, and 65; in some forms as a CAR including a single chain FV (ScFV) of the SX13-10 antibody, for example, having an amino acid sequence as set forth in SEQ ID NO:25, or a functional variant thereof having at least 80% sequence identity with SEQ ID NO:25, fused to “hinge” domain derived from human IgG4, or a spacer domain derived from human CD-28, fused to a transmembrane domain derived from human CD8 or from human CD28, fused to an extracellular domain including a cytoplasmic domain derived from human 4-1BB or human CD28, and a cytoplasmic domain derived from human CD3 zeta.

As set forth in the examples, it has been established that variant CAR constructs, including antigen binding components of an ENPP3 specific antibody, optionally ENPP3 specific antibody SX13-7 or ENPP3 specific antibody SX13-10, fused with a polypeptide derived from the extracellular (hinge) domain of human IgG4m, together with a polypeptide derived from the transmembrane region of the human CD8 alpha coreceptor, and a cytoplasmic region derived from the cytoplasmic component of 4-1BB and CD3 zeta is highly effective in multiple T cell donors, and resists exhaustion as well as maintains stemness (see, e.g., construct “PL203” in the Examples). Therefore, in some forms, the CAR includes the antigen binding components of ENPP3 specific antibody SX13-7, such as an ScFv having the amino acid sequence of SEQ ID NO:23, fused with a polypeptide derived from the extracellular (hinge) domain of human IgG4m, together with a polypeptide derived from the transmembrane region of the human CD8 alpha coreceptor, and a cytoplasmic region derived from the cytoplasmic component of 4-1BB and CD3 zeta.

In other forms, the CAR includes the antigen binding components of an ENPP3 specific antibody, optionally antibody SX13-10, such as an ScFv having the amino acid sequence of SEQ ID NO:25, fused with a polypeptide derived from the extracellular (hinge) domain of human IgG4m, together with a polypeptide derived from the transmembrane region of the human CD8 alpha coreceptor, and a cytoplasmic region derived from the cytoplasmic component of 4-1BB and CD3 zeta.

As set forth in the examples, it has been established that variant CAR constructs, including antigen binding components of an ENPP3 specific antibody, optionally ENPP3 specific antibody SX13-7 or ENPP3 specific antibody SX13-10, fused with a polypeptide derived from the extracellular (hinge) domain of human IgG4m, together with a polypeptide derived from the transmembrane region of the human CD28 coreceptor, and a cytoplasmic region derived from the cytoplasmic component the human CD28 coreceptor and a cytoplasmic region derived from CD3 zeta is highly effective in multiple T cell donors, and resists exhaustion as well as maintains stemness (see, e.g., construct “PL219” in the Examples).

Therefore, in some forms, the CAR includes the antigen binding components of an ENPP3 specific antibody, optionally antibody SX13-7, such as an ScFv having the amino acid sequence of SEQ ID NO:23, fused with a polypeptide derived from the extracellular (hinge) domain of human IgG4m, together with a polypeptide derived from the transmembrane region of the human CD28 coreceptor, and a cytoplasmic region derived from the cytoplasmic component of the human CD28 coreceptor and a cytoplasmic region derived from the cytoplasmic component of human CD3 zeta.

In other forms, the CAR includes the antigen binding components of an ENPP3 specific antibody, optionally antibody SX13-10, such as an ScFv having the amino acid sequence of SEQ ID NO:25, fused with a polypeptide derived from the extracellular (hinge) domain of human IgG4m, together with a polypeptide derived from the transmembrane region of the human CD28 coreceptor, and a cytoplasmic region derived from the cytoplasmic component of the human CD28 coreceptor and a cytoplasmic region derived from the cytoplasmic component of human CD3 zeta.

As set forth in the examples, it has been established that variant CAR constructs, including antigen binding components of an ENPP3 specific antibody, optionally antibody SX13-7 or ENPP3 specific antibody SX13-10, fused with a polypeptide derived from the extracellular (hinge) domain of human IgG4m, together with a polypeptide derived from the transmembrane region of the human CD8 coreceptor, and a cytoplasmic region derived from the cytoplasmic component of the human 4-1BB coreceptor and a cytoplasmic region derived from the cytoplasmic region of human CD3 zeta is highly effective in multiple T cell donors, and resists exhaustion as well as maintains stemness (see, e.g., construct “PL217” in the Examples).

Therefore, in some forms, the CAR includes the antigen binding components of an ENPP3 specific antibody, optionally antibody SX13-7, such as an ScFv having the amino acid sequence of SEQ ID NO:23, fused with a polypeptide derived from the extracellular (hinge) domain of human IgG4m, together with a polypeptide derived from the transmembrane region of the human CD8 coreceptor, and a cytoplasmic region derived from the cytoplasmic component of the human 4-1BB coreceptor and a cytoplasmic region derived from the cytoplasmic region of human CD3 zeta. In other forms, the CAR includes the antigen binding components of an ENPP3 specific antibody, optionally antibody SX13-10, such as an ScFv having the amino acid sequence of SEQ ID NO:25, fused with a polypeptide derived from the extracellular (hinge) domain of human IgG4m, together with a polypeptide derived from the transmembrane region of the human CD8 coreceptor, and a cytoplasmic region derived from the cytoplasmic component of the human 4-1BB coreceptor and a cytoplasmic region derived from the cytoplasmic region of human CD3 zeta. a.

a. CAR PL199 (Antibody SX13-7)

In some forms, a CAR including an scFv derived from the anti-ENPP3 antibody SX13-7 includes a spacer polypeptide derived from CD28, a transmembrane domain derived from CD28, and a cytoplasmic region derived from the cytoplasmic component of the human 4-1BB coreceptor and a from the cytoplasmic region of human CD3 zeta (see, e.g., “PL199” demonstrated in the Examples).

An exemplary nucleic acid sequence for a PL199 CAR
including an scFv derived from the anti-ENPP3 antibody
SX13-7 is:
(SEQ ID NO: 108)
ATGGCCTTACCAGTGACCGCCTTGCTCCTGCCGCTGGCCTTGCTGCTCCACGCCGCCAG
GCCGGATTACAAAGACGATGACGATAAGCAGCTGGTGGAGTCTGGGGGAGGCCTGGTCA
AGCCTGGGGGGTCCCTGAGACTCTCCTGTGCAGCCTCTGGATTCACCTTCAGTGACTAT
AGCATGAACTGGGTCCGCCAGGCTCCAGGGAAGGGGCTGGAGTGGGTCTCATCCATTAG
TAGTAGTAGTAGTTACAAAAAGTACGCAGACTCAGTGAAGGGCCGCCTCACCATCTCCA
GAGACAACGCCAAGAATTCATTGTATCTGCAGATGAACAGCCTGAGAGCCGAGGACACA
GCTGTGTATTACTGTGCGAGAGGGATTGACTACTTTGATTACTGGGGCCAGGGAACCCT
GGTCACCGTGTCCTCAAGTGGTGGCGGTGGCTCGGGCGGTGGTGGGTCGGGTGGCGGAG
GCTCTGACATCCAGTTGACCCAGTCTCCATCCTTCCTGTCTGCATCTGTAGGAGACAGA
GTCACCATCACTTGCCGGGCCAGTCAGGGCATTAGCAGTAACTTAGCCTGGTATCAGCA
GAAACCAGGGAAAGACCCTAAGGTCCTGATCTATGGTGCATCCACTTTGCAAAGTGGGG
TCCCATCAAGGTTCAGCGGCAGTGGATCTGGGACAGAATTCACTCTCACAATCAGCAGC
CTGCAGCCTGAAGATTTTGCAACTTATTACTGTCAACAGCTTAATAGTTACCCGAGGAC
GTTCGGCCAAGGGACCAAGGTGGAAATCAAAATTGAAGTTATGTATCCTCCTCCTTACC
TAGACAATGAGAAGAGCAATGGAACCATTATCCATGTGAAAGGGAAACACCTTTGTCCA
AGTCCCCTATTTCCCGGACCTTCTAAGCCCTTTTGGGTGCTGGTGGTGGTTGGTGGAGT
CCTGGCTTGCTATAGCTTGCTAGTAACAGTGGCCTTTATTATTTTCTGGGTGAAACGGG
GCAGAAAGAAACTCCTGTATATATTCAAACAACCATTTATGAGACCAGTACAAACTACT
CAAGAGGAAGATGGCTGTAGCTGCCGATTTCCAGAAGAAGAAGAAGGAGGATGTGAACT
GAGAGTGAAGTTCAGCAGGAGCGCAGACGCCCCCGCGTACCAGCAGGGCCAGAACCAGC
TCTATAACGAGCTCAATCTAGGACGAAGAGAGGAGTACGATGTTTTGGACAAGCGACGT
GGCCGGGACCCTGAGATGGGGGGAAAGCCGAGAAGGAAGAACCCTCAGGAAGGCCTGTA
CAATGAACTGCAGAAAGATAAGATGGCGGAGGCCTACAGTGAGATTGGGATGAAAGGCG
AGCGCCGGAGGGGCAAGGGGCACGATGGCCTTTACCAGGGTCTCAGTACAGCCACCAAG
GACACCTACGACGCCCTTCACATGCAGGCCCTGCCCCCTCGCTGA.
An exemplary amino acid sequence for a PL199 CAR including
an scFv derived from the anti-ENPP3 antibody SX13-7 is:
(SEQ ID NO: 109)
MALPVTALLLPLALLLHAARPDYKDDDDKQLVESGGGLVKPGGSLRLSCAASGFTFSDY
SMNWVRQAPGKGLEWVSSISSSSSYKKYADSVKGRLTISRDNAKNSLYLQMNSLRAEDT
AVYYCARGIDYFDYWGQGTLVTVSSSGGGGSGGGGSGGGGSDIQLTQSPSFLSASVGDR
VTITCRASQGISSNLAWYQQKPGKDPKVLIYGASTLQSGVPSRFSGSGSGTEFTLTISS
LQPEDFATYYCQQLNSYPRTFGQGTKVEIKIEVMYPPPYLDNEKSNGTIIHVKGKHLCP
SPLFPGPSKPFWVLVVVGGVLACYSLLVTVAFIIFWVKRGRKKLLYIFKQPFMRPVQTT
QEEDGCSCRFPEEEEGGCELRVKFSRSADAPAYQQGQNQLYNELNLGRREEYDVLDKRR
GRDPEMGGKPRRKNPQEGLYNELQKDKMAEAYSEIGMKGERRRGKGHDGLYQGLSTATK
DTYDALHMQALPPR.

In some forms, the disclosed CAR of SEQ ID NO: 109 binds to an epitope on a protein having an amino acid sequence set forth in SEQ ID NO:1 and/or SEQ ID NO:2, or a homologue, paralogue, or ortholog thereof. In some forms, the CAR is a variant having at least 70%, up to 99% identity to SEQ ID NO:109. For example, in some forms, the variant sequence has at least about 70%, 75%, 80%, 85%, 90%, or 95% identity to SEQ ID NO:109. Typically, the variant CAR has an amino acid sequence that has one or more amino acids different to SEQ ID NO:109, such as one or more substitutions, deletions or additions at any one of the amino acid positions of SEQ ID NO:109, but retains the ability to specifically bind to an epitope on a protein having an amino acid sequence set forth in SEQ ID NO:1 and/or SEQ ID NO:2.

b. CAR PL203 (Antibody SX13-7)

In some forms, a CAR including an scFv derived from the anti-ENPP3 antibody SX13-7 includes an extracellular (hinge) domain of human IgG4m, together with a polypeptide derived from the transmembrane region of the human CD8 coreceptor, and a cytoplasmic region derived from the cytoplasmic component of the human 4-1BB coreceptor and a cytoplasmic region derived from human CD3 zeta (see, e.g., “PL203” demonstrated in the Examples).

An exemplary nucleic acid sequence for a PL203 CAR
including an scFv derived from the anti-ENPP3 antibody
SX13-7 is:
(SEQ ID NO: 110)
ATGGCCTTACCAGTGACCGCCTTGCTCCTGCCGCTGGCCTTGCTGCTCCACGCCGCCAG
GCCGGATTACAAAGACGATGACGATAAGCAGCTGGTGGAGTCTGGGGGAGGCCTGGTCA
AGCCTGGGGGGTCCCTGAGACTCTCCTGTGCAGCCTCTGGATTCACCTTCAGTGACTAT
AGCATGAACTGGGTCCGCCAGGCTCCAGGGAAGGGGCTGGAGTGGGTCTCATCCATTAG
TAGTAGTAGTAGTTACAAAAAGTACGCAGACTCAGTGAAGGGCCGCCTCACCATCTCCA
GAGACAACGCCAAGAATTCATTGTATCTGCAGATGAACAGCCTGAGAGCCGAGGACACA
GCTGTGTATTACTGTGCGAGAGGGATTGACTACTTTGATTACTGGGGCCAGGGAACCCT
GGTCACCGTGTCCTCAAGTGGTGGCGGTGGCTCGGGCGGTGGTGGGTCGGGTGGCGGAG
GCTCTGACATCCAGTTGACCCAGTCTCCATCCTTCCTGTCTGCATCTGTAGGAGACAGA
GTCACCATCACTTGCCGGGCCAGTCAGGGCATTAGCAGTAACTTAGCCTGGTATCAGCA
GAAACCAGGGAAAGACCCTAAGGTCCTGATCTATGGTGCATCCACTTTGCAAAGTGGGG
TCCCATCAAGGTTCAGCGGCAGTGGATCTGGGACAGAATTCACTCTCACAATCAGCAGC
CTGCAGCCTGAAGATTTTGCAACTTATTACTGTCAACAGCTTAATAGTTACCCGAGGAC
GTTCGGCCAAGGGACCAAGGTGGAAATCAAAGAGTCCAAATATGGTCCCCCATGCCCAC
CCTGCCCAATCTACATCTGGGCGCCCTTGGCCGGGACTTGTGGGGTCCTTCTCCTGTCA
CTGGTTATCACCCTTTACTGCAAACGGGGCAGAAAGAAACTCCTGTATATATTCAAACA
ACCATTTATGAGACCAGTACAAACTACTCAAGAGGAAGATGGCTGTAGCTGCCGATTTC
CAGAAGAAGAAGAAGGAGGATGTGAACTGAGAGTGAAGTTCAGCAGGAGCGCAGACGCC
CCCGCGTACCAGCAGGGCCAGAACCAGCTCTATAACGAGCTCAATCTAGGACGAAGAGA
GGAGTACGATGTTTTGGACAAGCGACGTGGCCGGGACCCTGAGATGGGGGGAAAGCCGA
GAAGGAAGAACCCTCAGGAAGGCCTGTACAATGAACTGCAGAAAGATAAGATGGCGGAG
GCCTACAGTGAGATTGGGATGAAAGGCGAGCGCCGGAGGGGCAAGGGGCACGATGGCCT
TTACCAGGGTCTCAGTACAGCCACCAAGGACACCTACGACGCCCTTCACATGCAGGCCC
TGCCCCCTCGCTGA.
An exemplary amino acid sequence for a PL203 CAR including
an scFv derived from the anti-ENPP3 antibody SX13-7 is:
(SEQ ID NO: 111)
MALPVTALLLPLALLLHAARPDYKDDDDKQLVESGGGLVKPGGSLRLSCAASGFTFSDY
SMNWVRQAPGKGLEWVSSISSSSSYKKYADSVKGRLTISRDNAKNSLYLQMNSLRAEDT
AVYYCARGIDYFDYWGQGTLVTVSSSGGGGSGGGGSGGGGSDIQLTQSPSFLSASVGDR
VTITCRASQGISSNLAWYQQKPGKDPKVLIYGASTLQSGVPSRFSGSGSGTEFTLTISS
LQPEDFATYYCQQLNSYPRTFGQGTKVEIKESKYGPPCPPCPIYIWAPLAGTCGVLLLS
LVITLYCKRGRKKLLYIFKQPFMRPVQTTQEEDGCSCRFPEEEEGGCELRVKFSRSADA
PAYQQGQNQLYNELNLGRREEYDVLDKRRGRDPEMGGKPRRKNPQEGLYNELQKDKMAE
AYSEIGMKGERRRGKGHDGLYQGLSTATKDTYDALHMQALPPR.

In some forms, the disclosed CAR of SEQ ID NO: 111 binds to an epitope on a protein having an amino acid sequence set forth in SEQ ID NO:1 and/or SEQ ID NO:2, or a homologue, paralogue, or ortholog thereof. In some forms, the CAR is a variant having at least 70%, up to 99% identity to SEQ ID NO:111. For example in some forms, the variant sequence has at least about 70%, 75%, 80%, 85%, 90%, or 95% identity to SEQ ID NO:111. Typically, the variant CAR has an amino acid sequence that has one or more amino acids different to SEQ ID NO:111, such as one or more substitutions, deletions or additions at any one of the amino acid positions of SEQ ID NO:111, but retains the ability to specifically bind to an epitope on a protein having an amino acid sequence set forth in SEQ ID NO:1 and/or SEQ ID NO:2.

c. CAR PL205 (Antibody SX13-7)

In some forms, a CAR including an scFv derived from the anti-ENPP3 antibody SX13-7 includes an extracellular (hinge) domain of human IgG4m, together with a polypeptide derived from the transmembrane region of the human CD28, and a cytoplasmic region derived from the cytoplasmic component of the human 4-1BB coreceptor and a cytoplasmic region derived from human CD3 zeta (see, e.g., “PL205” demonstrated in the Examples).

An exemplary nucleic acid sequence for a PL205 CAR
including an scFv derived from the anti-ENPP3 antibody
SX13-7 is:
(SEQ ID NO: 112)
ATGGCCTTACCAGTGACCGCCTTGCTCCTGCCGCTGGCCTTGCTGCTCCACGCCGCCAG
GCCGGATTACAAAGACGATGACGATAAGCAGCTGGTGGAGTCTGGGGGAGGCCTGGTCA
AGCCTGGGGGGTCCCTGAGACTCTCCTGTGCAGCCTCTGGATTCACCTTCAGTGACTAT
AGCATGAACTGGGTCCGCCAGGCTCCAGGGAAGGGGCTGGAGTGGGTCTCATCCATTAG
TAGTAGTAGTAGTTACAAAAAGTACGCAGACTCAGTGAAGGGCCGCCTCACCATCTCCA
GAGACAACGCCAAGAATTCATTGTATCTGCAGATGAACAGCCTGAGAGCCGAGGACACA
GCTGTGTATTACTGTGCGAGAGGGATTGACTACTTTGATTACTGGGGCCAGGGAACCCT
GGTCACCGTGTCCTCAAGTGGTGGCGGTGGCTCGGGCGGTGGTGGGTCGGGTGGCGGAG
GCTCTGACATCCAGTTGACCCAGTCTCCATCCTTCCTGTCTGCATCTGTAGGAGACAGA
GTCACCATCACTTGCCGGGCCAGTCAGGGCATTAGCAGTAACTTAGCCTGGTATCAGCA
GAAACCAGGGAAAGACCCTAAGGTCCTGATCTATGGTGCATCCACTTTGCAAAGTGGGG
TCCCATCAAGGTTCAGCGGCAGTGGATCTGGGACAGAATTCACTCTCACAATCAGCAGC
CTGCAGCCTGAAGATTTTGCAACTTATTACTGTCAACAGCTTAATAGTTACCCGAGGAC
GTTCGGCCAAGGGACCAAGGTGGAAATCAAAGAGTCCAAATATGGTCCCCCATGCCCAC
CCTGCCCATTTTGGGTGCTGGTGGTGGTTGGTGGAGTCCTGGCTTGCTATAGCTTGCTA
GTAACAGTGGCCTTTATTATTTTCTGGGTGAAACGGGGCAGAAAGAAACTCCTGTATAT
ATTCAAACAACCATTTATGAGACCAGTACAAACTACTCAAGAGGAAGATGGCTGTAGCT
GCCGATTTCCAGAAGAAGAAGAAGGAGGATGTGAACTGAGAGTGAAGTTCAGCAGGAGC
GCAGACGCCCCCGCGTACCAGCAGGGCCAGAACCAGCTCTATAACGAGCTCAATCTAGG
ACGAAGAGAGGAGTACGATGTTTTGGACAAGCGACGTGGCCGGGACCCTGAGATGGGGG
GAAAGCCGAGAAGGAAGAACCCTCAGGAAGGCCTGTACAATGAACTGCAGAAAGATAAG
ATGGCGGAGGCCTACAGTGAGATTGGGATGAAAGGCGAGCGCCGGAGGGGCAAGGGGCA
CGATGGCCTTTACCAGGGTCTCAGTACAGCCACCAAGGACACCTACGACGCCCTTCACA
TGCAGGCCCTGCCCCCTCGCTGA.
An exemplary amino acid sequence for a PL205 CAR including
an scFv derived from the anti-ENPP3 antibody SX13-7 is:
(SEQ ID NO: 113)
MALPVTALLLPLALLLHAARPDYKDDDDKQLVESGGGLVKPGGSLRLSCAASGFTFSDY
SMNWVRQAPGKGLEWVSSISSSSSYKKYADSVKGRLTISRDNAKNSLYLQMNSLRAEDT
AVYYCARGIDYFDYWGQGTLVTVSSSGGGGSGGGGSGGGGSDIQLTQSPSFLSASVGDR
VTITCRASQGISSNLAWYQQKPGKDPKVLIYGASTLQSGVPSRFSGSGSGTEFTLTISS
LQPEDFATYYCQQLNSYPRTFGQGTKVEIKESKYGPPCPPCPFWVLVVVGGVLACYSLL
VTVAFIIFWVKRGRKKLLYIFKQPFMRPVQTTQEEDGCSCRFPEEEEGGCELRVKFSRS
ADAPAYQQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPR.

In some forms, the disclosed CAR of SEQ ID NO: 113 binds to an epitope on a protein having an amino acid sequence set forth in SEQ ID NO:1 and/or SEQ ID NO:2, or a homologue, paralogue, or ortholog thereof. In some forms, the CAR is a variant having at least 70%, up to 99% identity to SEQ ID NO:113. For example, in some forms, the variant sequence has at least about 70%, 75%, 80%, 85%, 90%, or 95% identity to SEQ ID NO:113. Typically, the variant CAR has an amino acid sequence that has one or more amino acids different to SEQ ID NO:113, such as one or more substitutions, deletions or additions at any one of the amino acid positions of SEQ ID NO:113, but retains the ability to specifically bind to an epitope on a protein having an amino acid sequence set forth in SEQ ID NO:1 and/or SEQ ID NO:2.

d. CAR PL216 (Antibody SX13-7)

In some forms, a CAR including an scFv derived from the anti-ENPP3 antibody SX13-7 includes an extracellular (hinge) domain of human CD28, together with a polypeptide derived from the transmembrane region of the human CD28, and a cytoplasmic region derived from the cytoplasmic component of the human CD28 coreceptor and a cytoplasmic region derived from human CD3 zeta (see, e.g., “PL16” demonstrated in the Examples).

An exemplary nucleic acid sequence for a PL216 CAR
including an scFv derived from the anti-ENPP3 antibody
SX13-7 is:
(SEQ ID NO: 114)
ATGGCCTTACCAGTGACCGCCTTGCTCCTGCCGCTGGCCTTGCTGCTCCACGCCGCCAG
GCCGGATTACAAAGACGATGACGATAAGCAGCTGGTGGAGTCTGGGGGAGGCCTGGTCA
AGCCTGGGGGGTCCCTGAGACTCTCCTGTGCAGCCTCTGGATTCACCTTCAGTGACTAT
AGCATGAACTGGGTCCGCCAGGCTCCAGGGAAGGGGCTGGAGTGGGTCTCATCCATTAG
TAGTAGTAGTAGTTACAAAAAGTACGCAGACTCAGTGAAGGGCCGCCTCACCATCTCCA
GAGACAACGCCAAGAATTCATTGTATCTGCAGATGAACAGCCTGAGAGCCGAGGACACA
GCTGTGTATTACTGTGCGAGAGGGATTGACTACTTTGATTACTGGGGCCAGGGAACCCT
GGTCACCGTGTCCTCAAGTGGTGGCGGTGGCTCGGGCGGTGGTGGGTCGGGTGGCGGAG
GCTCTGACATCCAGTTGACCCAGTCTCCATCCTTCCTGTCTGCATCTGTAGGAGACAGA
GTCACCATCACTTGCCGGGCCAGTCAGGGCATTAGCAGTAACTTAGCCTGGTATCAGCA
GAAACCAGGGAAAGACCCTAAGGTCCTGATCTATGGTGCATCCACTTTGCAAAGTGGGG
TCCCATCAAGGTTCAGCGGCAGTGGATCTGGGACAGAATTCACTCTCACAATCAGCAGC
CTGCAGCCTGAAGATTTTGCAACTTATTACTGTCAACAGCTTAATAGTTACCCGAGGAC
GTTCGGCCAAGGGACCAAGGTGGAAATCAAAATTGAAGTTATGTATCCTCCTCCTTACC
TAGACAATGAGAAGAGCAATGGAACCATTATCCATGTGAAAGGGAAACACCTTTGTCCA
AGTCCCCTATTTCCCGGACCTTCTAAGCCCTTTTGGGTGCTGGTGGTGGTTGGTGGAGT
CCTGGCTTGCTATAGCTTGCTAGTAACAGTGGCCTTTATTATTTTCTGGGTGAGGAGTA
AGAGGAGCAGGCTCCTGCACAGTGACTACATGAACATGACTCCCCGCCGCCCCGGGCCC
ACCCGCAAGCATTACCAGCCCTATGCCCCACCACGCGACTTCGCAGCCTATCGCTCCAG
AGTGAAGTTCAGCAGGAGCGCAGACGCCCCCGCGTACCAGCAGGGCCAGAACCAGCTCT
ATAACGAGCTCAATCTAGGACGAAGAGAGGAGTACGATGTTTTGGACAAGCGACGTGGC
CGGGACCCTGAGATGGGGGGAAAGCCGAGAAGGAAGAACCCTCAGGAAGGCCTGTACAA
TGAACTGCAGAAAGATAAGATGGCGGAGGCCTACAGTGAGATTGGGATGAAAGGCGAGC
GCCGGAGGGGCAAGGGGCACGATGGCCTTTACCAGGGTCTCAGTACAGCCACCAAGGAC
ACCTACGACGCCCTTCACATGCAGGCCCTGCCCCCTCGCTGA.
An exemplary amino acid sequence for a PL216 CAR including
an scFv derived from the anti-ENPP3 antibody SX13-7 is:
(SEQ ID NO: 115)
MALPVTALLLPLALLLHAARPDYKDDDDKQLVESGGGLVKPGGSLRLSCAASGFTFSDY
SMNWVRQAPGKGLEWVSSISSSSSYKKYADSVKGRLTISRDNAKNSLYLQMNSLRAEDT
AVYYCARGIDYFDYWGQGTLVTVSSSGGGGSGGGGSGGGGSDIQLTQSPSFLSASVGDR
VTITCRASQGISSNLAWYQQKPGKDPKVLIYGASTLQSGVPSRFSGSGSGTEFTLTISS
LQPEDFATYYCQQLNSYPRTFGQGTKVEIKIEVMYPPPYLDNEKSNGTIIHVKGKHLCP
SPLFPGPSKPFWVLVVVGGVLACYSLLVTVAFIIFWVRSKRSRLLHSDYMNMTPRRPGP
TRKHYQPYAPPRDFAAYRSRVKFSRSADAPAYQQGQNQLYNELNLGRREEYDVLDKRRG
RDPEMGGKPRRKNPQEGLYNELQKDKMAEAYSEIGMKGERRRGKGHDGLYQGLSTATKD
TYDALHMQALPPR.

In some forms, the disclosed CAR of SEQ ID NO: 115 binds to an epitope on a protein having an amino acid sequence set forth in SEQ ID NO: 1 and/or SEQ ID NO:2, or a homologue, paralogue, or ortholog thereof. In some forms, the CAR is a variant having at least 70%, up to 99% identity to SEQ ID NO:115. For example, in some forms, the variant sequence has at least about 70%, 75%, 80%, 85%, 90%, or 95% identity to SEQ ID NO:115. Typically, the variant CAR has an amino acid sequence that has one or more amino acids different to SEQ ID NO:115, such as one or more substitutions, deletions or additions at any one of the amino acid positions of SEQ ID NO:115, but retains the ability to specifically bind to an epitope on a protein having an amino acid sequence set forth in SEQ ID NO:1 and/or SEQ ID NO:2.

e. CAR PL217 (Antibody SX13-7)

In some forms, a CAR includes an scFv derived from the anti-ENPP3 antibody SX13-7 including an extracellular (hinge) domain of human IgG4, together with a polypeptide derived from the transmembrane region of the human CD8, and a cytoplasmic region derived from the cytoplasmic component of the human 4-1BB coreceptor and a cytoplasmic region derived from the human CD3 zeta (see, e.g., “PL217” demonstrated in the Examples).

An exemplary nucleic acid sequence for a PL217 CAR
including an scFv derived from the anti-ENPP3 antibody
SX13-7 is:
(SEQ ID NO: 116)
ATGGCCTTACCAGTGACCGCCTTGCTCCTGCCGCTGGCCTTGCTGCTCCACGCCGCCAG
GCCGGATTACAAAGACGATGACGATAAGCAGCTGGTGGAGTCTGGGGGAGGCCTGGTCA
AGCCTGGGGGGTCCCTGAGACTCTCCTGTGCAGCCTCTGGATTCACCTTCAGTGACTAT
AGCATGAACTGGGTCCGCCAGGCTCCAGGGAAGGGGCTGGAGTGGGTCTCATCCATTAG
TAGTAGTAGTAGTTACAAAAAGTACGCAGACTCAGTGAAGGGCCGCCTCACCATCTCCA
GAGACAACGCCAAGAATTCATTGTATCTGCAGATGAACAGCCTGAGAGCCGAGGACACA
GCTGTGTATTACTGTGCGAGAGGGATTGACTACTTTGATTACTGGGGCCAGGGAACCCT
GGTCACCGTGTCCTCAAGTGGTGGCGGTGGCTCGGGCGGTGGTGGGTCGGGTGGCGGAG
GCTCTGACATCCAGTTGACCCAGTCTCCATCCTTCCTGTCTGCATCTGTAGGAGACAGA
GTCACCATCACTTGCCGGGCCAGTCAGGGCATTAGCAGTAACTTAGCCTGGTATCAGCA
GAAACCAGGGAAAGACCCTAAGGTCCTGATCTATGGTGCATCCACTTTGCAAAGTGGGG
TCCCATCAAGGTTCAGCGGCAGTGGATCTGGGACAGAATTCACTCTCACAATCAGCAGC
CTGCAGCCTGAAGATTTTGCAACTTATTACTGTCAACAGCTTAATAGTTACCCGAGGAC
GTTCGGCCAAGGGACCAAGGTGGAAATCAAAGAGTCCAAATATGGTCCCCCATGCCCAT
CATGCCCAATCTACATCTGGGCGCCCTTGGCCGGGACTTGTGGGGTCCTTCTCCTGTCA
CTGGTTATCACCCTTTACTGCAAACGGGGCAGAAAGAAACTCCTGTATATATTCAAACA
ACCATTTATGAGACCAGTACAAACTACTCAAGAGGAAGATGGCTGTAGCTGCCGATTTC
CAGAAGAAGAAGAAGGAGGATGTGAACTGAGAGTGAAGTTCAGCAGGAGCGCAGACGCC
CCCGCGTACCAGCAGGGCCAGAACCAGCTCTATAACGAGCTCAATCTAGGACGAAGAGA
GGAGTACGATGTTTTGGACAAGCGACGTGGCCGGGACCCTGAGATGGGGGGAAAGCCGA
GAAGGAAGAACCCTCAGGAAGGCCTGTACAATGAACTGCAGAAAGATAAGATGGCGGAG
GCCTACAGTGAGATTGGGATGAAAGGCGAGCGCCGGAGGGGCAAGGGGCACGATGGCCT
TTACCAGGGTCTCAGTACAGCCACCAAGGACACCTACGACGCCCTTCACATGCAGGCCC
TGCCCCCTCGCTGA.
An exemplary amino acid sequence for a PL217 CAR including
an scFv derived from the anti-ENPP3 antibody SX13-7 is:
(SEQ ID NO: 117)
MALPVTALLLPLALLLHAARPDYKDDDDKQLVESGGGLVKPGGSLRLSCAASGFTFSDY
SMNWVRQAPGKGLEWVSSISSSSSYKKYADSVKGRLTISRDNAKNSLYLQMNSLRAEDT
AVYYCARGIDYFDYWGQGTLVTVSSSGGGGSGGGGSGGGGSDIQLTQSPSFLSASVGDR
VTITCRASQGISSNLAWYQQKPGKDPKVLIYGASTLQSGVPSRFSGSGSGTEFTLTISS
LQPEDFATYYCQQLNSYPRTFGQGTKVEIKESKYGPPCPSCPIYIWAPLAGTCGVLLLS
LVITLYCKRGRKKLLYIFKQPFMRPVQTTQEEDGCSCRFPEEEEGGCELRVKFSRSADA
PAYQQGQNQLYNELNLGRREEYDVLDKRRGRDPEMGGKPRRKNPQEGLYNELQKDKMAE
AYSEIGMKGERRRGKGHDGLYQGLSTATKDTYDALHMQALPPR.

In some forms, the disclosed CAR of SEQ ID NO: 117 binds to an epitope on a protein having an amino acid sequence set forth in SEQ ID NO:1 and/or SEQ ID NO:2, or a homologue, paralogue, or ortholog thereof. In some forms, the CAR is a variant having at least 70%, up to 99% identity to SEQ ID NO:117. For example, in some forms, the variant sequence has at least about 70%, 75%, 80%, 85%, 90%, or 95% identity to SEQ ID NO:117. Typically, the variant CAR has an amino acid sequence that has one or more amino acids different to SEQ ID NO:117, such as one or more substitutions, deletions or additions at any one of the amino acid positions of SEQ ID NO:117, but retains the ability to specifically bind to an epitope on a protein having an amino acid sequence set forth in SEQ ID NO:1 and/or SEQ ID NO:2.

f. CAR PL219 (Antibody SX13-7)

In some forms, a CAR includes an scFv derived from the anti-ENPP3 antibody SX13-7 including an extracellular (hinge) domain of human IgG4, together with a polypeptide derived from the transmembrane region of the human CD28, and a cytoplasmic region derived from the cytoplasmic component of the human CD28 coreceptor and a cytoplasmic region derived from human CD3 zeta (see, e.g., “PL219” demonstrated in the Examples).

An exemplary nucleic acid sequence for a PL219 CAR
including an scFv derived from the anti-ENPP3 antibody
SX13-7 is:
(SEQ ID NO: 118)
ATGGCCTTACCAGTGACCGCCTTGCTCCTGCCGCTGGCCTTGCTGCTCCACGCCGCCAG
GCCGGATTACAAAGACGATGACGATAAGCAGCTGGTGGAGTCTGGGGGAGGCCTGGTCA
AGCCTGGGGGGTCCCTGAGACTCTCCTGTGCAGCCTCTGGATTCACCTTCAGTGACTAT
AGCATGAACTGGGTCCGCCAGGCTCCAGGGAAGGGGCTGGAGTGGGTCTCATCCATTAG
TAGTAGTAGTAGTTACAAAAAGTACGCAGACTCAGTGAAGGGCCGCCTCACCATCTCCA
GAGACAACGCCAAGAATTCATTGTATCTGCAGATGAACAGCCTGAGAGCCGAGGACACA
GCTGTGTATTACTGTGCGAGAGGGATTGACTACTTTGATTACTGGGGCCAGGGAACCCT
GGTCACCGTGTCCTCAAGTGGTGGCGGTGGCTCGGGCGGTGGTGGGTCGGGTGGCGGAG
GCTCTGACATCCAGTTGACCCAGTCTCCATCCTTCCTGTCTGCATCTGTAGGAGACAGA
GTCACCATCACTTGCCGGGCCAGTCAGGGCATTAGCAGTAACTTAGCCTGGTATCAGCA
GAAACCAGGGAAAGACCCTAAGGTCCTGATCTATGGTGCATCCACTTTGCAAAGTGGGG
TCCCATCAAGGTTCAGCGGCAGTGGATCTGGGACAGAATTCACTCTCACAATCAGCAGC
CTGCAGCCTGAAGATTTTGCAACTTATTACTGTCAACAGCTTAATAGTTACCCGAGGAC
GTTCGGCCAAGGGACCAAGGTGGAAATCAAAGAGTCCAAATATGGTCCCCCATGCCCAT
CATGCCCATTTTGGGTGCTGGTGGTGGTTGGTGGAGTCCTGGCTTGCTATAGCTTGCTA
GTAACAGTGGCCTTTATTATTTTCTGGGTGAGGAGTAAGAGGAGCAGGCTCCTGCACAG
TGACTACATGAACATGACTCCCCGCCGCCCCGGGCCCACCCGCAAGCATTACCAGCCCT
ATGCCCCACCACGCGACTTCGCAGCCTATCGCTCCAGAGTGAAGTTCAGCAGGAGCGCA
GACGCCCCCGCGTACCAGCAGGGCCAGAACCAGCTCTATAACGAGCTCAATCTAGGACG
AAGAGAGGAGTACGATGTTTTGGACAAGCGACGTGGCCGGGACCCTGAGATGGGGGGAA
AGCCGAGAAGGAAGAACCCTCAGGAAGGCCTGTACAATGAACTGCAGAAAGATAAGATG
GCGGAGGCCTACAGTGAGATTGGGATGAAAGGCGAGCGCCGGAGGGGCAAGGGGCACGA
TGGCCTTTACCAGGGTCTCAGTACAGCCACCAAGGACACCTACGACGCCCTTCACATGC
AGGCCCTGCCCCCTCGCTGA.
An exemplary amino acid sequence for a PL219 CAR including
an scFv derived from the anti-ENPP3 antibody SX13-7 is:
(SEQ ID NO: 119)
MALPVTALLLPLALLLHAARPDYKDDDDKQLVESGGGLVKPGGSLRLSCAASGFTFSDY
SMNWVRQAPGKGLEWVSSISSSSSYKKYADSVKGRLTISRDNAKNSLYLQMNSLRAEDT
AVYYCARGIDYFDYWGQGTLVTVSSSGGGGSGGGGSGGGGSDIQLTQSPSFLSASVGDR
VTITCRASQGISSNLAWYQQKPGKDPKVLIYGASTLQSGVPSRFSGSGSGTEFTLTISS
LQPEDFATYYCQQLNSYPRTFGQGTKVEIKESKYGPPCPSCPFWVLVVVGGVLACYSLL
VTVAFIIFWVRSKRSRLLHSDYMNMTPRRPGPTRKHYQPYAPPRDFAAYRSRVKFSRSA
DAPAYQQGQNQLYNELNLGRREEYDVLDKRRGRDPEMGGKPRRKNPQEGLYNELQKDKM
AEAYSEIGMKGERRRGKGHDGLYQGLSTATKDTYDALHMQALPPR.

In some forms, the disclosed CAR of SEQ ID NO:119 binds to an epitope on a protein having an amino acid sequence set forth in SEQ ID NO:1 and/or SEQ ID NO:2, or a homologue, paralogue, or ortholog thereof. In some forms, the CAR is a variant having at least 70%, up to 99% identity to SEQ ID NO:119. For example, in some forms, the variant sequence has at least about 70%, 75%, 80%, 85%, 90%, or 95% identity to SEQ ID NO:119. Typically, the variant CAR has an amino acid sequence that has one or more amino acids different to SEQ ID NO:119, such as one or more substitutions, deletions or additions at any one of the amino acid positions of SEQ ID NO:119, but retains the ability to specifically bind to an epitope on a protein having an amino acid sequence set forth in SEQ ID NO:1 and/or SEQ ID NO:2.

### F. Nucleic Acids

Nucleic acids and vectors encoding or expressing the disclosed polypeptides, including but not limited to anti-ENPP3 antibodies and antigen binding domains thereof, and fusion constructs formed therefrom such as CAR are also described.

### 1. Isolated Nucleic Acid Molecules

Isolated nucleic acids encoding the disclosed polypeptides are disclosed. The term “isolated nucleic acid” refers to a nucleic acid that is separated from other nucleic acid molecules that are present in a mammalian genome, including nucleic acids that normally flank one or both sides of the nucleic acid in a mammalian genome. An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule independent of other sequences (e.g., a chemically synthesized nucleic acid, or a cDNA or genomic DNA fragment produced by PCR or restriction endonuclease treatment), as well as recombinant DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, a cDNA library or a genomic library, or a gel slice containing a genomic DNA restriction digest, is not to be considered an isolated nucleic acid.

Nucleic acids can be in sense or antisense orientation, or can be complementary to a reference sequence encoding a disclosed polypeptide. Thus, nucleic acids encoding each of the disclosed polypeptide sequences, and fragments and variants thereof, in sense and antisense, and in single stranded and double stranded forms, are provided. The nucleic acids can be DNA, RNA, or nucleic acid analogs. Nucleic acid analogs can be modified at the base moiety, sugar moiety, or phosphate backbone. Such modification can improve, for example, stability, hybridization, or solubility of the nucleic acid. Modifications at the base moiety can include deoxyuridine for deoxythymidine, and 5-methyl-2′-deoxycytidine or 5-bromo-2′-deoxycytidine for deoxycytidine. Modifications of the sugar moiety can include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, for example, Summerton and Weller (1997) Antisense Nucleic Acid Drug Dev. 7:187-195; and Hyrup et al. (1996) Bioorgan. Med. Chem. 4:5-23. In addition, the deoxyphosphate backbone can be replaced with, for example, a phosphorothioate or phosphorodithioate backbone, a phosphoroamidite, or an alkyl phosphotriester backbone.

### 2. Vectors Expressing or Encoding ENPP3 Antibodies and CAR

In some forms, nucleic acids encoding disclosed molecules are present within vectors. Vectors including an isolated polynucleotide encoding a disclosed polypeptide for the expression of the polypeptide within a host cell are described.

The term “vector” is a nucleic acid molecule used to carry genetic material into another cell, where it can be replicated and/or expressed. Any vector known to those skilled in the art in view of the present disclosure can be used. Examples of vectors include, but are not limited to, plasmids, viral vectors (bacteriophage, animal viruses, and plant viruses), cosmids, and artificial chromosomes (e.g., YACs). A vector can be a DNA vector or an RNA vector. In some forms, a vector is a DNA plasmid. One of ordinary skill in the art can construct a vector of the application through standard recombinant techniques in view of the present disclosure.

In some forms, the vector including nucleic acids encoding a polypeptide is an expression vector. The term “expression vector” refers to any type of genetic construct including a nucleic acid coding for an RNA capable of being transcribed. Expression vectors include, but are not limited to, vectors for recombinant protein expression, such as a DNA plasmid or a viral vector, and vectors for delivery of nucleic acid into a subject for expression in a tissue of the subject, such as a DNA plasmid or a viral vector. It will be appreciated by those skilled in the art that the design of the expression vector can depend on such factors as the choice of the host cell to be transformed, the level of expression of protein desired, etc.

In some forms, vectors contain one or more regulatory sequences. The term “regulatory sequence” refers to any sequence that allows, contributes or modulates the functional regulation of the nucleic acid molecule, including replication, duplication, transcription, splicing, translation, stability and/or transport of the nucleic acid or one of its derivative (i.e. mRNA) into the host cell or organism. In the context of the disclosure, this term encompasses promoters, enhancers and other expression control elements (e.g., polyadenylation signals and elements that affect mRNA stability).

In some forms, the vector is a non-viral vector. Examples of non-viral vectors include, but are not limited to, DNA plasmids, bacterial artificial chromosomes, yeast artificial chromosomes, bacteriophages, etc. Examples of non-viral vectors include, but are not limited to, RNA replicon, mRNA replicon, modified mRNA replicon or self-amplifying mRNA, closed linear deoxyribonucleic acid, e.g., a linear covalently closed DNA, e.g., a linear covalently closed double stranded DNA molecule. Preferably, a non-viral vector is a DNA plasmid. A “DNA plasmid”, which is used interchangeably with “DNA plasmid vector,” “plasmid DNA” or “plasmid DNA vector,” refers to a double-stranded and generally circular DNA sequence that is capable of autonomous replication in a suitable host cell. DNA plasmids used for expression of an encoded polynucleotide typically include an origin of replication, a multiple cloning site, and a selectable marker, which for example, can be an antibiotic resistance gene. Examples of suitable DNA plasmids that can be used include, but are not limited to, commercially available expression vectors for use in well-known expression systems (including both prokaryotic and eukaryotic systems), such as pSE420 (Invitrogen, San Diego, Calif.), which can be used for production and/or expression of protein in Escherichia coli; pYES2 (Invitrogen, Thermo Fisher Scientific), which can be used for production and/or expression in Saccharomyces cerevisiae strains of yeast; MAXBAC®. complete baculovirus expression system (Thermo Fisher Scientific), which can be used for production and/or expression in insect cells; pcDNA™. or pcDNA3™ (Life Technologies, Thermo Fisher Scientific), which can be used for high level constitutive protein expression in mammalian cells; and pVAX or pVAX-1 (Life Technologies, Thermo Fisher Scientific), which can be used for high-level transient expression of a protein of interest in most mammalian cells. The backbone of any commercially available DNA plasmid can be modified to optimize protein expression in the host cell, such as to reverse the orientation of certain elements (e.g., origin of replication and/or antibiotic resistance cassette), replace a promoter endogenous to the plasmid (e.g., the promoter in the antibiotic resistance cassette), and/or replace the polynucleotide sequence encoding transcribed proteins (e.g., the coding sequence of the antibiotic resistance gene), by using routine techniques and readily available starting materials. (See e.g., Sambrook et al., Molecular Cloning a Laboratory Manual, Second Ed. Cold Spring Harbor Press (1989)).

Preferably, a DNA plasmid is an expression vector suitable for protein expression in mammalian host cells. Expression vectors suitable for protein expression in mammalian host cells include, but are not limited to, pcDNA™, pcDNA3™, pVAX, pVAX-1, ADVAX, NTC8454, etc. In some forms, an expression vector is based on pVAX-1, which can be further modified to optimize protein expression in mammalian cells. pVAX-1 is a commonly used plasmid in DNA vaccines, and contains a strong human immediate early cytomegalovirus (CMV-IE) promoter followed by the bovine growth hormone (bGH)-derived polyadenylation sequence (pA). pVAX-1 further contains a pUC origin of replication and a kanamycin resistance gene driven by a small prokaryotic promoter that allows for bacterial plasmid propagation.

In some forms the vector is a viral vector. In general, viral vectors are genetically engineered viruses carrying modified viral DNA or RNA that has been rendered non-infectious, but still contains viral promoters and transgenes, thus allowing for translation of the transgene through a viral promoter. Because viral vectors are frequently lacking infectious sequences, they require helper viruses or packaging lines for large-scale transfection. Examples of viral vectors that can be used include, but are not limited to, adenoviral vectors, adeno-associated virus vectors, pox virus vectors, enteric virus vectors, Venezuelan Equine Encephalitis virus vectors, Semliki Forest Virus vectors, Tobacco Mosaic Virus vectors, lentiviral vectors, arenavirus viral vectors, replication-deficient arenavirus viral vectors or replication-competent arenavirus viral vectors, bi-segmented or tri-segmented arenavirus, infectious arenavirus viral vectors, nucleic acids which include an arenavirus genomic segment wherein one open reading frame of the genomic segment is deleted or functionally inactivated (and replaced by a nucleic acid encoding a disclosed polypeptide or another therapeutic polypeptide as described herein), arenavirus such as lymphocytic choriomeningitidis virus (LCMV), e.g., clone 13 strain or MP strain, and arenavirus such as Junin virus e.g., Candid #1 strain, etc.

In some forms, the viral vector is an adenovirus vector, e.g., a recombinant adenovirus vector. A recombinant adenovirus vector can for instance be derived from a human adenovirus (HAdV, or AdHu), or a simian adenovirus such as chimpanzee or gorilla adenovirus (ChAd, AdCh, or SAdV) or rhesus adenovirus (rhAd). Preferably, an adenovirus vector is a recombinant human adenovirus vector, for instance a recombinant human adenovirus serotype 26, or any one of recombinant human adenovirus serotype 5, 4, 35, 7, 48, etc. In other forms, an adenovirus vector is a rhAd vector, e.g. rhAd51, rhAd52 or rhAd53. In some forms, a recombinant viral vector is prepared using methods known in the art in view of the present disclosure. For example, in view of the degeneracy of the genetic code, several nucleic acid sequences can be designed that encode the same polypeptide. In some forms, a polynucleotide encoding a disclosed polypeptide is codon-optimized to ensure proper expression in the host cell (e.g., bacterial or mammalian cells). Codon-optimization is a technology widely applied in the art, and methods for obtaining codon-optimized polynucleotides will be well known to those skilled in the art in view of the present disclosure.

In some forms, the vectors, e.g., a DNA plasmid or a viral vector (particularly an adenoviral vector), include any regulatory elements to establish conventional function(s) of the vector, including but not limited to replication and expression of a disclosed polypeptide encoded by the polynucleotide sequence of the vector.

In some forms, the vector is adeno-associated viral vector (AAV). AAV vector used in the compositions and methods can be a naturally occurring serotype of AAV or an artificial variant. In preferred forms, the serotype of the AAV vector is AAV6 or AAV9.

In some forms, the vector for inclusion in the gene editing compositions or for providing elements of the gene editing compositions (e.g., transposon) is a viral vector such as a vesicular stomatitis (VSV) vector, a Bocavirus vector, such as a human bocavirus 1 (HBoV1) vector, a Herpes simplex virus (HSV) vector, or an adenovirus vector (AdV).

In some forms, the viral vector is a Herpes simplex virus (HSV) vector. Herpes simplex viruses (HSV) are large, enveloped dsDNA viruses characteristic of their lytic and latent nature of infection, which result in life-long latent infection of neurons and allows for long-term transgene expression. Deletion of HSV genes has generated expression vectors with low toxicity and an excellent packaging capacity of >30 kb foreign DNA. In some forms, the viral vector is a Vesicular stomatitis virus (VSV) vector. Vesicular stomatitis virus is a non-segmented, negative-stranded RNA virus that belongs to the family Rhabdoviridae, genus Vesiculovirus. VSV infects a broad range of animals, including cattle, horses, and swine. The genome of the virus codes for five major proteins, glycoprotein (G), matrix protein (M), nucleoprotein (N), large protein (L), and phosphoprotein (P). The G protein mediates both viral binding and host cell fusion with the endosomal membrane following endocytosis. The L and P proteins are subunits of the viral RNA-dependent RNA polymerase. The simple structure and rapid high-titer growth of VSV in mammalian and many other cells has made recombinant VSV a useful tool in the fields of cellular and molecular biology and virology.

In some forms, the viral vector is a human Bocavirus vector (HBoV). Exemplary human bocavirus vectors include human bocaviruses 1-4 (HBoV1-4), As well as Gorilla BoV.

In other forms, the viral vector is an adenovirus vector. In some forms, the vector is a chimeric vector, such as a vector that is based on a chimeric virus formed from a combination of one or more components from two or more different viral vectors. An exemplary chimeric viral vector is a chimeric bocavirus/adeno-associated virus vector. Therefore, in some forms, the vector is a chimeric HBoV1/AAV2 vector (e.g., rAAV2/HBoV1 chimeras).

### 3. Transposons and Genome Engineering Systems

In some forms, the vector is an AAV vector that can transduce diverse cell types with minimal cellular toxicity, leading to highly efficient and stable genomic modifications.

An exemplary method for introducing a disclosed polypeptide into a cell includes introducing to the cell a viral vector including a transposon encoding the polypeptide and a sequence that encodes one or more transposase enzymes configured to specifically mediate targeted integration of the transposon into the cellular genome.

Also disclosed are systems for introducing a disclosed polypeptide into a cell, where the system includes a viral vector including a transposon encoding the disclosed polypeptide and a sequence that encodes one or more transposase enzymes configured to specifically mediate targeted integration of the transposon into the cellular genome.

In some forms, the expression vector also includes one or more additional functional elements, for example, for genetic modification of the host cell by removal or silencing of one or more of the host genes.

In some forms, the vector provides combinations of simultaneous multiplexed knockout and knock-in genomic modifications in the host cell. In some forms, the compositions include an RNA-guided endonuclease and one or more AAV vectors containing a sequence (e.g., a crRNA array) that encodes one or more crRNAs that collectively direct the endonuclease to one or more target genes. Optionally, at least one of the AAV vectors contains or further contains one or more HDR templates. The crRNA array can encode two or more crRNAs each of which direct the endonuclease to a different target gene. In some forms, the method can involve introducing two AAV vectors. In the foregoing method, the one or more HDR templates include (a) a sequence that encodes a reporter gene and/or a disclosed polypeptide, and (b) one or more sequences homologous to one or more target sites. The HDR template can further include a promoter and/or polyadenylation signal operationally linked to each reporter gene, disclosed polypeptide, or combination thereof.

In some forms, the RNA-guided endonuclease is capable of disruption of the target genes and/or the one or more HDR templates can mediate targeted integration of the reporter gene, the disclosed polypeptide, or combinations thereof at the target sites. A target site can be within the locus of the disrupted gene or at a locus different from the disrupted gene. Exemplary target genes or target sites include, but are not limited to PDCD1, TRAC, CTLA4, B2M, CIITA, TRBC1, and TRBC2. A preferred target gene is TRAC. In some forms, the PDCD1 and/or TRAC gene can be disrupted; one or more reporter genes, and/or a disclosed polypeptide, can be integrated in the PDCD1 and/or TRAC gene; the PDCD1 gene can be disrupted and the one or more reporter genes, and/or disclosed polypeptide can be integrated in the TRAC gene; or the TRAC gene can disrupted and the one or more reporter genes, and/or the disclosed polypeptide can be integrated in the PDCD1 gene.

In preferred embodiments, the disclosed polypeptide includes an ENPP3 antigen binding domain, for example an ENPP3-targeting CAR fusion protein.

### 4. Exemplary Nucleic Acid Constructs

Nucleic acids that express or encode antibodies or CAR constructs, including the antigen binding components of any of the described ENPP3 binding antibodies are provided. In exemplary forms, a nucleic acid encodes a polypeptide that includes the CDRs of the SX13-7 antibody, or the SX13-10 antibody. In some forms, the nucleic acid expresses or encodes a CAR that includes the CDRs of the SX13-7 antibody, or the SX13-10 antibody together with a spacer domain from human IgG4, and/or the transmembrane domain of CD8 alpha, fused to the intracellular domain of 4-1BB and CD3 zeta.

In some forms, a nucleic acid encodes a CAR including the antigen binding components of ENPP3 specific antibody SX13-7, such as an ScFv having the amino acid sequence of SEQ ID NO:23, fused with a polypeptide derived from the extracellular (hinge) domain of human IgG4m, together with a polypeptide derived from the transmembrane region of the human CD8 alpha coreceptor, and a cytoplasmic region derived from the cytoplasmic component of 4-1BB and CD3 zeta.

In other forms, a nucleic acid encodes a CAR including the antigen binding components of ENPP3 specific antibody SX13-7, such as an ScFv having the amino acid sequence of SEQ ID NO:23, fused with a polypeptide derived from the extracellular (hinge) domain of human IgG4m, together with a polypeptide derived from the transmembrane region of the human CD28 coreceptor, and a cytoplasmic region derived from the cytoplasmic component of the human CD28 coreceptor and a cytoplasmic region derived from the cytoplasmic component of human CD3 zeta.

In other forms, a nucleic acid encodes a CAR including the antigen binding components of ENPP3 specific antibody SX13-7, such as an ScFv having the amino acid sequence of SEQ ID NO:23, fused with a polypeptide derived from the extracellular (hinge) domain of human IgG4m, together with a polypeptide derived from the transmembrane region of the human CD8 coreceptor, and a cytoplasmic region derived from the cytoplasmic component of the human 4-1BB coreceptor and a cytoplasmic region derived from the cytoplasmic region of human CD3 zeta.

In other forms, the CAR includes the antigen binding components of ENPP3 specific antibody SX13-10, such as an ScFv having the amino acid sequence of SEQ ID NO: 25, fused with a polypeptide derived from the extracellular (hinge) domain of human IgG4m, together with a polypeptide derived from the transmembrane region of the human CD8 coreceptor, and a cytoplasmic region derived from the cytoplasmic component of the human 4-1BB coreceptor and a cytoplasmic region derived from the cytoplasmic region of human CD3 zeta.

### G. Cells

In some forms, polypeptides, nucleic acids, or vectors encoding the disclosed polypeptides are present within a host cell. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques.

Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Host cells (e.g., a prokaryotic cell or a eukaryotic cell) can be used to, for example, produce the polypeptides described herein.

In some forms, the cell is a eukaryotic cell, such as a mammalian cell. In some forms the mammalian cell is derived from or is designed to be administered into a non-human mammal, e.g., primate, bovine, ovine, porcine, canine, rodent, Leporidae such as monkey, cow, sheep, pig, dog, rabbit, rat or mouse cell. In some forms, the cell is derived from a non-mammalian eukaryotic cell such as poultry bird (e.g., chicken), vertebrate fish (e.g., salmon) or shellfish (e.g., oyster, claim, lobster, shrimp) cell. In some forms, the cell is a plant cell. For example, in some forms, the cell is derived from a monocot or dicot of a crop or grain plant, such as cassava, corn, sorghum, soybean, wheat, oat or rice, or from a tree or production plant, fruit or vegetable (e.g., trees such as citrus trees, e.g., orange, grapefruit or lemon trees; peach or nectarine trees; apple or pear trees; nut trees such as almond or walnut or pistachio trees; nightshade plants; plants of the genus Brassica; plants of the genus Lactuca; plants of the genus Spinacia; plants of the genus Capsicum; cotton, tobacco, asparagus, carrot, cabbage, broccoli, cauliflower, tomato, eggplant, pepper, lettuce, spinach, strawberry, blueberry, raspberry, blackberry, grape, coffee, cocoa, etc.).

In some forms, the cell is from an established cell line, or a primary cell. The term “primary cell,” refers to cells and cell cultures derived from a subject and allowed to grow in vitro for a limited number of passages, i.e. splitting, of the culture. In exemplary forms, the introduction of a transposase and the viral vector encoding a disclosed polypeptide into a host cell is performed ex vivo.

In some forms, the cell is a T cell, hematopoietic stem cell (HSC), macrophage (MA), natural killer cell (NK), or dendritic cell (DC). Thus, CAR T cells, CAR MA cells, CAR NK, and CAR DC cells are all expressly provided. For example, in some forms, the T cell is a CD8+ T cell selected from the group including effector T cells, memory T cells, central memory T cells, and effector memory T cells. In other forms, the T cell is a CD4+ T cell selected from the group including Th1 cells, Th2 cells, Th17 cells, and Treg cells. In some forms, the cell is an alpha-beta T cell that has been genetically modified to remove or diminish the expression of one or more expression products that are expressed in the wild-type cell.

In some forms, cells are obtained from a human subject. For example, in some forms, the cells are autologous cells, i.e., cells obtained from a subject prior to introduction of the disclosed polypeptides, and/or nucleic acids, or vectors encoding the disclosed polypeptides, and re-introduction to the same subject following modification. In other forms, the cells are heterologous cells, i.e., cells obtained from a different subject than the intended recipient. In some forms, the cells are frozen prior to or after introduction of the disclosed polypeptides, and/or nucleic acids, or vectors encoding disclosed polypeptides. Methods and compositions for freezing and thawing viable eukaryotic cells are known in the art. In some forms, the cells are autologous immune cells, such as T cells or progenitor cells/stem cells.

In some forms, cells are obtained from a healthy subject. In other forms, cells are obtained from a subject identified as having or at risk of having a disease or disorder, such as cancer and/or an auto-immune disease.

In preferred forms, the introduction of the disclosed polypeptides to the cells occurs through genetic modification of the cells. In some forms, genetic modification of the cell includes introduction of nucleic acids, or vectors encoding polypeptides to the cell for expression of the disclosed polypeptides within the cell and presentation at the cell surface. In some forms, genetic modification of the cell includes transduction with a transposon encoding a disclosed polypeptide, particularly CAR polypeptides. In an exemplary form, a disclosed polypeptide is introduced into a cell in vitro by transduction of the cell with a nucleic acid encoding a transposon including the disclosed polypeptide. Therefore, genetically modified (Transgenic) cells including disclosed proteins according to the described compositions are described. In some forms, T cells are engineered for endogenous gene editing on genes such as TRAC, TRBC, B2M and CIITA.

In some forms, the cells are human immune cells, such as T cells. Therefore, in some forms, prior to expansion and genetic modification, T cells are obtained from a diseased or healthy subject. In some forms, the CAR cells are autologous T cells obtained from a subject prior to ex vivo genetic modification (i.e., to express CAR) and re-introduction to the same subject in vivo as CAR T-cells.

T cells can be obtained from a number of samples, including peripheral blood mononuclear cells, bone marrow, lymph node tissue, cord blood, thymus tissue, tissue from a site of infection, ascites, pleural effusion, spleen tissue, and tumors. In some forms, T cells are obtained from a unit of blood collected from a subject using any number of techniques known to the skilled artisan, such as FICOLL™ separation. In one preferred form, cells from the circulating blood of an individual are obtained by apheresis. The apheresis product typically contains lymphocytes, including T cells, monocytes, granulocytes, B cells, other nucleated white blood cells, red blood cells, and platelets. The cells collected by apheresis can be washed to remove the plasma fraction and to place the cells in an appropriate buffer or media for subsequent processing steps. In some forms, the cells are washed with phosphate buffered saline (PBS). In some forms, the wash solution lacks calcium and can lack magnesium or can lack many if not all divalent cations. After washing, the cells can be resuspended in a variety of biocompatible buffers, such as, for example, Ca2+-free, Mg2+-free PBS, PLASMALYTE A, or other saline solution with or without buffer. Alternatively, the undesirable components of the apheresis sample are removed and the cells directly resuspended in culture media.

In some forms, T cells are isolated from peripheral blood lymphocytes by lysing the red blood cells and depleting the monocytes, for example, by centrifugation through a PERCOLL™ gradient or by counterflow centrifugal elutriation. In specific forms, a specific subpopulation of T cells, such as CD3+, CD28+, CD4+, CD8+, CD45RA+, and CD45RO+ T cells, is further isolated by positive or negative selection techniques. For example, in some forms, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. Therefore, T cells expressing heterologous CAR (e.g., anti-ENPP3 at the surface of the T cells are provided.

In certain forms, the T cells are genetically modified T cells. For example, in some forms, the T cells are genetically modified to reduce, prevent or otherwise alter the expression of one or more genes within the “wild-type” T cell, in addition to the inclusion and expression of the CAR within the same T cell. In some forms, the T cell is modified to reduce or prevent expression of one or more surface receptors that may interfere with the function or structure of the CAR. For example, in some forms, the T cell is modified by silencing of one or more genes such as TCR alpha and/or TCR beta genes, i.e., to prevent expression of non-CAR alpha-beta TCR proteins at the surface of the T cells. In some forms, the TRAC gene is targeted for removal or ablation. Thus, in some forms, the T cell does not express endogenous alpha-beta TCRs at the surface.

### 1. Exemplary Cells and CAR-T Cells

Cells, such as recombinant or engineered cells that express or include antibodies or CAR constructs, including the antigen binding components of any of the described ENPP3 binding antibodies are also provided.

In some forms, the cells are immune cells, such as T cells. For example, in some forms, T cells are engineered to express one or more of the described ENPP3-specific antibodies or CARs. In exemplary forms, an engineered T cell is a CAR T cell that expresses or includes a polypeptide that includes the CDRs of the SX13-7 antibody, or the SX13-10 antibody. In some forms, an engineered T cell is a CAR T cell that expresses or includes a CAR having the CDRs of the SX13-7 antibody, or the SX13-10 antibody, together with a spacer domain from human IgG4, and/or the transmembrane domain of CD8 alpha, fused to the intracellular domain of 4-1BB and CD3 zeta.

In some forms, an engineered T cell is a CART cell that expresses or includes a CAR including the antigen binding components of ENPP3 specific antibody SX13-7, such as an ScFv having the amino acid sequence of SEQ ID NO:23, fused with a polypeptide derived from the extracellular (hinge) domain of human IgG4m, together with a polypeptide derived from the transmembrane region of the human CD8 alpha coreceptor, and a cytoplasmic region derived from the cytoplasmic component of 4-1BB and CD3 zeta.

In some forms, an engineered T cell is a CART cell that expresses or includes a CAR including the antigen binding components of ENPP3 specific antibody SX13-7, such as an ScFv having the amino acid sequence of SEQ ID NO:23, fused with a polypeptide derived from the extracellular (hinge) domain of human IgG4m, together with a polypeptide derived from the transmembrane region of the human CD28 coreceptor, and a cytoplasmic region derived from the cytoplasmic component of the human CD28 coreceptor and a cytoplasmic region derived from the cytoplasmic component of human CD3 zeta.

In some forms, an engineered T cell is a CART cell that expresses or includes a CAR including the antigen binding components of ENPP3 specific antibody SX13-7, such as an ScFv having the amino acid sequence of SEQ ID NO:23, fused with a polypeptide derived from the extracellular (hinge) domain of human IgG4m, together with a polypeptide derived from the transmembrane region of the human CD8 coreceptor, and a cytoplasmic region derived from the cytoplasmic component of the human 4-1BB coreceptor and a cytoplasmic region derived from the cytoplasmic region of human CD3 zeta.

In some forms, an engineered T cell is a CART cell that expresses or includes a CAR including the antigen binding components of ENPP3 specific antibody SX13-10, such as an ScFv having the amino acid sequence of SEQ ID NO:25, fused with a polypeptide derived from the extracellular (hinge) domain of human IgG4m, together with a polypeptide derived from the transmembrane region of the human CD8 coreceptor, and a cytoplasmic region derived from the cytoplasmic component of the human 4-1BB coreceptor and a cytoplasmic region derived from the cytoplasmic region of human CD3 zeta.

### H. Delivery Vehicles

Any of the disclosed compositions including, but not limited to the disclosed proteins and/or nucleic acids, can be delivered to target cells using a delivery vehicle. The delivery vehicles can be, for example, polymeric particles, inorganic particles, silica particles, liposomes, micelles, multilamellar vesicles, etc.

Delivery vehicles may be microparticles or nanoparticles. Nanoparticles are often utilized for inter-tissue application, penetration of cells, and certain routes of administration. The nanoparticles may have any desired size for the intended use. The nanoparticles may have any diameter from 10 nm up to about 1,000 nm. The nanoparticle can have a diameter from 10 nm to 900 nm, from 10 nm to 800 nm, from 10 nm to 700 nm, from 10 nm to 600 nm, from 10 nm to 500 nm, from 20 nm from 500 nm, from 30 nm to 500 nm, from 40 nm to 500 nm, from 50 nm to 500 nm, from 50 nm to 400 nm, from 50 nm to 350 nm, from 50 nm to 300 nm, or from 50 nm to 200 nm. In some forms the nanoparticles can have a diameter less than 400 nm, less than 300 nm, or less than 200 nm. The range can be between 50 nm and 300 nm.

Thus, in some forms, the delivery vehicles are nanoscale compositions, for example, 10 nm up to, but not including, about 1 micron. However, it will be appreciated that in some forms, and for some uses, the particles can be smaller, or larger (e.g., microparticles, etc.). Although many of the compositions disclosed herein are referred to as nanoparticle or nanocarrier compositions, it will be appreciated that in some forms and for some uses the carrier can be somewhat larger than nanoparticles. Such compositions can be referred to as microparticulate compositions. For example, a nanocarriers according to the present disclosure may be a microparticle. Microparticles can a diameter between, for example, 0.1 and 100 μm in size.

### I. Pharmaceutical Compositions

Pharmaceutical compositions including the disclosed compositions, e.g., a disclosed ENPP3 binding polypeptide, or a genetically modified cell or a population of genetically modified cells expressing ENPP3-targeting CAR proteins.

In some forms, the pharmaceutical compositions include one or more of a pharmaceutically acceptable buffer, carrier, diluent or excipients. In some forms, the pharmaceutical compositions include a specific amount of a provided compound or specific number or population of cells, for example, expanded by culturing and expanding an isolated genetically modified cell, e.g., a homogenous population. Therefore, in some forms, pharmaceutical compositions include a homogenous population of modified cells including and/or expressing a CAR polypeptide. In other forms, the pharmaceutical compositions include populations of cells that contain variable or different genetically modified cells, e.g., a heterogeneous population. In some forms, the pharmaceutical compositions include cells that are bispecific or multi-specific. Any of the compositions can additionally or alternatively include one or more anti-ENPP3 antibodies. In other forms, the compositions include no cells, but include one or more species of an antibody, for example, targeting ENPP3.

In some forms, the cells have been isolated from a diseased or healthy subject prior to genetic modification to express an ENPP3 CAR.

The term “pharmaceutically acceptable carrier” describes a pharmaceutically acceptable material, composition, or vehicle that is involved in carrying or transporting a compound of interest from one tissue, organ, or portion of the body to another tissue, organ, or portion of the body. For example, in some forms the carrier is a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

In some forms, pharmaceutical compositions include buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. The pharmaceutical compositions can be formulated for delivery via any route of administration. The term “Route of administration” can refer to any administration pathway known in the art, including but not limited to aerosol, nasal, oral, intravenous, intramuscular, intraperitoneal, inhalation, transmucosal, transdermal, parenteral, implantable pump, continuous infusion, topical application, capsules and/or injections. The pharmaceutical compositions are preferably formulated for intravenous administration.

Typically, the disclosed pharmaceutical compositions are administered in a manner appropriate to a disease to be treated (or prevented). The quantity and frequency of administration is typically determined by such factors as the condition of the patient, and the type and severity of the patient's disease, although appropriate dosages can be determined by clinical trials.

The disclosed pharmaceutical compositions can be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

### III. Methods of Treatment

Methods of treatment including a ENPP3-binding polypeptide, ENPP3-targeting CAR cells, and other therapeutic agents disclosed herein are provided. In some embodiments, the methods include Adoptive Cell Therapy (ACT) employing ENPP-3 targeting CAR cells. The CAR cells can be CAR immune cells, e.g., CAR T cells, CAR MA cells, CAR NK, and CAR DC cells are all expressly provided.

An exemplary method involves treating a subject (e.g., a human) having a disease, disorder, or condition by administering to the subject an effective amount of a disclosed pharmaceutical composition. In some forms, the methods treat a subject having a disease, disorder, or condition associated with an elevated expression or specific expression of an ENPP3 antigen by administering to the subject an effective amount of a disclosed pharmaceutical composition. In some forms, the methods treat a subject having a disease, disorder, or condition associated with an elevated expression or specific expression of an ENPP3 antigen by administering to the subject an effective amount of a pharmaceutical composition including an ENPP3-binding polypeptide or an cell, e.g., an immune cell, modified to contain a CAR that targets the antigen ENPP3. The cell can have been isolated from the subject having the disease, disorder, or condition, or from a healthy donor, prior to genetic modification.

In some forms, the methods administer a molecule or antibody, or cell including or expressing a molecule or antibody having an antigen binding domain including the light chain variable domain and heavy chain variable domain of an antibody clone selected from SX13-7, SX13-10, SX13-1, SX13-2, SX13-5, SX13-6, SX13-8.1, or SX13-12. In an exemplary form, the molecule or antibody includes an antigen binding domain including six complementarity-determining regions (CDRs), whereby the CDRs include at least one consensus CDR of the CDRs of a heavy chain variable domain having an amino acid sequence of SEQ ID NO:78, and a light chain variable domain having an amino acid sequence of SEQ ID NO:79. In some forms of the method, the antigen binding domain includes heavy chain CDR1, heavy chain CDR2 and heavy chain CDR3 of SEQ ID NO:78, and light chain CDR1, light chain CDR2 and light chain CDR3 of SEQ ID NO: 79. In some forms of the method, the heavy chain CDR1 includes an amino acid sequence of SEQ ID NO:46, heavy chain CDR2 includes an amino acid sequence of SEQ ID NO:36, and heavy chain CDR3 includes an amino acid sequence of SEQ ID NO:31, light chain CDR1 includes an amino acid sequence of SEQ ID NO:52, light chain CDR2 includes an amino acid sequence of SEQ ID NO:53 and light chain CDR3 includes an amino acid sequence of SEQ ID NO:34. In some forms of the method, the molecule includes an amino acid sequence of SEQ ID NO:23. In some forms of the method, the molecule is a chimeric antigen receptor (CAR), optionally whereby the CAR includes one or more of an extracellular antigen binding domain, a spacer domain, a transmembrane domain, and intracellular signaling domain. In some forms, the methods administer one or more cells, such as a population of cells, including or expressing a CAR having an amino acid sequence of any one of SEQ ID NOs: 28, 99, 109, 111, 113, 115, 117 or 119.

### A. Diseases to be Treated

Methods of treating a subject having a disease or disorder are provided. The subject to be treated can have a disease, disorder, or condition such as but not limited to, cancer. The disease, disorder, or condition can be associated with an elevated expression or specific expression of an ENPP3 antigen. In some forms, the methods treat or prevent cancer in a subject in need thereof.

In some forms, the methods treat or prevent a cancer in a subject.

Cancer is a disease of genetic instability, allowing a cancer cell to acquire the hallmarks proposed by Hanahan and Weinberg, including (i) self-sufficiency in growth signals; (ii) insensitivity to anti-growth signals; (iii) evading apoptosis; (iv) sustained angiogenesis; (v) tissue invasion and metastasis; (vi) limitless replicative potential; (vii) reprogramming of energy metabolism; and (viii) evading immune destruction (Cell., 144:646-674, (2011)).

Tumors, which can be treated in accordance with the disclosed methods, are classified according to the embryonic origin of the tissue from which the tumor is derived. Carcinomas are tumors arising from endodermal or ectodermal tissues such as skin or the epithelial lining of internal organs and glands. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

The described compositions and methods are useful for treating, or alleviating subjects having benign or malignant tumors by delaying or inhibiting the growth/proliferation or viability of tumor cells in a subject, reducing the number, growth or size of tumors, inhibiting or reducing metastasis of the tumor, and/or inhibiting or reducing symptoms associated with tumor development or growth.

The disclosed compositions and methods of treatment thereof are generally suited for treatment of carcinomas, sarcomas, lymphomas and leukemias expressing ENPP3.

In a particular embodiment the cancer or tumor to be treated is a kidney cancer such as renal cell carcinoma (RCC). Renal cell carcinoma (RCC), also known as renal cell cancer or renal cell adenocarcinoma, is the most common type of kidney cancer. About 9 out of 10 kidney cancers are renal cell carcinomas. The RCC can be clear cell RCC or a non-clear cell RCC such as papillary renal cell carcinoma, chromophobe renal cell carcinoma, collecting duct RCC, multilocular cystic RCC, medullary carcinoma, mucinous tubular and spindle cell carcinoma, or neuroblastoma-associated RCC.

In some embodiments, the cancer includes one or more solid tumors.

Other types of cancer that can be treated with the provided compositions and methods include, but are not limited to, cancers such as vascular cancer such as multiple myeloma, adenocarcinomas and sarcomas, of bone, bladder, brain, breast, cervical, colorectal, esophageal, kidney, liver, lung, nasopharangeal, pancreatic, prostate, skin, stomach, and uterine. In some forms, the compositions are used to treat multiple cancer types concurrently. The compositions can also be used to treat metastases or tumors at multiple locations.

Exemplary tumor cells include, but are not limited to, tumor cells of cancers, including leukemias including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as, but not limited to, chronic myelocytic (granulocytic) leukemia, chronic lymphocytic leukemia, hairy cell leukemia; polycythemia vera; lymphomas such as, but not limited to, Hodgkin's disease, non-Hodgkin's disease; multiple myelomas such as, but not limited to, smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as, but not limited to, bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors including, but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including, but not limited to, pheochromocytom and adrenocortical carcinoma; thyroid cancer such as but not limited to papillary or follicular thyroid cancer, medullary thyroid cancer and anaplastic thyroid cancer; pancreatic cancer, including, but not limited to, insulinoma, gastrinoma, glucagonoma, vipoma, somatostatin-secreting tumor, and carcinoid or islet cell tumor; pituitary cancers including, but not limited to, Cushing's disease, prolactin-secreting tumor, acromegaly, and diabetes insipius; eye cancers including, but not limited to, ocular melanoma such as iris melanoma, choroidal melanoma, and ciliary body melanoma, and retinoblastoma; vaginal cancers, including, but not limited to, squamous cell carcinoma, adenocarcinoma, and melanoma; vulvar cancer, including, but not limited to, squamous cell carcinoma, melanoma, adenocarcinoma, basal cell carcinoma, sarcoma, and Paget's disease; cervical cancers including, but not limited to, squamous cell carcinoma, and adenocarcinoma; uterine cancers including, but not limited to, endometrial carcinoma and uterine sarcoma; ovarian cancers including, but not limited to, ovarian epithelial carcinoma, borderline tumor, germ cell tumor, and stromal tumor; esophageal cancers including, but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers including, but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers including, but not limited to, hepatocellular carcinoma and hepatoblastoma, gallbladder cancers including, but not limited to, adenocarcinoma; cholangiocarcinomas including, but not limited to, papillary, nodular, and diffuse; lung cancers including, but not limited to, non-small cell lung cancer, squamous cell carcinoma (epidermoid carcinoma), adenocarcinoma, large-cell carcinoma and small-cell lung cancer; testicular cancers including, but not limited to, germinal tumor, seminoma, anaplastic, classic (typical), spermatocytic, nonseminoma, embryonal carcinoma, teratoma carcinoma, choriocarcinoma (yolk-sac tumor), prostate cancers including, but not limited to, adenocarcinoma, leiomyosarcoma, and rhabdomyosarcoma; penal cancers; oral cancers including, but not limited to, squamous cell carcinoma; basal cancers; salivary gland cancers including, but not limited to, adenocarcinoma, mucoepidermoid carcinoma, and adenoidcystic carcinoma; pharynx cancers including, but not limited to, squamous cell cancer, and verrucous; skin cancers including, but not limited to, basal cell carcinoma, squamous cell carcinoma and melanoma, superficial spreading melanoma, nodular melanoma, lentigo malignant melanoma, acral lentiginous melanoma; kidney cancers including, but not limited to, renal cell cancer, adenocarcinoma, hypernephroma, fibrosarcoma, transitional cell cancer (renal pelvis and/or uterer); Wilms' tumor; bladder cancers including, but not limited to, transitional cell carcinoma, squamous cell cancer, adenocarcinoma, and carcinosarcoma. For a review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

### B. Effective Amounts

The effective amount or therapeutically effective amount of a pharmaceutical compositions including for example an ENPP3 binding polypeptide or modified cells, such as therapeutic T cells, can be a dosage sufficient to treat, inhibit, or alleviate one or more symptoms of a disease or disorder, such as a cancer, or to otherwise provide a desired pharmacologic and/or physiologic effect, for example, reducing, inhibiting, or reversing one or more of the underlying pathophysiological mechanisms underlying a disease or disorder, such as cancer or autoimmune disease.

In some forms, when administrating the pharmaceutical composition, the amount administered can be expressed as the amount effective to achieve a desired anti-cancer effect in the recipient. For example, in some forms, the amount of the pharmaceutical composition, is effective to inhibit the viability or proliferation of cancer cells in the recipient. In some forms, the amount of the pharmaceutical composition including modified cells, such as therapeutic T cells, is effective to reduce the tumor burden in the recipient, or reduce the total number of cancer cells, and combinations thereof. In other forms, the amount of the pharmaceutical compositions including modified cells, such as therapeutic T cells, is effective to reduce one or more symptoms or signs of cancer in a cancer patient, or signs of an autoimmune disease in a patient having an autoimmune disease or disorder.

The effective amount of the pharmaceutical composition will vary based on the active agent and from subject to subject, depending on the species, age, weight and general condition of the subject, the severity of the disorder being treated, and its mode of administration. Thus, it is not possible to specify an exact amount for every pharmaceutical composition. However, an appropriate amount can be determined by one of ordinary skill in the art using only routine experimentation given the teachings herein. For example, effective dosages and schedules for administering the pharmaceutical composition can be determined empirically. In some forms, the dosage ranges for the administration of the composition are those large enough to effect reduction in cancer cell proliferation or viability, or to reduce tumor burden for example.

Preferably, the dosage is not so large as to cause adverse side effects, such as unwanted cross-reactions, anaphylactic reactions, and the like. Generally, the dosage will vary with the age, condition, and sex of the patient, route of administration, whether other drugs are included in the regimen, and the type, stage, and location of the disease to be treated. The dosage can be adjusted by the individual physician in the event of any counter-indications. It will also be appreciated that the effective dosage of the composition can increase or decrease over the course of a particular treatment. Changes in dosage can result and become apparent from the results of diagnostic assays.

Dosage can vary, and can be administered in one or more dose administrations daily, for one or several days. Guidance can be found in the literature for appropriate dosages for given classes of pharmaceutical products. Optimal dosing schedules can be calculated from measurements of drug accumulation in the body of the subject or patient. Persons of ordinary skill can determine optimum dosages, dosing methodologies and repetition rates. Optimum dosages can vary depending on the relative potency of individual pharmaceutical compositions, and can generally be estimated based on EC50S found to be effective in in vitro and in vivo animal models.

For example, some embodiments, antibodies are packaged in a hermetically sealed container, such as an ampoule or sachette, indicating the quantity of antibody. In some embodiments, the antibodies are supplied as a dry sterilized lyophilized powder or water free concentrate in a hermetically sealed container and can be reconstituted, e.g., with water or saline to the appropriate concentration for administration to a subject. Preferably, the antibodies of the invention are supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, or at least 75 mg. The lyophilized antibodies can be stored at between 2 and 8° C. in their original container and the antibodies can be administered within 12 hours, preferably within 6 hours, within 5 hours, within 3 hours, or within 1 hour after being reconstituted. In an alternative embodiment, antibodies can be supplied in liquid form in a hermetically sealed container indicating the quantity and concentration of the antibody, fusion protein, or conjugated molecule. Preferably, the liquid form of the antibodies are supplied in a hermetically sealed container at least 1 mg/ml, more preferably at least 2.5 mg/ml, at least 5 mg/ml, at least 8 mg/ml, at least 10 mg/ml, at least 15 mg/ml, at least 25 mg/ml, at least 50 mg/ml, at least 100 mg/ml, at least 150 mg/ml, at least 200 mg/ml of the antibodies.

For antibodies encompassed by the invention, the dosage administered to a patient is typically 0.01 mg/kg to 100 mg/kg of the patient's body weight. Preferably, the dosage administered to a patient is between 0.01 mg/kg and 20 mg/kg, 0.01 mg/kg and 10 mg/kg, 0.01 mg/kg and 5 mg/kg, 0.01 and 2 mg/kg, 0.01 and 1 mg/kg, 0.01 mg/kg and 0.75 mg/kg, 0.01 mg/kg and 0.5 mg/kg, 0.01 mg/kg to 0.25 mg/kg, 0.01 to 0.15 mg/kg, 0.01 to 0.10 mg/kg, 0.01 to 0.05 mg/kg, or 0.01 to 0.025 mg/kg of the patient's body weight. In particular, the invention contemplates that the dosage administered to a patient is 0.2 mg/kg, 0.3 mg/kg, 1 mg/kg, 3 mg/kg, 6 mg/kg or 10 mg/kg. A dose as low as 0.01 mg/kg may show appreciable pharmacodynamic effects. Dose levels of 0.10-1 mg/kg are predicted to be most appropriate. Higher doses (e.g., 1-30 mg/kg) are also contemplated. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies may be reduced by enhancing uptake and tissue penetration of the antibodies by modifications such as, for example, lipidation.

It can generally be stated that a pharmaceutical composition containing CAR cells described herein can be administered at a dosage of 10⁴ to 10⁹ cells/kg body weight, preferably 10⁵ to 10⁷ cells/kg body weight, including all integer values within those ranges. In some forms, patients can be treated by infusing a disclosed pharmaceutical composition containing CAR expressing cells (e.g., T cells) in the range of about 10⁴ to 10¹² or more cells per square meter of body surface (cells/m).

Injections and infusion of the disclosed compositions can be repeated as often and as many times as the patient can tolerate until the desired response is achieved. Thus, antibodies and CAR cell compositions can also be administered once or multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can be determined by one skilled in the art of medicine by monitoring the patient for signs of disease and adjusting the treatment accordingly. In some forms, the unit dosage is in a unit dosage form for intravenous injection. In some forms, the unit dosage is in a unit dosage form for oral administration. In some forms, the unit dosage is in a unit dosage form for inhalation. In some forms, the unit dosage is in a unit dosage form for intra-tumoral injection.

Treatment can be continued for an amount of time sufficient to achieve one or more desired therapeutic goals, for example, a reduction of the amount of cancer cells relative to the start of treatment, or complete absence of cancer cells in the recipient. Treatment can be continued for a desired period of time, and the progression of treatment can be monitored using any means known for monitoring the progression of anti-cancer treatment in a patient. In some forms, administration is carried out every day of treatment, or every week, or every fraction of a week. In some forms, treatment regimens are carried out over the course of up to two, three, four or five days, weeks, or months, or for up to 6 months, or for more than 6 months, for example, up to one year, two years, three years, or up to five years.

The efficacy of administration of a particular dose of the pharmaceutical compositions including modified cells, such as therapeutic T cells, according to the methods described herein can be determined by evaluating the aspects of the medical history, signs, symptoms, and objective laboratory tests that are known to be useful in evaluating the status of a subject in need for the treatment of cancer or other diseases and/or conditions. These signs, symptoms, and objective laboratory tests will vary, depending upon the particular disease or condition being treated or prevented, as will be known to any clinician who treats such patients or a researcher conducting experimentation in this field. For example, if, based on a comparison with an appropriate control group and/or knowledge of the normal progression of the disease in the general population or the particular individual: (1) a subject's physical condition is shown to be improved (e.g., a tumor has partially or fully regressed), (2) the progression of the disease or condition is shown to be stabilized, or slowed, or reversed, or (3) the need for other medications for treating the disease or condition is lessened or obviated, then a particular treatment regimen will be considered efficacious. In some forms, efficacy is assessed as a measure of the reduction in tumor volume and/or tumor mass at a specific time point (e.g., 1-5 days, weeks, or months) following treatment.

### C. Modes of Administration

Any of the disclosed compositions can be used therapeutically in combination with a pharmaceutically acceptable carrier. The compositions described herein can be conveniently formulated into pharmaceutical compositions composed of one or more of the compounds in association with a pharmaceutically acceptable carrier. See, e.g., Remington's Pharmaceutical Sciences, latest edition, by E. W. Martin Mack Pub. Co., Easton, PA, which discloses typical carriers and conventional methods of preparing pharmaceutical compositions that can be used in conjunction with the preparation of formulations of the therapeutics described herein and which is incorporated by reference herein. These most typically would be standard carriers for administration of compositions to humans. In one aspect, for humans and non-humans, these include solutions such as sterile water, saline, and buffered solutions at physiological pH. Other therapeutics can be administered according to standard procedures used by those skilled in the art.

The pharmaceutical compositions including antibodies or modified cells, such as therapeutic T cells, described herein can include, but are not limited to, carriers, thickeners, diluents, buffers, preservatives, surface active agents and the like in addition to the therapeutic(s) of choice.

Pharmaceutical compositions containing antibodies or modified cells, such as therapeutic T cells, and optionally one or more additional therapeutic agents can be administered to the subject in a number of ways depending on whether local or systemic treatment is desired, and on the area to be treated. Thus, for example, a pharmaceutical composition can be administered as an intravenous infusion, or directly injected into a specific site, for example, into or surrounding a tumor. Moreover, a pharmaceutical composition can be administered to a subject as an ophthalmic solution and/or ointment to the surface of the eye, vaginally, rectally, intranasally, orally, by inhalation, or parenterally, for example, by intradermal, subcutaneous, intramuscular, intraperitoneal, intrarectal, intraarterial, intralymphatic, intravenous, intrathecal and intratracheal routes. In some forms, the compositions are administered directly into a tumor or tissue, e.g., stereotactically.

Parenteral administration, if used, is generally characterized by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. Suitable parenteral administration routes include intravascular administration (e.g., intravenous bolus injection, intravenous infusion, intra-arterial bolus injection, intra-arterial infusion and catheter instillation into the vasculature); peri- and intra-tissue injection (e.g., intraocular injection, intra-retinal injection, or sub-retinal injection); subcutaneous injection or deposition including subcutaneous infusion (such as by osmotic pumps); direct application by a catheter or other placement device (e.g., an implant including a porous, non-porous, or gelatinous material).

Preparations for parenteral administration include sterile aqueous or non-aqueous solutions, suspensions, and emulsions which can also contain buffers, diluents and other suitable additives. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Administration of the pharmaceutical compositions containing antibodies or genetically modified cells (e.g., CAR cells) can be localized (i.e., to a particular region, physiological system, tissue, organ, or cell type) or systemic.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

### D. Combination Therapy

Any of the disclosed pharmaceutical compositions can be used alone, or in combination with other therapeutic agents or treatment modalities, for example, chemotherapy or stem-cell transplantation. As used herein, “combination” or “combined” refer to either concomitant, simultaneous, or sequential administration of the therapeutics.

In some forms, the pharmaceutical compositions and other therapeutic agents are administered separately through the same route of administration. In other forms, the pharmaceutical compositions and other therapeutic agents are administered separately through different routes of administration. The combinations can be administered either concomitantly (e.g., as an admixture), separately but simultaneously (e.g., via separate intravenous lines into the same subject; one agent is given orally while the other agent is given by infusion or injection, etc.,), or sequentially (e.g., one agent is given first followed by the second).

Examples of preferred additional therapeutic agents include other conventional therapies known in the art for treating the desired disease, disorder or condition. In some forms, the therapeutic agent is one or more other targeted therapies (e.g., a targeted cancer therapy) and/or immune-checkpoint blockage agents (e.g., anti-CTLA-4, anti-PD1, and/or anti-PDL1 agents such as antibodies).

The compositions and methods described herein may be used as a first therapy, second therapy, third therapy, or combination therapy with other types of therapies known in the art, such as chemotherapy, surgery, radiation, gene therapy, immunotherapy, bone marrow transplantation, stem cell transplantation, targeted therapy, cryotherapy, ultrasound therapy, photodynamic therapy, radio-frequency ablation or the like, in an adjuvant setting or a neoadjuvant setting.

The disclosed pharmaceutical compositions and/or other therapeutic agents, procedures or modalities can be administered during periods of active disease, or during a period of remission or less active disease. The pharmaceutical compositions can be administered before the additional treatment, concurrently with the treatment, post-treatment, or during remission of the disease or disorder. When administered in combination, the disclosed pharmaceutical compositions and the additional therapeutic agents (e.g., second or third agent), or all, can be administered in an amount or dose that is higher, lower or the same than the amount or dosage of each agent used individually, e.g., as a monotherapy. In certain forms, the administered amount or dosage of the disclosed pharmaceutical composition, the additional therapeutic agent (e.g., second or third agent), or all, is lower (e.g., at least 20%, at least 30%, at least 40%, or at least 50%) than the amount or dosage of each agent used individually, e.g., as a monotherapy (e.g., required to achieve the same therapeutic effect).

In some forms, the methods administer one or more additional anti-cancer agents to a subject. In the context of cancer, targeted therapies are therapeutic agents that block the growth and spread of cancer by interfering with specific molecules (“molecular targets”) that are involved in the growth, progression, and spread of cancer. Many different targeted therapies have been approved for use in cancer treatment. These therapies include hormone therapies, signal transduction inhibitors, gene expression modulators, apoptosis inducers, angiogenesis inhibitors, immunotherapies, and toxin delivery molecules. Numerous antineoplastic drugs can be used in combination with the disclosed pharmaceutical compositions. In some forms, the additional therapeutic agent is a chemotherapeutic or antineoplastic drug. The majority of chemotherapeutic drugs can be divided into alkylating agents, antimetabolites, anthracyclines, plant alkaloids, topoisomerase inhibitors, monoclonal antibodies, and other anti-tumor agents.

### IV. Kits

The compositions, reagents, and other materials for the disclosed compounds and cells can be packaged together in any suitable combination as a kit useful for performing, or aiding in the performance of, the methods. It is useful if the components in a given kit are designed and adapted for use together in the method. For example, kits with one or more compositions for administration to a subject, may include a pre-measured dosage of the composition in a sterile needle, ampule, tube, container, or other suitable vessel. The kits may include instructions for dosages and dosing regimens.

Instructional material can include a publication, a recording, a diagram, or any other medium of expression which can be used to communicate the usefulness of the compositions and methods of the kit. Compositions can include antibodies in a solid (i.e., dry powder or lyophilized) form, or as a solution, such as an aqueous solution.

The disclosed compositions and methods can be further understood through the following numbered paragraphs.
1. A molecule or antibody including an antigen binding domain of an antibody that immune-specifically binds to the Ectonucleotide pyrophosphatase/phosphodiesterase 3 (ENPP3) of SEQ ID NOS: 1 and/or 2.
2. A molecule or antibody including an antigen binding domain of an antibody that immuno-specifically binds to ENPP3, the antigen binding domain including six complementarity determining regions (CDRs), wherein the CDRs include at least one consensus CDR of the CDRs of anti-ENPP3 antibodies SX13-1, SX13-2, SX13-5, SX13-6, SX13-7, SX13-8.1, SX13-10 and SX13-12 with all remaining CDRs selected from:
   (A) the three light chain and the three heavy chain CDRs of anti-ENPP3 antibody SX13-1;
   (B) the three light chain and the three heavy chain CDRs of anti-ENPP3 antibody SX13-2;
   (C) the three light chain and the three heavy chain CDRs of anti-ENPP3 antibody SX13-5;
   (D) the three light chain and the three heavy chain CDRs of anti-ENPP3 antibody SX13-6;
   (E) the three light chain and the three heavy chain CDRs of anti-ENPP3 antibody SX13-7;
   (F) the three light chain and the three heavy chain CDRs of anti-ENPP3 antibody SX13-8.1;
   (G) the three light chain and the three heavy chain CDRs of anti-ENPP3 antibody SX13-10; or
   (H) the three light chain and the three heavy chain CDRs of anti-ENPP3 antibody SX13-12.
3. The molecule or antibody of paragraphs 1 or 2, wherein the six CDRs are:
   (A) the three light chain and the three heavy chain CDRs of anti-ENPP3 antibody SX13-1;
   (B) the three light chain and the three heavy chain CDRs of anti-ENPP3 antibody SX13-2;
   (C) the three light chain and the three heavy chain CDRs of anti-ENPP3 antibody SX13-5;
   (D) the three light chain and the three heavy chain CDRs of anti-ENPP3 antibody SX13-6;
   (E) the three light chain and the three heavy chain CDRs of anti-ENPP3 antibody SX13-7;
   (F) the three light chain and the three heavy chain CDRs of anti-ENPP3 antibody SX13-8.1;
   (G) the three light chain and the three heavy chain CDRs of anti-ENPP3 antibody SX13-10; or
   (H) the three light chain and the three heavy chain CDRs of anti-ENPP3 antibody SX13-12.
4. The molecule or antibody of any one of paragraphs 1-3, wherein the antigen binding domain includes one or both of the light and heavy chain variable domain(s) of clone SX13-1, SX13-2, SX13-5, SX13-6, SX13-7, SX13-8.1, SX13-10 or SX13-12.
5. The molecule or antibody of any one of paragraphs 1-4, wherein the antigen binding domain includes both of the light and heavy chain variable domains of clone SX13-1, SX13-2, SX13-5, SX13-6, SX13-7, SX13-8.1, SX13-10 or SX13-12.
6. A molecule or antibody including an antigen binding domain of an antibody that immune-specifically binds to ENPP3, the antigen binding domain including six complementarity-determining regions (CDRs), wherein the CDRs include at least one consensus CDR of the CDRs of the amino acid sequence of SEQ ID NOS: 3 or 4, SEQ ID NOS: 5 or 6, SEQ ID NOS: 7 or 8, SEQ ID NOS: 9 or 10, SEQ ID NOS: 11 or 12, SEQ ID NOS: 13 or 14, SEQ ID NOS: 15 or 16, and SEQ ID NOS: 17 or 18, with all remaining CDRs selected from:
   (A) the three light chain and the three heavy chain CDRs of the amino acid sequence of SEQ ID NOS: 4 or 3;
   (B) the three light chain and the three heavy chain CDRs of the amino acid sequence of SEQ ID NOS: 6 or 5;
   (C) the three light chain and the three heavy chain CDRs of the amino acid sequence of SEQ ID NOS: 8 or 7;
   (D) the three light chain and the three heavy chain CDRs of the amino acid sequence of SEQ ID NOS: 10 or 9;
   (E) the three light chain and the three heavy chain CDRs of the amino acid sequence of SEQ ID NOS: 12 or 11;
   (F) the three light chain and the three heavy chain CDRs of the amino acid sequence of SEQ ID NOS: 14 or 13;
   (G) the three light chain and the three heavy chain CDRs of the amino acid sequence of SEQ ID NOS: 16 or 15; or
   (H) the three light chain and the three heavy chain CDRs of the amino acid sequence of SEQ ID NOS: 18 or 17.
7. The molecule or antibody of any one of paragraphs 1-6, wherein the six CDRs are:
   (A) the three light chain and the three heavy chain CDRs of the amino acid sequence of SEQ ID NOS: 4 and 3 or SEQ ID NOS: 71 and 70, respectively;
   (B) the three light chain and the three heavy chain CDRs of the amino acid sequence of SEQ ID NOS: 6 and 5 or SEQ ID NOS: 73 and 72, respectively;
   (C) the three light chain and the three heavy chain CDRs of the amino acid sequence of SEQ ID NOS: 8 and 7 or SEQ ID NOS: 75 and 74, respectively;
   (D) the three light chain and the three heavy chain CDRs of the amino acid sequence of SEQ ID NOS: 10 and 9 with or SEQ ID NOS: 77 and 76, respectively;
   (E) the three light chain and the three heavy chain CDRs of the amino acid sequence of SEQ ID NOS: 12 and 11 or SEQ ID NOS: 79 and 78, respectively;
   (F) the three light chain and the three heavy chain CDRs of the amino acid sequence of SEQ ID NOS: 14 and 13 or SEQ ID NOS: 81 and 80, respectively;
   (G) the three light chain and the three heavy chain CDRs of the amino acid sequence of SEQ ID NOS: 16 and 15 or SEQ ID NOS: 83 and 82, respectively; or
   (H) the three light chain and the three heavy chain CDRs of the amino acid sequence of SEQ ID NOS: 18 and 17 or SEQ ID NOS: 85 and 84. respectively.
8. The molecule or antibody of any one of paragraphs 1-7, wherein the antigen binding domain includes one or both of the light and heavy chain variable domain(s) of the amino acid sequence of SEQ ID NOS: 4 and 3 or SEQ ID NOS: 71 and 70 respectively, SEQ ID NOS: 6 and 5 or SEQ ID NOS: 73 and 72 respectively, SEQ ID NOS: 8 and 7 or SEQ ID NOS: 75 and 74 respectively, SEQ ID NOS: 10 and 9 or SEQ ID NOS: 77 and 76 respectively, SEQ ID NOS: 12 and 11 or SEQ ID NOS: 79 and 78 respectively, SEQ ID NOS: 14 and 13 or SEQ ID NOS: 81 and 80 respectively, SEQ ID NOS: 16 and 15 or SEQ ID NOS: 83 and 82 respectively, SEQ ID NOS: 18 and 17 or SEQ ID NOS: 85 and 84 respectively, or variants of the foregoing with at least 75% sequence identity thereto, optionally wherein the variant includes the CDRs of SEQ ID NOS: 4 and 3, respectively, SEQ ID NOS: 6 and 5, respectively, SEQ ID NOS: 8 and 7, respectively, SEQ ID NOS: 10 and 9, respectively, SEQ ID NOS: 12 and 11, respectively, SEQ ID NOS: 14 and 13, respectively, SEQ ID NOS: 16 and 15, respectively or SEQ ID NOS: 18 and 17, respectively optionally without variation.
9. The molecule or antibody of any one of paragraphs 1-8, wherein the antigen binding domain includes both of the light and heavy chain variable domains of the amino acid sequence of SEQ ID NOS: 4 and 3 or SEQ ID NOS: 71 and 70 respectively, SEQ ID NOS: 6 and 5 or SEQ ID NOS: 73 and 72 respectively, SEQ ID NOS: 8 and 7 or SEQ ID NOS: 75 and 74 respectively, SEQ ID NOS: 10 and 9 or SEQ ID NOS: 77 and 76 respectively, SEQ ID NOS: 12 and 11 or SEQ ID NOS: 79 and 78 respectively, SEQ ID NOS: 14 and 13 or SEQ ID NOS: 81 and 80 respectively, SEQ ID NOS: 16 and 15 or SEQ ID NOS: 83 and 82 respectively, or SEQ ID NOS: 18 and 17 or SEQ ID NOS: 85 and 84 respectively.
10. The molecule or antibody of any one of paragraphs 1-9, wherein the molecule or antibody includes the amino acid sequence of any one of SEQ ID NOS: 19-26 with or without the signal sequence, or variant thereof with at least 75% sequence identity thereto, optionally wherein the variant includes the CDRs of SEQ ID NOS: 4 and 3, respectively, SEQ ID NOS: 6 and 5, respectively, SEQ ID NOS: 8 and 7, respectively, SEQ ID NOS: 10 and 9, respectively, SEQ ID NOS: 12 and 11, respectively, SEQ ID NOS: 14 and 13, respectively, SEQ ID NOS: 16 and 15, respectively or SEQ ID NOS: 18 and 17, respectively optionally without variation.
11. The molecule or antibody of any one of paragraphs 1-10 wherein the molecule or antibody is an antibody.
12. The molecule or antibody of paragraph 11, wherein the antibody is an intact antibody and functional antibody fragment or fusion protein.
13. The molecule or antibody of paragraph 12, wherein the functional fragment or fusion protein is selected from Fab fragments, F(ab′)2 fragments, Fab′ fragments, Fv fragments, recombinant IgG (rlgG) fragments, single chain antibody fragments optionally single chain variable fragments (scFv), and single domain antibodies optionally selected from sdAb, sdFv, and nanobody fragments.
14. The molecule or antibody of any one of paragraphs 11-13, wherein the antibody is selected from intrabodies, peptibodies, chimeric antibodies, fully human antibodies, humanized antibodies, and heteroconjugate antibodies, and multispecific antibodies optionally selected bispecific antibodies, diabodies, triabodies, tetrabodies, tandem di-scFv, and tandem tri-scFv.
15. The molecule or antibody of any one of paragraphs 11-14 wherein the antibody is an IgM, IgE, IgA, IgD, or IgG optionally an IgG1, IgG2, IgG3, or IgG4.
16. The molecule or antibody of any one of paragraphs 1-15, wherein the molecule or antibody is detectably labeled or includes a conjugated toxin, drug, receptor, enzyme, receptor ligand.
17. A fusion protein including the molecule or antibody of any one of paragraphs 1-16 and heterologous amino acid sequence.
18. A chimeric antigen receptor (CAR) polypeptide including the molecule or antibody of any one of paragraphs 1-17.
19. The chimeric antigen receptor of paragraph 18 including an extracellular antigen binding domain, a spacer domain, a transmembrane domain, and intracellular signaling domain.
20. The chimeric antigen receptor of paragraphs 18 and 19 including a structure including the molecule or antibody of any one of paragraphs 1-16 linked to CD8hinge linked to CD8TM linked to 41BBcostim linked to CD3z optionally including the amino acid sequence of SEQ ID NO:28.
21. A nucleic acid or acids encoding the molecule or antibody of any one of paragraphs 1-16, fusion protein of paragraphs 17, or CAR of any one of paragraphs 18-20.
22. The nucleic acid or acids of paragraph 21, including an expression control sequence operably linked thereto, optionally wherein the expression control sequence includes a promoter optionally selected SFFV promoter or EFS promotor.
23. A vector including the nucleic acid or acids of paragraphs 21 or 22.
24. A host cell including the molecule or antibody of any one of paragraphs 1-16, fusion protein of paragraphs 17, CAR of any one of paragraphs 18-20, or nucleic acid or acids(s) of paragraphs 21-23.
25. A CAR immune cell including the CAR of any one of paragraphs 18-20, optionally wherein the immune cell is a T cell, natural killer (NK) cell, or macrophage (MA).
26. The CAR immune cell of paragraph 25, wherein the CAR immune cell maintains the ability to kill cancer ENPP3+ cancer cells after 1, 2, 3, 4 or more stimulations.
27. The CAR immune cell of paragraphs 25 or 26, wherein the CAR immune cell is resistant to exhaustion.
28. The CAR immune cell of any one of paragraphs 25-27, wherein the antigen binding domain of the CAR includes the CDRs and optionally heavy and light chain variable regions with or without the signal sequence of SX13-1, SX13-7, SX13-10, or SX13-12.
29. The CAR immune cell of any one of paragraphs 25-28 including the scFv of any one of SEQ ID NOS: 19-26.
30. The CAR immune cell of any one of paragraphs 25-29 including SEQ ID NO:28, optionally linked at the N-terminus to the scFv of any one of SEQ ID NOS: 19-26.
31. A pharmaceutical composition including the CAR T cells of any one of paragraphs 25-30 or the molecule or antibody of any one of paragraphs 1-16 optionally wherein the molecule or antibody includes a drug conjugated thereto and/or has antibody-dependent cell-mediated cytotoxicity (ADCC) activity, or complement-dependent cytotoxicity activity (CDC), and/or is a bispecific engager optionally a T cell engager or NK cell engager.
32. A method of treating cancer, including administering a subject in need thereof an effective amount of pharmaceutical composition of paragraph 31.
33. The method of paragraph 32, wherein the subject has a cancer including cancer cells including expression of ENPP3.
34. The method of paragraph 33, wherein the expression of ENPP3 on the surface of the cancer cells is elevated compared to non-cancer cells, optionally non-cancer cells of the same cell type.
35. The method of any one of paragraphs 31-34, wherein the cancer is a renal cell carcinoma (RCC), optionally wherein the RCC is clear cell RCC or a non-clear cell RCC optionally selected papillary renal cell carcinoma, chromophobe renal cell carcinoma, collecting duct RCC, multilocular cystic RCC, medullary carcinoma, mucinous tubular and spindle cell carcinoma, and neuroblastoma-associated RCC.

### EXAMPLES

### Example 1: Design and Construction of ENPP3-Binders, CARs, and CAR T Cells

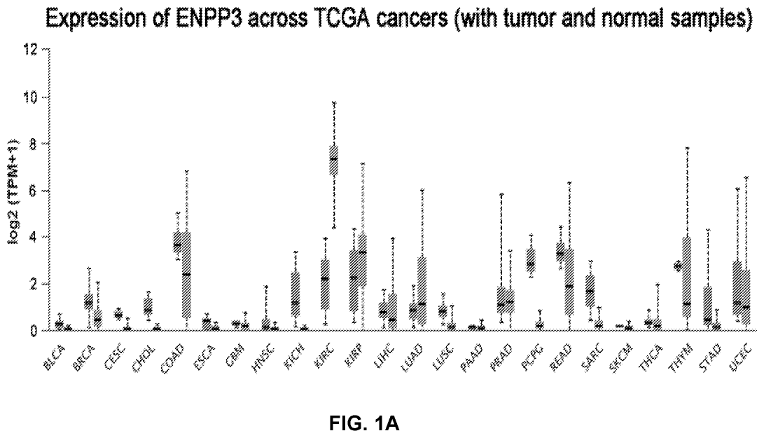

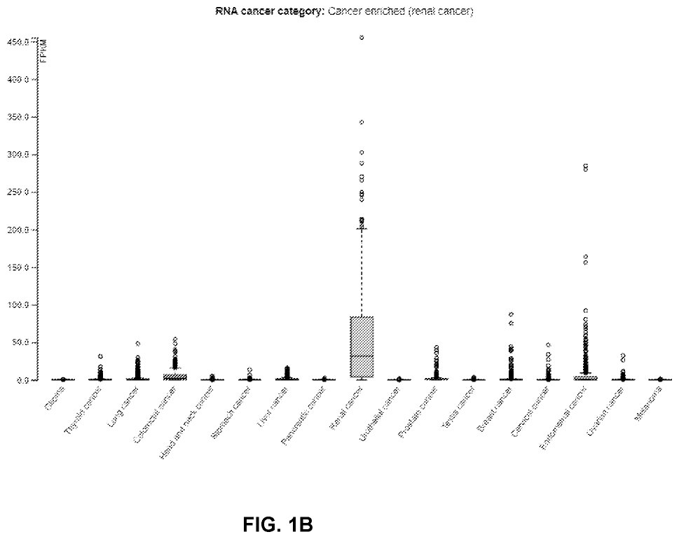

To identify suitable tumor-associated antigens (TAA), an explorative assessment of genes differentially expressed in kidney cancer was carried out. Gene expression analysis of patient data was performed using cancer and healthy donor genomics databases including TCGA and GTEX. Results identified ENPP3 to be highly expressed in RCC and had minimal expression in normal tissues. See, e.g., FIGS. 1A and 1B, KICH (Kidney Chromophobe), KIRC (Kidney Renal Clear Cell Carcinoma), KRIP (Kidney Renal Papillary Cell Carcinoma).

mRNA antigen was used to immunize B6 and ATX-GK humanized mice. NGS and identified multiple single BCR pairs and cloned them into human IgG form to produce antibodies (new binders).

Flow analysis was performed.

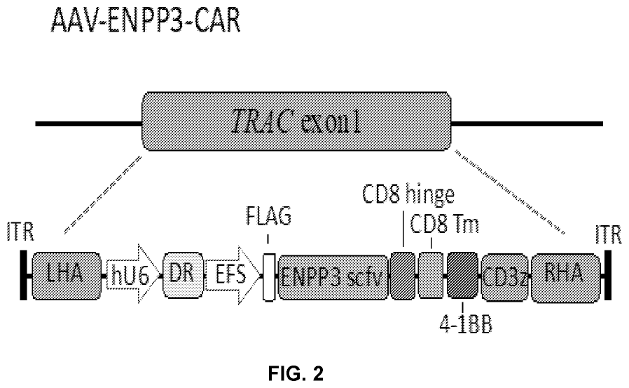

Eight of these binders, See clones 1, 2, 5, 6, 7, 8.1, 10 and 12 of FIGS. 2A-2S having the paired light and heavy chain variable regions according to SEQ ID NOS: 2-18 were selected as high binders for cell surface ENPP.

These binders were then converted into scFv forms according to SEQ ID NOS: 19-26.

Several scFv forms were used to prepare ENPP3-targeting chimeric antigen receptor fusion proteins having the structure: ENPP3-scFv-CD8hinge-CD8TM-41BBcostim-CD3z. See FIG. 2. Transformed CAR T cells could be detected by FLAG (e.g., using anti-FLAG antibody).

Lentiviral and AAV vectors were used to generate CAR-T cells from healthy donor cells. Lentivirus was produced in Lenti-X™ 293T Cell Line (Takara). Briefly, on day 1 (eighteen to twenty-four hours prior to transfection), 8-9E+6 cells were seeded in 100 cell culture petri dish. On day 2, the cells were transfected using PEI-PRO with packaging and expressing vectors (see manufacturer protocol), following with media replacement on day 3. Viruses were collected on day 4 from supernatants. The viruses got concentrated after mixing with PEG-LV concentrator (Takara Bio) according to manufacturer's instructions.

AAV viruses were produced using AAVpro® 293T Cell Line (Takara). The same number of cells were seeded as described above followed by transfection with AAV packaging vectors and expression vectors using PEI MAX reagent on day 2 and changing the media 12 h after transfection (day 3). AAVs were collected 72 h after media change (both supernatant and cells). Briefly, cell Pellets were resuspended in 1× gradient buffer (10× gradient buffer includes 7.5 ml 1M Tris pH 7.5, 6.57 g NaCl, 1.52 g 1M MgCl₂ 6H₂O, ddH₂O to 50 ml), followed by PEG/NaCl precipitation and then cell lysis (freeze/thaw method) and clearing the prep using 10% sodium deoxycholate and NaCl. After building iodixanol gradient (i.e. 15%, 25%, 40% and 58%), the virus prep was added to the gradient followed by ultracentrifuge (48,000 rpm, 2 h 10 min at 18 C). The fraction with AAV (between 40% and 58% gradient buffer) was collected after ultra centrifuge by a syringe, followed by filtering an dialysis (Millipore). The MOI was calculated using qRT-PCR.

For AAV viruses, Cas12a mRNA/TRAC gRNA (TRAC gRNA is expressing in the AAV vector under U6 promotor, FIG. 2) was used to knockout TRAC and CARs were inserted (Knock-in) into TRAC locus precisely (KIKO for Knock-in/Knock-out). Briefly, T cells were activated using 1:1 ratio of CD3/CD28 Dynabeads for 2.5 days. On day 3, T cells (2 million cells) were debeaded and electroporated (Lonza) with 10 μg cpf1 (Cas12a mRNA) and AAV viruses were added 2 hours post-electroporation (final MOI of 5). Knock-in efficiency in TRAC locus was tested after 5 days using flow cytometer (CD3 negative and CAR positive population).

For lentiviruses, the viruses were added to the activated/debeaded T cells (final MOI of 5). Briefly, 3×E5 T cells activated with 1:1.5 CD3/28 imuuno magnetic beads were plated in 200 μl complete T cells media [Optimizer, 200 IU IL2, 5 ng/ml IL-7 and 5 ng/mL IL15, 5% AB+ human serum] in a 24 well plate and mixed/thoroughly resuspended with the appropriate amount of the lentiviral suspension at MOI equal to or below 5. T cells were supplemented with additional 0.5 ml of the complete media for expansion for up to 3-5 days in 16-24 hour later. Lentivirus-delivered transgene expression was assessed by FACs 3-5 days later (FLAG expression).

### Example 2: ENPP3 CAR T Cells Displayed Robust Antitumor Activity Against RCC Cell Lines

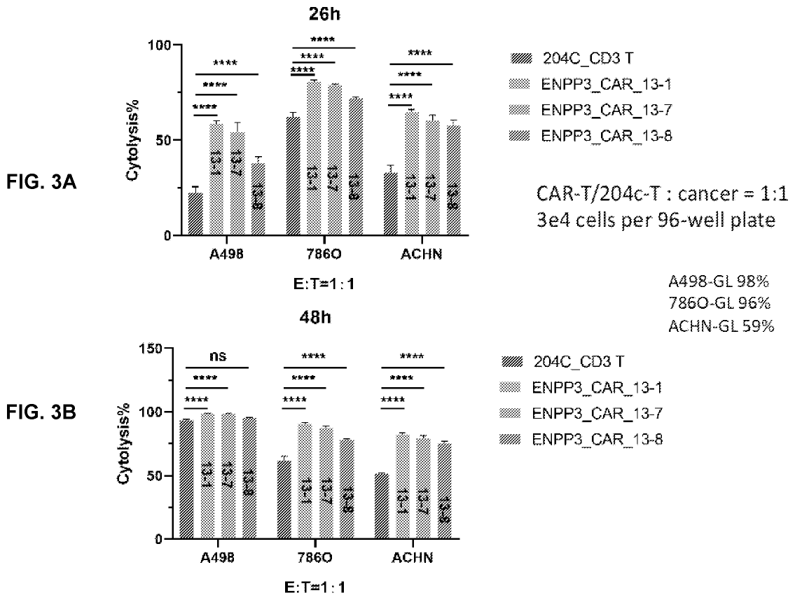

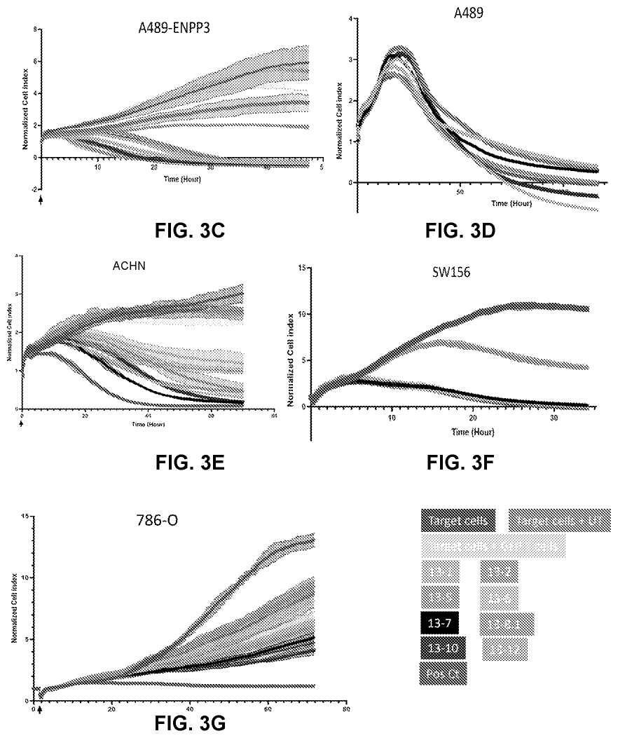

ENPP3 CAR T cells displayed robust antitumor activity against RCC cell lines and xenograft mouse models. Briefly, to evaluate CAR functionality two methods were used: cytotoxicity assay using target cells with luciferase expression and RTCA (real time killing assay) using xCelligencen. FIGS. 3A and 3B showed SX13-1, 13-7 and 13-8 cytotoxicity against A498, 786-O and ACHN cells. In addition, RTCA was performed against cell lines (A498, A498-ENPP3, SW156, ACHN and 786-0). These cells feature range of native and lentiviral generated ENPP3 expression levels with A498-ENPP3 (transduced with lenti virus to over-express ENPP3) the highest and 786-O the lowest ENPP3 expression.

FIGS. 3C-3G show SX13-1, 2, 5, 6, 7, 8.1, 10, 12 and positive control (form patent US20200317814A1) ENPP3 CARs in vitro real time killing assay using xCelligence (Agilent) at ratio of E:T 1:1. Cancer cells were initially seeded at the density of 10,000 per 96 well. Un-transduced (UT)/CAR T cells were added after 24 hours, equalizing expression of different ENPP3-CAR candidates by % un-transduced cells (the same CAR-T cells number and UT in each CAR).

### Killing Assays Featured:

A498 parental cell line: Sensitive to ENPP3-CAR killing
786-O (low ENPP3 expression level) cells less sensitive (i.e., resilient) to ENPP3-CAR killing
Lenti-A498 (medium ENPP3 express level) lentiviral viral cell line: Sensitive to ENPP3-CAR killing
ACHN cells are ENPP3 medium expressing cells.

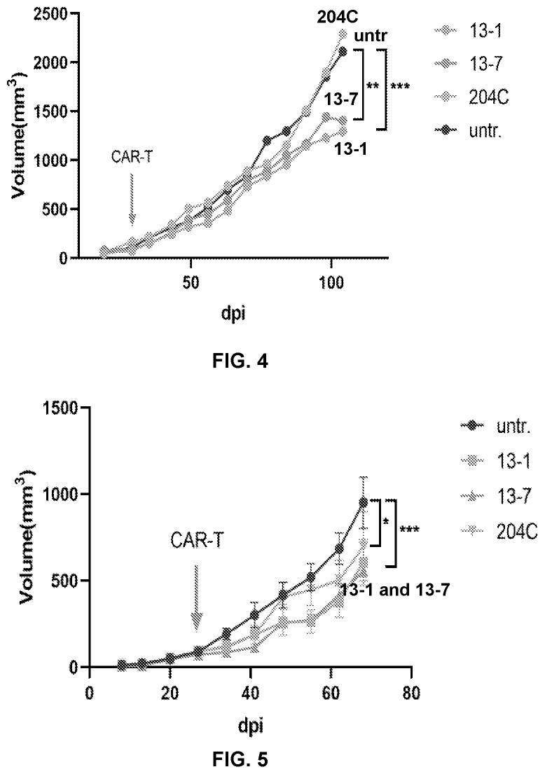

Together, these data showed that all the experimental ENPP3 CARs showed selective cytotoxic activity against kidney cancer cell lines. FIG. 4 shows SX13-1 and SX13-7 ENPP3 CARs in an in vivo tumor elimination assay. The control groups of 204 C (untransduced T cells from 204C donor) and PBS were used in all in vivo studies. The 786-O cells were injected into NSG mice subcutaneously followed by CAR T cells injection intravenously. Tumor size was measured weekly. FIG. 5 also shows SX13-1 and SX13-7 ENPP3 CARs in an in vivo tumor elimination assay. 786-O cells were injected into NBSGW mice subcutaneously followed by CAR T cells injection intravenously. Tumor size was measured weekly.

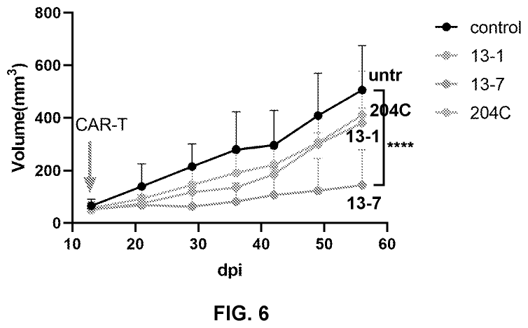

FIG. 6 also shows SX13-1 and SX13-7 ENPP3 CARs in an in vivo tumor elimination assay. ACHN cells were injected into NSG mice subcutaneously followed by CAR T cells injection intravenously. Tumor size was measured weekly.

In sum, results show ENPP3 CARs were highly effective and lysed target cells across the antigen expression range. The in vivo experiments also displayed preliminary pre-clinical efficacy of ENPP3 CAR-Ts in RCC tumors in mice. Taken together, these efficacy data support ENPP3 CAR-T cells for the treatment of RCC and other ENPP3+ cancer indications.

### Example 3: CAR T are Effective for Serial Killing

### Materials and Methods

ENPP3-CAR T cells were generated using lentivector pSFFV-FLAG-ENPP3-CAR and yielded candidates 13-1, 13-5, 13-7, 13-8.1, 13-10,13-12 and positive control.

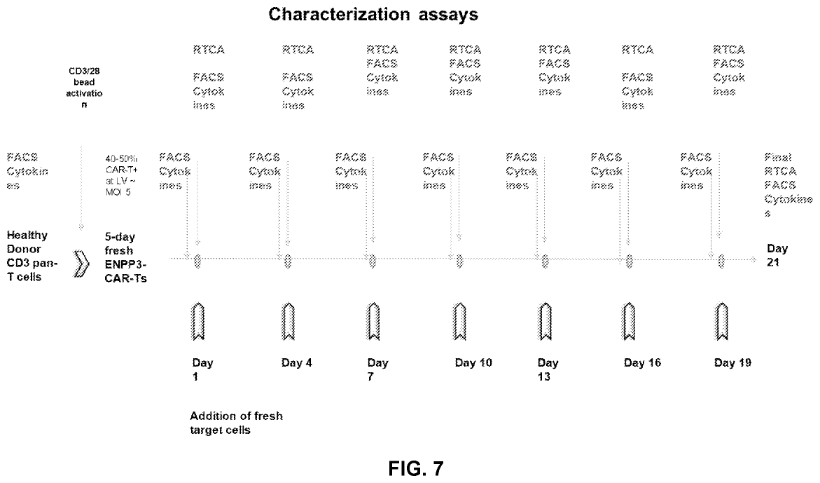

A CAR T evolution in a serial co-culture assay is illustrated in FIG. 7.

### Samples in the Study:

1. CAR-Ts co-cultured with target cells.
2. CAR-T expanded without stimulation with target cells. Note: this will represent CAR-T for assessment of CAR-autoactivation

### Methods and Readouts:

1. FACS assessment of T cell biomarkers for antigen induced activation, stemness and exhaustion
2. FACS assessment of CAR expression/density on T cell
3. T cell killing efficiency using RTCA killing assay
4. Cytokine expression profiling using ELISA and Luminex multiplex assay

### Results

Results show that SFFV promoter is stronger than EFS promotor.

Serial co-culture assay results show the expression level of donor 3 ENPP3 CARS begins to decline after stimulation #3. CAR 13-8.1 and 13-5 lost their capability of killing after 3 times stimulation. CARs 13-1, 13-12 and positive control can not kill efficiently after 4 times stimulation. However, CAR 13-7 and 13-10 (specially 13-10) are very good in killing even after 4 times stimulation with target cells (A498 cells). None of the ENPP3 CARs can efficiently kill after 6 times stimulation.

For donor 4 CARs, most of the CARs lost their killing ability after just two simulations. CAR 13-10 and the positive control were still killing. After 4 stimulations with A498 target cells on donor 4 CARs, CAR 13-10 was the most efficient killer, doing even better than the positive control.

A summarized results of single killing and the serial killing assays are shown in Table 1 below:

| | | | | | | | | | | Positive |
|---|---|---|---|---|---|---|---|---|---|---|
| Target cells, donor, ratio [symbol] T | Exp [symbol] | SX13-1 | SX13-2 | SX13- [symbol] | SX13-6 | SX13-7 | SX [symbol] | SX13-10 | SX13-12 | [symbol] |
| [symbol] , donor [symbol] ratio | 1 | [symbol] | [symbol] | [symbol] | N/A | [symbol] | [symbol] | [symbol] | [symbol] | [symbol] |
| [symbol] , donor [symbol] ratio | 1 | [symbol] | [symbol] | [symbol] | N/A | [symbol] | [symbol] | [symbol] | [symbol] | [symbol] |
| A [symbol] , donor 3, [symbol] ratio | 2 | [symbol] | [symbol] | [symbol] | N/A | [symbol] | [symbol] | [symbol] | [symbol] | [symbol] |
| A [symbol] , donor 3, [symbol] ratio | 2 | [symbol] | [symbol] | [symbol] | N/A | [symbol] | [symbol] | [symbol] | [symbol] | [symbol] |
| 7 [symbol] -O, donor3, [symbol] ratio | 2 | [symbol] | [symbol] | [symbol] | N/A | [symbol] | [symbol] | [symbol] | [symbol] | [symbol] |
| 7 [symbol] -O, donor3, [symbol] ratio | 2 | [symbol] | [symbol] | [symbol] | N/A | [symbol] | [symbol] | [symbol] | [symbol] | [symbol] |
| A498, donor 3, 1:1 ratio, serial [symbol] St 3 | | [symbol] | N/A | N/A | N/A | N/A | N/A | [symbol] | [symbol] | N/A |
| A498, donor 4, 1:1 ratio | 3 | [symbol] | [symbol] | [symbol] | N/A | [symbol] | [symbol] | [symbol] | [symbol] | [symbol] |
| 786-O, donor 4, 1:1 ratio | 3 | [symbol] | [symbol] | [symbol] | N/A | [symbol] | [symbol] | [symbol] | [symbol] | [symbol] |
| ACHN, donor 4, 1:1 | 4 | [symbol] | [symbol] | [symbol] | [symbol] | [symbol] | [symbol] | [symbol] | [symbol] | [symbol] |
| ACHN, donor 4, 1:5 | 4 | [symbol] | [symbol] | [symbol] | [symbol] | [symbol] | [symbol] | [symbol] | [symbol] | [symbol] |
| 786-O, donor 4, 1:1 | 4 | [symbol] | [symbol] | [symbol] | [symbol] | [symbol] | [symbol] | [symbol] | [symbol] | [symbol] |
| 786-O, donor 4, 1:5 | 4 | [symbol] | [symbol] | [symbol] | [symbol] | [symbol] | [symbol] | [symbol] | [symbol] | [symbol] |
| A498, donor 4, 0.5:1 ratio | 4 | [symbol] | [symbol] | [symbol] | [symbol] | [symbol] | [symbol] | [symbol] | [symbol] | [symbol] |
| A498, donor 4, 2:1 ratio | 4 | [symbol] | [symbol] | [symbol] | [symbol] | [symbol] | [symbol] | [symbol] | [symbol] | [symbol] |
| A498, donor 3, 1:1 ratio, serial#2 St 3 | | [symbol] | N/A | [symbol] | N/A | [symbol] | [symbol] | [symbol] | [symbol] | [symbol] |
| A498, donor 3, 1:1 ratio, serial#2 St 4 | | [symbol] | N/A | [symbol] | N/A | [symbol] | [symbol] | [symbol] | [symbol] | [symbol] |
| A498, donor 3, 1:1 ratio, serial#2 St 6 | | [symbol] | N/A | [symbol] | N/A | [symbol] | [symbol] | [symbol] | [symbol] | [symbol] |
| A498, donor 4, 1:1 ratio, serial# [symbol] St 2 | | [symbol] | N/A | [symbol] | [symbol] | [symbol] | [symbol] | [symbol] | [symbol] | [symbol] |
| A498, donor 4, 1:1 ratio, serial# [symbol] St 4 | | [symbol] | N/A | [symbol] | [symbol] | [symbol] | [symbol] | [symbol] | [symbol] | [symbol] |
| A498, donor 4, 1:1 ratio, serial# [symbol] St 6 | | [symbol] | N/A | [symbol] | [symbol] | [symbol] | [symbol] | [symbol] | [symbol] | [symbol] |

[symbol]

indicates data missing or illegible when filed

The 13-7, 13-10 and 13-12 CARs are the winners for serial killing on donor 3 (specially 13-10) and CAR 13-10 is the winner for donor 4. In general donor 3 CARs are better killers than donor 4. Please not positive control has a scFv from the following patent: US20200317814A1.

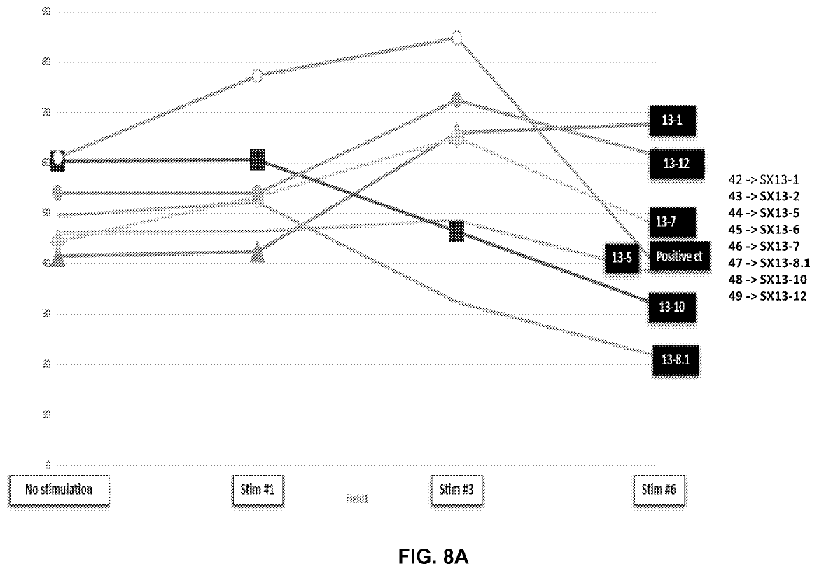

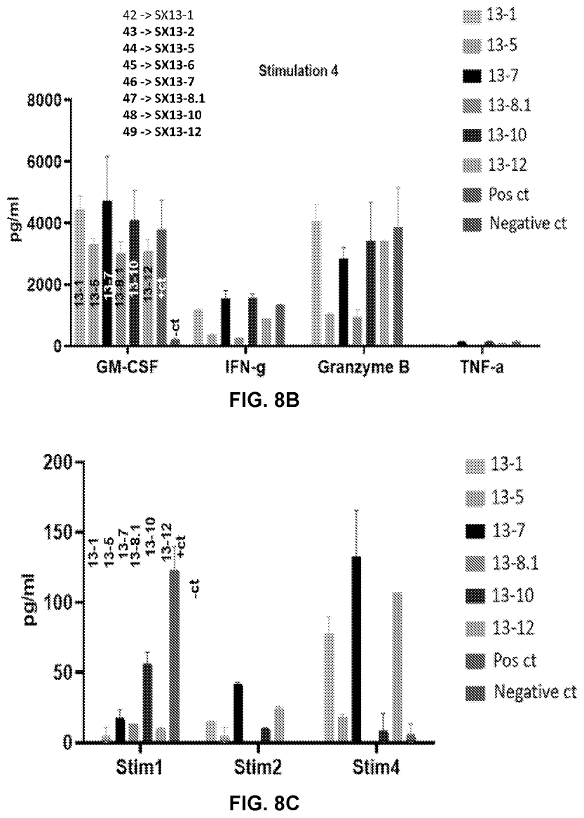

FIG. 8A shows CAR expression in different CARs in serial killing. FIG. 8B shows a cytokine profile (Luminex) of ENPP3 CARs in the serial killing assay (donor 3), specifically after 4 stimulations. CAR 13-7, 13-10, and 13-12 showed as good as positive control CAR for GM-CSF secretion and Granzyme B.

FIG. 8C shows the IL-4 profile after various stimulations. A co-culture of the most efficient killer, CAR 13-10, with A498 kidney cancer cells (stimulation) showed the lowest IL-4 production, indicating Treg population depletion. IL-4 is a regulatory cytokine.

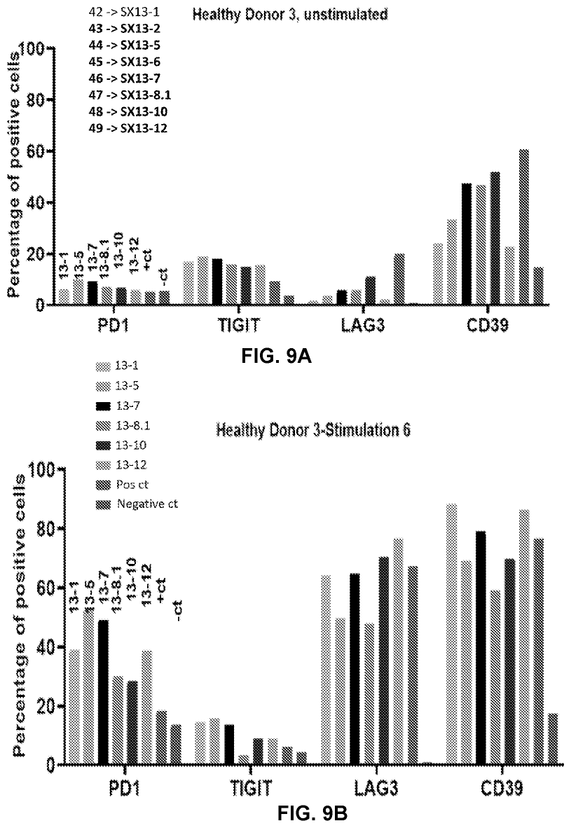

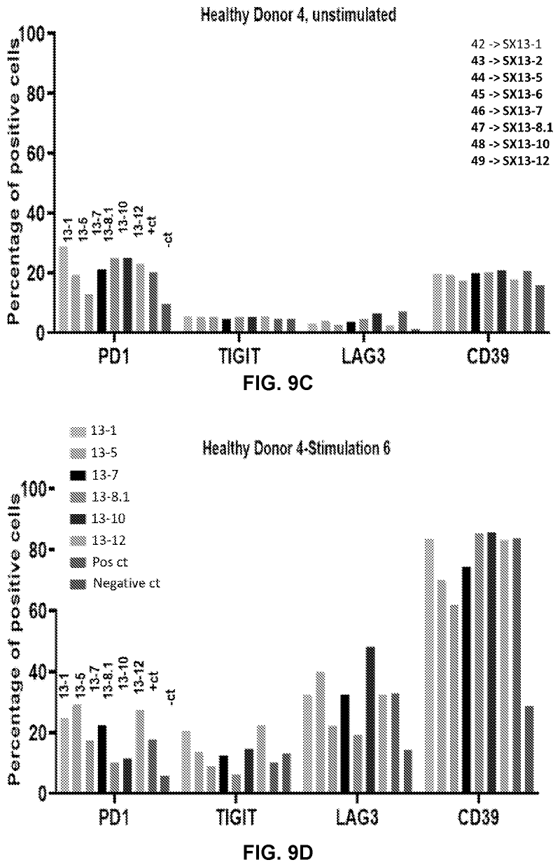

FIGS. 9A-9D show the profiles of four exhaustion markers (PD1, TIGIT, KAG3 and CD39) for the donor 3 ENPP3 CARs (FIGS. 11A, 11B) and the donor 4 ENPP3 CARs (FIGS. 11C, 11D) unstimulated (FIGS. 11A, 11C) after 6 stimulations (FIGS. 11B, 11D) with A498 kidney cancer cells. The following fluorophores were used for the staining: CD39-FITC, LAG3-PerCP-eFluor 710, TIGIT-PE, PD1-PE-Cy7. Donor 3 has 55.45% CD4 and 29.39% CD8. As discussed above, the most efficient killers after serial killing for donor 3 were CARs 13-7, 13-10 and the positive control CAR (from patent), and for donor 4 was CAR 13-10.

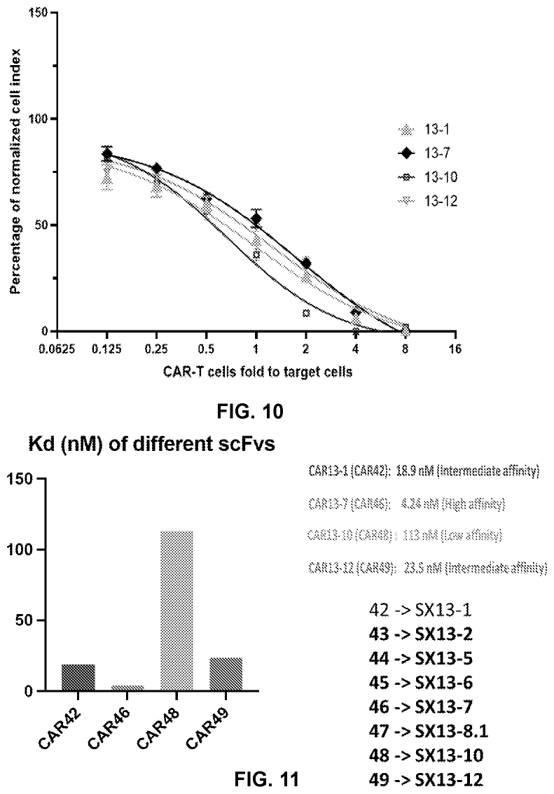

FIG. 10 shows the EC50 for killing (single killing, just one time stimulation) for preferred final candidate ENPP3 CARs (13-1, 13-7, 13-10 and 13-12) at 24 h time point (24 h after adding CARs to target cells which is A498 kidney cancer cells). The EC50 was calculated as following 0.97, 1.76, 0.66 and 1.26 for 13-1, 13-7, 13-10 and 13-12, respectively.

### Example 4: Further Analysis, Selection and Validation of Effective ENPP3-Specific CAR T Cells

### Materials and Methods

ENPP3 expression is present in different cancers and normal tissues, with ENPP3 highly expressed in RCC (positivity in 92.3% and high expression in 83.9% of samples), downstream of the oncogenic VHL/HIF pathway. Because ENPP3 has restricted expression in normal tissues, and generally in non-T cell accessible luminal surfaces, a panel of CAR T cells that selectively recognize ENPP3 was prepared. The CARs were prepared as fusion proteins including antigen-binding components of each of the anti-ENPP3 antibodies: SX13-1, SX13-2, SX13-5, SX13-6, SX13-7, SX13-8.1, SX13-10 and SX13-12.

Corresponding CAR T cell including CARs having the antigen binding components of each of these antibodies (as an ScFv domain) were prepared and characterized as follows: CAR42 (SX13-1); CAR43 (SX13-2); CAR44 (SX13-5); CAR45 (SX13-6); CAR 46 (SX13-7); CAR 47 (SX13-8.1); CAR 48 (SX13-10); and CAR 49 (SX13-12). A positive control and negative control (no T cells) were also prepared.

### Results

A binding affinity assay was carried out using each of these CARTs (FIG. 11) to identify the CARs having the highest affinity for ENPP3. The data demonstrated that CAR 42 (13-1) had 18.9 nM (intermediate affinity); CAR46 (13-7) had 4.24 nM (High affinity), CAR48 (13-10) had 113 nM (Low affinity); and CAR40 (13-12) had 23.5 nM (Intermediate affinity).

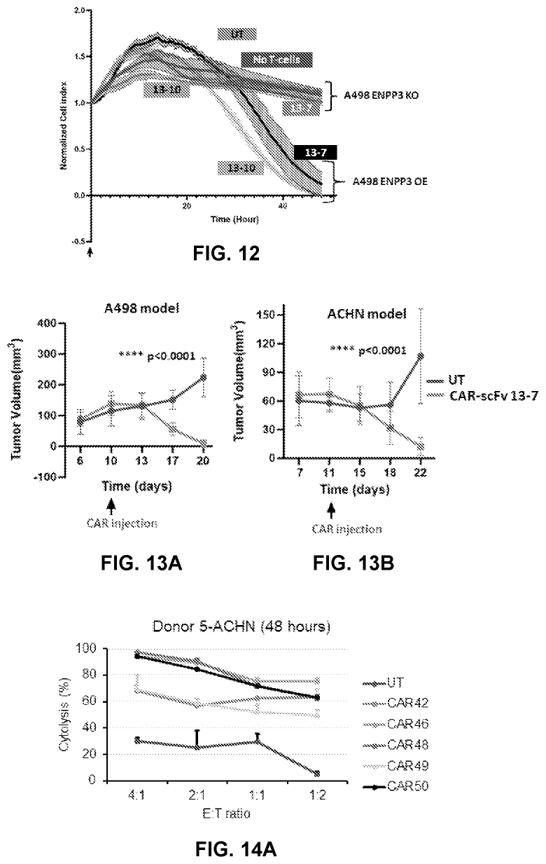

Next, an RTCA assay (killing assay) was carried out using ENPP3 Knock Out (KO) A498 and ENPP3 over-expressing (OE) A498 cells using ENPP3 CARsc Fv 13-7 and 13-10 (FIG. 12). The data demonstrate high specificity of the scFvs, as evidenced by a lack of cytolytic activity against A498 ENPP3 KO cells.

To test the in vivo efficacy of the CAR Ts, tumor growth of A498 and ACHN human kidney tumor was assessed with ENPP3 CAR 13-7 treatment. Mice were first subcutaneously injected with 4×10⁶ tumor cells; five million ENPP3 CAR 13-7 cells, or non-transduced CD3+ T cells were infused into mice at day 10. Two-way ANOVA was used to assess significance. **** p<0.0001. By comparing CAR scFv 13-7 in vivo in an A498 and ACHN xenograft models, a superior anti-tumor response was observed for CAR scFv 13-7 (FIGS. 13A-13B). Finally, ENPP3 protein expression in ACHN Xenograft Tumors was investigate. Together, the data confirmed ENPP3 protein expression in ACHN Xenograft Tumors and demonstrated superior anti-tumor response for CAR T cells including a CAR having the scFv from antibody SX13-7.

### Example 5: ENPP3-Specific CAR-46 T Cells Produce Anti-Tumor Cytokine in Different Donors, Maintain Better Stemness and Central Memory Features and Overcome CAR-T Exhaustion Upon Target Cell Exposure Compared to Other scFV

### Materials and Methods

To further characterize anti-tumor efficacy of ENPP3-specific scFv, a panel of assays, including cytolysis assays, and cytokine induction assay, survival assay was carried out with fresh CAR T cells, including CAR T cells produced using CAR42 (SX13-1); CAR 46 (SX13-7); 48 (SX13-10); and CAR 49 (SX13-12), in a variety of donor cell backgrounds, as well as CAR50 and UT (Control).

### Results

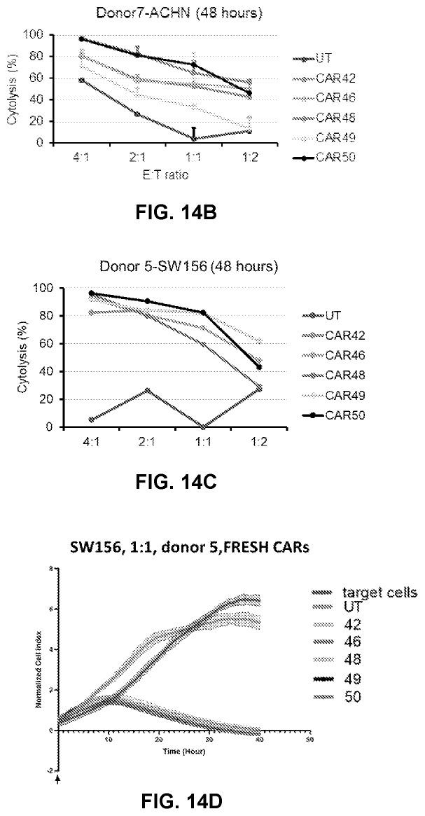

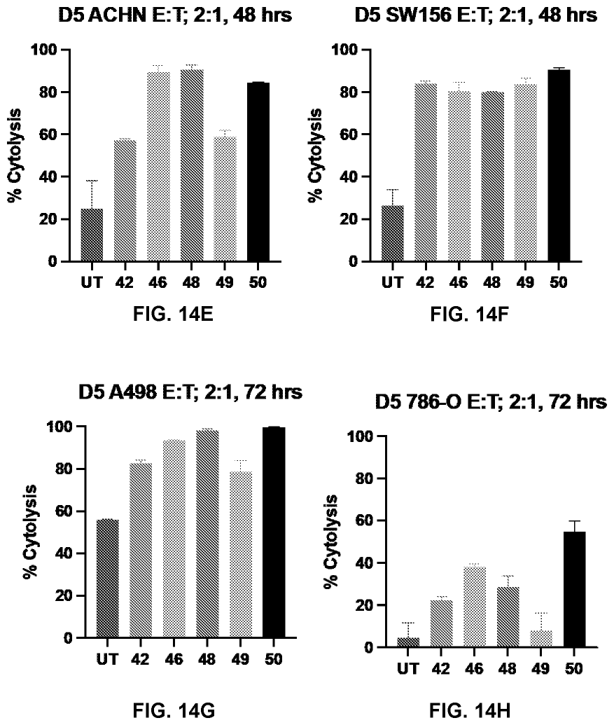

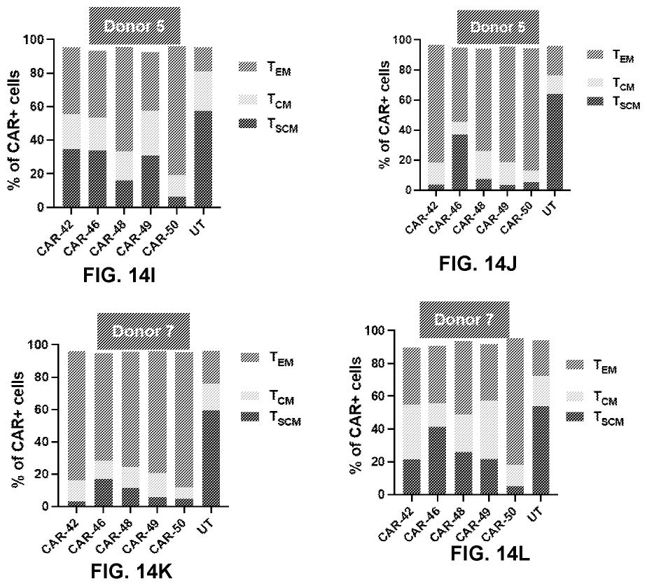

Cytolysis assay with fresh CARs demonstrated that CAR46 (SX13-7); and CAR48 (SX13-10) showed better killing efficacy against ACHN, while all CARs showed strong elimination of SW156 (FIGS. 14A-14D).

FIGS. 14E-14H are plots showing quantification of Enpp3 CAR killing efficiency across different kidney cancer cell lines, E:T 2:1 at 48 h (FIGS. 14E and 14F) and 72 h (FIGS. 14G and 14H), after adding the CAR-Ts using Luc assay.

FIGS. 14I-14L are plots showing phenotyping of ENPP3 CAR-T in two different donors (donor 5 and 7), after three times stimulations with ACHN (FIG. 14I and FIG. 14L) and two times stimulations with SW156 (FIGS. 14J-14K).

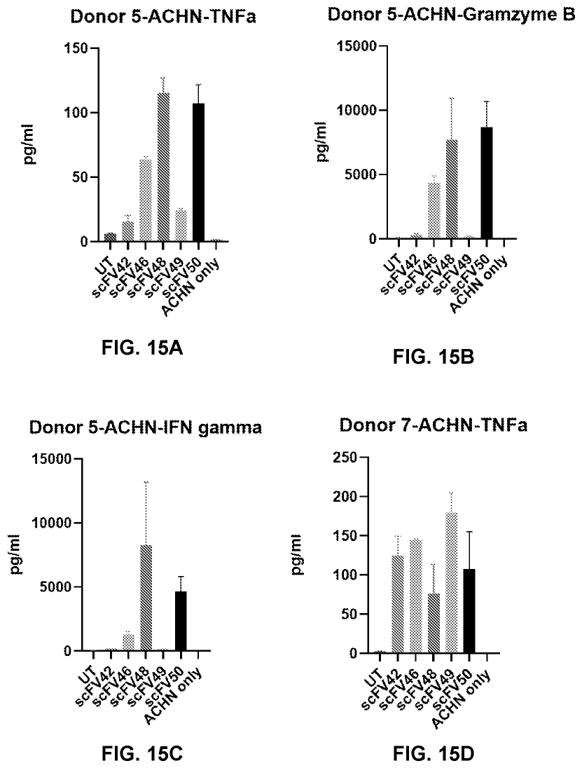

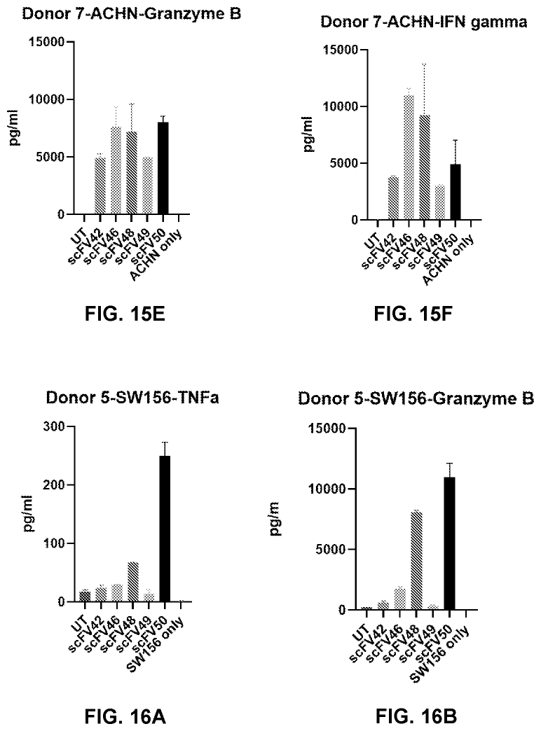

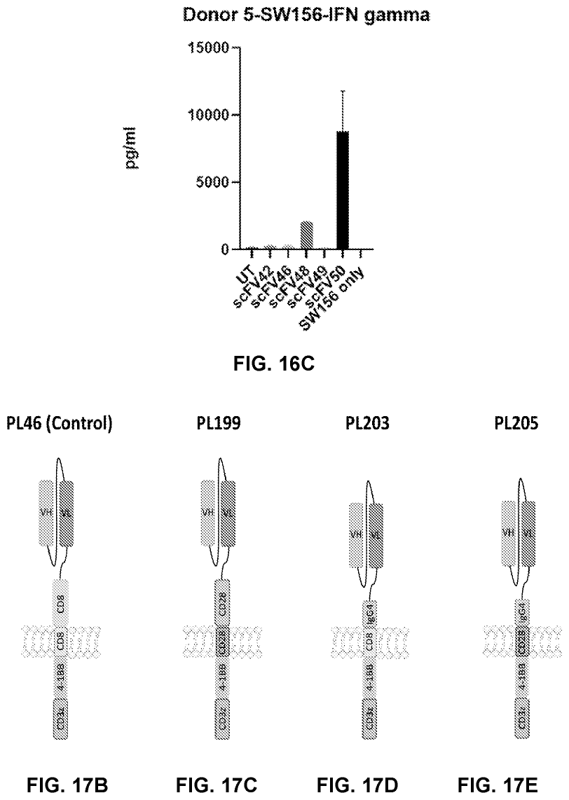

Quantification of Enpp3 CAR killing efficiency across different kidney cancer cell lines and reduced killing from exhausted CAR-Ts, with scFV48 showed more than 40% cytolysis. scFV46 and scFV48 showed strong anti-tumor cytokine induction in different donors, as demonstrated in FIGS. 15A-15F and 16A-16C). Data from these studies are summarized in Table 2, below.

Taken together, the data demonstrated that CAR-46 maintains better stemness and central memory features compared to other scFV, and that CAR-46 Fitness is evident in multiple donors (Donor 5 and Donor 7). The data also show that CAR-46 overcomes CAR-T Exhaustion upon target cell exposure, as compared to other scFV.

TABLE 2
Summary of cytolysis, cell killing, serial killing, exhaustion and fitness assays

| Assays | CAR42 | CAR46 | CAR48 | CAR49 | CAR50 |
|---|---|---|---|---|---|
| E:T (Cytolysis) % of killing | | | | | |
| normalized with UT | | | | | |
| SW156 | 60 | 57 | 57 | 60 | 70 |
| ACHN | 25 | 65 | 65 | 25 | 60 |
| 786-O | 17 | 35 | 25 | 5 | 48 |
| A498 | 26 | 35 | 40 | 24 | 45 |
| RTCA killing | | | | | |
| SW156 | +++ | +++ | +++ | +++ | +++ |
| Serial killing (Cytolysis) | | | | | |
| ACHN | + | ++ | ++ | + | ++ |
| Overcoming CAR-T Exhaustion | ++ | +++ | + | ++ | − |
| CAR-T cell Fitness (Stemness and | ++ | +++ | + | + | − |
| memory Potential) | | | | | |
| Anti-tumor cytokines | + | ++ | +++ | + | +++ |
| No harmful cytokines | TBD | TBD | TBD | TBD | TBD |
| Kd (nM) | 18.9 | 4.24 | 113 | 23.5 | NA |
| Retrogenix | NA | No Major | No Major | NA | NA |
| | | Concern | Concern | | |
| In vivo efficacy | TBD | TBD | TBD | TBD | TBD |
| In vivo safety | TBD | TBD | TBD | TBD | TBD |

### Example 6: CAR Improvement Using scFv of Antibody SX13-7

### Materials and Methods

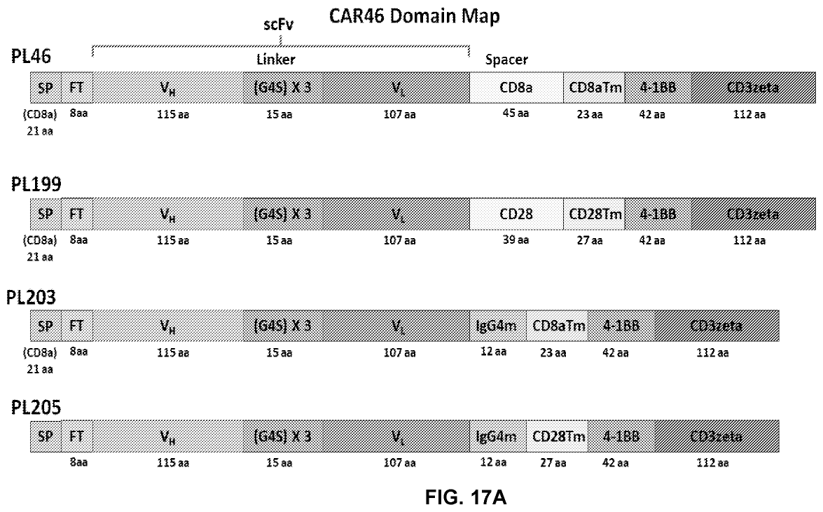

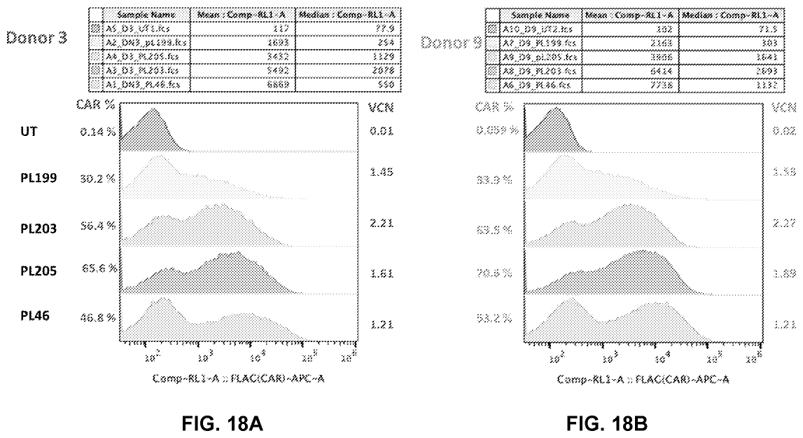

To characterize and improve anti-tumor efficacy of ENPP3-specific CARs having different coreceptor components, various structural variant CARs were engineered based on the CAR46 antigen binding domain. An overview of CAR variants that were prepared is depicted in FIGS. 17A-17B. All variants, including PL46 (WT CAR); PL199 (CD28 Hinge+CD28Tm); PL203 (IgG4m Hinge+CD8 Tm); and PL205 (IgG4m Hinge+CD28 Tm) were expressed in each of two donor cells (Donors 3 and 9).

A luciferase assay for cytolysis was carried out with fresh CARs using SW156 cells, and 786-O cells, and A498 cells and ACHN cells

### Results

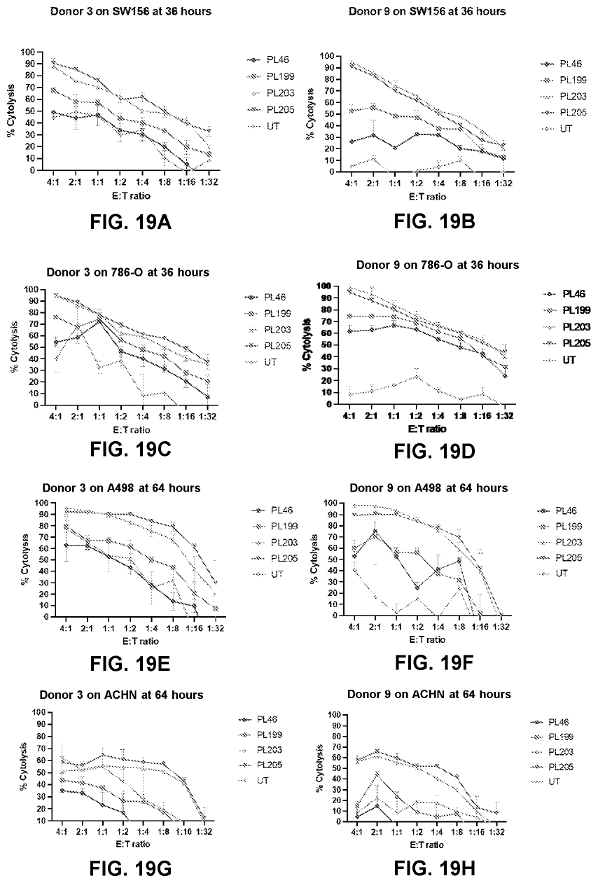

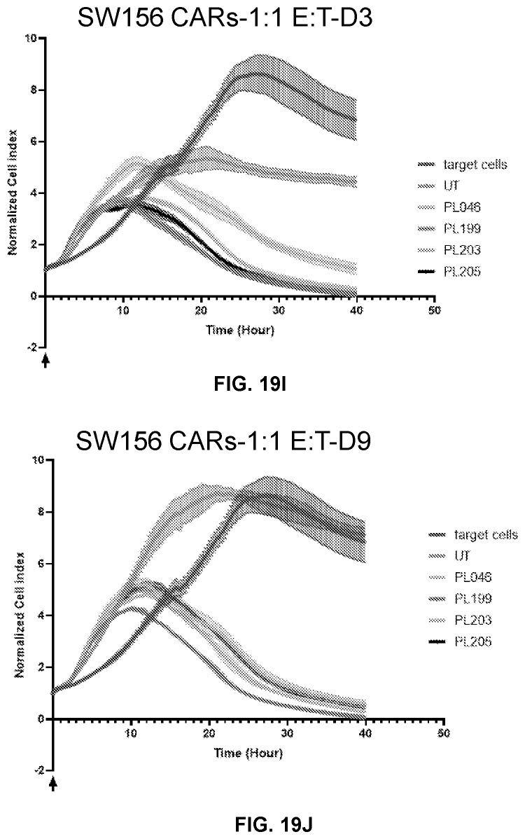

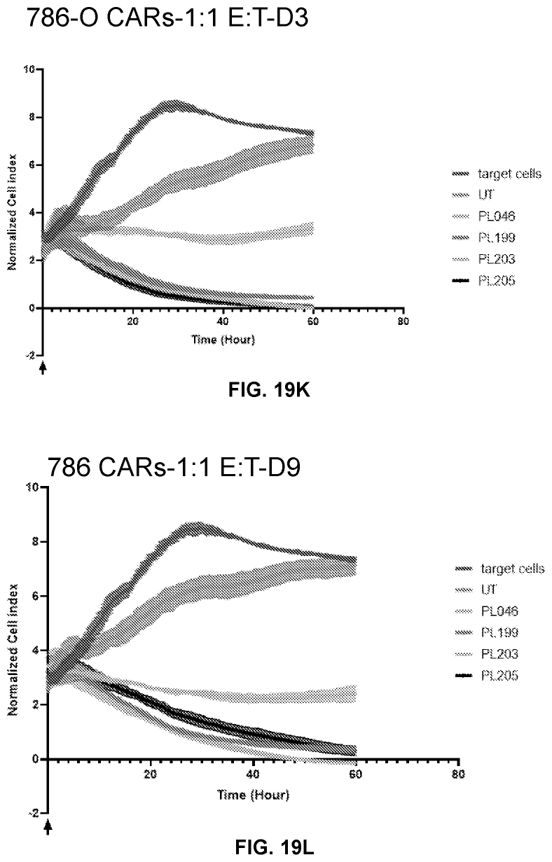

Data from the Luciferase assays identified PL203 (IgG4m Hinge+CD8 Tm); and PL205 (IgG4m Hinge+CD28 Tm) as the most effective candidate molecules (FIGS. 19A-19H). FIGS. 19I-19L are plots showing RTCA killing assay of ENPP3 CARs (all using scFv 46) on SW156 cells in two different donors (D3 and D9) at E:T 1:1 (FIGS. 19I-19J) and on 786-O cells in two different donors (D3 and D9) at E:T 1:1 (FIGS. 19K-19L).

CAR expression after ST1 (stimulation 1 with SW156 target cells) was observed in both donor cell lines.

### Example 7: Functional Assessment and Screening of Spacer and Co-Stimulation Domain Variants of CARs Using scFv of Antibody SX13-7

### Materials and Methods

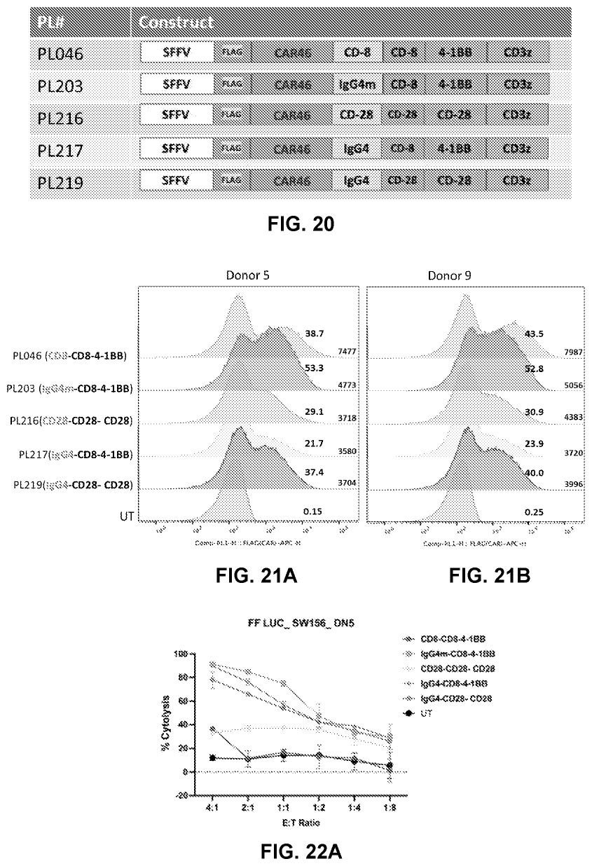

To further improve anti-tumor efficacy of ENPP3-specific CARs having different cytoplasmic components, various structural variant CARs were engineered based on the CAR46 antigen binding domain. An overview of CAR variants that were prepared is depicted in FIG. 20. All variants, including PL46 (WT CAR); PL203 (IgG4m Hinge+CD8 Tm; 4-1BB cytoplasmic); PL216 (CD28 Hinge+CD28 Tm; CD28 cytoplasmic); PL217 (IgG4m Hinge+CD8 Tm; 4-1BB cytoplasmic); and PL219 (IgG4m Hinge+CD28 Tm; CD28 cytoplasmic) were expressed in each of two donor cells (Donors 5 and 9).

A luciferase assay for cytolysis was carried out with fresh CARs using SW156 cells, and 786-O cells, and A498 cells in each of the two donor backgrounds.

### Results

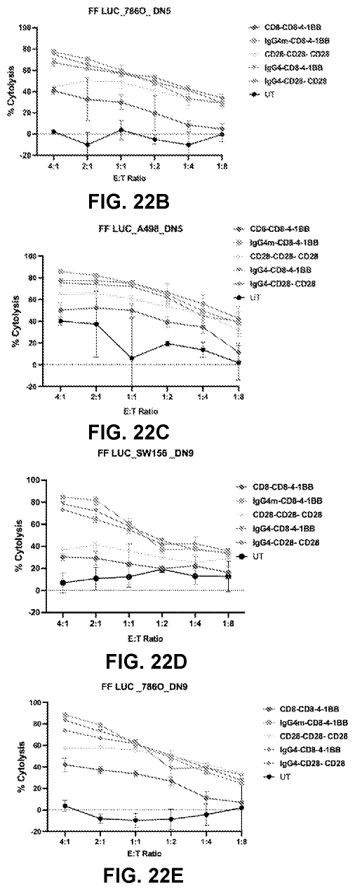

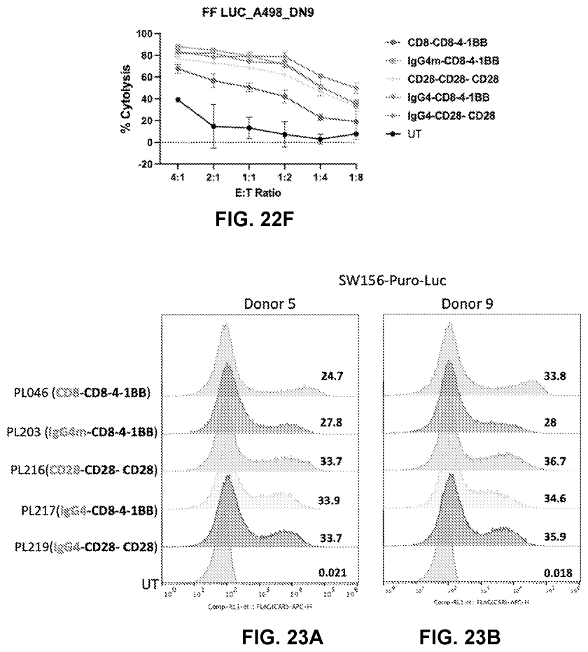

Data from the Luciferase assays identified PL203 (IgG4m Hinge+CD8 Tm; 4-1BB cytoplasmic); PL219 (IgG4m Hinge+CD28 Tm; CD28 cytoplasmic); and PL217 (IgG4m Hinge+CD8 Tm; 4-1BB cytoplasmic) as the most effective candidates. molecules (FIGS. 22A-22F).

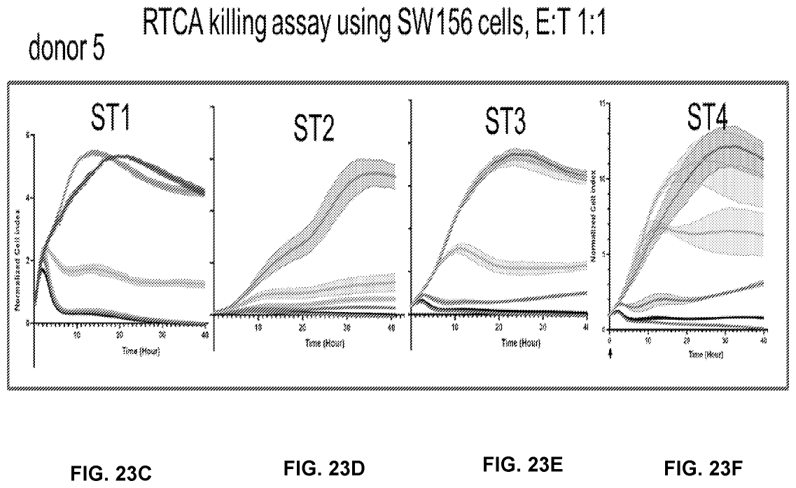

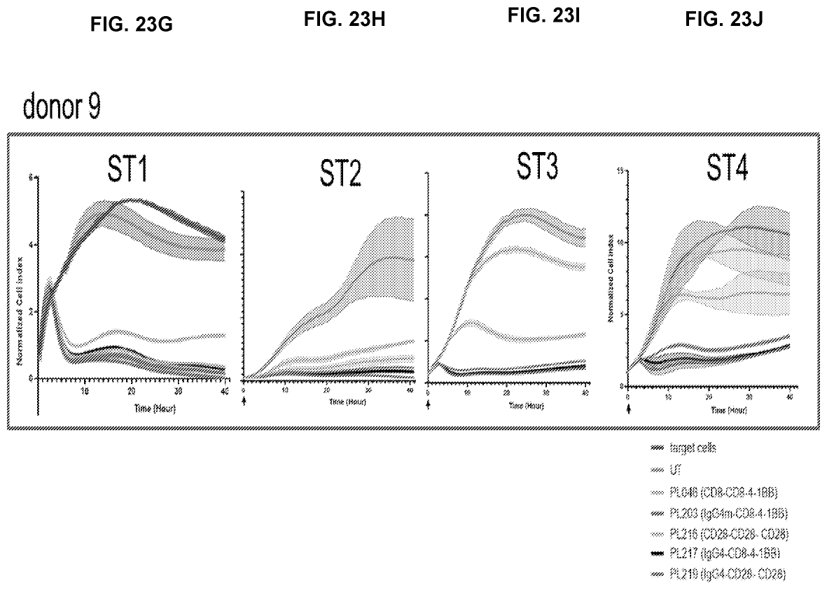

FIGS. 23A-23B show CAR expression profile after ST1 (stimulation 1) with SW156 target cells for PL046 (CD8-CD8-4-1BB), PL203 (IgG4m-CD8-4-1BB), PL216 (CD28-CD28-CD28), PL217 (IgG4-CD8-4-1BB), and PL219 (IgG4-CD28-CD28) from donor 5 (FIG. 23A) and donor 9 (FIG. 23B). FIGS. 23C-23J are plots showing RTCA killing assay of ENPP3 CARs (all using scFv 46) on SW156 cells in two different donors D5 (FIGS. 23C-23F) and D9 (FIGS. 23G-23J) at E:T 1:1 in serial killing assay: Stimulation 1, 2, 3 and 4.

1. A molecule or antibody comprising an antigen binding domain of an antibody that immuno-specifically binds to Ectonucleotide pyrophosphatase/phosphodiesterase 3 (ENPP3),
wherein the antigen binding domain comprises six complementarity determining regions (CDRs),
wherein the CDRs include at least one consensus CDR of the CDRs of an anti-ENPP3 antibody selected from the group consisting of SX13-7, SX13-10, SX13-1, SX13-2, SX13-5, SX13-6, SX13-8.1, and SX13-12.

2.-3. (canceled)

4. The molecule or antibody of claim 1, wherein all remaining CDRs are selected from:
(A) the three light chain CDRs and the three heavy chain CDRs of anti-ENPP3 antibody SX13-7;
(B) the three light chain CDRs and the three heavy chain CDRs of anti-ENPP3 antibody SX13-10;
(C) the three light chain CDRs and the three heavy chain CDRs of anti-ENPP3 antibody SX13-1;
(D) the three light chain CDRs and the three heavy chain CDRs of anti-ENPP3 antibody SX13-2;
(E) the three light chain CDRs and the three heavy chain CDRs of anti-ENPP3 antibody SX13-5;
(F) the three light chain CDRs and the three heavy chain CDRs of anti-ENPP3 antibody SX13-6;
(G) the three light chain CDRs and the three heavy chain CDRs of anti-ENPP3 antibody SX13-8.1; and
(H) the three light chain CDRs and the three heavy chain CDRs of anti-ENPP3 antibody SX13-12.

5. The molecule or antibody of claim 1 comprising six CDRs,
wherein the six CDRs are:
the three light chain CDRs and the three heavy chain CDRs of anti-ENPP3 antibody SX13-7;
the three light chain CDRs and the three heavy chain CDRs of anti-ENPP3 antibody SX13-10;
the three light chain CDRs and the three heavy chain CDRs of anti-ENPP3 antibody SX13-1;
the three light chain CDRs and the three heavy chain CDRs of anti-ENPP3 antibody SX13-2;
the three light chain CDRs and the three heavy chain CDRs of anti-ENPP3 antibody SX13-5;
the three light chain CDRs and the three heavy chain CDRs of anti-ENPP3 antibody SX13-6;
the three light chain CDRs and the three heavy chain CDRs of anti-ENPP3 antibody SX13-8.1; or
the three light chain CDRs and the three heavy chain CDRs of anti-ENPP3 antibody SX13-12.

6.-24. (canceled)

25. The molecule of claim 1, wherein the molecule comprises a chimeric antigen receptor (CAR),
optionally wherein the CAR comprises one or more of an extracellular antigen binding domain, a spacer domain, a transmembrane domain, and intracellular signaling domain.

26.-68. (canceled)

69. The molecule of claim 1, wherein the molecule comprises a chimeric antigen receptor (CAR), and
wherein the CAR comprises a framework comprising any one of SEQ ID NOs: 28, 120, 121, 122, 123, 124, or 125.

70. A CAR comprising (i) an antigen binding domain, optionally an anti-ENPP3 antigen binding domain, optionally an scFv or other construct, optionally of claim 1, and (ii) a variant spacer domain, transmembrane domain, and/or intracellular/costimulatory (“costim”) domains.

71. The CAR of claim 70, comprising the amino acid sequence of any one of SEQ ID NOS: 120-125.

72. The CAR of claim 70 comprising the amino acid sequence of any one of SEQ ID NOS: 91, 93, 95, 97, 99, 101, 103, 105, 107, 109, 111, 113, 115, 117, and 119 without SEQ ID NO:126, any one of SEQ ID NOS: 91, 93, 95, 97, 99, 101, 103, 105, 107, 109, 111, 113, 115, 117, and 119, or a fragment or variant thereof with at least 70% sequence identity thereto.

73. (canceled)

74. A nucleic acid encoding or expressing the molecule or antibody of claim 1 and/or the amino acid sequence of any one of SEQ ID NOS: 91, 93, 95, 97, 99, 101, 103, 105, 107, 109, 111, 113, 115, 117, and 119.

75. The nucleic acid of claim 74, further comprising an expression control sequence operably linked thereto,
optionally wherein the expression control sequence comprises a promoter.

76. The nucleic acid of claim 75, wherein the promoter is an SFFV promoter, or an EFS promotor.

77. (canceled)

78. A vector comprising the nucleic acid of claim 74.

79. A host cell comprising the molecule or antibody of claim 1.

80. A CAR immune cell comprising the CAR of any one of claim 25.

81. The CAR immune cell of claim 80, wherein the immune cell is a T cell, natural killer (NK) cell, or macrophage (MA).

82. The CAR immune cell of claim 81, wherein the CAR immune cell maintains the ability to kill cancer ENPP3+ cancer cells after 1, 2, 3, 4 or more stimulations.

83. The CAR immune cell of claim 80, wherein the CAR immune cell is resistant to exhaustion.

84. A population of cells derived from expansion of the CAR immune cell of claim 80.

85. A pharmaceutical composition comprising the molecule or antibody of claim 1, optionally wherein the molecule or antibody comprises a drug conjugated thereto and/or has antibody-dependent cell-mediated cytotoxicity (ADCC) activity, or complement-dependent cytotoxicity activity (CDC), and/or is a bispecific engager optionally a T cell engager or NK cell engager.

86. A method of treating cancer comprising administering a subject in need thereof an effective amount of pharmaceutical composition of claim 85.

87.-89. (canceled)


SEQUENCE LISTING (126 sequences)

SEQ ID NO: 1 - protein, 875 aa, Homo sapiens [sequence omitted]

SEQ ID NO: 2 - protein, 845 aa, Homo sapiens
LLVIMSLGLGLGLGLRKLEKOGSCRKKCFDASFRGLENCRCDVACKDRGDCCWDFEDTCVESTRIWMCNKFRCGETRLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNKYTIVTGLYPESHGIIDNNMYDVNLNKNFSLSSKEQNNPAWWHGQPMWLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKORNLHNCVNIILLADHGMDQTYCNKMEYMTDYFPRINFFYMYEGPAPRIRAHNIPHDFFSENSEEIVRNLSCRKPDQHFKPYLTPDLPKRLHYAKNVRIDKVHLEVDQQWLAVRSKSNTNCGGGNAGYNNEFRSMEAIFLAHGPSFKEKTEVEPFENIEVYNLMCDLLRIQPAPNNGTHGSLNHLLKVPFYEPSHAEEVSKESVCGFANPLPTESLDCFCPHLONSTQLEQVNOMLNLTQEEITATVKVNLPFGRPRVLQKNVDHCLLYHREYVSGFGKAMRMPMWSSYTVPOLGDTSPLPPTVPDCLRADVRVPPSESOKCSFYLADKNITHGFLYPPASNRTSDSQYDALITSNLVPMYEEFRKMWDYFHSVLLIKHATERNGVNVVSGPIFDYNYDGHEDAPDEITKHLANIDVPIPTHYFVVLISCKNKSHTPENCPGWLDVLPFIIPHRPTNVESCPEGKPEALWVEERFTAHIARVRDVELLTGLDFYQDKVOPVSBILQLKTYLPTFETTI

SEQ ID NO: 3 - protein, 135 aa, Homo sapiens
MELGLRWVFLVAILEGVQCEVQLVESGGGLVKPGGSLRLSCAASGFTFSDYSMNWVRQAPGKGLEWVSSISSSSRYKYYADSMKGRFTISRDNAKNSLYLQMNSLRVEDTAVYYCTRGIDYFDYWGQGTLVTVSS

SEQ ID NO: 4 - protein, 129 aa, Homo sapiens
MDMRVPAQLLGLLLLWLPGARCDIQLTQSPSFLSASVGDRVTITCRASQDINNYLAWYQQKPGKAPKFLIHVASTLQSGVPSRFSGSGSGTEFTLTISSLQPEDSATYYCQQLNSYPRTFGQGTKVEIK

SEQ ID NO: 5 - protein, 138 aa, Homo sapiens
MELGLRWVFLVAILEGVQCEVQLVESGGGLVKPGGSLRLSCAASGFTFSSYSMNWVRQAPGKGLEWVTYISSSSSYRYYADSVKGRFTISRDNAKNSLNLQMNTLRPEDTAVYYCARVGAITGYFDFWGQGTLVTVSS

SEQ ID NO: 6 - protein, 129 aa, Homo sapiens
MDMRVPAQLLGLLLLWFPGARCDIQMTQSPSSLSASVGDRVTITCRASQGIRNDLDWFQQKPGKAPKRLIYAASSLQSGVPSRFSGSGSGTEFTLTVSSLQPEDFATYYCLQYNGYPLTFGGGTKVEIK

SEQ ID NO: 7 - protein, 138 aa, Homo sapiens
MELGLRWVFLVAILEGVQCEVQLVESGGGLVKPGGSLRLSCAASGFTFSTFSMNWVRQAPGKGLEWVSYISSYSSYIYYADSVKGRFTISRDNAKKSLYLQMNSLRAEDTAVYYCSRVGAITGYFDYWGQGTLVTVSS

SEQ ID NO: 8 - protein, 136 aa, Homo sapiens
MDMRVPAQLLGLLLLWFPGARCGIQMTQSPSSLSASVGDRVTITCRASQGIRNDLGWYQQTPGKAPKRLMYTASSLHSGVPSRFSGSGSGTEFTLTISSLQPEDFATYYCLQYNGYPLTFGGGTKVEIKSEQIDNO

SEQ ID NO: 9 - protein, 137 aa, Homo sapiens
MEFGLSWVFLVAIIKGVQCQVQLVESGGDLVKPGGSLRLSCAASGFTFSDYYMNWIRQAPEKGLEWISYISSSGFIMDSADSVKGRFTISRDNAKNSLFLQMNSLRAEDTAVYYCARGNWNYPFDYWGQGTLVTVSS

SEQ ID NO: 10 - protein, 129 aa, synthetic construct
MDMRVPAQLLGLLLLWLPATRCDIQMTQSPSSLSASIGDRVTITCRASQGISHYLAWYQQRPGKVPKFLIYAASTLQSGVPSRFSGRGSGTDFTLTISSLQPEDIATYYCQMYKNVPFTFGPGTKVDIK

SEQ ID NO: 11 - protein, 135 aa, Homo sapiens
MELGLRWVFLVAILEGVQCEVQLVESGGGLVKPGGSLRLSCAASGFTFSDYSMNWVRQAPGKGLEWVSSISSSSSYKKYADSVKGRLTISRDNAKNSLYLQMNSLRAEDTAVYYCARGIDYFDYWGQGTLVTVSS

SEQ ID NO: 12 - protein, 129 aa, Homo sapiens
MDMRVPAQLLGLLLLWLPGARCDIQLTQSPSFLSASVGDRVTITCRASQGISSNLAWYQQKPGKDPKVLIYGASTLQSGVPSRFSGSGSGTEFTLTISSLQPEDFATYYCQQLNSYPRTFGQGTKVEIK

SEQ ID NO: 13 - protein, 145 aa, Homo sapiens
MSVSFLIFLPVLGLPWGVLSQVQLQQSGPGLVKPSQTLSLTCAISGDSVSSNRAAWNWIRQPPSRGLEWLGRTYYRSNWYNDYAVSVKSRITIKPDTSKNQFSLQLNSVTPEDTAVYYCARDRGTYYYYYYMDVWDKGTTVTVSS

SEQ ID NO: 14 - protein, 127 aa, Homo sapiens
METPAQLLFLLLLWLPDTTGEIVLTQSPGTLSLSPGERATLSCRASQSVSSSYLVWYQQKAGQAPRILIYGASNRATDIPVRFSGSGSGTDFTLTISRLEPEDFGVYFCQHYGGSYTFGQGTKLEIK

SEQ ID NO: 15 - protein, 147 aa, Homo sapiens
MELGLRWVFLVAILEGVQCEVQLLESGGGLVKPGGSLRLSCAASGFTFSRYNMNWVRQAPGKGLEWVSFISGSSSYVYYADSVKGRFTISKDNAKNSLYLQMNSLRAEDTAVYYCARSGYCTSTTCYNYYYYYMDVWGKGTTVTVSS

SEQ ID NO: 16 - protein, 128 aa, Homo sapiens
METPAQLLFLLLLWLPDTTGEIVLMQSPGTLSLSPGERATLSCRASQSVSNSYLAWYQQKPGQAPRLLIYGASSRATGIPDRFSGSGSGTDFTLTISRLEPEDFAVFYCQQFGSSPLTFGGGTKVEIK

SEQ ID NO: 17 - protein, 135 aa, Homo sapiens
MELGLRWVFLVAILEGVQCELQLVESGGGLVKPGGSLRLSCAASGFTFSDYSMNWVRQAPGKGLNWVASISSSSHYKYYADSVKGRFTISRDNAKNSLYLQMNSLRAEDTAVYYCARGIDFFDIWGQGTMVTVSS

SEQ ID NO: 18 - protein, 129 aa, Homo sapiens
MDMRVPAQLLGLLLLWLPGARCDIQLTQSPSFLSASVGDRVTITCRASQDINSYLVWYQQKPGKAPKLLIYTASTLQSGVPSRFSGSGSGTEFTLTIRSLQPEDFATYYCQQLNSYPRTFGQGTKVEIK

SEQ ID NO: 19 - protein, 237 aa, Homo sapiens
QLVESGGGLVKPGGSLRLSCAASGFTFSDYSMNWVRQAPGKGLEWVSSISSSSRYKYYADSMKGRFTISRDNAKNSLYLQMNSLRVEDTAVYYCTRGIDYFDYWGQGTLVTVSSSGGGGSGGGGSGGGGSDIQLTQSPSFLSASVGDRVTITCRASQDINNYLAWYQQKPGKAPKFLIHVASTLQSGVPSRFSGSGSGTEFTLTISSLQPEDSATYYCQQLNSYPRTFGQGTKVEIK

SEQ ID NO: 20 - protein, 240 aa, Homo sapiens
QLVESGGGLVKPGGSLRLSCAASGFTFSSYSMNWVRQAPGKGLEWVTYISSSSSYRYYADSVKGRFTISRDNAKNSLNLQMNTLRPEDTAVYYCARVGAITGYFDFWGQGTLVTVSSSGGGGSGGGGSGGGGSDIQMTQSPSSLSASVGDRVTITCRASQGIRNDLDWFQQKPGKAPKRLIYAASSLQSGVPSRFSGSGSGTEFTLTVSSLQPEDFATYYCLQYNGYPLTFGGGTKVEIK

SEQ ID NO: 21 - protein, 240 aa, Homo sapiens
QLVESGGGLVKPGGSLRLSCAASGFTFSTFSMNWVRQAPGKGLEWVSYISSYSSYIYYADSVKGRFTISRDNAKKSLYLQMNSLRAEDTAVYYCSRVGAITGYFDYWGQGTLVTVSSSGGGGSGGGGSGGGGSGIQMTQSPSSLSASVGDRVTITCRASQGIRNDLGWYQQTPGKAPKRLMYTASSLHSGVPSRFSGSGSGTEFTLTISSLQPEDFATYYCLQYNGYPLTFGGGTKVEIK

SEQ ID NO: 22 - protein, 239 aa, Homo sapiens
QLVESGGDLVKPGGSLRLSCAASGFTFSDYYMNWIRQAPEKGLEWISYISSSGFIMDSADSVKGRFTISRDNAKNSLFLQMNSLRAEDTAVYYCARGNWNYPFDYWGQGTLVTVSSSGGGGSGGGGSGGGGSDIQMTQSPSSLSASIGDRVTITCRASQGISHYLAWYQQRPGKVPKFLIYAASTLQSGVPSRFSGRGSGTDFTLTISSLQPEDIATYYCQMYKNVPFTFGPGTKVDIK

SEQ ID NO: 23 - protein, 237 aa, Homo sapiens
QLVESGGGLVKPGGSLRLSCAASGFTFSDYSMNWVRQAPGKGLEWVSSISSSSSYKKYADSVKGRLTISRDNAKNSLYLQMNSLRAEDTAVYYCARGIDYFDYWGQGTLVTVSSSGGGGSGGGGSGGGGSDIQLTQSPSFLSASVGDRVTITCRASQGISSNLAWYQQKPGKDPKVLIYGASTLQSGVPSRFSGSGSGTEFTLTISSLQPEDFATYYCQQLNSYPRTFGQGTKVEIK

SEQ ID NO: 24 - protein, 247 aa, Homo sapiens
VQLQQSGPGLVKPSQTLSLTCAISGDSVSSNRAAWNWIRQPPSRGLEWLGRTYYRSNWYNDYAVSVKSRITIKPDTSKNQFSLQLNSVTPEDTAVYYCARDRGTYYYYYYMDVWDKGTTVTVSSSGGGGSGGGGSGGGGSEIVLTQSPGTLSLSPGERATLSCRASQSVSSSYLVWYQQKAGQAPRILIYGASNRATDIPVRFSGSGSGTDFTLTISRLEPEDFGVYFCQHYGGSYTFGQGTKLEIK

SEQ ID NO: 25 - protein, 250 aa, Homo sapiens
QLLESGGGLVKPGGSLRLSCAASGFTFSRYNMNWVRQAPGKGLEWVSFISGSSSYVYYADSVKGRFTISKDNAKNSLYLQMNSLRAEDTAVYYCARSGYCTSTTCYNYYYYYMDVWGKGTTVTVSSSGGGGSGGGGSGGGGSEIVLMQSPGTLSLSPGERATLSCRASQSVSNSYLAWYQQKPGQAPRLLIYGASSRATGIPDRFSGSGSGTDFTLTISRLEPEDFAVFYCQQFGSSPLTFGGGTKVEIK

SEQ ID NO: 26 - protein, 237 aa, Homo sapiens
QLVESGGGLVKPGGSLRLSCAASGFTFSDYSMNWVRQAPGKGLNWVASISSSSHYKYYADSVKGRFTISRDNAKNSLYLQMNSLRAEDTAVYYCARGIDFFDIWGQGTMVTVSSSGGGGSGGGGSGGGGSDIQLTQSPSFLSASVGDRVTITCRASQDINSYLVWYQQKPGKAPKLLIYTASTLQSGVPSRFSGSGSGTEFTLTIRSLQPEDFATYYCQQLNSYPRTFGQGTKVEIK

SEQ ID NO: 27 - protein, 18 aa, Homo sapiens
SSSGGGGSGGGGSGGGGS

SEQ ID NO: 28 - protein, 226 aa, Homo sapiens
AKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 6 aa, Homo sapiens
FTFSDY

SEQ ID NO: 30 - protein, 6 aa, Homo sapiens
SSSSRY

SEQ ID NO: 31 - protein, 7 aa, Homo sapiens
GIDYFDY

SEQ ID NO: 32 - protein, 11 aa, Homo sapiens
RASQDINNYLA

SEQ ID NO: 33 - protein, 7 aa, Homo sapiens
VASTLQS

SEQ ID NO: 34 - protein, 9 aa, Homo sapiens
QQLNSYPRT

SEQ ID NO: 35 - protein, 7 aa, Homo sapiens
GFTFSSY

SEQ ID NO: 36 - protein, 6 aa, Homo sapiens
SSSSSY

SEQ ID NO: 37 - protein, 10 aa, Homo sapiens
VGAITGYFDF

SEQ ID NO: 38 - protein, 11 aa, Homo sapiens
RASQGIRNDLD

SEQ ID NO: 39 - protein, 7 aa, Homo sapiens
AASSLQS

SEQ ID NO: 40 - protein, 9 aa, Homo sapiens
LQYNGYPLT

SEQ ID NO: 41 - protein, 7 aa, Homo sapiens
GFTFSTF

SEQ ID NO: 42 - protein, 6 aa, Homo sapiens
SSYSSY

SEQ ID NO: 43 - protein, 10 aa, Homo sapiens
VGAITGYFDY

SEQ ID NO: 44 - protein, 11 aa, Homo sapiens
RASQGIRNDLG

SEQ ID NO: 45 - protein, 7 aa, Homo sapiens
TASSLHS

SEQ ID NO: 46 - protein, 7 aa, Homo sapiens
GFTFSDY

SEQ ID NO: 47 - protein, 6 aa, Homo sapiens
SSSGFI

SEQ ID NO: 48 - protein, 9 aa, Homo sapiens
GNWNYPFDY

SEQ ID NO: 49 - protein, 11 aa, Homo sapiens
RASQGISHYLA

SEQ ID NO: 50 - protein, 7 aa, Homo sapiens
AASTLQS

SEQ ID NO: 51 - protein, 9 aa, Homo sapiens
QMYKNVPFT

SEQ ID NO: 52 - protein, 11 aa, Homo sapiens
RASQGISSNLA

SEQ ID NO: 53 - protein, 7 aa, Homo sapiens
GASTLQS

SEQ ID NO: 54 - protein, 9 aa, Homo sapiens
GDSVSSNRA

SEQ ID NO: 55 - protein, 7 aa, Homo sapiens
YYRSNWY

SEQ ID NO: 56 - protein, 12 aa, Homo sapiens
RGTYYYYYYMDV

SEQ ID NO: 57 - protein, 12 aa, Homo sapiens
RASQSVSSSYLV

SEQ ID NO: 58 - protein, 7 aa, Homo sapiens
GASNRAT

SEQ ID NO: 59 - protein, 8 aa, Homo sapiens
QHYGGSYT

SEQ ID NO: 60 - protein, 7 aa, Homo sapiens
GFTFSRY

SEQ ID NO: 61 - protein, 6 aa, Homo sapiens
SGSSSY

SEQ ID NO: 62 - protein, 19 aa, Homo sapiens
SGYCTSTTCYNYYYYYMDV

SEQ ID NO: 63 - protein, 12 aa, Homo sapiens
RASQSVSNSYLA

SEQ ID NO: 64 - protein, 7 aa, Homo sapiens
GASSRAT

SEQ ID NO: 65 - protein, 9 aa, Homo sapiens
QQFGSSPLT

SEQ ID NO: 66 - protein, 6 aa, Homo sapiens
SSSSHY

SEQ ID NO: 67 - protein, 7 aa, Homo sapiens
GIDFFDI

SEQ ID NO: 68 - protein, 11 aa, Homo sapiens
RASQDINSYLV

SEQ ID NO: 69 - protein, 7 aa, Homo sapiens
TASTLQS

SEQ ID NO: 70 - protein, 114 aa, Homo sapiens
QLVESGGGLVKPGGSLRLSCAASGFTFSDYSMNWVRQAPGKGLEWVSSISSSSRYKYYADSMKGRFTISRDNAKNSLYLQMNSLRVEDTAVYYCTRGIDYFDYWGQGTLVTVSS

SEQ ID NO: 71 - protein, 107 aa, Homo sapiens
DIQLTQSPSFLSASVGDRVTITCRASQDINNYLAWYQQKPGKAPKFLIHVASTLQSGVPSRFSGSGSGTEFTLTISSLQPEDSATYYCQQLNSYPRTFGQGTKVEIK

SEQ ID NO: 72 - protein, 117 aa, Homo sapiens
QLVESGGGLVKPGGSLRLSCAASGFTFSSYSMNWVRQAPGKGLEWVTYISSSSSYRYYADSVKGRFTISRDNAKNSLNLQMNTLRPEDTAVYYCARVGAITGYFDFWGQGTLVTVSS

SEQ ID NO: 73 - protein, 107 aa, Homo sapiens
DIQMTQSPSSLSASVGDRVTITCRASQGIRNDLDWFQQKPGKAPKRLIYAASSLQSGVPSRFSGSGSGTEFTLTVSSLQPEDFATYYCLQYNGYPLTFGGGTKVEIK

SEQ ID NO: 74 - protein, 117 aa, Homo sapiens
QLVESGGGLVKPGGSLRLSCAASGFTFSTFSMNWVRQAPGKGLEWVSYISSYSSYIYYADSVKGRFTISRDNAKKSLYLQMNSLRAEDTAVYYCSRVGAITGYFDYWGQGTLVTVSS

SEQ ID NO: 75 - protein, 107 aa, Homo sapiens
GIQMTQSPSSLSASVGDRVTITCRASQGIRNDLGWYQQTPGKAPKRLMYTASSLHSGVPSRFSGSGSGTEFTLTISSLQPEDFATYYCLQYNGYPLTFGGGTKVEIK

SEQ ID NO: 76 - protein, 116 aa, Homo sapiens
QLVESGGDLVKPGGSLRLSCAASGFTFSDYYMNWIRQAPEKGLEWISYISSSGFIMDSADSVKGRFTISRDNAKNSLFLQMNSLRAEDTAVYYCARGNWNYPFDYWGQGTLVTVSS

SEQ ID NO: 77 - protein, 107 aa, Homo sapiens
DIQMTQSPSSLSASIGDRVTITCRASQGISHYLAWYQQRPGKVPKFLIYAASTLQSGVPSRFSGRGSGTDFTLTISSLQPEDIATYYCQMYKNVPFTFGPGTKVDIK

SEQ ID NO: 78 - protein, 114 aa, Homo sapiens
QLVESGGGLVKPGGSLRLSCAASGFTFSDYSMNWVRQAPGKGLEWVSSISSSSSYKKYADSVKGRLTISRDNAKNSLYLQMNSLRAEDTAVYYCARGIDYFDYWGQGTLVTVSS

SEQ ID NO: 79 - protein, 107 aa, Homo sapiens
DIQLTQSPSFLSASVGDRVTITCRASQGISSNLAWYQQKPGKDPKVLIYGASTLQSGVPSRFSGSGSGTEFTLTISSLQPEDFATYYCQQLNSYPRTFGQGTKVEIK

SEQ ID NO: 80 - protein, 124 aa, Homo sapiens
VQLQQSGPGLVKPSQTLSLTCAISGDSVSSNRAAWNWIRQPPSRGLEWLGRTYYRSNWYNDYAVSVKSRITIKPDTSKNQFSLQLNSVTPEDTAVYYCARDRGTYYYYYYMDVWDKGTTVTVSS

SEQ ID NO: 81 - protein, 107 aa, Homo sapiens
EIVLTQSPGTLSLSPGERATLSCRASQSVSSSYLVWYQQKAGQAPRILIYGASNRATDIPVRFSGSGSGTDFTLTISRLEPEDFGVYFCQHYGGSYTFGQGTKLEIK

SEQ ID NO: 82 - protein, 126 aa, Homo sapiens
QLLESGGGLVKPGGSLRLSCAASGFTFSRYNMNWVRQAPGKGLEWVSFISGSSSYVYYADSVKGRFTISKDNAKNSLYLQMNSLRAEDTAVYYCARSGYCTSTTCYNYYYYYMDVWGKGTTVTVSS

SEQ ID NO: 83 - protein, 108 aa, Homo sapiens
EIVLMQSPGTLSLSPGERATLSCRASQSVSNSYLAWYQQKPGQAPRLLIYGASSRATGIPDRFSGSGSGTDFTLTISRLEPEDFAVFYCQQFGSSPLTFGGGTKVEIK

SEQ ID NO: 84 - protein, 114 aa, Homo sapiens
QLVESGGGLVKPGGSLRLSCAASGFTFSDYSMNWVRQAPGKGLNWVASISSSSHYKYYADSVKGRFTISRDNAKNSLYLQMNSLRAEDTAVYYCARGIDFFDIWGQGTMVTVSS

SEQ ID NO: 85 - protein, 107 aa, Homo sapiens
DIQLTQSPSFLSASVGDRVTITCRASQDINSYLVWYQQKPGKAPKLLIYTASTLQSGVPSRFSGSGSGTEFTLTIRSLQPEDFATYYCQQLNSYPRTFGQGTKVEIK

SEQ ID NO: 86 - protein, 45 aa, Homo sapiens
TTTPAPRPPTPAPTIASQPLSLRPEACRPAAGGAVHTRGLDFACD

SEQ ID NO: 87 - protein, 23 aa, Homo sapiens
IYIWAPLAGTCGVLLLSLVITLY

SEQ ID NO: 88 - protein, 43 aa, Homo sapiens
CKRGRKKLLYIFKQPFMRPVQTTQEEDGCSCRFPEEEEGGCEL

SEQ ID NO: 89 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 90 - DNA, 1479 bp, synthetic construct
  source 1..1479: mol_type = other DNA
atggccttaccagtgaccgccttgctcctgccgctggccttgctgctccacgccgccaggccggattacaaagacgatgacgataagcaactggtggagtctgggggaggcctggtcaagcctggggggtccctgagactctcctgtgcagcctctggattcaccttcagtgactatagcatgaactgggtccgccaggctccaggaaaggggctggagtgggtctcatccattagtagtagtagtagatacaaatactacgcagactcaatgaagggccgattcaccatctccagagacaacgccaagaactcactgtatctgcaaatgaacagcctgagagtcgaggacacagctgtatattactgtacgagagggattgactactttgactactggggccagggaaccctggtcaccgtctcctcaagtggtggcggtggctcgggcggtggtgggtcgggtggcggaggctctgacatccagttgacccagtctccatccttcctgtctgcatctgtaggagacagagtcaccatcacttgccgggccagtcaggacattaacaattatttagcctggtatcagcaaaaaccagggaaagcccctaagttcctgatccatgttgcatccactttgcaaagtggggtcccatcaaggttcagcggcagtggatctgggacagagttcactctcacaatcagcagtttgcagcctgaagattctgcaacttattactgtcaacagcttaatagttacccgcggacgttcggccaagggaccaaggtggagattaaagccaagcctaccacgacgccagcgccgcgaccaccaacaccggcgcccaccatcgcgtcgcagcccctgtccctgcgcccagaggcatgccggccagcagcagggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 91 - protein, 492 aa, synthetic construct
MALPVTALLLPLALLLHAARPDYKDDDDKQLVESGGGLVKPGGSLRLSCAASGFTFSDYSMNWVRQAPGKGLEWVSSISSSSRYKYYADSMKGRFTISRDNAKNSLYLQMNSLRVEDTAVYYCTRGIDYFDYWGQGTLVTVSSSGGGGSGGGGSGGGGSDIQLTQSPSFLSASVGDRVTITCRASQDINNYLAWYQQKPGKAPKFLIHVASTLQSGVPSRFSGSGSGTEFTLTISSLQPEDSATYYCQQLNSYPRTFGQGTKVEIKAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 92 - DNA, 1488 bp, synthetic construct
  source 1..1488: mol_type = other DNA
atggccttaccagtgaccgccttgctcctgccgctggccttgctgctccacgccgccaggccggattacaaagacgatgacgataagcagctggtggagtctgggggaggcctggtcaagcctggggggtccctgagactctcctgtgcagcctctggattcaccttcagtagttatagcatgaactgggtccgccaggctccagggaaggggctggagtgggtcacatatattagtagtagtagtagttacagatactacgcagactcagtgaagggccggtttaccatctccagagacaacgccaagaactcactgaatctgcaaatgaacaccctgagacccgaggacacagctgtgtactactgtgcgagagtgggagctattacggggtactttgacttttggggccagggaaccctggtcaccgtctcctcaagtggtggcggtggctcgggcggtggtgggtcgggtggcggaggctctgacatccagatgacccagtctccatcctccctgtctgcatctgtaggagacagagtcaccatcacttgccgggcaagtcagggcattagaaatgatttagactggtttcagcagaaaccagggaaagcccctaagcgcctgatctatgctgcatccagtttgcaaagtggggtcccatcaaggttcagcggcagtggatctgggacagaattcactctcacagtcagcagcctgcagcctgaagattttgcaacttattactgtctacagtataatggttatccgctcactttcggcggagggaccaaggtggagatcaaagccaagcctaccacgacgccagcgccgcgaccaccaacaccggcgcccaccatcgcgtcgcagcccctgtccctgcgcccagaggcatgccggccagcagcagggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 93 - protein, 495 aa, synthetic construct
MALPVTALLLPLALLLHAARPDYKDDDDKQLVESGGGLVKPGGSLRLSCAASGFTFSSYSMNWVRQAPGKGLEWVTYISSSSSYRYYADSVKGRFTISRDNAKNSLNLQMNTLRPEDTAVYYCARVGAITGYFDFWGQGTLVTVSSSGGGGSGGGGSGGGGSDIQMTQSPSSLSASVGDRVTITCRASQGIRNDLDWFQQKPGKAPKRLIYAASSLQSGVPSRFSGSGSGTEFTLTVSSLQPEDFATYYCLQYNGYPLTFGGGTKVEIKAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 94 - DNA, 1488 bp, synthetic construct
  source 1..1488: mol_type = other DNA
atggccttaccagtgaccgccttgctcctgccgctggccttgctgctccacgccgccaggccggattacaaagacgatgacgataagcaattggtggagtctgggggaggcctggtcaagcctggggggtccctgagactctcctgtgcagcctctggattcaccttcagtacctttagcatgaactgggtccgccaggctccagggaaggggctggaatgggtttcatacattagtagttatagtagttacatatactacgcagactcagtgaagggccgattcaccatctccagagacaacgccaagaaatcactgtatctgcaaatgaacagcctgagagccgaagacacagctgtctattactgttcgcgagtgggagctattacggggtactttgactattggggccagggaaccctggtcaccgtctcctcaagtggtggcggtggctcgggcggtggtgggtcgggtggcggaggctctggcatccagatgacccagtctccatcctccctgtctgcatctgtaggagacagagtcaccatcacttgccgggcaagtcagggcattagaaatgatttaggctggtatcagcagacaccaggaaaagcccctaagcgcctgatgtatactgcatccagtttgcacagtggggtcccctcaaggttcagcggcagtggatctgggacagaattcactctcacaatcagcagcctgcagcctgaagattttgcaacttattactgtctacagtataatggttacccgctcactttcggcggagggaccaaggtggagatcaaggccaagcctaccacgacgccagcgccgcgaccaccaacaccggcgcccaccatcgcgtcgcagcccctgtccctgcgcccagaggcatgccggccagcagcagggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 95 - protein, 495 aa, synthetic construct
MALPVTALLLPLALLLHAARPDYKDDDDKQLVESGGGLVKPGGSLRLSCAASGFTFSTFSMNWVRQAPGKGLEWVSYISSYSSYIYYADSVKGRFTISRDNAKKSLYLQMNSLRAEDTAVYYCSRVGAITGYFDYWGQGTLVTVSSSGGGGSGGGGSGGGGSGIQMTQSPSSLSASVGDRVTITCRASQGIRNDLGWYQQTPGKAPKRLMYTASSLHSGVPSRFSGSGSGTEFTLTISSLQPEDFATYYCLQYNGYPLTFGGGTKVEIKAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 96 - DNA, 1485 bp, synthetic construct
  source 1..1485: mol_type = other DNA
atggccttaccagtgaccgccttgctcctgccgctggccttgctgctccacgccgccaggccggattacaaagacgatgacgataagcagctggtggagtctgggggagacttggtcaagcctggagggtccctgagactctcctgtgcagcctctggattcaccttcagtgactattacatgaactggatccgccaggctccagagaagggactggagtggatttcatacattagtagtagtggttttatcatggactccgcagactctgtgaagggccgattcaccatctccagggacaacgccaagaactcactgtttctgcaaatgaacagcctgagagccgaggacacggccgtgtattactgtgcgagaggtaactggaactacccttttgactactggggccagggaaccctggtcaccgtctcctcaagtggtggcggtggctcgggcggtggtgggtcgggtggcggaggctctgacatccagatgacccagtctccatcctccctgtctgcatctataggagacagagtcaccatcacttgccgggcgagtcagggcattagccattatttagcctggtatcagcagagaccagggaaagttcctaagttcctgatctatgctgcatccactttacaatcaggggtcccatctcggttcagtggccgtggatctgggacagatttcactctcaccatcagcagcctgcagcctgaagatattgcgacttattactgtcagatgtataaaaatgtcccattcactttcggccctgggaccaaagtggatatcaaagccaagcctaccacgacgccagcgccgcgaccaccaacaccggcgcccaccatcgcgtcgcagcccctgtccctgcgcccagaggcatgccggccagcagcagggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 97 - protein, 494 aa, synthetic construct
MALPVTALLLPLALLLHAARPDYKDDDDKQLVESGGDLVKPGGSLRLSCAASGFTFSDYYMNWIRQAPEKGLEWISYISSSGFIMDSADSVKGRFTISRDNAKNSLFLQMNSLRAEDTAVYYCARGNWNYPFDYWGQGTLVTVSSSGGGGSGGGGSGGGGSDIQMTQSPSSLSASIGDRVTITCRASQGISHYLAWYQQRPGKVPKFLIYAASTLQSGVPSRFSGRGSGTDFTLTISSLQPEDIATYYCQMYKNVPFTFGPGTKVDIKAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 98 - DNA, 1479 bp, synthetic construct
  source 1..1479: mol_type = other DNA
atggccttaccagtgaccgccttgctcctgccgctggccttgctgctccacgccgccaggccggattacaaagacgatgacgataagcagctggtggagtctgggggaggcctggtcaagcctggggggtccctgagactctcctgtgcagcctctggattcaccttcagtgactatagcatgaactgggtccgccaggctccagggaaggggctggagtgggtctcatccattagtagtagtagtagttacaaaaagtacgcagactcagtgaagggccgcctcaccatctccagagacaacgccaagaattcattgtatctgcagatgaacagcctgagagccgaggacacagctgtgtattactgtgcgagagggattgactactttgattactggggccagggaaccctggtcaccgtctcctcaagtggtggcggtggctcgggcggtggtgggtcgggtggcggaggctctgacatccagttgacccagtctccatccttcctgtctgcatctgtaggagacagagtcaccatcacttgccgggccagtcagggcattagcagtaacttagcctggtatcagcagaaaccagggaaagaccctaaggtcctgatctatggtgcatccactttgcaaagtggggtcccatcaaggttcagcggcagtggatctgggacagaattcactctcacaatcagcagcctgcagcctgaagattttgcaacttattactgtcaacagcttaatagttacccgaggacgttcggccaagggaccaaggtggaaatcaaagccaagcctaccacgacgccagcgccgcgaccaccaacaccggcgcccaccatcgcgtcgcagcccctgtccctgcgcccagaggcatgccggccagcagcagggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 99 - protein, 492 aa, synthetic construct
MALPVTALLLPLALLLHAARPDYKDDDDKQLVESGGGLVKPGGSLRLSCAASGFTFSDYSMNWVRQAPGKGLEWVSSISSSSSYKKYADSVKGRLTISRDNAKNSLYLQMNSLRAEDTAVYYCARGIDYFDYWGQGTLVTVSSSGGGGSGGGGSGGGGSDIQLTQSPSFLSASVGDRVTITCRASQGISSNLAWYQQKPGKDPKVLIYGASTLQSGVPSRFSGSGSGTEFTLTISSLQPEDFATYYCQQLNSYPRTFGQGTKVEIKAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 100 - DNA, 1509 bp, synthetic construct
  source 1..1509: mol_type = other DNA
atggccttaccagtgaccgccttgctcctgccgctggccttgctgctccacgccgccaggccggattacaaagacgatgacgataaggtacagctgcagcagtcaggtccaggactggtgaagccctcgcagaccctctcactcacctgtgccatctccggggacagtgtctctagcaacagagctgcttggaactggatcaggcagcccccatcgagaggccttgagtggctgggaaggacatactacaggtccaactggtataatgattatgcagtttctgtgaagagtcgaataaccatcaaaccagacacatccaagaaccagttctccctgcagctgaactctgtgactcccgaggacacggctgtttattactgtgcaagagatcgtgggacctactactactactactacatggacgtctgggacaaagggaccacggtcaccgtctcctcaagtggtggcggtggctcgggcggtggtgggtcgggtggcggaggctctgaaattgtgttgacgcagtctccaggcaccctgtctttgtctccaggggaaagagccaccctctcctgcagggccagtcagagtgttagtagcagctacttagtctggtaccaacagaaagctggccaggctcccaggatcctcatctatggtgcctccaacagggccactgacatcccagtcaggttcagtggcagtgggtctgggacagacttcactctcaccatcagcagactggagcctgaggattttggagtgtatttctgtcagcattatggtggctcatacacttttggccaggggaccaagctggagatcaaagccaagcctaccacgacgccagcgccgcgaccaccaacaccggcgcccaccatcgcgtcgcagcccctgtccctgcgcccagaggcatgccggccagcagcagggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 101 - protein, 502 aa, synthetic construct
MALPVTALLLPLALLLHAARPDYKDDDDKVQLQQSGPGLVKPSQTLSLTCAISGDSVSSNRAAWNWIRQPPSRGLEWLGRTYYRSNWYNDYAVSVKSRITIKPDTSKNQFSLQLNSVTPEDTAVYYCARDRGTYYYYYYMDVWDKGTTVTVSSSGGGGSGGGGSGGGGSEIVLTQSPGTLSLSPGERATLSCRASQSVSSSYLVWYQQKAGQAPRILIYGASNRATDIPVRFSGSGSGTDFTLTISRLEPEDFGVYFCQHYGGSYTFGQGTKLEIKAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 102 - DNA, 1518 bp, synthetic construct
  source 1..1518: mol_type = other DNA
atggccttaccagtgaccgccttgctcctgccgctggccttgctgctccacgccgccaggccggattacaaagacgatgacgataagcagctgctggagtctgggggaggcctggtcaagcctggggggtccctgagactctcctgtgcggcctctggattcacgttcagtagatataacatgaactgggtccgccaggctccagggaaggggctggagtgggtctcattcatcagtggcagtagtagttacgtatactacgcagactcagtgaagggccgattcaccatctccaaagacaacgccaagaactcactgtatctgcaaatgaacagcctgagagccgaggacacagctgtatattattgtgcgagatcggggtattgtactagtaccacctgctataattactactactactacatggacgtctggggcaaagggaccacggtcaccgtctcctcaagtggtggcggtggctcgggcggtggtgggtcgggtggcggaggctctgaaattgtgttgatgcagtctccaggcaccctgtctttgtctccaggggaaagagccaccctctcctgcagggccagtcagagtgttagcaacagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgttttactgtcagcagtttggtagctcaccgctcactttcggcggagggaccaaggtggagatcaaagccaagcctaccacgacgccagcgccgcgaccaccaacaccggcgcccaccatcgcgtcgcagcccctgtccctgcgcccagaggcatgccggccagcagcagggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 103 - protein, 505 aa, synthetic construct
MALPVTALLLPLALLLHAARPDYKDDDDKQLLESGGGLVKPGGSLRLSCAASGFTFSRYNMNWVRQAPGKGLEWVSFISGSSSYVYYADSVKGRFTISKDNAKNSLYLQMNSLRAEDTAVYYCARSGYCTSTTCYNYYYYYMDVWGKGTTVTVSSSGGGGSGGGGSGGGGSEIVLMQSPGTLSLSPGERATLSCRASQSVSNSYLAWYQQKPGQAPRLLIYGASSRATGIPDRFSGSGSGTDFTLTISRLEPEDFAVFYCQQFGSSPLTFGGGTKVEIKAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 104 - DNA, 1479 bp, synthetic construct
  source 1..1479: mol_type = other DNA
atggccttaccagtgaccgccttgctcctgccgctggccttgctgctccacgccgccaggccggattacaaagacgatgacgataagcagctggtggagtctgggggaggcctggtcaagcctggggggtccctgagactctcctgtgcagcctctggattcaccttcagtgactacagcatgaactgggtccgccaggctccaggaaaggggctgaattgggtcgcatccattagtagtagtagtcattacaaatactacgcagactcagtgaagggccgattcaccatctccagagacaacgccaagaactcactgtatctgcaaatgaacagcctgagagccgaggacacagctgtgtattattgtgcgagagggattgatttttttgatatttggggccaagggacaatggtcaccgtctcttcaagtggtggcggtggctcgggcggtggtgggtcgggtggcggaggctctgacatccagttgacccagtctccatccttcctgtctgcatctgtaggagacagagtcaccatcacttgccgggccagtcaggacattaacagttatttagtttggtatcagcaaaaaccagggaaagcccctaaactcctgatctatactgcatccactttgcaaagcggggtcccatcaaggttcagcggcagtggatctgggacagaattcactctcacaatcagaagcctgcagcctgaagattttgcaacttattactgtcaacagcttaatagttaccctcggacgttcggccaagggaccaaggtggaaatcaaagccaagcctaccacgacgccagcgccgcgaccaccaacaccggcgcccaccatcgcgtcgcagcccctgtccctgcgcccagaggcatgccggccagcagcagggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 105 - protein, 492 aa, synthetic construct
MALPVTALLLPLALLLHAARPDYKDDDDKQLVESGGGLVKPGGSLRLSCAASGFTFSDYSMNWVRQAPGKGLNWVASISSSSHYKYYADSVKGRFTISRDNAKNSLYLQMNSLRAEDTAVYYCARGIDFFDIWGQGTMVTVSSSGGGGSGGGGSGGGGSDIQLTQSPSFLSASVGDRVTITCRASQDINSYLVWYQQKPGKAPKLLIYTASTLQSGVPSRFSGSGSGTEFTLTIRSLQPEDFATYYCQQLNSYPRTFGQGTKVEIKAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 106 - DNA, 1503 bp, synthetic construct
  source 1..1503: mol_type = other DNA
atggccttaccagtgaccgccttgctcctgccgctggccttgctgctccacgccgccaggccggattacaaagacgatgacgataagatggaggtgctggtggagagcggcggcggcctggtgcagcccggcggcagcctgaggctgagctgcgccgccagcggcttcgccttcagcaactacggcatgagctgggtgaggcaggcccccggccagaggctggagtgggtgagctacatcagcagcggcggcggcaccacctactacctggacagcgtgaccggcaggttcaccatcagcagggacaacgccaagaacagcctgtacctgcagatgaacagcctgagggccgaggacaccgccgtgtactactgcgccaggcacaggagggtgtactacggctactacttcgactactggggccagggcaccctggtgaccgtgagcagcagtggtggcggtggctcgggcggtggtgggtcgggtggcggaggctctatggacatcctgctgacccagagccccgacttccagagcgtgacccccaaggagaaggtgaccatcacctgcagggccagccagagcatcggcaccagcatccactggtaccagcagaagcccgaccagagccccaagctgctgatcgagtacgccagcgagagcatcagcggcatccccagcaggttcagcggcagcggcagcggcaccgacttcaccctgaccatcaacagcctggaggccgaggacgccgccacctactactgccagcagagcaacagctggccctacaccttcggcggcggcaccaagctggagatcaaggccaagcctaccacgacgccagcgccgcgaccaccaacaccggcgcccaccatcgcgtcgcagcccctgtccctgcgcccagaggcatgccggccagcagcagggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 107 - protein, 500 aa, synthetic construct
MALPVTALLLPLALLLHAARPDYKDDDDKMEVLVESGGGLVQPGGSLRLSCAASGFAFSNYGMSWVRQAPGQRLEWVSYISSGGGTTYYLDSVTGRFTISRDNAKNSLYLQMNSLRAEDTAVYYCARHRRVYYGYYFDYWGQGTLVTVSSSGGGGSGGGGSGGGGSMDILLTQSPDFQSVTPKEKVTITCRASQSIGTSIHWYQQKPDQSPKLLIEYASESISGIPSRFSGSGSGTDFTLTINSLEAEDAATYYCQQSNSWPYTFGGGTKLEIKAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 108 - DNA, 1461 bp, synthetic construct
  source 1..1461: mol_type = other DNA
atggccttaccagtgaccgccttgctcctgccgctggccttgctgctccacgccgccaggccggattacaaagacgatgacgataagcagctggtggagtctgggggaggcctggtcaagcctggggggtccctgagactctcctgtgcagcctctggattcaccttcagtgactatagcatgaactgggtccgccaggctccagggaaggggctggagtgggtctcatccattagtagtagtagtagttacaaaaagtacgcagactcagtgaagggccgcctcaccatctccagagacaacgccaagaattcattgtatctgcagatgaacagcctgagagccgaggacacagctgtgtattactgtgcgagagggattgactactttgattactggggccagggaaccctggtcaccgtgtcctcaagtggtggcggtggctcgggcggtggtgggtcgggtggcggaggctctgacatccagttgacccagtctccatccttcctgtctgcatctgtaggagacagagtcaccatcacttgccgggccagtcagggcattagcagtaacttagcctggtatcagcagaaaccagggaaagaccctaaggtcctgatctatggtgcatccactttgcaaagtggggtcccatcaaggttcagcggcagtggatctgggacagaattcactctcacaatcagcagcctgcagcctgaagattttgcaacttattactgtcaacagcttaatagttacccgaggacgttcggccaagggaccaaggtggaaatcaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 109 - protein, 486 aa, synthetic construct
MALPVTALLLPLALLLHAARPDYKDDDDKQLVESGGGLVKPGGSLRLSCAASGFTFSDYSMNWVRQAPGKGLEWVSSISSSSSYKKYADSVKGRLTISRDNAKNSLYLQMNSLRAEDTAVYYCARGIDYFDYWGQGTLVTVSSSGGGGSGGGGSGGGGSDIQLTQSPSFLSASVGDRVTITCRASQGISSNLAWYQQKPGKDPKVLIYGASTLQSGVPSRFSGSGSGTEFTLTISSLQPEDFATYYCQQLNSYPRTFGQGTKVEIKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 110 - DNA, 1371 bp, synthetic construct
  source 1..1371: mol_type = other DNA
atggccttaccagtgaccgccttgctcctgccgctggccttgctgctccacgccgccaggccggattacaaagacgatgacgataagcagctggtggagtctgggggaggcctggtcaagcctggggggtccctgagactctcctgtgcagcctctggattcaccttcagtgactatagcatgaactgggtccgccaggctccagggaaggggctggagtgggtctcatccattagtagtagtagtagttacaaaaagtacgcagactcagtgaagggccgcctcaccatctccagagacaacgccaagaattcattgtatctgcagatgaacagcctgagagccgaggacacagctgtgtattactgtgcgagagggattgactactttgattactggggccagggaaccctggtcaccgtgtcctcaagtggtggcggtggctcgggcggtggtgggtcgggtggcggaggctctgacatccagttgacccagtctccatccttcctgtctgcatctgtaggagacagagtcaccatcacttgccgggccagtcagggcattagcagtaacttagcctggtatcagcagaaaccagggaaagaccctaaggtcctgatctatggtgcatccactttgcaaagtggggtcccatcaaggttcagcggcagtggatctgggacagaattcactctcacaatcagcagcctgcagcctgaagattttgcaacttattactgtcaacagcttaatagttacccgaggacgttcggccaagggaccaaggtggaaatcaaagagtccaaatatggtcccccatgcccaccctgcccaatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 111 - protein, 456 aa, synthetic construct
MALPVTALLLPLALLLHAARPDYKDDDDKQLVESGGGLVKPGGSLRLSCAASGFTFSDYSMNWVRQAPGKGLEWVSSISSSSSYKKYADSVKGRLTISRDNAKNSLYLQMNSLRAEDTAVYYCARGIDYFDYWGQGTLVTVSSSGGGGSGGGGSGGGGSDIQLTQSPSFLSASVGDRVTITCRASQGISSNLAWYQQKPGKDPKVLIYGASTLQSGVPSRFSGSGSGTEFTLTISSLQPEDFATYYCQQLNSYPRTFGQGTKVEIKESKYGPPCPPCPIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 112 - DNA, 1380 bp, synthetic construct
  source 1..1380: mol_type = other DNA
atggccttaccagtgaccgccttgctcctgccgctggccttgctgctccacgccgccaggccggattacaaagacgatgacgataagcagctggtggagtctgggggaggcctggtcaagcctggggggtccctgagactctcctgtgcagcctctggattcaccttcagtgactatagcatgaactgggtccgccaggctccagggaaggggctggagtgggtctcatccattagtagtagtagtagttacaaaaagtacgcagactcagtgaagggccgcctcaccatctccagagacaacgccaagaattcattgtatctgcagatgaacagcctgagagccgaggacacagctgtgtattactgtgcgagagggattgactactttgattactggggccagggaaccctggtcaccgtgtcctcaagtggtggcggtggctcgggcggtggtgggtcgggtggcggaggctctgacatccagttgacccagtctccatccttcctgtctgcatctgtaggagacagagtcaccatcacttgccgggccagtcagggcattagcagtaacttagcctggtatcagcagaaaccagggaaagaccctaaggtcctgatctatggtgcatccactttgcaaagtggggtcccatcaaggttcagcggcagtggatctgggacagaattcactctcacaatcagcagcctgcagcctgaagattttgcaacttattactgtcaacagcttaatagttacccgaggacgttcggccaagggaccaaggtggaaatcaaagagtccaaatatggtcccccatgcccaccctgcccattttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 113 - protein, 459 aa, synthetic construct
MALPVTALLLPLALLLHAARPDYKDDDDKQLVESGGGLVKPGGSLRLSCAASGFTFSDYSMNWVRQAPGKGLEWVSSISSSSSYKKYADSVKGRLTISRDNAKNSLYLQMNSLRAEDTAVYYCARGIDYFDYWGQGTLVTVSSSGGGGSGGGGSGGGGSDIQLTQSPSFLSASVGDRVTITCRASQGISSNLAWYQQKPGKDPKVLIYGASTLQSGVPSRFSGSGSGTEFTLTISSLQPEDFATYYCQQLNSYPRTFGQGTKVEIKESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 114 - DNA, 1458 bp, synthetic construct
  source 1..1458: mol_type = other DNA
atggccttaccagtgaccgccttgctcctgccgctggccttgctgctccacgccgccaggccggattacaaagacgatgacgataagcagctggtggagtctgggggaggcctggtcaagcctggggggtccctgagactctcctgtgcagcctctggattcaccttcagtgactatagcatgaactgggtccgccaggctccagggaaggggctggagtgggtctcatccattagtagtagtagtagttacaaaaagtacgcagactcagtgaagggccgcctcaccatctccagagacaacgccaagaattcattgtatctgcagatgaacagcctgagagccgaggacacagctgtgtattactgtgcgagagggattgactactttgattactggggccagggaaccctggtcaccgtgtcctcaagtggtggcggtggctcgggcggtggtgggtcgggtggcggaggctctgacatccagttgacccagtctccatccttcctgtctgcatctgtaggagacagagtcaccatcacttgccgggccagtcagggcattagcagtaacttagcctggtatcagcagaaaccagggaaagaccctaaggtcctgatctatggtgcatccactttgcaaagtggggtcccatcaaggttcagcggcagtggatctgggacagaattcactctcacaatcagcagcctgcagcctgaagattttgcaacttattactgtcaacagcttaatagttacccgaggacgttcggccaagggaccaaggtggaaatcaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 115 - protein, 485 aa, synthetic construct
MALPVTALLLPLALLLHAARPDYKDDDDKQLVESGGGLVKPGGSLRLSCAASGFTFSDYSMNWVRQAPGKGLEWVSSISSSSSYKKYADSVKGRLTISRDNAKNSLYLQMNSLRAEDTAVYYCARGIDYFDYWGQGTLVTVSSSGGGGSGGGGSGGGGSDIQLTQSPSFLSASVGDRVTITCRASQGISSNLAWYQQKPGKDPKVLIYGASTLQSGVPSRFSGSGSGTEFTLTISSLQPEDFATYYCQQLNSYPRTFGQGTKVEIKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 116 - DNA, 1371 bp, synthetic construct
  source 1..1371: mol_type = other DNA
atggccttaccagtgaccgccttgctcctgccgctggccttgctgctccacgccgccaggccggattacaaagacgatgacgataagcagctggtggagtctgggggaggcctggtcaagcctggggggtccctgagactctcctgtgcagcctctggattcaccttcagtgactatagcatgaactgggtccgccaggctccagggaaggggctggagtgggtctcatccattagtagtagtagtagttacaaaaagtacgcagactcagtgaagggccgcctcaccatctccagagacaacgccaagaattcattgtatctgcagatgaacagcctgagagccgaggacacagctgtgtattactgtgcgagagggattgactactttgattactggggccagggaaccctggtcaccgtgtcctcaagtggtggcggtggctcgggcggtggtgggtcgggtggcggaggctctgacatccagttgacccagtctccatccttcctgtctgcatctgtaggagacagagtcaccatcacttgccgggccagtcagggcattagcagtaacttagcctggtatcagcagaaaccagggaaagaccctaaggtcctgatctatggtgcatccactttgcaaagtggggtcccatcaaggttcagcggcagtggatctgggacagaattcactctcacaatcagcagcctgcagcctgaagattttgcaacttattactgtcaacagcttaatagttacccgaggacgttcggccaagggaccaaggtggaaatcaaagagtccaaatatggtcccccatgcccatcatgcccaatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 117 - protein, 456 aa, synthetic construct
MALPVTALLLPLALLLHAARPDYKDDDDKQLVESGGGLVKPGGSLRLSCAASGFTFSDYSMNWVRQAPGKGLEWVSSISSSSSYKKYADSVKGRLTISRDNAKNSLYLQMNSLRAEDTAVYYCARGIDYFDYWGQGTLVTVSSSGGGGSGGGGSGGGGSDIQLTQSPSFLSASVGDRVTITCRASQGISSNLAWYQQKPGKDPKVLIYGASTLQSGVPSRFSGSGSGTEFTLTISSLQPEDFATYYCQQLNSYPRTFGQGTKVEIKESKYGPPCPSCPIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 118 - DNA, 1377 bp, synthetic construct
  source 1..1377: mol_type = other DNA
atggccttaccagtgaccgccttgctcctgccgctggccttgctgctccacgccgccaggccggattacaaagacgatgacgataagcagctggtggagtctgggggaggcctggtcaagcctggggggtccctgagactctcctgtgcagcctctggattcaccttcagtgactatagcatgaactgggtccgccaggctccagggaaggggctggagtgggtctcatccattagtagtagtagtagttacaaaaagtacgcagactcagtgaagggccgcctcaccatctccagagacaacgccaagaattcattgtatctgcagatgaacagcctgagagccgaggacacagctgtgtattactgtgcgagagggattgactactttgattactggggccagggaaccctggtcaccgtgtcctcaagtggtggcggtggctcgggcggtggtgggtcgggtggcggaggctctgacatccagttgacccagtctccatccttcctgtctgcatctgtaggagacagagtcaccatcacttgccgggccagtcagggcattagcagtaacttagcctggtatcagcagaaaccagggaaagaccctaaggtcctgatctatggtgcatccactttgcaaagtggggtcccatcaaggttcagcggcagtggatctgggacagaattcactctcacaatcagcagcctgcagcctgaagattttgcaacttattactgtcaacagcttaatagttacccgaggacgttcggccaagggaccaaggtggaaatcaaagagtccaaatatggtcccccatgcccatcatgcccattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 119 - protein, 458 aa, synthetic construct
MALPVTALLLPLALLLHAARPDYKDDDDKQLVESGGGLVKPGGSLRLSCAASGFTFSDYSMNWVRQAPGKGLEWVSSISSSSSYKKYADSVKGRLTISRDNAKNSLYLQMNSLRAEDTAVYYCARGIDYFDYWGQGTLVTVSSSGGGGSGGGGSGGGGSDIQLTQSPSFLSASVGDRVTITCRASQGISSNLAWYQQKPGKDPKVLIYGASTLQSGVPSRFSGSGSGTEFTLTISSLQPEDFATYYCQQLNSYPRTFGQGTKVEIKESKYGPPCPSCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 120 - protein, 221 aa, synthetic construct
KIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 121 - protein, 191 aa, synthetic construct
KESKYGPPCPPCPIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 122 - protein, 194 aa, synthetic construct
KESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - protein, 220 aa, synthetic construct
KIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 124 - protein, 191 aa, synthetic construct
KESKYGPPCPSCPIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 125 - protein, 193 aa, synthetic construct
KESKYGPPCPSCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 126 - protein, 29 aa, synthetic construct
MALPVTALLLPLALLLHAARPDYKDDDDK